(19) United States
(12) Patent Application Publication
(10) Pub. No.: US 2003/0139374 A1
(43) Pub. Date: Jul. 24, 2003

(54) Combined methods for tumor vasculature coagulation and treatment

(75) Inventors: Philip E. Thorpe, Dallas, TX (US); Steven W. King, Rancho Santa Margarita, CA (US); Claudia Gottstein, Dallas, TX (US)

Correspondence Address: Shelley P.M. Fussey, Williams, Morgan & Amerson, P.C., Suite 250, 7676 Hillmont, Houston, TX, 77040, US

(73) Assignee: Board of Regents, The University of Texas System and Peregrine Pharmaceuticals, Inc.

(21) Appl. No.: 10/259,236

(22) Filed: Sep. 27, 2002

Related U.S. Application Data
(60) Provisional application No. 60/325,532, filed on Sep. 27, 2001.

(51) Int. Cl. A61K 31/739

(52) U.S. Cl. 514/54

(57) ABSTRACT

Disclosed are various defined combinations of agents for use in improved anti-vascular therapies and coagulative tumor treatment. Particularly provided are combined treatment methods, and associated compositions, pharmaceuticals, medicaments, kits and uses, which together function surprisingly effectively in the treatment of vascularized tumors. The invention preferably involves a component or treatment step that enhances the effectiveness of therapy using targeted or non-targeted coagulants to cause tumor vasculature thrombosis.

Applicants claim priority to U.S. provisional application Serial No. 60/325,532, filed Sep. 27, 2001, the specification, claims and drawings of which application are specifically incorporated herein by reference without disclaimer.

### BACKGROUND OF THE INVENTION

1. Field of the Invention

The present invention relates generally to the fields of blood vessels, coagulation and tumor therapy. More particularly, it provides various specified combined treatment methods, and associated compositions, pharmaceuticals, medicaments, kits and uses, which together function surprisingly effectively in the treatment of vascularized tumors. The combination methods, uses and compositions of the invention preferably include a component or treatment that enhances the effectiveness of targeted or non-targeted coagulants in causing tumor vasculature thrombosis.

2. Description of the Related Art

Tumor cell resistance to various chemotherapeutic agents represents a major problem in clinical oncology. Therefore, although many advances in the chemotherapy of neoplastic disease have been realized during the last 30 years, many of the most prevalent forms of human cancer still resist effective chemotherapeutic intervention.

A significant underlying problem that must be addressed in any treatment regimen is the concept of “total cell kill.” This concept holds that in order to have an effective treatment regimen, whether it be a surgical or chemotherapeutic approach or both, there must be a total cell kill of all so-called “clonogenic” malignant cells, that is, cells that have the ability to grow uncontrolled and replace any tumor mass that might be removed. Due to the ultimate need to develop therapeutic agents and regimens that will achieve a total cell kill, certain types of tumors have been more amenable than others to therapy. For example, the soft tissue tumors (e.g., lymphomas), and tumors of the blood and blood-forming organs (e.g., leukemias) have generally been more responsive to chemotherapeutic therapy than have solid tumors such as carcinomas.

One reason for the susceptibility of soft and blood-based tumors to chemotherapy is the greater physical accessibility of lymphoma and leukemic cells to chemotherapeutic intervention. Simply put, it is much more difficult for most chemotherapeutic agents to reach all of the cells of-a solid tumor mass than it is the soft tumors and blood-based tumors, and therefore much more difficult to achieve a total cell kill. Increasing the dose of chemotherapeutic agents most often results in toxic side effects, which generally limits the effectiveness of conventional anti-tumor agents.

It has long been clear that a significant need exists for the development of novel strategies for the treatment of solid tumors. One such strategy is the use of “immunotoxins”, in which an anti-tumor cell antibody is used to deliver a toxin to the tumor cells. However, in common with the chemotherapeutic approach described above, this also suffers from certain drawbacks. For example, antigen-negative or antigen-deficient cells can survive and repopulate the tumor or lead to further metastases. Also, in the treatment of solid tumors, the tumor mass is generally impermeable to molecules of the size of the antibodies and immunotoxins. Therefore, the development of immunotoxins alone did not lead to particularly significant improvements in cancer treatment.

Certain investigators then developed the approach of targeting the vasculature of solid tumors. Targeting the blood vessels of the tumors has certain advantages in that it is not likely to lead to the development of resistant tumor cells or populations thereof. Furthermore, delivery of targeted agents to the vasculature does not have problems connected with accessibility, and destruction of the blood vessels should lead to an amplification of the anti-tumor effect as many tumor cells rely on a single vessel for their oxygen and nutrient supplies. Exemplary intratumoral vascular targeting strategies are described in U.S. Pat. Nos. 5,855,866 and 6,051,230.

Another approach for the targeted destruction of tumor vasculature is described in U.S. Pat. Nos. 6,093,399 and 6,004,555, in which antibodies and ligands against tumor vascular and stromal markers are used to deliver coagulants to solid tumors. The targeted delivery of coagulants in this manner has the advantage that significant toxic side effects are not likely to result from any background mis-targeting that may result due to any low level cross-reactivity of the targeting antibodies with the cells of normal tissues. The antibody-coagulant constructs for use in such directed anti-tumor therapy have been termed “coaguligands”.

Exemplary components for use in such targeted coaguligands are coagulants based on Tissue Factor (TF) and Tissue Factor derivatives. As disclosed in U.S. Pat. No. 5,877,289, a preferred derivative is a truncated version of human Tissue Factor (truncated Tissue Factor. “tTF”, or soluble Tissue Factor, “sTF”). Treatment of tumor-bearing mice with such coaguligands results in significant tumor necrosis and even complete tumor regression in many animals (U.S. Pat. Nos. 5,877,289, 6,004,555 and 6,093,399; Huang et al., 1997).

Coagulation-impaired TF compositions were later surprisingly shown to be capable of specifically localizing to the blood vessels within a vascularized tumor and exerting anti-tumor effects in the absence of any targeting agent (U.S. Pat. Nos. 6,156,321, 6,132.729 and 6,132.730). These self-localizing TF derivatives, and the therapies associated therewith, became known as “naked Tissue Factor” compositions and therapies. Such naked Tissue Factors can be further modified to improve their biological half-life, e.g., by conjugation to inert (non-targeting) carriers.

Although the targeted delivery of coagulation factors and the use of naked Tissue Factor coagulants represent significant advances in tumor treatment protocols, there is still a need for improved anti-vascular tumor therapies. The identification of additional agents capable of increasing the effectiveness of both targeted and non-targeted anti-vascular coagulant therapies would provide significant benefits. e.g., in expanding the number of agents for use and broadening the patient selection criteria. Developing combination therapies to allow the targeted or non-targeted coagulants to be used at lower doses, thus further reducing any concerns regarding side effects, would represent another important advance in the development of safe and effective therapeutic products.

### SUMMARY OF THE INVENTION

The present invention addresses the needs of the prior art by providing new combined methods and compositions for improved tumor treatment using coagulant-based tumor therapeutics. The invention particularly provides various defined combinations that increase the effectiveness of both targeted and non-targeted coagulant therapies that act on tumor vasculature to induce thrombosis and tumor necrosis. The combined treatment methods and uses, and related compositions, pharmaceuticals, medicaments and kits of the invention. preferably comprise one or more components or treatments that function to sensitize tumor vasculature to the coagulant-based treatment, typically achieved by enhancing the procoagulant status of the tumor vasculature, thus making coagulant-based tumor therapy more effective.

Increasing the sensitivity of the vasculature in the tumor towards coagulation using the combined approaches of the present invention broadens the range of procoagulant agents that may be effectively used in tumor treatment, meaning that agents of only marginal effectiveness when used alone can now be employed in combined therapies to achieve specific tumor thrombosis. Equally, the sensitization, activation and/or enhancement achieved by the sensitizing component or treatment step allows existing coagulant-based anti-tumor agents, whether tumor-targeted or non-targeted, to be administered at lower doses and still achieve significant anti-tumor effects.

In all approaches of the invention, the sensitization or activation steps or agents, in combination with the coagulant-based tumor therapeutics, function to cause thrombosis in the tumor vasculature, and do not cause significant thrombosis in normal vasculature, such that the overall combined treatment achieves significant anti-tumor effects with no, minimal or reduced toxicity. Thus, any potential or actual side effects of coagulant-based tumor therapies can be reduced across the spectrum of cancer patients.

In addition, as the invention operates to sensitize tumor vasculature to coagulant-based therapies, typically by enhancing its procoagulant state, these discoveries expand the types of tumors and numbers of patients that can be effectively treated by such methods. For example, it is known that certain tumors are more resistant to coagulation than others, and the present invention therefore expands the application of coagulant-based therapies to patients having one of the more coagulation-resistant tumors.

In an overall sense, the invention thus provides methods for treating animals and patients having a vascularized tumor, comprising (a) subjecting the animal or patient to at least a first sensitizing treatment in a manner effective to enhance the procoagulant status of the tumor vasculature; and (b) treating the animal or patient with a coagulant-based tumor therapy in an manner effective to induce tumor vasculature coagulation. The “treatment” or “coagulant-based therapy” step is preferably achieved by administering to the animal or patient at least a first tumor vasculature coagulative agent in an amount effective to induce coagulation in the vasculature of the tumor.

Although, conceptually, the “sensitizing component” of the combined methods is viewed as “enhancing the procoagulant status of tumor vasculature” or “predisposing the tumor vasculature to coagulation”, there is no requirement for the sensitizing step to be “a pre-treatment”. Accordingly, the sensitizing component and the coagulant-based treatment may be performed together, such as by the combined administration of sensitizing agents and tumor vasculature coagulative agents, as validated by successful tumor treatment data herein. However, the one or more “sensitizing or activating” components or steps may indeed be performed as “a pre-treatment”, which enhances the effectiveness of targeted or non-targeted coagulants when subsequently administered.

The invention has a number of combined sensitizing embodiments. In certain cases, the invention combines one or more sensitizing agents effective to enhance the procoagulant status of tumor vasculature with one or more tumor vasculature coagulative agents to provide a combination, kit or cocktail not previously taught in the art. In such embodiments, the doses of the sensitizing agents and tumor vasculature coagulative agents are not critical, the contribution of the invention resting in the surprising combinations made possible by the insight and reasoning of the present inventors, validated by the in vivo data in the present application and further supplemented by new mechanistic understandings. In many such embodiments, sensitizing agents will be used that have not been previously used or suggested for use in connection with tumor therapy.

However, in many embodiments, the present invention provides surprisingly effective combinations and treatments using sensitizing agents or steps that have some existing connection with tumor therapy. In certain embodiments, the surprising applications of the invention are in using sensitizing agents or steps in connection with coagulative tumor therapy, as opposed to a distant branch of tumor therapy. In such embodiments, the use of lower doses of one or more of the sensitizing agents and tumor vasculature coagulative agents is an important advantage of the invention.

In still further embodiments, the invention brings together sensitizing agents or steps and tumor vasculature coagulative agents in a manner wherein the important advance rests either in the dosing of one or more agents or in the application to particular patient groups within the wide cancer field, or both. In many preferred aspects, therefore, the invention uses either low, sensitizing doses of the sensitizing agents or steps, or low, treatment doses of the tumor vasculature coagulative agents. In certain aspects, low doses of both categories of agents are preferred.

Accordingly, many of the “sensitizing dose(s)” of agents and “sensitizing level(s)” of non-invasive techniques will be “sensitizing, low” doses and levels. The sensitizing, low doses or levels are effective to enhance the procoagulant status of tumor vasculature when administered to an animal having a vascularized tumor, i.e., such that administration of a tumor vasculature coagulative agent is effective to induce coagulation in the vasculature of the tumor. Equally, many of the “treatment” doses of tumor vasculature coagulative agents are “effective low treatment doses”, i.e., low doses that are still effective to induce coagulation in tumor vasculature when administered to an animal in combination with at least a first sensitizing agent or step.

In certain embodiments, low/standard combinations may be used, such that either the sensitizing agent or the coagulative tumor therapeutic is present or used at a low dose, while the other is present or used at a standard dose. Low dose sensitizing agents and standard dose tumor vasculature coagulative agents are one aspect; and low dose tumor vasculature coagulative agents in conjunction with standard doses of sensitizing agents are the counterpart. However, in certain embodiments, both the sensitizing agent and the tumor vasculature coagulative agent may be provided at reduced doses.

Irrespective of the dosing issues, in light of the present disclosure, including the mechanism of action elucidated by the inventors, certain preferred combinations of agents are provided. For example, one of ordinary skill in the art will now appreciate that certain of the sensitizing agents function selectively in the tumor environment, such as endotoxin and TNFα. Such “tumor vasculature-selective” sensitizing agents are equally suitable for combined use with both tumor targeted coagulants (coaguligands) and non-tumor-targeted therapeutics, such as naked Tissue Factor. Other sensitizing agents and methods, which are either not so selective for tumor vasculature, or function as “non-selective vascular sensitizers”, are preferably used at low doses and in combination with targeted coagulants or targeted coagulant-drug combinations.

As used throughout the entire application, the terms “a” and “an” are used in the sense that they mean “at least one”, “at least a first”, “one or more” or “a plurality” of the referenced components or steps, except in instances wherein an upper limit is thereafter specifically stated or would be understood by one of ordinary skill in the art. The operable limits and parameters of combinations, as with the amounts of any single agent, will be known to those of ordinary skill in the art in light of the present disclosure.

The “a” and “an” terms are also used to mean “at least one”, “at least a first”, “one or more” or “a plurality” of steps in the recited methods, except where specifically stated. Thus, not only may different doses be employed in the methods of the present invention, but different numbers of doses, e.g., injections, may be used, up to and including multiple administrations.

Certain compositions of the invention comprise:

(a) an amount of a sensitizing agent effective to enhance the procoagulant status of tumor vasculature when administered to an animal having a vascularized tumor; and

(b) an amount of a tumor vasculature coagulative agent effective to induce coagulation in tumor vasculature when administered to an animal in combination with the at least a first sensitizing agent.

Similarly, certain kits of the invention comprise, in at least a first container:

(a) an amount of a sensitizing agent effective to enhance the procoagulant status of tumor vasculature when administered to an animal having a vascularized tumor; and

(b) an amount of a tumor vasculature coagulative agent effective to induce coagulation in tumor vasculature when administered to an animal in combination with the at least a first sensitizing agent.

The kits may further comprise a therapeutically effective amount of a third therapeutic agent, such as a third therapeutic agent selected from the group consisting of a chemotherapeutic agent, radiotherapeutic agent, anti-angiogenic agent, anti-tubulin drug and apoptosis-inducing agent.

Kits can further comprise at least one tumor diagnostic component.

Written instructions for using the sensitizing agent and the tumor vasculature coagulative agent in combined tumor treatment may be further provided as part of the kit, including electronic and written instructions and dosing information.

Representative methods of the invention are those for treating an animal or human patient having a vascularized tumor, comprising:

(a) subjecting the animal or patient to at least a first sensitizing treatment in a manner effective to enhance the procoagulant status of the vasculature of the vascularized tumor; and

(b) administering to the animal or patient at least a first tumor vasculature coagulative agent in an amount effective to induce coagulation in the vasculature of the tumor.

One use of the invention is the use of a tumor vasculature coagulative agent for the manufacture of a medicament for treating an animal having a vascularized tumor, the animal having previously been subjected to a sensitizing treatment in a manner effective to enhance the procoagulant status of the vasculature of the vascularized tumor.

Another use of the invention is the use of a sensitizing agent that enhances the procoagulant status of tumor vasculature for the manufacture of a medicament for treating an animal having a vascularized tumor, the animal having tumor vasculature that is not sufficiently prothrombotic to support tumor vasculature coagulative therapy in the absence of the sensitizing agent.

A further use of the invention is the use of a tumor vasculature coagulative agent for the manufacture of a medicament for treating an animal having a vascularized tumor by simultaneously subjecting the animal to a sensitizing treatment in a manner effective to enhance the procoagulant status of the vasculature of the vascularized tumor and administering the tumor vasculature coagulative agent.

Still another use of the invention is the use of a sensitizing agent that enhances the procoagulant status of tumor vasculature and a tumor vasculature coagulative agent for the manufacture of a medicament for sequential application for treating an animal having a vascularized tumor.

Yet a further use of the invention is the use of a tumor vasculature coagulative agent for the manufacture of a medicament for treating an animal having a vascularized tumor by sequential, separate or simultaneous administration of a sensitizing agent that enhances the procoagulant status of tumor vasculature and the tumor vasculature coagulative agent.

In certain of the compositions, kits, methods and uses of the invention, the tumor vasculature coagulative agent will be one or more or a plurality of non-targeted coagulation-deficient Tissue Factor compounds, i e.. “naked” Tissue Factors. Co-pending U.S. patent application Ser. No. 09/573.835. filed May 18. 2000, is specifically incorporated herein by reference in regard to even further supplementing the disclosure of such non-targeted coagulation-deficient Tissue Factor compounds.

The non-targeted coagulation-deficient Tissue Factor compounds are generally between about 100-fold and about 1,000,000-fold less active in coagulation than full length, native Tissue Factor, such as being at least about 1.000-fold less active, or at least about 10,000-fold less active, or at least about 100,000-fold less active in coagulation than full length, native Tissue Factor.

Preferred non-targeted coagulation-deficient Tissue Factor compounds are human Tissue Factor compounds, which may be prepared by recombinant means.

It is preferred that the non-targeted coagulation-deficient Tissue Factor compounds be deficient in binding to a phospholipid surface, such as may be achieved using a truncated Tissue Factor, such as a Tissue Factor compound of about 219 amino acids in length. Dimeric and polymeric Tissue Factors may also be used.

In certain embodiments, the non-targeted coagulation-deficient Tissue Factor compound will be modified to increase its biological half life, other than by attachment to a binding region that binds to a component of a tumor cell, tumor vasculature or tumor stroma. Such coagulation-deficient Tissue Factor compounds are preferably at least 100-fold less active in coagulation than full length, native Tissue Factor and have been modified to increase the biological half life; wherein the coagulation-deficient Tissue Factor compound is not attached to a targeting moiety, i.e., a targeting moiety.

Such non-targeted coagulation-deficient Tissue Factor compounds may be operatively linked to an inert carrier molecule that increases the biological half life of the coagulation-deficient Tissue Factor compound, including an inert protein carrier molecule, such as an albumin or a globulin. Other inert carrier molecules are polysaccharides or synthetic polymer carrier molecules.

Another suitable inert carrier molecule is an antibody or portion thereof, such as an IgG antibody or an Fc portion of an antibody, wherein the antibody does not specifically bind to a component of a tumor cell, tumor vasculature or tumor stroma. The non-targeted coagulation-deficient Tissue Factor compound may also be introduced into an IgG molecule in place of the C_{H}3 domain to create an inert IgG carrier molecule that comprises the non-targeted coagulation-deficient Tissue Factor compound.

In other of the compositions, kits, methods and uses of the invention, the tumor vasculature coagulative agent will be one or more or a plurality of tumor targeted coagulants, which comprise a first binding region that binds to a component expressed, accessible to binding or localized on the surface of a tumor cell, intratumoral vasculature or tumor stroma, wherein the first binding region is operatively linked to a coagulation factor or to an antibody, or antigen binding region thereof, that binds to a coagulation factor. Co-pending U.S. patent application Ser. No. 09/483,679, filed Jan. 14, 2000, is specifically incorporated herein by reference in regard to even further supplementing the disclosure of such tumor targeted coagulants.

The first binding region of the tumor targeted coagulant may be an antibody, or antigen-binding region thereof, such as a monoclonal, recombinant, human, humanized, part-human or chimeric antibody or antigen-binding region thereof. Exemplary first binding regions are an scFv, Fv, Fab′, Fab, diabody, linear antibody or F(ab′)₂ antigen-binding region of an antibody.

Other first binding regions of the tumor targeted coagulant are ligands, growth factors or receptors, a preferred example of which is VEGF.

The first binding region of the tumor targeted coagulant may bind to a component expressed, accessible to binding or localized on the surface of intratumoral blood vessels of a vascularized tumor, such as to an intratumoral vasculature cell surface receptor or to a ligand or growth factor that binds to an intratumoral vasculature cell surface receptor.

Exemplary targets include a VEGF receptor, an FGF receptor, a TGFβ receptor, a TIE, VCAM-1, ICAM-1, P-selectin, E-selectin, PSMA, αᵥβ₃ integrin, pleiotropin, endosialin or endoglin, and also VEGF, FGF, TGFβ, a ligand that binds to a TIE, a tumor-associated fibronectin isoform, scatter factor/hepatocyte growth factor (HGF), platelet factor 4 (PF4), PDGF or TIMP.

The first binding region of the tumor targeted coagulant may bind to a component expressed, accessible to binding or localized on the surface of a tumor cell or to a component released from a necrotic tumor cell, or to a component expressed, accessible to binding, inducible or localized on tumor stroma.

The tumor targeted coagulant may be one in which the first binding region is operatively linked to the coagulation factor, or where it is operatively linked to a second binding region that binds to the coagulation factor.

Human coagulation factors are preferred for use. Tissue Factor or Tissue Factor derivatives may be used, including all those described above for non-targeted use, such as truncated Tissue Factor.

Other coagulants for use in the tumor targeted coagulant are Factor II/IIa, Factor VII/VIIa, Factor IX/IXa or Factor X/Xa; and also Russell's viper venom Factor X activator, thromboxane A₂, thromboxane A₂ synthase or α2-antiplasmin.

Irrespective of the tumor vasculature coagulative agent, the compositions, kits, methods and uses of the invention may be used with a range of sensitizing treatments. Certain sensitizing treatments are applied as an external stimulus. e.g. to alter tumor blood flow or tumor vascular endothelial cell activation. These include subjecting the animal or patient to a sensitizing amount of irradiation, such as irradiation with γ-irradiation, X-rays, UV-irradiation or electrical pulses, or exposing the animal to hyperthermia or ultrasound.

Aside from the tumor vasculature coagulative agent, the compositions, kits, methods and uses of the invention may be used with a sensitizing treatment that comprises administering a sensitizing dose of one or more or a plurality of sensitizing agents. Certain sensitizing agents alter the blood flow through the vasculature in the vascularized tumor, or alter tumor vasculature permeability or structural integrity.

The sensitizing agent may enhance the procoagulant status of the tumor vasculature by inducing tissue factor on tumor vascular endothelial cells via CD14 activation, or independent of CD14 activation. The sensitizing agent may induce tissue factor on monocytes or macrophages via CD14 and K-channel activation, or independent of CD14 activation. The sensitizing agent may induce CD14/TLR expression, or activate CD14 or toll-like receptors on monocytes or macrophages.

Other sensitizing agents may induce a sensitizing amount of tumor vascular endothelial cells apoptosis; or may induce phosphatidylserine externalization on tumor vascular endothelial cells independent of apoptosis. The sensitizing agent may also induce a sensitizing amount of necrosis in tumor vascular endothelial cells. Certain sensitizing agents ligate CD40 on tumor vascular endothelial cells.

Certain preferred sensitizing agents are endotoxin or detoxified endotoxin derivatives, such as monophosphoryl lipid A (MPL).

Other preferred sensitizing agents are activating antibodies that bind to the cell surface activating antigen CD14 and that do not bind to a tumor antigen on the cell surface of a tumor cell. Exemplary antibodies are selected from the group consisting of UCHM-1, 18E12, My-4, WT14 and RoMo-1.

Certain cytokines are effective sensitizing agents, such as those selected from the group consisting of monocyte chemoattractant protein-1 (MCP-1), platelet-derived growth factor-BB (PDGF-BB) and C-reactive protein (CRP).

Tumor necrosis factor-α (TNFα) and inducers of TNFα, such as endotoxin, a Rac1 antagonist, DMXAA, CM101 or thalidomide, are preferred sensitizing agents.

Other suitable sensitizing agents are muramyl dipeptide or tripeptide peptidoglycan or a derivative thereof, synthetic lipopeptide P3CSK4, a glycosylphosphatidylinositol (GPI), a glycoinositolphospholipid (GIPL), a peptidoglycan monomer (PGM), Prevotella glycoprotein (PGP), muramyl dipeptide (MDP), threonyl-MDP or MTPPE.

Sensitizing doses of an anti-angiogenic agent may be used, such as an anti-angiogenic agent selected from the group consisting of vasculostatin, canstatin and maspin. Sensitizing doses of VEGF inhibitors are further preferred, such as an anti-VEGF blocking antibody, a soluble VEGF receptor construct (sVEGF-R), a tyrosine kinase inhibitor, an antisense VEGF construct, an anti-VEGF RNA aptamer or an anti-VEGF ribozyme.

The sensitizing agent may be an activating antibody that binds to the cell surface activating antigen CD40 or sCD40-Ligand (sCD153), such as the antibodies G28-5, mAb89, EA-5 and S2C6.

Thalidomide is another preferred sensitizing agent.

Sensitizing doses of combretastatins are also preferred, including prodrug or tumor-targeted forms thereof. Combretastatins A-1, A-2, A-3, A-4, A-5, A-6, B-1, B-2, B-3, B-4, D-1 or D-2, or a prodrug or tumor-targeted form thereof, are included.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Removal of endotoxin from recombinant truncated Tissue Factor (tTF). Endotoxin content in recombinant tTF after subsequent purification steps. 1: after Ni-NTA affinity column; 2: after gel filtration column; 3: after endotoxin affinity gel purification. Shown are the endotoxin amounts given as ng/ml protein solution (black bars) or as ng/mg specific protein (gray bars). 1 endotoxin unit equals 30-100 pg. The y-axis is on a logarithmic scale.

FIG. 2. Coagulation activity of truncated Tissue Factor (tTF) before and after depyrogenation. Coagulation activity of recombinant tTF at different concentrations was measured before (solid circles) and after (open circles) endotoxin affinity gel purification in a two stage cell free coagulation assay. Factor Xa activation as a measure of Tissue Factor activity was measured as increase of absorption at 405 nm. Values are means of duplicate data points in a representative study.

FIG. 3. Quantification of tumor necrosis in mice treated with truncated Tissue Factor (tTF) and/or LPS (endotoxin). Percentage of tumor tissue necrosis was calculated after densitometric analysis of representative tumor sections dividing the total area by the necrotic area and multiplying with 100. The statistical significance was p=0.001 for tTF treatment vs. tTF plus LPS and p=0.04 for LPS treatment vs. tTF plus LPS.

FIG. 4. Model of coagulation induction by tTF (sTF) in vivo. Intravenously injected sensitizing agents such as LPS (endotoxin) stimulates either directly, or via tumor necrosis factor-α (TNFα), the upregulation of endogenous tissue factor (TF) on the surface of endothelial cells. A synergism of TNFα with VEGF, secreted from tumor cells, exists for tissue factor upregulation. Intravenously injected tTF (sTF) associates with factor VIIa, which is present in minute amounts in the blood and binds to the endothelial cells via the Gla domain of VIIa. Both sTF-VIIa and endogenous TF increase the surface density of tissue factor resulting in the formation of dimers or dimer-like molecules. These dimers are able to support activation of factor VII to VIIa. The newly formed VIIa allows more sTF to adhere to the surface of the endothelial cells, thereby further increasing the tissue factor density. Both sTF-VIIa and endogenous TF support coagulation induction via the so-called extrinsic pathway.

### DESCRIPTION OF ILLUSTRATIVE EMBODIMENTS

Solid tumors and carcinoma account for more than 90% of all cancers in man (Shockley et al. 1991). The therapeutic uses of monoclonal antibodies and immunotoxins have been investigated in the therapy of lymphomas and leukemias (Lowder et al., 1987; Vitetta et al., 1991), but have been disappointingly ineffective in clinical trials against carcinomas and other solid tumors (Byers and Baldwin, 1988; Abrams and Oldham, 1985).

A principal reason for the ineffectiveness of antibody-based treatments is that macromolecules are not readily transported into solid tumors (Sands, 1988; Epenetos et al, 1986). Even when these molecules get into the tumor mass, they fail to distribute evenly due to the presence of tight junctions between tumor cells (Dvorak et al., 1991), fibrous stroma (Baxter et al, 1991), interstitial pressure gradients (Jain, 1990) and binding site barriers (Juweid et al. 1992).

In developing new strategies for treating solid tumors, the methods that involve targeting the vasculature of the tumor, rather than the tumor cells themselves, offer distinct advantages (U.S. Pat. Nos. 5,855,866 and 6,051,230). Inducing a blockade of the blood flow through the tumor, e.g., through tumor vasculature specific fibrin formation, interferes with the influx and efflux processes in a tumor site, thus resulting in anti-tumor effect.

Arresting the blood supply to a tumor may be accomplished through shifting the procoagulant-fibrinolytic balance in the tumor-associated vessels in favor of the coagulating processes by specific exposure to coagulating agents. Accordingly, antibody-coagulant constructs and bispecific antibodies have been generated and used in the specific delivery of coagulants to the tumor environment (U.S. Pat. Nos. 6,093,399 and 6,004,555). A preferred coagulant that has been delivered in this manner is Tissue Factor and Tissue Factor derivatives.

Tissue Factor (Factor III) is the key initiator of the extrinsic coagulation cascade. It is a transmembrane glycoprotein containing 263 residues with a molecular weight of approximately 47 kDa and belongs to the cytokine receptor family group 2. In addition to its role in the coagulation system, it can also function as a signaling receptor (Morrissey, 2001; Siegbahn, 200 1). The cDNA was cloned in 1987 by four groups (Morrissey et al., 1987; Spicer et al, 1987; Scarpati et al., 1987; Fisher et al., 1987), and the crystal structure of the extracellular domain was solved in 1994 (Harlos et al., 1994; Muller et al., 1994).

The extracellular domain of Tissue Factor is comprised of the first 219 amino acids and has been named soluble Tissue Factor (sTF) or, in later publications, truncated Tissue Factor (tTF), which is the terminology preferably employed in the present application. tTF is detectable in plasma under various conditions, e.g., in patients with unstable angina (Santucci et al., 2000), but its function is still unknown.

The ability of tTF to induce coagulation in comparison to full length TF is greatly reduced. Despite this difference in activity, truncated Tissue Factor has been exploited in inducing coagulation in selected blood vessels, particularly those within tumors. In one approach, Tissue Factor derivatives are linked to an antibody or other targeting moiety, such as growth factors or peptides. Such targeting agents home to tumor vasculature antigens, e.g to markers at the surface of tumor vascular endothelial cells, and immobilize tTF close to the membrane surface, allowing the assembly of coagulation factors on the lipid membrane similar to the physiological coagulation process (U.S. Pat. Nos. 6,093,399 and 6,004,555; Huang et al., 1997).

Coagulant-deficient Tissue Factors alone, such as tTF, can also achieve specific coagulation in tumor blood vessels, despite the fact that they lack any recognized tumor targeting component. tTF localization to blood vessels within vascularized tumors and anti-tumor effects in the absence of targeting agents are described in U.S. Pat. Nos. 6,156,321, 6,132,729 and 6,132,730. Although these non-targeted or so-called “naked” Tissue Factor therapies are widely applicable, certain tumor models do not respond well to naked Tissue Factor. For example, when mice bearing L540 human Hodgkin's disease tumors were treated with a non-targeted tTF-immunoglobulin conjugate alone, the mice showed little reduction in tumor growth relative to control.

A. Combination Therapies to Enhance Procoagulant Tumor Treatment

In order to increase the effectiveness of both targeted and non-targeted coagulation-based tumor therapies, the present inventors developed the unifying strategy of increasing the procoagulant status of tumor vascular endothelium, thus rendering the tumor vasculature more sensitive to thrombosis by coaguligands or naked Tissue Factor. Tumor endothelium typically already provides a procoagulant milieu, as compared to the vasculature of normal organs (U.S. Pat. No. 6.093,399; Ran et al, 1998; Nawroth et al., 1988). Therefore, the concept of increasing the procoagulant activity in this manner needed to be validated in animal models in vivo. The present application achieves this validation, showing that tumor vasculature can indeed be rendered even more sensitive to thrombosis by procoagulant tumor therapy without initiating unwanted activation of normal vascular endothelial cells, which would have led to thrombosis in normal organs and associated side-effects.

The inventors chose to use endotoxin in initial studies designed to increase the procoagulant activity of tumor vasculature in vivo. Endotoxin, or lipopolysaccharide (LPS), is a constitutive component of the outer membrane of gram-negative bacteria and is released when the bacteria die or multiply (Rietschel et al., 1993). Endotoxins are made of a polar heteropolysaccharide chain, covalently linked to a non-polar lipid moiety (lipid A), which anchors the molecule in the bacterial outer membrane. The molecular weight of endotoxin monomers is 10-20 kDa, but it also occurs in the form of micelles (up to 1000 kDa) or vesicles (particles of sizes up to 100 nm).

Endotoxins play a central role in the pathogenesis of gram-negative sepsis with symptoms including fever, shock, vascular leak syndrome and respiratory distress syndrome (Glauser et al., 1991; Ten Cate, 2000; Martin & Silverman, 1992). Many of the endotoxin effects involve endotoxin-induced release of cytokines, e.g., TNFα, by cells of the immune system, but direct effects on endothelial cells have also been reported (Bannermann & Goldblum, 1999).

The sensitivity to endotoxin is very much dependant on the respective species, and humans are one of the most sensitive species. McKay and Shapiro applied endotoxin in 1958 to induce disseminated intravascular coagulation in rabbits (McKay & Shapiro, 1958). In that study, a Sanarelli-Shwartzman phenomenon, i.e. glomerular thrombosis with subsequent renal cortical necrosis, was provoked in rabbits by intravenous endotoxin injections spaced 24 hours apart (McKay and Shapiro, 1958). Possible mechanisms for the observed effects include the damage of endothelial cells and leukocytes, a decreased fibrinolytic potential, blockade of the reticuloendothelial system, activation of Hageman factor and release of catecholamines and glucocorticoids during the first episode (McKay, 1973). Mice are much less sensitive than rabbits to endotoxin effects and most murine models of endotoxin shock require co-administration of additional factors (Galanos et al, 1979; Becker & Rudbach, 1978; Pieroniet et al, 1970).

In the studies disclosed herein, it was confirmed that endotoxin is able to function synergistically with tTF in the induction of coagulation on tumor endothelial cells, without causing similar effects in the endothelial cells of normal organ vasculature. The inventors were able to use low, nontoxic doses of endotoxin and still greatly enhance the thrombosis-inducing effect of tTF in tumor vasculature. Importantly, the enhanced coagulation in tumor vasculature was not observed in normal vasculature, meaning that these studies can be readily translated to the clinic.

The form of these studies involved generating recombinant tTF in E. coli and removing the contaminating endotoxin to unmeasurable levels. The recombinant, endotoxin-free tTF (depyrogenated tTF) was then spiked with defined amounts of E. coli endotoxin, and the effect on tumor vessel thrombosis was evaluated in vivo in mice bearing L540 human Hodgkin's disease tumors. Tumor-bearing mice treated with tTF alone or with low dose endotoxin showed 0% and 12% tumor tissue necrosis, respectively, but the combination of low dose endotoxin and tTF resulted in 28% necrosis. Endotoxin alone at high doses (20 μg) induced 47% tumor tissue necrosis. In mice treated with tTF alone, a slight systemic activation of the coagulation system could be measured: thrombin antithrombin-levels increased from 7.9 ng/ml to 25.4 ng/ml.

Although understanding the precise mechanism of action is not required to practice the present invention, subsequent in vitro analyses investigating the molecular mechanism of action indicate that tTF can associate in vivo with Factor VIIa, and adhere to tumor endothelial cells via the Gla domain of Factor VIIa. The tTF-VIIa complex then increases the net procoagulant effect of endothelial cells both by activating Factor X to Xa and Factor VII to VIIa. These studies are the first to describe the molecular mechanisms of coagulation induction by soluble tissue factor in vivo.

In earlier studies using L540 human Hodgkin's disease tumors, tumor-bearing mice given a non-targeted tTF-immunoglobulin conjugate showed little reduction in tumor growth relative to control. In contrast, when mice with L540 tumors were treated with the same non-targeted tTF-immunoglobulin conjugate in combination with a conventional dose of the chemotherapeutic agent, etoposide, an enhanced anti-tumor response was observed. The mechanism underlying the combined effects of routine doses of tTF and etoposide was not delineated. However, in light of the studies herein, and the new understanding provided, there is now a clearer scientific basis for these results. Moreover, the present invention describes, for the first time, the combined use of a range of agents at low or “sensitizing” doses, not suggested in earlier work, to achieve more effective and/or more widely applicable tumor treatment.

Importantly, the present invention confirms the procoagulant status of tumor vessels versus normal vessels, and shows that low, nontoxic doses of agents that activate tumor vascular endothelium in vivo can be used to increase the effectiveness of procoagulant tumor therapy without causing adverse effects in healthy tissues. These studies particularly show that naked Tissue Factor used in conjunction with low dose endotoxin can induce tumor vessel thrombosis and subsequent necrosis to a similar extent as achieved with coaguligands.

A significant point to emerge from the present invention is that the use of low dose endothelial cell activators or “coagulation sensitizers” render tumor blood vessels sensitive to thrombosis induction in vivo, whereas no thrombosis is seen in normal blood vessels. This means that the combination methods of the invention can be applied to achieve tumor blood vessel thrombosis using coagulative agents that are inactive when used alone. It also means that agents that are able to coagulate tumor vasculature when used alone may now be used at lower doses in combination with a pre-treatment step, which predisposes only the tumor vessels to additional thrombosis, leaving normal blood vessels unaltered.

The invention thus provides surprisingly effective means of safely treating tumors, which are supported by a new mechanistic understanding. An interaction between the hemostatic system and malignant diseases has been proposed by Trousseau as early as in 1872 (Trousseau, 1872). Since then, many clinicians observed thrombotic complications in cancer patients (Lip et al., 2002). However, an understanding of the ability of tumor endothelial cells to promote coagulation more readily than normal endothelium has proven elusive until recently (Ran et al. 1988, U.S. Pat. Nos. 6,406,693 and 6,312,694).

An important difference that distinguishes tumor vessels from normal vessels is the presentation of phosphatidylserine on the luminal surface of the endothelial lining, which is a key factor in the induction of thrombosis in tumor vessels using coaguligands (U.S. Pat. Nos. 6,406,693 and 6.312,694; Ran et al., 1998). The present inventors show that endotoxin and other sensitizing agents are able to further increase the procoagulant activity of tumor endothelium, rendering tumor vasculature more sensitive to thrombosis induction by coagulant-based tumor therapeutics, such as tTF and coaguligands, and that this can be achieved without upsetting the balance in normal blood vessels, and without causing thrombosis in normal tissues.

The present observations made in mice are highly applicable to humans, particularly due to the commonality of tumor blood vessels. For example, in humans tumor vessels show similar differential prothrombotic activity, which would be supported by the notion that cancer patients have a higher number of thrombotic events than the normal population. Accordingly, the present studies in animal models, coupled with the dosing and treatment regimen guidance presented herein, means that the use of sensitizing agents in combination with targeted or non-targeted coagulants will constitute a safe and effective form of tumor therapy in human patients.

The lack of evident thrombosis in normal vasculature, whilst important for the safety of clinical therapy, does not necessarily mean that there is no systemic activation of the coagulation system at all. For example, in analyzing plasma samples for coagulation parameters (thrombin-anti-thrombin complexes, antithrombin III and thrombin) three days past the inducing event, increased levels of TAT, and to a slight extent decrease of ATIII, were found after treatment with tTF. This means that there is a general activation of the coagulation system, but the levels are low and would not require clinical intervention in a human treatment setting. ATIII-levels were only very slightly decreased, with ATIII being a much less sensitive marker than TAT.

There are a number of possible mechanisms by which endotoxin could act on tumor endothelium to facilitate thrombosis induction by coagulants such as tTF. Tumor necrosis induced by injection of endotoxin or bacterial extracts has been described (Coley, 1893); Gratia & Linz, 1931; Shear, 1944; Nowotny, 1969. Old & Boyse, 1973), although not proposed as a sensitizing pre-treatment prior to treatment using coagulate-based tumor therapeutics. A connection between endotoxin and TF in endotoxin-induced thrombosis has been deduced from the fact that endotoxin effects on the coagulation system could be partially or completely blocked by inhibitors of TF (Warr et al., 1990; Elsayed et al., 1996; Ten Cate, 2000). One important aspect of the present invention is that it exploits low levels of endotoxin and other sensitizing agents to induce thrombosis selectively in tumor vasculature, whilst leaving normal vessels unaffected.

In the present studies, serum TNFα levels in mice treated with LPS were markedly elevated. TNFα is upregulated in macrophages upon stimulation with LPS (Beutler et al. 1985; Watanabe et al, 1988). Both TNFα and LPS have been reported to upregulate tissue factor in endothelial cells, macrophages and monocytes (Bevilacqua et al., 1986; Bierhaus et al., 1995; Parry et al., 1995; Moll et al., 1995; Drake et al, 1993). Using FACS analysis, the present studies also confirm the upregulation of tissue factor on murine endothelial cells by TNFα. A strong synergistic effect of VEGF with TNFα was observed on the tissue factor production of these cells. Since tumor cells are a major source of VEGF, part of the coagulation selectivity for tumor vasculature could arise from this TNFα-VEGF synergism on TF expression.

Another cause for tumor selectivity of the coagulation induction could be the high density of macrophages in tumor tissues, which produce both tissue factor and TNFα upon stimulation. Tumors are rich in macrophages, and L540 tumors are particularly so, as was demonstrated immunohistologically by the present inventors: The TNFα produced would result in tissue factor expression on the local endothelial cells, increasing the density of tissue factor molecules on the endothelial surface within the tumor (Zhang et al, 1996). Another factor contributing to the selectivity of the untargeted coagulation induction could be venous stasis in certain areas of the tumor, which has been known to predispose to thrombosis.

The extracellular domain of tissue factor, as demonstrated in this study, cannot adhere to the surface of endothelial cells per se, nor does it form homodimers with other tissue factor molecules. As to the molecular mechanism of coagulation induction by tTF, the inventors postulate that tTF captures factor VIIa, which is present in small amounts in the blood, and then adheres via the Gla domain of VIIa to the endothelial cells. In an in vitro coagulation assay using endothelial cells, it was shown that the tTF-VIIa complex indeed could adhere to the surface of endothelial cells stimulated with LPS or TNFα, thereby increasing the net procoagulant effect. Using a similar assay, it was also shown that, not only was factor Xa generated, which was the readout for procoagulant activity, part of the coagulation activity seemed to be due to de novo generation of factor VIIa.

Translating the events observed in vitro to the in vivo situation, one would expect that treatment of mice with tTF precomplexed with factor VIIa would also result in thrombosis. This was tested in 5 mice, when an average tumor necrosis rate of 33% (range 0-85%) was found. In these mice, however, side effects were more pronounced, and in ⅘ mice thromboses were seen in lung and heart, resulting in a transmural myocardial infarction in one case. This supports the notion that in the mice treated with LPS plus tTF, where such side effects were not seen, factor VIIa production occurred locally, at the site of the tumor vessels.

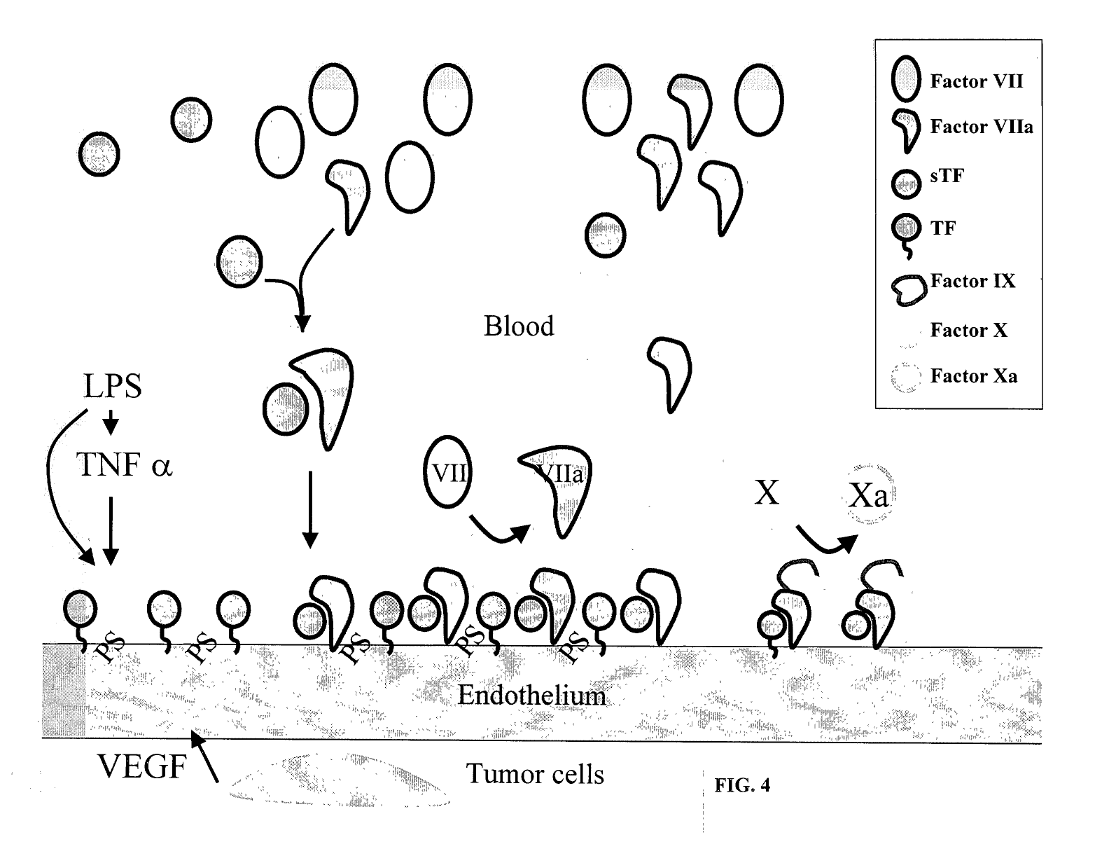

Based on the collective data of the present invention and the insight of the inventors, a model describing the molecular mechanisms of coagulation induction by tTF in vivo is provided (FIG. 4), which is particularly applicable to the sensitizing pre-treatments described herein. The sequence of events is as follows: intravenously injected sensitizing agents, such as LPS, result in upregulation of TNFα in endothelial cells and macrophages. TNFα (or LPS) synergizes with VEGF and other cytokines secreted by tumor cells (Moon & Geczy, 1988; Zuckerman et al., 1989) in the upregulation of tissue factor in tumor endothelial cells and macrophages. This increases the surface density of tissue factor molecules in tumor vasculature, increasing the difference in the expression profile over that in normal vasculature.

Intravenously injected tTF captures factor VIIa, which is present in the blood in minute amounts. The tTF-VIIa complex then adheres preferentially to activated endothelial cells, present at high numbers in the tumor (tTF-VIIa complexes can also adhere to other endothelial cells, as demonstrated by injecting precomplexed tTF-VIIa complexes into tumor bearing mice). In the tumor vasculature, the preexisting high tissue factor surface density on tumor endothelial cells is then further increased by additional binding of tTF-VIIa. This leads to an increased generation of factor Xa and increases the probability of dimers or dimer-like structure formation. The latter then induces activation of factor VII to VIIa (Donate et al., 2000).

Therefore, the local concentration of factor VIIa is increased and allows more tTF, circulating in the blood, to adhere to tumor endothelial cells. This further increases the surface density of tissue factor molecules in tumor endothelium, and more factor VIIa gets activated. Both, endogenous TF and tTF-VIIa complex will then promote the downstream events of the coagulation cascade (FIG. 4). For simplicity, several other components of the coagulation system, like platelets, neutrophils and coagulation inhibitory molecules, are not depicted in FIG. 4. Although somewhat simplified as depicted, the model is effective to explain the observations made in the present invention.

In addition to the data obtained from the L540-Hodgkin's lymphoma model, which is a preferred model due to the lack of spontaneous necrosis, the present inventors have also performed studies in mice (n=9) with a syngeneic F9-fibrosarcoma. Although spontaneous necrosis in these tumors was high (40-50% of tumor tissue), the amount of tumor tissue necrosis by treatment with endotoxin or tTF plus endotoxin was increased to 70-80%. This further strengthens the value of the present invention and its wide applicability in the treatment of a range of tumors.

Additional applications of the invention include not only the elucidation of molecular mechanisms of action of coagulation induction in vivo, but the rational drug design of coagulation inducing drugs. When normal organs of mice were carefully analyzed by light microscopy, surprisingly few side effects were observed. Phosphatidylserine expression on the luminal side of tumor vasculature is a limiting factor for coagulation induction via the tissue factor pathway (Ran et al., 1998). The present inventors further suggest that, in addition, the local factor VIIa production is another limiting factor, and the surface density of tissue factor on the luminal side of the endothelium seems to play an important role in this aspect. Care should be taken not to make factor VIIa available to the systemic circulation in the presence of tTF. The invention thus provides the opportunity to integrate these newly understood features into the design of specific coagulation inducing (or inhibiting) drugs.

Irrespective of the mechanistic understanding, and in addition to the drug design opportunities provided by the invention, it is evident that sensitizing agents such as endotoxin can now be used in combination with targeted or non-targeted coagulants as safe and effective tumor therapies. The inventors have therefore developed new sensitizing treatment methods in which a range of agents can be used to advantage in combination with vascular targeting and other procoagulant tumor therapies, such as coaguligand and naked Tissue Factor treatments. Although the invention cleverly exploits the properties of known agents, the combined use of such agents at low, sensitizing doses represents an important advance not suggested in the art.

A1. Sensitization

In terms of the “sensitizing agents” and “steps” for use in the present invention, the discoveries disclosed herein allow many agents not previously connected with tumor treatment to now be used in successful combination tumor therapy. In such aspects, any dose or level of the sensitizing agents or steps effective to enhance the procoagulant state of the tumor vasculature may be used, in which the overall treatment will involve any dose of a tumor vasculature coagulative agent effective to induce tumor vasculature coagulation.

However, certain other categories of, or individual, sensitizing agents and sensitizing steps include components already used, or suggested for use, in conventional tumor treatment. While this is an advantage of the invention for regulatory approval and safety aspects, the invention represents a new and important development over the prior art in that such sensitizing agents and/or steps are used in “low dose coagulative tumor therapy”.

In these “low dose coagulative tumor therapies”, the sensitizing agents and/or steps may be used at “sensitizing amounts, doses and/or regimens”, rather than at their “conventional therapeutic” amounts, doses and/or regimens. The “sensitizing amounts, doses and/or regimens” are lower than the counterpart “therapeutic” amounts, doses and/or regimens when such agents are used in tumor therapy, either alone or in therapies unconnected with procoagulant intervention (such as in standard combined chemotherapeutic regimens).

In other aspects, the “low dose” component of the “low dose coagulative tumor therapies” is primarily contributed by the tumor vasculature coagulative agent itself. That is, the execution of any sensitizing step, whether or not previously used or suggested for use in a conventional tumor treatment, may be combined with a dose of the tumor vasculature coagulative agent lower than previously described for therapies without a sensitizing step. Thus, the sensitizing component of the invention can be seen as facilitating the use of surprisingly low doses of coagulant-based tumor therapeutics, such as coaguligands and non-targeted Tissue Factors.

The endotoxin and tTF studies disclosed herein are instructive to highlight the application of the sensitizing treatments of the invention to lowering the dose of tumor vasculature coagulative agents. In the in vivo studies using L540 human Hodgkin's disease tumors, no anti-tumor effect has been observed using tTF alone at doses of from 4 μg tTF to 16 μg tTF. At 100 μg, anti-tumor effects begin to appear. In the sensitizing studies using a total dose of 4 μg of tTF, an effective anti-tumor response was obtained with an endotoxin dose of 500 ng. The dose was then lowered to 10 ng endotoxin, wherein similar effective anti-tumor results were obtained.

Therefore, it has already been proven that (1) low doses of a sensitizing agent can convert an ineffective coagulant therapy into an effective anti-tumor therapy; and (2) that a type of tumor unresponsive to coagulant-based therapies can be rendered sensitive to such therapies. The wider range of coagulant-based agents that may now be used effectively in tumor treatment is an evident advantage of the invention. Equally, the invention expands the patient population for coagulant-based tumor treatment, such that patients with tumors in which the blood vessels were not sufficiently prothrombotic for inclusion in these treatments can now be added to the treatment groups. Thus, the invention is applicable to a new population group.

As the 16 μg dose of tTF alone was ineffective in the L540 tumors studies, the reduction in tTF dose made possible by the use of a sensitizing agent cannot be readily quantitated from these data alone. Preliminary data using 50 and 100 μg doses of tTF with sensitizing agents suggests that at least 12-fold to 20-fold reductions are achievable, and that 50-fold to 100-fold lower doses can be used. These reductions apply equally well to coaguligands. Moreover, given the wide range of sensitizing agents and steps disclosed herein, the inventors reason that reductions in coaguligand or naked Tissue Factor doses of 100-fold, 200-fold, 500-fold or even about a 1,000-fold are within the scope of the invention.

It will be understood by those of skill in the art that the combination therapies of the present invention should be tested in an in vivo setting prior to use in a human subject. Such pre-clinical testing in animals is routine in the art. To conduct such confirmatory tests, all that is required is an art-accepted animal model of the disease in question, such as an animal bearing a solid tumor. Any animal may be used in such a context, such as, e.g., a mouse, rat, guinea pig, hamster, rabbit, dog, chimpanzee, or such like. In the context of cancer treatment, studies using small animals such as mice are widely accepted as being predictive of clinical efficacy in humans, and such animal models are therefore preferred in the context of the present invention as they are readily available and relatively inexpensive, at least in comparison to other experimental animals.

The manner of conducting an experimental animal test will be straightforward to those of ordinary skill in the art. All that is required to conduct such a test is to establish equivalent treatment groups, and to administer the combined test compounds to one group while various control studies are conducted in parallel on the equivalent animals in the remaining group or groups. Control studies using each agent alone, in addition to absolute negative controls, will generally be employed in the context of the present invention. One monitors the animals during the course of the study and, ultimately, one sacrifices the animals to analyze the effects of the treatment.

One of the most useful features of the present invention is its application to the treatment of vascularized tumors. Accordingly, anti-tumor studies can be conducted to determine the specific thrombosis within the tumor vasculature and the anti-tumor effects of the combined therapy. As part of such studies, the specificity of the effects should also be monitored, including evidence of coagulation in other vessels and tissues and the general well being of the animals should be carefully monitored.

In the context of the treatment of solid tumors, it is contemplated that effective combinations of agents and doses will be those agents and doses that generally result in at least about 10% of the vessels within a vascularized tumor exhibiting thrombosis, in the absence of significant thrombosis in non-tumor vessels; preferably, thrombosis will be observed in at least about 20%, about 30%, about 40%, or about 50% also of the blood vessels within the solid tumor mass, without significant non-localized thrombosis. At least about 60%, about 70%, about 80%, about 85%, about 90%, about 95% or even up to and including about 99% of the tumor vessels may be thrombotic. Naturally, the more vessels that exhibit thrombosis, the more preferred is the treatment, so long as the effect remains specific, relatively specific or preferential to the tumor-associated vasculature and so long as coagulation is not apparent in other tissues to a degree sufficient to cause significant harm to the animal.

Following the induction of thrombosis within the tumor blood vessels, the surrounding tumor tissues become necrotic. The successful use of the combinations of agents and doses of the invention, can thus also be assessed in terms of the expanse of the necrosis induced specifically in the tumor. Again, the expanse of cell death in the tumor will be assessed relative to the maintenance of healthy tissues in all other areas of the body. Combinations of agents and doses will have therapeutic utility in accordance with the present invention when their administration results in at least about 10% of the tumor tissue becoming necrotic (10% necrosis). Again, it is preferable to elicit at least about 20%, about 30%, about 40% or about 50% necrosis in the tumor region, without significant, adverse side-effects. Combinations of agents and doses may induce at least about 60%, about 70%, about 80%, about 85%, about 90%, about 95% up to and including 99% tumor necrosis, so long as the constructs and doses used do not result in significant side effects or other untoward reactions in the animal.

All of the above determinations can be readily made and properly assessed by those of ordinary skill in the art. For example, attendant scientists and physicians can utilize such data from experimental animals in the optimization of appropriate doses for human treatment. In subjects with advanced disease, a certain degree of side effects can be tolerated. However, patients in the early stages of disease can be treated with more moderate doses in order to obtain a significant therapeutic effect in the absence of side effects. The effects observed in such experimental animal studies should preferably be statistically significant over the control levels and should be reproducible from study to study.

Essentially each of the sensitizing agents may be used in combination with essentially each of the tumor vasculature coagulative agents, particularly wherein one or both of the sensitizing and tumor vasculature coagulative agents are used at low doses. However, in light of the detailed disclosure herein, including the mechanism of action elucidated by the inventors (FIG. 4), and the knowledge in the art, those of ordinary skill in the art will now be able to select particular combinations of sensitizing agents and tumor vasculature coagulative agents that function effectively together in tumor treatment.

For example, sensitizing agents that function selectively in the tumor environment, such as endotoxin and TNFα, may be widely used with coaguligands and naked Tissue Factor constructs. Other sensitizing agents and methods with mechanisms that are not so restricted to the tumor vasculature, or that are essentially pan-vascular sensitizers, will preferably be used at low doses and in combination with tumor-targeted coagulants. In this manner, as the coagulant-based therapeutic is targeted to the tumor, any sensitization or activation of the vasculature in normal tissues will not lead to significant side effects. In light of these and other considerations disclosed herein, and without being bound by any mechanistic theories, the inventors provide the following guidance concerning groups of agents or steps, and particular examples thereof, which may be used to advantage as sensitizing components of the present invention.

A2. Induction of Tissue Factor

The present inventors have envisioned a number of mechanisms by which the sensitizing treatments of the invention may be operating. These include enhancing the procoagulant status of the tumor vasculature by inducing tissue factor on tumor vascular endothelial cells, either via CD14 activation or independent of CD14 activation.

Preferred agents for inducing tissue factor on tumor vascular endothelial cells via CD14 activation include endotoxin, defined parts of endotoxin, lipid A and like structures, and CD14 activating antibodies. Preferred agents for inducing tissue factor on tumor vascular endothelial cells independent of CD14 activation include inflammatory cytokines, such as TNFα and IL-1; other cytokines, such as MCP-1, PDGF-BB, CRP; and VEGF. The standard and sensitizing doses of these agents are discussed below.

Tissue factor may also be induced on monocytes or macrophages via CD14 and K-channel activation, or independent of CD14 activation. Preferred agents for inducing tissue factor on monocytes or macrophages via CD14 and K-channel activation include endotoxin, defined parts of endotoxin, lipid A and like structures, and CD14 activating antibodies. The standard and sensitizing doses of these agents are discussed below. These and other agents may be used in combination with antibodies or other molecules neutralizing sCD14, to inhibit transfer of a CD14 activating structure to plasma lipoproteins.

Preferred agents for inducing tissue factor on monocytes or macrophages independent of CD14 activation include inflammatory cytokines, such as TNFα and IL-1; other cytokines, such as MCP-1, PDGF-BB, CRP; and VEGF. The standard and sensitizing doses of these agents are discussed below.

CD14/TLR expression may also be induced as part of the mechanism, and agents that induce CD14/TLR expression can be used as sensitizing agents in the invention. 22 oxyacalcitriol (OCT) is one such example, which induces CD14, but its use should be undertaken with care as it also downregulates TF and TNF, and upregulates TM. Preferred agents that induces CD14 are endotoxin, cytokines, such as GM-CSF, IL-1, IL-10 and lysophosphatidic acid (LPA). The standard and sensitizing doses of endotoxin, GM-CSF, IL-1, IL-10 are discussed below. The standard doses of LPA are those that produce effective local concentrations of about 2.5 μm, as correlated with in vitro studies (Jersmann et al., 2001). Doses for use in the sensitizing aspects of the invention in humans will be 10- to 1000-fold lower than standard.

Activating CD14 and/or toll-like receptors on monocytes or macrophages may also be used in the invention. Certain agents for use in these embodiments include endotoxin, defined parts of endotoxin, lipid A and like structures, and CD14 activating antibodies, the standard and sensitizing doses of which are discussed below. These and other agents may also be used in combination with antibodies or other molecules neutralizing sCD14, to inhibit transfer of a CD14 activating structure to plasma lipoproteins.

Additional agents that activate CD14 and/or toll-like receptors on monocytes or macrophages include muramyl dipeptide (MDP) and cytokine-inducing derivatives; synthetic lipopeptides, such as P3CSK4, which induces TLR4 independent Erk1/2 activation; glycosylphosphatidylinositol (GPI) anchors and glycoinositol-phospholipids (GIPLs) from typanosoma cruzi; peptidoglycan monomer (PGM); Prevotella glycoprotein (PGP); and lipoteichoic acid. The standard and sensitizing doses of these agents are discussed below. A TLR4 activating antibody may also be used in these embodiments, which can be used as a sensitizing agent at 10-100 fold lower than for other therapies.

A3. TNFα and Inducers of TNFα

A sub-set of agents that enhance the procoagulant status of the tumor vasculature by inducing tissue factor on tumor vascular endothelial cells are TNFα, inducers of TNFα and other cytokines that result in TF production. Preferred examples of these include endotoxin, Rac1 antagonists, such as an attenuated or engineered adenovirus, DMXAA (and FAA), CM101 and thalidomide, Endotoxin is discussed below.

Rac1 antagonists have not been previously proposed for use in cancer treatment, but may now be used in the combined treatment of the present invention, as about 5000 DNA particles per cell cause TNF upregulation independent of CD14 (Sanlioglu et al, 2001). CM101 and thalidomide can be used as sensitizing agents at up to 50-fold lower levels than when employed in conventional treatments.

The standard doses of DMXAA are 25 mg/kg in mice and 3.1 mg/m² in humans (Ching et al., 2002). The inventors reason that preferred sensitizing, low doses of DMXAA for use in the invention will be 200 ng to 10 μg. i.e.. 10 μg/kg to 500 μg/kg in mice, based on the fact that DMXAA is 20-fold less effective than endotoxin in inducing TNFα (Philpott). The lower limits contemplated for use are 10 ng, i.e.. 500 ng/kg, and the high limit 400 μg. i.e., 20 mg/kg. For human treatment, the estimated effective dose will also be about 1,000-fold lower then typically employed, i.e., about 3 μg /m².

A4. Induction of Endothelial Cell Apoptosis

Further mechanisms of enhancing the procoagulant status of the tumor vasculature include inducing a sensitizing amount of tumor vascular endothelial cell apoptosis. Any apoptosis-inducing agent can therefore be used at a low dose as a sensitizing agent of the present invention.

Angiogenesis inhibitors, such as VEGF-inhibitors, including anti-VEGF neutralizing antibodies, soluble receptor constructs, small molecule inhibitors, antisense, RNA aptamers, ribozymes, sNRP-1 and anti-VEGF Receptor antibodies, may all be employed. The standard and sensitizing doses of these agents are discussed below. Despite being slow acting, endostatin, angiostatin, thrombospondin-1, thrombospondin-2 and platelet factor-4 may be used, preferably in selected embodiments where the time of action is not a limitation.

Other suitable apoptosis-inducing agents are angiopoietin-2, used in the absence of growth factors or in presence of growth factor inhibitors; angiotensin II in presence of AT(1) inhibitors, preferably in the presence of AT(2); and apoptosis-inducing chemotherapeutic agents, such as doxorubicin.

When using angiopoietin-2, in the absence of growth factors or in presence of growth factor inhibitors, significantly reduced levels can be employed. As determined from in vitro studies, instead of 35-1250 ng/ml (Maisonpierre et al., 1997), the inventors reason that doses effective to produce as low as 0.5 ng/ml will be suitable, with 50-200 ng/ml being useful and doses effective to produce about 400 ng/ml being the upper limit.

Angiotensin II is used at a standard dose in rats of 3.5 mg/kg, and a suitable AT1 inhibitor, losartan, is typically used at 10 mg/kg (Li et al., 1997). As not previously proposed for cancer therapy, these agents can be used at the same doses in all embodiments of the present invention. However, lower doses are also useful, such as at least 10-fold lower.

The standard dose of doxorubicin in human treatment is 60 mg/M². When used in the present invention as sensitizing agents, apoptosis-inducing chemotherapeutic agents, such as doxorubicin, can be used at significantly reduced levels, as only submicromolar concentrations are required for the sensitizing effects.

A5. Phosphatidylserine Externalization

In addition to overt tumor vascular endothelial cell apoptosis, the sensitizing aspects of the invention can function by inducing activation of tumor vascular endothelial cell membranes, as represented by externalization of phosphatidylserine (PS) independent of apoptosis. Apoptosis induction is sometimes reversible and PS externalization occurs in the mid phase of apoptotic events. As PS externalization is a goal of sensitization in itself, and not just the definite death of the cells, this permits even lower doses of apoptosis-inducing agents, such as those described herein, to be used as sensitizing agents.

In these aspects of the sensitizing treatments, reactive oxygen (RO) may be involved, including nitric oxide (NO), such that NO synthases can be used. In other embodiments, depending on the agent for combined use, nitric oxide synthase (NOS) inhibitors may be used (Parkins et al., 2000). Exemplary NOS inhibitors are L-NAME, L-NNA, NLA and L-NMMA. Typically, these are used at about 1-10 mg/kg, Arsenic trioxide may also be used as a sensitizing agent, e.g., at about 10 mg/kg (Roboz et al., 2000; Lew et al., 1999).

Hydrogen peroxide, thrombin and cytokines, such as TNFα, IFNγ, IL1α, IL1β and the like, may be employed or exploited in the sensitizing step, NFκ-B activation may also be involved. Other than the cytokines, which are discussed below, the standard doses in the art will be useful for certain embodiments; however, lower doses are typically preferred, and these agents can be used at least at 10-fold lower levels than conventionally used.

A6. Endothelial Cell Necrosis

The sensitizing treatment may also induce a sensitizing amount of necrosis in tumor vascular endothelial cells. During endothelial cell necrosis, the reactive invasion of macrophages into the tumor could provide an additional source of cells to produce tissue factor and therefore generate a more procoagulant milieu. Such treatments could also have three components: 1) the necrosis induction, resulting in additional macrophage infiltration into the tumor; 2) a sensitizing agent that induces macrophages to produce tissue factor, which would be a local effect, because the density of macrophages is increased in the tumor; and 3) the coagulation inducing substance.

With the proviso that they are used at low, sensitizing doses, angiogenesis inhibitors, VEGF-inhibitors, endostatin, angiostatin and the like may be used as a sensitizing treatment of the invention to induce endothelial cell necrosis. Tumor-targeted toxins, including vascular-targeted and stromal-targeted toxins, may also be used at low doses as a sensitizing treatment of the invention.

Although generally described as agents for tertiary use with the present invention, tumor vascular immunotoxins are described in detail hereinbelow, and may be adapted for use as sensitizing agents simply by use at low doses, not previously taught. In light of the knowledge in the art regarding anti-endothelial cell immunotoxins, and the sensitizing data in the present application, the inventors reason that doses effective for sensitizing effects are half of the dose, preferably one tenth of the dose, and more preferably a 1/20 of the dose for use in a non-sensitizing context. These figures are particularly defined in terms of the ability to recruit a sufficient number of macrophages for a sensitizing effect.

A7. Inhibiting Fibrinolysis

Other methods of sensitizing treatments include activating Factor XII, as can be achieved using endotoxin, inhibiting the fibrinolytic system, activating platelets and/or neutralizing coagulation inhibitors in the tumor.

In certain embodiments, inhibition of the fibrinolytic system, which is increased in tumors, is contemplated. In these aspects, the sensitizing agent may be protamine, which inhibits heparin.

Sensitizing doses of other inhibitors of fibrinolysis may also be employed. For example, an inhibitor of fibrinolysis selected from the group consisting of α₂-antiplasmin. ε-aminocapronic acid (EACA), tranexam acid (AMCHA), trans-AMCHA, racemat of cis- and trans-AMCHA, p-aminomethylbenzoe acid (PAMBA), PAI-1 (plasmin activator inhibitor-1), PAI-2, and a neutralizing antibody or bispecific antibody against plasmin. Sensitizing doses of platelet-activating compounds may be used, such as thromboxane A₂ or thromboxane A₂ synthase. Further sensitizing agents are neutralizing antibodies against tissue factor pathway inhibitor (TFPI).

The administration of limiting coagulation factors may also be used as a sensitizing treatment of the invention. These aspects include the provision of inactive coagulation factors, plus activators thereof, the provision of the active coagulation factors alone; and the provision of the activator alone, RES blockade may also be employed to inhibit the removal of coagulation factors.

A8. CD40 Ligation

Further sensitizing mechanisms are to induce the cell surface activating antigen, CD40 and/or to ligate CD40. on tumor vascular endothelial cells. To induce CD40, cytokines such as TNFα, IFN γ and IL-1 may be used. The standard and sensitizing doses of these agents are discussed below.

To ligate CD40 on tumor vascular endothelial cells, the sensitizing agent may be an activating antibody that binds to CD40 or a CD40L activating antibody. Exemplary activating antibodies that bind to CD40 are include, but are not limited to, the anti-CD40 monoclonal antibodies mAb89 and EA-5 (Buske et al., 1997a), 17:40 and S2C6 (Bjorck et al., 1994), G28-5 (Ledbetter et al., 1994), G28-5 sFv (Ledbetter et al., 1997), as well as those disclosed in U.S. Pat. Nos. 5,801,227, 5,677,165 and 5,874.082, each incorporated herein by reference. A number of these antibodies are also commercially available, from sources such as Alexis Corporation (San Diego, Calif.) and Pharmingen (San Diego, Calif.).

Another suitable CD40 activating antibody is BL-C4 (Pradier et al., 1996). It has been reported that 100-1500 ng/ml of this activating antibody is required to induce procoagulant activity on monocytes in vitro (Pradier et al., 1996). From this information and the detailed insight of the operation of the present invention, the inventors reason that effective in vivo sensitizing doses are 400 ng-20 μg in the mouse and 100-300 ng/kg for humans. The values for use in the invention are between 10-fold and 100-fold lower than could have been envisioned prior to the present invention.

sCD40-ligand (sgp39 or sCD153) may also be used to activate CD40. CD40-ligand nucleic acid and amino acid sequences are disclosed in U.S. Pat. Nos. 5.565,321 and 5,540,926, incorporated herein by reference. Soluble versions of CD40 ligand can be made from the extracellular region, or a fragment thereof, and a soluble CD40 ligand has been found in culture supernatants from cells that express a membrane-bound version of CD40 ligand, such as EL-4 cells. sCD40-ligand at a dose of 80 ng to 4 μg would be used in the mouse. In humans, 20-60 ng/kg are contemplated for use, which are 10-fold to 100-fold lower than could have been suggested prior to the present invention.

A9. Altering Blood Flow The sensitizing step of the invention may involve altering the blood flow through tumor vasculature. This can be achieved using external, non-invasive techniques, or by administering an agent that alters tumor blood flow or tumor vasculature permeability or structural integrity. In aspects where an agent is administered, drugs that affect tumor blood flow, function, permeability and/or structural integrity are used at low, sensitizing doses, not thought to be useful prior to the present invention.

Examples of such drugs are combretastatin and analogues thereof, ZD6126 and analogues thereof, thalidomide, angiostatin and endostatin. The sensitizing doses of endostatin, angiostatin and thalidomide are contemplated to be 10- to 1000-fold lower than standard doses. Combretastatins are used in the clinic, typically at 60 mg/m² once every 3 weeks. When used as a sensitizing agent, this dose can be reduced by 10- to 1000-fold. Similar standard and sensitizing doses are applicable for ZD6126 and analogues thereof.

A10. Non-Invasive Treatments

The procoagulant status of the tumor vasculature can be enhanced using external or non-invasive stimuli. Sensitizing amounts of irradiation are used, such as sensitizing amounts of γ-irradiation, X-rays, UV-irradiation or electrical pulses. Exposing the animal or patient to hyperthermia or ultrasound may also be employed.

Certain of the external or non-invasive methods also function, at least in part, by altering the blood flow through the vasculature in the tumor, and/or by altering tumor vasculature permeability or structural integrity. Hyperthermia (ultrasound), electrical pulses and X-rays are particularly contemplated as non-invasive means to alter tumor blood flow. Standard “doses” or “levels” are >40° for 40 min for hyperthermia; greater than 1200 V of electrical pulses for growth delay of tumors; and for X-rays, 24 Gy (3×8) in mice (Edwards et al., 2002) and 40-45 Gy in humans, e.g. 10 Gy/week.

For use as sensitizing pre-treatments, the time of hyperthermia can be shorter, particularly where the second treatment is given before recovery. Rather than the standard 1200 V (Sersa et al., 1999), electrical pulses can be applied at as low as 760 V, up to about 1040 V, and achieve a decrease in perfusion. For sensitizing treatment with X-rays, the low dose of about 2.46 Gy is particularly contemplated.

A11. Endotoxin and Derivatives

Where the sensitizing treatment comprises administering a sensitizing agent, preferably at a sensitizing dose, a wide variety of agents is provided for use in the invention. Certain preferred embodiments concern the use of endotoxin or a detoxified endotoxin derivative. Endotoxin (LPS) has a polar heteropolysaccharide chain, covalently linked to a non-polar lipid moiety termed “lipid A”. Lipid A itself may be used, but this is preferably used in animals. Various detoxified endotoxins are available, which are preferred for use in animals and particularly for use in humans. Detoxified and refined endotoxins, and combinations thereof, are described in U.S. Pat. Nos. 4,866,034; 4,435,386; 4,505,899; 4,436,727; 4,436,728; 4,505,900, each specifically incorporated herein by reference.

The non-toxic derivative monophosphoryl lipid A (MPL) is one example of a detoxified endotoxin. MPL has comparable biological activities to lipid A, including B cell mitogenicity, adjuvanticity, activation of macrophages and induction of interferon synthesis. MPL-stimulated T cells enhance IL-1 secretion by macrophages. The effects of MPL on T cells include the endogenous production of factors such as TNF (Bennett et al., 1988). MPL derivatives and synthetic MPLs may thus be used in the present invention. MPL is known to be safe; clinical trials using MPL as an adjuvant have shown MPL to be safe for humans. Indeed, 100 μg/m² is known to be safe for human use, even on an outpatient basis.

Endotoxin is typically used at 100-500 μg plus enhancer for toxicity studies in mice (Becker & Rudbach, 1978; Galanos et al., 1979; Lehmann et al., 1987). In contrast, the range of sensitizing doses for use in the present invention is from 500 pg to 20 μg in mice, and generally from 10-50 μg. In humans, doses of 4 ng/kg can be used (Franco et al., 2000), but the invention provides for reduced doses of at least about 10-fold lower.

For other lipid A and defined endotoxin structures and derivatives, 3 μg-4.5 mg have been used in antitumor studies, e.g., by the Ribi group, In the present case, the inventors reason that doses as low as 10 ng to 100 ng can be employed, as shown in the mouse studies herein. In certain embodiments, particularly depending on the treatment agent, doses from 1 ng to 200 μg can be used. Human treatment will benefit from similarly reduced sensitizing doses.

A12. Peptidoglycans and Glycolipids

Further sensitizing agents are muramyl dipeptide or tripeptide peptidoglycans or derivatives thereof, synthetic lipopeptide P3CSK4. glycosylphosphatidylinositols (GPIs), glycoinositolphospholipids (GIPLs), peptidoglycan monomer (PGM) and Prevotella glycoprotein (PGP). Muramyl dipeptide (MDP) and tripeptide peptidoglycans derivatives include threonyl-MDP, fatty acid derivatives, such as MTPPE, and the derivatives described in U.S. Pat. No. 4,950,645, incorporated herein by reference.

MDP is used as an adjuvant, e.g, at 25 mg/kg (Chedid et al., 1982) and at 0.1-10 mg/kg (Chomel et al., 1987) in mice. The doses for human treatment can be reduced by about 10-fold, although similar doses can also be employed in combination with particular coagulative anti-tumor agents.

The synthetic lipopeptide P3CSK4 has been used in vitro at 10 ng/ml to 10 μg/ml. GPI anchors and glycoinositol-phospholipids GIPLs) from typanosoma cruzi have been used in vitro at 10 ng/ml (Campos et al., 2001). Each of these categories of agents are proposed for use in the sensitizing aspects of the invention at 10-100 fold lower than could have been suggested prior to the present invention.

PGM is used in vitro at 1-100 μg/ml. In mice, it has been used at 600 μg, i.e., 30 mg/kg (Gabrilovac et al., 1989) and at 10 mg/kg (Ravlic-Gulan et al., 1999; Valinger et al., 1987). PGP is used in vitro at 10 μg/ml and TLR 4 activating antibodies are used in vitro at 5 μg/ml. Each of these agents can be used as sensitizing agents at lower doses, e.g, at 100 μg/kg, and at correspondingly lower doses in humans. However, doses from 10 mg/kg up to 100 mg/kg can be employed, e.g, where other agents are used at low doses instead.

A13. CD14 Activating Antibodies

Other sensitizing agents are activating antibodies that bind to CD14. As these aspects of the invention are not intended for antigen induction, the activating antibodies will preferably not bind to a tumor antigen on the cell surface of a tumor cell. Exemplary antibodies are those selected from the group consisting of UCHM-1, 18E12, My-4, WT14 and RoMo-1. Inhibitory antibodies, such as IC14 (Verbon et al., 2001), should be avoided, as will be understood by those of ordinary skill in the art. Combinations with antibodies or other molecules neutralizing sCD14 may also be used to inhibit transfer of a CD14 activating structure to plasma lipoproteins.

From the concentration of 10 μg/ml used in vitro (Chu & Prasad, 1998), in vivo doses of about 1.5 mg are considered standard. In contrast, the present inventors reason that from 1.5 ng to 60 μg will be useful in the invention, and preferably from 30 ng to 1.5 μg, with corresponding significant reductions in the sensitizing treatments for use in humans.

A14. Inflammatory Cytokines

A range of inflammatory cytokines may be used in the present invention, preferably at sensitizing doses lower than used in other anti-tumor therapies. Such cytokines include TNFα, IL-1α, IL-1β, IL-10, GM-CSF, IFNγ and the like. More preferred cytokines are those selected from the group consisting of TNFα, and TNFα inducers, monocyte chemoattractant protein-1 (MCP-1), platelet-derived growth factor-BB (PDGF-BB) and C-reactive protein (CRP).

TNFα is used at standard doses of 4-6 μg in mice (Krosnick et al., 1989) and at 3×10⁵ U/m²/24 hour in humans (Bauer et al., 1989). Sensitizing doses suitable for use in the invention are 1 ng to 1 μg in mice, with 20-100 ng being preferred. In human treatment, in light of the mechanisms deduced by the present inventors, including the synergism with VEGF, doses of 6×10³ U/m²/24 hour will be effective, 50 fold lower than used in the art. In patients with VEGF-producing tumors, low doses of 500 U/m²/24 hour can be used. However, with certain second agents, the doses can be increased up to about 2×10⁵ U/m²/24 hour.

IL-1 is used in vitro at about 15 pg/ml. IL-1 has been used in humans as an adjuvant in vaccination protocols, including against cancer. The standard dose is 0.3-0.5 μg/m²/24 h×8 (Woodlock et al., 1999). For the sensitizing treatments of the invention, the doses for use in mice range from 1 pg to 100 ng, with about 100 pg being preferred. The doses for human treatment can be reduced by 10- to 1000-fold, in comparison to protocols available before the present invention.

IL-10 is typically used at I mg/kg in the mouse. In vitro, IL-10 is used at 1 pg/ml. For the sensitizing treatments of the invention, the doses for mice and humans are similar to those for IL-1, with dose reductions of 10- to 1000-fold being provided by the invention.

GM-CSF is used in humans at 250 μg/m²/day times 8, but this dose can be reduced by 10- to 1000-fold for use in the sensitizing aspects of the invention. Other inflammatory cytokines such as MCP-1, PDGF-BB and CRP, and VEGF, could also be used, with significant reductions in doses in contrast to other uses prior to the present invention.

A15. VEGF Inhibitors

VEGF is a multifunctional cytokine that is induced by hypoxia and oncogenic mutations. VEGF is a primary stimulant of the development and maintenance of a vascular network in embryogenesis. It functions as a potent permeability-inducing agent, an endothelial cell chemotactic agent, an endothelial survival factor, and endothelial cell proliferation factor. Its activity is required for normal embryonic development, as targeted disruption of one or both alleles of VEGF results in embryonic lethality.

The use of one or more VEGF inhibition methods is a preferred aspect of the sensitization embodiments of the invention. The recognition of VEGF as a primary stimulus of angiogenesis in pathological conditions has led to various methods to block VEGF activity, although none suggested for use as sensitizing mechanisms for combined tumor coagulative treatment. Any of the VEGF inhibitors developed may be advantageously employed in the invention at a low dose. Accordingly, any one or more of the following neutralizing anti-VEGF antibodies, soluble receptor constructs, antisense strategies, RNA aptamers and tyrosine kinase inhibitors designed to interfere with VEGF signaling may thus be used in the invention at doses 10- to 1000-fold lower than previously thought.

Suitable agents thus include neutralizing antibodies (Kim et al., 1992; Presta et al., 1997; Sioussat et al., 1993; Kondo et al., 1993; Asano et al., 1995), soluble receptor constructs (Kendall and Thomas, 1993; Aiello et al., 1995; Lin et al., 1998; Millauer et al., 1996), tyrosine kinase inhibitors (Siemeister et al., 1998), antisense strategies, RNA aptamers and ribozymes against VEGF or VEGF receptors (Saleh et al., 1996; Cheng et al., 1996). Variants of VEGF with antagonistic properties may also be employed, as described in WO 98/16551. Each of the foregoing references are specifically incorporated herein by reference.

Blocking antibodies against VEGF will be preferred in certain embodiments, particularly for simplicity, Monoclonal antibodies against VEGF have been shown to inhibit human tumor xenograft growth and ascites formation in mice (Kim et al., 1993; Mesiano et al., 1998; Luo et al., 1998a; 1998b; Borgstrom et al., 1996; 1998; each incorporated herein by reference). The antibody A4.6.1 is a high affinity anti-VEGF antibody capable of blocking VEGF binding to both VEGFR1 and VEGFR2 (Kim et al., 1992; Wiesmann et al, 1997; Muller et al.,1998; Keyt et al., 1996; each incorporated herein by reference). A4.6.1 has recently been humanized by monovalent phage display techniques and is currently in Phase I clinical trials as an anti-cancer agent (Brem, 1998; Baca et al., 1997; Presta et al, 1997; each incorporated herein by reference).

Alanine scanning mutagenesis and X-ray crystallography of VEGF bound by the Fab fragment of A4.6.1 showed that the epitope on VEGF that A4.6.1 binds is centered around amino acids 89-94. This structural data demonstrates that A4.6.1 competitively inhibits VEGF from binding to VEGFR2, but inhibits VEGF from binding to VEGFR1 most likely by steric hindrance (Muller et al.,1998; Keyt et al., 1996; each incorporated herein by reference)

A4.6.1 may be used in combination with the present invention. However, a new antibody termed 2C3 is currently preferred, which selectively blocks the interaction of VEGF with only one of the two VEGF receptors, 2C3 inhibits VEGF-mediated growth of endothelial cells, has potent anti-tumor activity and selectively blocks the interaction of VEGF with VEGFR2 (KDR/Flk-1), but not VEGFR1 (FLT-1). In contrast to A4.6.1, 2C3 allows specific inhibition of VEGFR2-induced angiogenesis, without concomitant inhibition of macrophage chemotaxis (mediated by VEGFR1), and is thus contemplated to be a safer therapeutic, U.S. Pat. Nos. 6.342,219, 6,342,221 and 6,416,758, are specifically incorporated herein by reference for the purposes of even further describing the 2C3 antibody and its uses in anti-angiogenic therapy and VEGF inhibition.

A16. Other Angiogenesis Inhibitors

Other anti-angiogenic agents used at “sensitizing” or low doses can be used with the present invention. The anti-angiogenic therapies may be based upon the provision of an anti-angiogenic agent or the inhibition of an angiogenic agent. Inhibition of angiogenic agents may be achieved by one or more of the methods described for inhibiting VEGF, including neutralizing antibodies, soluble receptor constructs, small molecule inhibitors, antisense, RNA aptamers and ribozymes may all be employed. For example, antibodies to angiogenin may be employed, as described in U.S. Pat. No. 5,520,914, specifically incorporated herein by reference.

In that FGF is connected with angiogenesis. FGF inhibitors may also be used, Certain examples are the compounds having N-acetylglucosamine alternating in sequence with 2-O-sulfated uronic acid as their major repeating units, including glycosaminoglycans, such as archaran sulfate. Such compounds are described in U.S. Pat. No. 6,028,061, specifically incorporated herein by reference, and may be used in combination herewith.

Certain sensitizing components of the invention are low doses of anti-angiogenic agents selected from the group consisting of endostatin, angiostatin, thrombospondin-1. thrombospondin-2, platelet factor-4, vasculostatin, canstatin and maspin, Angiopoietin-2 may also be used in a growth factor deficient environment or in a growth factor inhibitor rich environment. Angiotensin II may further be used in the presence of an AT(1) or AT(2) inhibitor.

Numerous tyrosine kinase inhibitors useful for the treatment of angiogenesis, as manifest in various diseases states, are now known. These include, for example, the 4-aminopyrrolo[2,3-d]pyrimidines of U.S. Pat. No. 5,639,757, specifically incorporated herein by reference, which may also be used in combination with the present invention. Further examples of organic molecules capable of modulating tyrosine kinase signal transduction via the VEGFR2 receptor are the quinazoline compounds and compositions of U.S. Pat. No. 5,792,771, which is specifically incorporated herein by reference for the purpose of describing further combinations for use with the present invention.

Compounds of other chemical classes have also been shown to inhibit angiogenesis and may be used in combination with the present invention. For example, steroids such as the angiostatic 4,9(11)-steroids and C21-oxygenated steroids, as described in U.S. Pat. No. 5,972,922, specifically incorporated herein by reference, may be employed in combined therapy, U.S. Pat. Nos. 5,712,291 and 5,593,990, each specifically incorporated herein by reference, describe thalidomide and related compounds, precursors, analogs, metabolites and hydrolysis products, which may also be used in combination with the present invention to inhibit angiogenesis. Thalidomide compounds can be used at low levels as sensitizing agents. The compounds in U.S. Pat. Nos. 5,712,291 and 5,593,990 can be administered orally. Further exemplary anti-angiogenic agents that are useful in connection with combined therapy are listed in the following Table A. Each of the agents listed therein are exemplary and by no means limiting.

TABLE A
Inhibitors and Negative Regulators of Angiogenesis

| Substances | References |
|---|---|
| Angiostatin | O'Reilly et al., 1994 |
| Endostatin | O'Reilly et al., 1997 |
| 16 kDa prolactin fragment | Ferrara et al., 1991; Clapp et al., 1993; D'Angelo et al., |
| | 1995; Lee et al., 1998 |
| Laminin peptides | Kleinman et al., 1993; Yamamura et al., 1993; |
| | Iwamoto et al., 1996; Tryggvason. 1993 |
| Fibronectin peptides | Grant et al., 1998; Sheu et al., 1997 |
| Tissue metalloproteinase | Sang, 1998 |
| inhibitors (TIMP 1, 2, 3, 4) | |
| Plasminogen activator inhibitors | Soff et al., 1995 |
| (PAI-1, −2) | |
| Tumor necrosis factor α (high | Frater-Schroder et al., 1987 |
| dose, in vitro) | |
| TGF-β1 | RayChadhury and D'Amore, 1991; Tada et al., 1994 |
| Interferons (IFN-α, -β, γ) | Moore et al., 1998; Lingen et al., 1998 |
| ELR-CXC Chemokines: | Moore et al. 1998; Hiscox and Jiang, 1997; Coughlin |
| IL-12; SDF-1: MIG; Platelet | et al., 1998; Tanaka et al., 1997 |
| factor 4 (PF-4); IP-10 | |
| Thrombospondin (TSP) | Good et al, 1990: Frazier, 1991; Bomstein, 1992; |
| | Toisma et al., 1993: Sheibani and Frazier, 1995; |
| | Volpert et al., 1998 |
| SPARC | Hasselaar and Sage, 1992; Lane et al., 1992; |
| | Jendraschak and Sage, 1996 |
| 2-Methoxyoestradiol | Fotsis et al., 1994 |
| Proliferin-related protein | Jackson et al., 1994 |
| Suramin | Gagliardi et al., 1992; Takano et al., 1994; |
| | Waltenberger et al., 1996; Gagliardi et al., 1998; |
| | Manetti et al., 1998 |
| Thalidomide | D'Amato et al., 1994; Kenyon et al., 1997 Wells, 1998 |
| Cortisone | Thorpe et al., 1993 Folkman et al., 1983 Sakamoto |
| | et al. 1986 |
| Linomide | Vukanovic et al., 1993; Ziche et al., 1998; Nagler |
| | et al., 1998 |
| Fumagillin (AGM-1470: TNP- | Sipos et al., 1994; Yoshida et al., 1998 |
| 470) | |
| Tamoxifen | Gagliardi and Collins, 1993; Lindner and Borden, |
| | 1997; Haran et al., 1994 |
| Korean mistletoe extract | Yoon et al., 1995 |
| (Viscum album coloratum) | |
| Retinoids | Oikawa et al., 1989; Lingen et al., 1996; Majewski |
| | et al. 1996 |
| CM101 | Hellerqvist et al., 1993; Quinn et al., 1995; Wamil |
| | et al., 1997; DeVore et al., 1997 |
| Dexamethasone | Hori et al., 1996; Wolff et al., 1997 |
| Leukemia inhibitory factor (LIF) | Pepper et al., 1995 |

Other components for use in inhibiting angiogenesis are angiostatin, endostatin. vasculostatin, canstatin and maspin. The protein named “angiostatin” is disclosed in U.S. Pat. Nos. 5,776,704; 5,639,725 and 5,733,876, each incorporated herein by reference. Angiostatin is a protein having a molecular weight of between about 38 kD and about 45 kD, as determined by reducing polyacrylamide gel electrophoresis, which contains approximately Kringle regions 1 through 4 of a plasminogen molecule. Angiostatin generally has an amino acid sequence substantially similar to that of a fragment of murine plasminogen beginning at amino acid number 98 of an intact murine plasminogen molecule.

The amino acid sequence of angiostatin varies slightly between species. For example, in human angiostatin, the amino acid sequence is substantially similar to the sequence of the above described murine plasminogen fragment, although an active human angiostatin sequence may start at either amino acid number 97 or 99 of an intact human plasminogen amino acid sequence. Further, human plasminogen may be used, as it has similar anti-angiogenic activity, as shown in a mouse tumor model.

Certain anti-angiogenic therapies have already been shown to cause tumor regressions, and angiostatin is one such agent. Endostatin, a 20 kDa COOH-terminal fragment of collagen XVIII, the bacterial polysaccharide CM101, and the antibody LM609 also have angiostatic activity. However, in light of their other properties, they are referred to as anti-vascular therapies or tumor vessel toxins, as they not only inhibit angiogenesis but also initiate the destruction of tumor vessels through mostly undefined mechanisms. Their delivery according to the present invention is clearly envisioned.

Angiostatin and endostatin have become the focus of intense study, as they are the first angiogenesis inhibitors that have demonstrated the ability to not only inhibit tumor growth but also cause tumor regressions in mice. There are multiple proteases that have been shown to produce angiostatin from plasminogen including elastase, macrophage metalloelastase (MME), matrilysin (MMP-7), and 92 kDa gelatinase B/type IV collagenase (MMP-9).

MME can produce angiostatin from plasminogen in tumors and granulocyte-macrophage colony-stimulating factor (GMCSF) upregulates the expression of MME by macrophages inducing the production of angiostatin. The role of MME in angiostatin generation is supported by the finding that MME is in fact expressed in clinical samples of hepatocellular carcinomas from patients. Another protease thought to be capable of producing angiostatin is stromelysin-1 (MMP-3). MMP-3 has been shown to produce angiostatin-like fragments from plasminogen in vitro.

The mechanism of action for angiostatin is currently unclear, it is hypothesized that it binds to an unidentified cell surface receptor on endothelial cells inducing endothelial cell to undergo programmed cell death or mitotic arrest. Endostatin appears to be an even more powerful anti-angiogenesis and anti-tumor agent although its biology is less clear. Endostatin is effective at causing regressions in a number of tumor models in mice. Tumors do not develop resistance to endostatin and, after multiple cycles of treatment, tumors enter a dormant state during which they do not increase in volume. In this dormant state, the percentage of tumor cells undergoing apoptosis was increased, yielding a population that essentially stays the same size. Endostatin is thought to bind an unidentified endothelial cell surface receptor that mediates its effect. Endostatin and angiostatin are thus contemplated for sensitization according to the present invention.

CM101 is a bacterial polysaccharide that has been well characterized in its ability to induce neovascular inflammation in tumors. CM101 binds to and cross-links receptors expressed on dedifferentiated endothelium that stimulates the activation of the complement system. It also initiates a cytokine-driven inflammatory response that selectively targets the tumor. It is a uniquely antipathoangiogenic agent that downregulates the expression VEGF and its receptors. CM101 is currently in clinical trials as an anti-cancer drug, and can now be used at low levels in the combination aspects of this invention.

Thrombospondin (TSP-1) and platelet factor 4 (PF4) may also be used in the present invention. These are both angiogenesis inhibitors that associate with heparin and are found in platelet α-granules. TSP-1 is a large 450 kDa multi-domain glycoprotein that is constituent of the extracellular matrix. TSP-1 binds to many of the proteoglycan molecules found in the extracellular matrix including. HSPGs, fibronectin, laminin, and different types of collagen. TSP-1 inhibits endothelial cell migration and proliferation in vitro and angiogenesis in vivo. TSP-1 can also suppress the malignant phenotype and tumorigenesis of transformed endothelial cells. The tumor suppressor gene p53 has been shown to directly regulate the expression of TSP-1 such that, loss of p53 activity causes a dramatic reduction in TSP-1 production and a concomitant increase in tumor initiated angiogenesis.

PF4 is a 70aa protein that is member of the CXC ELR-family of chemokines that is able to potently inhibit endothelial cell proliferation in vitro and angiogenesis in vivo. PF4 administered intratumorally or delivered by an adenoviral vector is able to cause an inhibition of tumor growth.

Interferons and metalloproteinase inhibitors are two other classes of naturally occurring angiogenic inhibitors that can be delivered according to the present invention. The anti-endothelial activity of the interferons has been known since the early 1980s, however, the mechanism of inhibition is still unclear. It is known that they can inhibit endothelial cell migration and that they do have some anti-angiogenic activity in vivo that is possibly mediated by an ability to inhibit the production of angiogenic promoters by tumor cells. Vascular tumors in particular are sensitive to interferon, for example, proliferating hemangiomas can be successfully treated with IFNα.

Tissue inhibitors of metalloproteinases (TIMPs) are a family of naturally occurring inhibitors of matrix metalloproteases (MMPs) that can also inhibit angiogenesis and can be used in the treatment protocols of the present invention. MMPs play a key role in the angiogenic process as they degrade the matrix through which endothelial cells and fibroblasts migrate when extending or remodeling the vascular network. In fact, one member of the MMPs, MMP-2, has been shown to associate with activated endothelium through the integrin αvβ3 presumably for this purpose. If this interaction is disrupted by a fragment of MMP-2, then angiogenesis is downregulated and in tumors growth is inhibited.

There are a number of pharmacological agents that inhibit angiogenesis, any one or more of which may be used as part of the present invention. These include AGM-1470/TNP-470. thalidomide, and carboxyamidotriazole (CAI). Fumagillin was found to be a potent inhibitor of angiogenesis in 1990. and since then the synthetic analogues of fumagillin, AGM-1470 and TNP-470 have been developed. Both of these drugs inhibit endothelial cell proliferation in vitro and angiogenesis in vivo. TNP-470 has been studied extensively in human clinical trials with data suggesting that long-term administration is optimal.

Thalidomide was originally used as a sedative but was found to be a potent teratogen and was discontinued. In 1994 it was found that thalidomide is an angiogenesis inhibitor. Thalidomide is currently in clinical trials as an anti-cancer agent as well as a treatment of vascular eye diseases, and can now be used at low levels in the combination aspects of this invention.

CAI is a small molecular weight synthetic inhibitor of angiogenesis that acts as a calcium channel blocker that prevents actin reorganization, endothelial cell migration and spreading on collagen IV. CAI inhibits neovascularization at physiological attainable concentrations and is well tolerated orally by cancer patients. Clinical trials with CAI have yielded disease stabilization in 49% of cancer patients having progressive disease before treatment.

Cortisone in the presence of heparin or heparin fragments was shown to inhibit tumor growth in mice by blocking endothelial cell proliferation. The mechanism involved in the additive inhibitory effect of the steroid and heparin is unclear although it is thought that the heparin may increase the uptake of the steroid by endothelial cells. The mixture has been shown to increase the dissolution of the basement membrane underneath newly formed capillaries and this is also a possible explanation for the additive angiostatic effect. Heparin-cortisol conjugates also have potent angiostatic and anti-tumor effects activity in vivo.

Further specific angiogenesis inhibitors may be delivered to tumors using the tumor targeting methods of the present invention. These include, but are not limited to, Anti-Invasive Factor, retinoic acids and paclitaxel (U.S. Pat. No. 5,716,981; incorporated herein by reference). AGM-1470 (Ingber et al., 1990; incorporated herein by reference); shark cartilage extract (U.S. Pat. No. 5,618,925; incorporated herein by reference); anionic polyamide or polyurea oligomers (U.S. Pat. No. 5,593,664; incorporated herein by reference); oxindole derivatives (U.S. Pat. No. 5,576,330; incorporated herein by reference); estradiol derivatives (U.S. Pat. No. 5,504,074; incorporated herein by reference); and thiazolopyrimidine derivatives (U.S. Pat. No. 5,599,813; incorporated herein by reference) are also contemplated for use as anti-angiogenic compositions for the combined uses of the present invention.

Compositions comprising an antagonist of an αᵥβ₃ integrin may also be used to inhibit angiogenesis as part of the present invention. As disclosed in U.S. Pat. No. 5,766,591 (incorporated herein by reference), RGD-containing polypeptides and salts thereof, including cyclic polypeptides, are suitable examples of αᵥβ₃ integrin antagonists.

The antibody LM609 against the αᵥβ₃ integrin also induces tumor regressions. Integrin αᵥβ₃ antagonists, such as LM609, induce apoptosis of angiogenic endothelial cells leaving the quiescent blood vessels unaffected. LM609 or other αᵥβ₃ antagonists may also work by inhibiting the interaction of αᵥβ₃ and MMP-2, a proteolytic enzyme thought to play an important role in migration of endothelial cells and fibroblasts.

Apoptosis of the angiogenic endothelium by LM609 may have a cascade effect on the rest of the vascular network. Inhibiting the tumor vascular network from completely responding to the tumor's signal to expand may, in fact, initiate the partial or full collapse of the network resulting in tumor cell death and loss of tumor volume. It is possible that endostatin and angiostatin function in a similar fashion. The fact that LM609 does not affect quiescent vessels but is able to cause tumor regressions suggests strongly that not all blood vessels in a tumor need to be targeted for treatment in order to obtain an anti-tumor effect.

As angiopoietins are ligands for Tie2, other methods of therapeutic intervention based upon altering signaling through the Tie2 receptor can also be used in combination herewith. For example, a soluble Tie2 receptor capable of blocking Tie2 activation (Lin et al., 1998a) can be employed. Delivery of such a construct using recombinant adenoviral gene therapy has been shown to be effective in treating cancer and reducing metastases (Lin et al, 1998a).

A17. Further Apoptosis Inducers

Sensitization treatment may also be achieved using agents that induce apoptosis in any cells within the tumor, including tumor cells, but preferably in tumor vascular endothelial cells. Although many anti-cancer agents may have, as part of their mechanism of action, an apoptosis-inducing effect, certain agents have been discovered, designed or selected with this as a primary mechanism, as described below. These may now be used to advantage in the low doses of the present invention.

A number of oncogenes have been described that inhibit apoptosis, or programmed cell death. Exemplary oncogenes in this category include, but are not limited to, bcr-abl, bcl-2 (distinct from bcl-1, cyclin D1; GenBank accession numbers M14745, X06487; U.S. Pat. Nos. 5,650,491; and 5,539,094; each incorporated herein by reference) and family members including Bcl-x1, Mcl-1, Bak, A1, A20. Overexpression of bcl-2 was first discovered in T cell lymphomas, bcl-2 functions as an oncogene by binding and inactivating Bax, a protein in the apoptotic pathway. Inhibition of bcl-2 function prevents inactivation of Bax, and allows the apoptotic pathway to proceed. Thus, inhibition of this class of oncogenes, e.g., using antisense nucleotide sequences, is contemplated for use in the present invention in aspects wherein enhancement of apoptosis is desired (U.S. Pat. Nos. 5,650,491; 5,539,094; and 5,583,034; each incorporated herein by reference).

Many forms of cancer have reports of mutations in tumor suppressor genes, such as p53. Inactivation of p53 results in a failure to promote apoptosis. With this failure, cancer cells progress in tumorigenesis, rather than become destined for cell death. Thus, provision of tumor suppressors is also contemplated for use in the present invention to stimulate cell death. Exemplary tumor suppressors include, but are not limited to, p53, Retinoblastoma gene (Rb). Wilm's tumor (WT1), bax alpha, interleukin-1b-converting enzyme and family, MEN-1 gene, neurofibromatosis, type 1 (NF1), cdk inhibitor p16, colorectal cancer gene (DCC), familial adenomatosis polyposis gene (FAP), multiple tumor suppressor gene (MTS-1), BRCA1 and BRCA2.

Preferred for use are the p53 (U.S. Pat. Nos. 5,747,469; 5,677,178; and 5,756,455; each incorporated herein by reference), Retinoblastoma, BRCA1 (U.S. Pat. Nos. 5,750,400; 5,654,155; 5,710,001; 5,756,294; 5,709,999; 5,693,473; 5,753,441; 5,622,829; and 5,747,282; each incorporated herein by reference). MEN-1 (GenBank accession number U93236) and adenovirus E1A (U.S. Pat. Nos. 5,776,743; incorporated herein by reference) genes.

Other compositions that may be used include genes encoding the tumor necrosis factor related apoptosis inducing ligand termed TRAIL, and the TRAIL polypeptide (U.S. Pat. No. 5,763,223; incorporated herein by reference); the 24 kD apoptosis-associated protease of U.S. Pat. No. 5,605,826 (incorporated herein by reference); Fas-associated factor 1, FAF1 (U.S. Pat. No. 5,750,653; incorporated herein by reference). Also contemplated for use in these aspects of the present invention is the provision of interleukin-1β-converting enzyme and family members, which are also reported to stimulate apoptosis.

Compounds such as carbostyril derivatives (U.S. Pat. Nos. 5,672,603; and 5,464,833; each incorporated herein by reference); branched apogenic peptides (U.S. Pat. No. 5,591,717; incorporated herein by reference); phosphotyrosine inhibitors and non-hydrolyzable phosphotyrosine analogs (U.S. Pat. Nos. 5,565,491; and 5,693,627; each incorporated herein by reference); agonists of RXR retinoid receptors (U.S. Pat. No. 5,399,586; incorporated herein by reference); and even antioxidants (U.S. Pat. No. 5,571,523; incorporated herein by reference) may also be used. Tyrosine kinase inhibitors, such as genistein, may also be linked to ligands that target a cell surface receptor (U.S. Pat. No. 5,587,459; incorporated herein by reference).

A18. Combretastatins

When used at sensitizing, low doses, a combretastatin, or a prodrug or tumor-targeted form thereof, may be used in the present invention. As described in U.S. Pat. Nos. 5,892,069, 5,504,074 and 5,661,143, each specifically incorporated herein by reference, combretastatins are estradiol derivatives that generally inhibit cell mitosis. Exemplary combretastatins that may be used in conjunction with the invention include those based upon combretastatin A, B and/or D and those described in U.S. Pat. Nos. 5,892,069, 5,504,074 and 5,661,143. Combretastatins A-1, A-2, A-3, A-4, A-5, A-6, B-1, B-2, B-3, B-4, D-1 or D2 are exemplary of the foregoing types.

U.S. Pat. Nos. 5,569,786 and 5,409,953, are incorporated herein by reference for purposes of describing the isolation, structural characterization and synthesis of each of combretastatin A-1, A2, A-3, B-1, B-2, B-3 and B-4 and formulations and methods of using such combretastatins to treat neoplastic growth. Any one or more of such combretastatins may be used in conjunction with the present invention, but at lower doses.

Combretastatin A-4, as described in U.S. Pat. Nos. 5,892,069, 5,504,074, 5,661,143 and 4,996,237, each specifically incorporated herein by reference, may also be used herewith. U.S. Pat. No. 5,561,122 is further incorporated herein by reference for describing suitable combretastatin A-4 prodrugs, which are contemplated for combined use with the present invention, but at lower doses.

U.S. Pat. No. 4,940,726, specifically incorporated herein by reference, particularly describes macrocyclic lactones denominated combretastatin D-1 and Combretastatin D-2, each of which may be used in combination with the compositions and methods of the present invention. U.S. Pat. No. 5,430,062, specifically incorporated herein by reference, concerns stilbene derivatives and combretastatin analogues with anti-cancer activity that may be used in combination with the present invention, preferably at low doses.

B. Non-Targeted (Naked) Tissue Factor

Whichever therapeutic agent is selected for use in the sensitizing step of the combination treatments of the present invention, the “coagulative tumor therapy” may be achieved using a “non-targeted coagulant”, i.e., a coagulant that is not associated with a targeting agent. Preferably, the “non-targeted coagulants” are based upon “non-targeted, coagulant-deficient tissue factor constructs”. These agents are also herein termed “naked tissue factor”, wherein the “naked” simply means “in the absence of a targeting agent or moiety”, preferably in the absence of a tumor-targeting agent or moiety.

Coagulant-deficient Tissue Factor was earlier discovered to specifically promote coagulation in tumor vasculature despite the lack of any recognized tumor targeting component. Any such coagulation-impaired TF may thus be used in the “non-sensitizing” or “treatment” step of the present invention, including non-targeted TF conjugates with improved half-life. Suitable non-targeted, coagulant-deficient tissue factor constructs are disclosed in U.S. Pat. Nos. 6,156,321, 6,132,729 and 6,132,730 (and WO 98/31394), each of which are specifically incorporated herein by reference for the purpose of even further describing and enabling these embodiments of the overall invention.

The intact TF polypeptide precursor is 295 amino acids in length, which includes a peptide leader with alternative cleavage sites, which is now known to lead to the formation of a protein of 263 amino acids in length.

A recombinant form of TF has been constructed that contains only the cell surface or extracellular domain (Stone, et al, 1995) and lacks the transmembrane and cytoplasmic regions of TF. This truncated TF (tTF) is 219 amino acids in length and is a soluble protein with approximately 10⁵ times less factor X-activating activity relative to native transmembrane TF in an appropriate phospholipid membrane environment (Ruf, et al, 1991b). This difference in activity is because the TF:VIIa complex binds and activates Factors IX and X far more efficiently when associated with a negatively charged phospholipid surface (Ruf, et al, 1991b; Paborsky, et al, 1991).

Despite the significant impairment of coagulative capacity of the tTF, tTF can promote blood coagulation when tethered or functionally associated by some other means with a phospholipid or membrane environment. This underlies the development of “coaguligands” to localize the coagulant within the tumor, exerting thrombosis and tumor necrosis.

tTF has also been proposed for possible use in treating a limited number of disorders when used in combination with other accessory molecules necessary for restoration of sufficient activity (U.S. Pat. No. 5,374,617). This possibility was exploited in certain limited circumstances by combining the use of tTF with the administration of the clotting factor, Factor VIIa. The combined use of Factor VIIa with tTF results in restoration of sufficient coagulant activity for this combination to be of use in treating bleeding disorders, such as hemophilia, in patients wherein coagulation is impaired (U.S. Pat. Nos. 5,374,617; 5,504,064; and 5,504,067).

The group of patients most readily identified with such impaired coagulation mechanisms are hemophiliacs, including those suffering from hemophilia A and hemophilia B, and those that have high titers of antibodies directed to clotting factors. In addition, this combined tTF and Factor VIIa treatment has been proposed for use in connection with patients suffering from severe trauma, post-operative bleeding or even cirrhosis (U.S. Pat. Nos. 5,374,617; 5,504,064; and 5,504,067). Both systemic administration by infusion and topical application have been proposed as useful in such therapies. These therapies can thus be seen as supplementing the body with two clotting type “factors” in order to overcome any natural limitations in these or other related molecules in the coagulation cascade in order to arrest bleeding at a specific site.

U.S. Pat. Nos. 6,156,321, 6,132,729 and 6,132,730 (and WO 98/31394) demonstrated that when tTF was systematically administered to animals with solid tumors, it was able to induce specific coagulation of the tumor's blood supply, resulting in tumor regression. Such naked tissue factor compositions may thus be used in the non-sensitizing or treatment aspects of the combination therapies of the present invention.

Various “coagulation-deficient” TF constructs may be employed, including many different forms of tTF, longer but still impaired TFs, mutants TFs, any truncated, variant or mutant TFs modified or otherwise conjugated to improve their half-life, and all such functional equivalents thereof. As detailed herein below, there are various structural considerations that may be employed in the design of candidate coagulation-deficient TFs, and various assays are available for confirming that the candidate TFs are indeed suitable for use in the treatment aspects of the present invention. Given that the technological skills for creating a variety of compounds, e.g, using molecular biology, are routine to those of ordinary skill in the art, and given the extensive structural and functional guidance provided herein, the ordinary artisan will be readily able to make and use a number of different coagulation-deficient TFs in the context of the present invention.

B1. Structural Considerations for Coagulation-Deficient TF

Those of skill in the art will readily appreciate that the TF molecules for use in the present invention cannot be substantially native TF. This is evident as natural TF and close variants thereof are particularly active in promoting coagulation. Therefore, upon administration to an animal or patient, this would lead to widespread coagulation and would be lethal. Therefore, formulations of intact, natural TF should be avoided.

Suitable TF molecules do not, alone, substantially associate with the plasma membrane. Naturally, truncation of the molecule is the most direct manner in which to achieve a modified TF that does not bind to the membrane. However, actual truncation or shortening of the molecule is not the only mechanism by which operative TF variants may be created. By way of example only, mutations may be introduced into the C-terminal region of the molecule that normally traverses the membrane in order to prevent proper membrane insertion. It is contemplated that the insertion of various additional amino acids, or the mutation of those residues already present, may be used to effect such membrane expulsion. Therefore, modifications that may be considered in this regard are those that reduce the hydrophobicity of the C-terminal portion of the molecule so that the thermodynamic properties of this region are no longer favorable to membrane insertion.

In considering making structural modifications to the native TF molecule, those of skill in the art will be aware of the need to maintain significant portions of the molecule sufficient for the resultant TF variant to be able to function to promote at least some coagulation. An important consideration is that the TF molecule should substantially retain its ability to bind to Factor VII or Factor VIIa, The VII/VIIa binding region is generally central to the molecule and such region should therefore be substantially maintained in all TF variants proposed for use in the present invention.

Nonetheless, certain sequence portions from the N-terminal region of the native TF are also contemplated to be dispensable. Therefore, one may introduce mutations into this region or may employ deletion mutants (N-terminal truncations) into the candidate TF molecules for use herewith. Given these guidelines, those of skill in the art will appreciate that the following exemplary truncated, dimeric, multimeric and mutant TF constructs are by no means limiting and that many other functionally equivalent molecules may be readily prepared and used. The following exemplary Tissue Factor compositions, including the truncated, dimeric, multimeric and mutated versions, may exist as distinct polypeptides or may be conjugated to inert carriers, such as immunoglobulins, as described herein below.

B2. Truncated Tissue Factor

As used herein, the term “truncated” when used in connection with TF means that the particular TF construct is lacking certain amino acid sequences. The term truncated thus means Tissue Factor constructs of shorter length, and differentiates these compounds from other Tissue Factor constructs that have reduced membrane association or binding. Although modified but substantially full-length TFs may thus be considered as functional equivalents of truncated TFs (“functionally truncated”), the term “truncated” is used herein in its classical sense to mean that the TF molecule is rendered membrane-binding deficient by removal of sufficient amino acid sequences to effect this change in property.

Accordingly, a truncated TF protein or polypeptide is one that differs from native TF in that a sufficient amount of the transmembrane amino acid sequence has been removed from the molecule, as compared to the native Tissue Factor. A “sufficient amount” in this context is an amount of transmembrane amino acid sequence originally sufficient to enter the TF molecule in the membrane, or otherwise mediate functional membrane binding of the TF protein. The removal of such a “sufficient amount of transmembrane spanning sequence” therefore creates a truncated Tissue Factor protein or polypeptide deficient in phospholipid membrane binding capacity, such that the protein is substantially a soluble protein that does not significantly bind to phospholipid membranes, and that substantially fails to convert Factor VII to Factor VIIa in a standard TF assay, and yet retains so-called catalytic activity including activating Factor X in the presence of Factor VIIa. U.S. Pat. No. 5,504,067 is specifically incorporated herein by reference for the purposes of further describing such truncated Tissue Factor proteins.

The preparation of particular truncated Tissue Factor constructs is described herein below. Preferably, the Tissue Factors for use in the present invention will generally lack the transmembrane and cytosolic regions of the protein. However, there is no need for the truncated TF molecules to be limited to molecules of the length of 219 amino acids. Therefore, constructs of between about 210 and about 230 amino acids in length may be used. In particular, the constructs may be about 210, 211, 212, 213, 214, 215, 216, 217, 218, 219, 220, 221, 222, 223, 224, 225, 226, 227, 228, 229, or about 230 amino acids in length.

Naturally, it will be understood that the intention is to substantially delete the transmembrane region of about 23 amino acids from the truncated molecule. Therefore, in truncated TF constructs that are longer than about 218-222 amino acids in length, the significant sequence portions thereafter will generally be comprised of about the 21 amino acids that form the cytosolic domain of the native TF molecule. In this regard, the truncated TF constructs may be between about 231, 232, 233, 234, 235, 236, 237, 238, 239, 240, or about 241 amino acids in length.

In certain preferred embodiments, tTF may be designated as the extracellular domain of mature Tissue Factor protein. Therefore, in exemplary preferred embodiments, tTF may comprise residues 1-219 of the mature protein.

B3. Dimeric Tissue Factor Constructs

Previously it has been shown that it is possible for native Tissue Factor on the surface of J82 bladder carcinoma cells to exist as a dimer (Fair et al., 1987). The binding of one Factor VII or Factor VIIa molecule to one Tissue Factor molecule may also facilitate the binding of another Factor VII or Factor VIIa to another Tissue Factor (Fair et al, 1987; Bach et al., 1986). Furthermore, Tissue Factor shows structural homology to members of the cytokine receptor family (Edgington et al., 1991) some of which dimerize to form active receptors (Davies and Wlodawer, 1995). As such it is contemplated that the truncated Tissue Factor compositions of the present invention may be useful as dimers.

Accordingly, any of the truncated, mutated or otherwise coagulation-deficient Tissue Factor constructs disclosed herein, or an equivalent thereof, may be prepared in a dimeric form for use in the present invention. As will be known to those of ordinary skill in the art, such TF dimers may be prepared by employing the standard techniques of molecular biology and recombinant expression, in which two coding regions are prepared in-frame and expressed from an expression vector. Equally, various chemical conjugation technologies may be employed in connection with the preparation of TF dimers. The individual TF monomers may be derivatized prior to conjugation. All such techniques would be readily known to those of skill in the art.

If desired, the Tissue Factor dimers or multimers may be joined via a biologically-releasable bond, such as a selectively-cleavable linker or amino acid sequence. For example, peptide linkers that include a cleavage site for an enzyme preferentially located or active within a tumor environment are contemplated. Exemplary forms of such peptide linkers are those that are cleaved by urokinase, plasmin, thrombin, Factor IXa, Factor Xa, or a metalloproteinase, such as collagenase, gelatinase or stromelysin.

In certain embodiments, the Tissue Factor dimers may further comprise a hindered hydrophobic membrane insertion moiety, to later encourage the functional association of the Tissue Factor with the phospholipid membrane, but only under certain defined conditions. As described in the context of the truncated Tissue Factors, hydrophobic membrane-association sequences are generally stretches of amino acids that promote association with the phospholipid environment due to their hydrophobic nature. Equally, fatty acids may be used to provide the potential membrane insertion moiety.

Such membrane insertion sequences may be located either at the N-terminus or the C-terminus of the TF molecule, or generally appended at any other point of the molecule so long as their attachment thereto does not hinder the functional properties of the TF construct. The intent of the hindered insertion moiety is that it remains non-functional until the TF construct localizes within the tumor environment, and allows the hydrophobic appendage to become accessible and even further promote physical association with the membrane. Again, it is contemplated that biologically-releasable bonds and selectively-cleavable sequences will be particularly useful in this regard, with the bond or sequence only being cleaved or otherwise modified upon localization within the tumor environment and exposure to particular enzymes or other bioactive molecules.

B4. Tri and Multimeric Tissue Factor Constructs

In other embodiments the tTF constructs of the present invention may be multimeric or polymeric. In this context a “polymeric construct” contains 3 or more Tissue Factor constructs of the present invention. A “multimeric or polymeric TF construct” is a construct that comprises a first TF molecule or derivative operatively attached to at least a second and a third TF molecule or derivative, and preferably, wherein the resultant multimeric or polymeric construct is still deficient in coagulating activity as compared to wild-type TF. In preferred embodiments, the multimeric and polymeric TF constructs for use in this invention are multimers or polymers of truncated TF molecules, which may be optionally combined with other coagulation-deficient TF constructs or variants.

The multimers may comprise between about 3 and about 20 such TF molecules, with between about 3 and about 15 or about 10 being preferred and between about 3 and about 10 being most preferred. Naturally, TF multimers of at least about 3, 4, 5, 6, 7, 8, 9 or 10 or so are included within the present invention. The individual TF units within the multimers or polymers may also be linked by selectively-cleavable peptide linkers or other biological-releasable bonds as desired. Again, as with the TF dimers discussed above, the constructs may be readily made using either recombinant manipulation and expression or using standard synthetic chemistry.

B5. Factor VII Activation Mutants

Even further TF constructs useful in context of the present invention are those mutants deficient in the ability to activate Factor VII. The basis for the utility of such mutants lies in the fact that they are also “coagulation-deficient”. Such “Factor VII activation mutants” are generally defined herein as TF mutants that bind functional Factor VII/VIIa, proteolytically activate Factor X, but are substantially free from the ability to proteolytically activate Factor VII. Accordingly, such constructs are TF mutants that lack Factor VII activation activity.

The ability of such Factor VII activation mutants to function in promoting tumor-specific coagulation is based upon both the localization of the TF construct to tumor vasculature, and the presence of Factor VIIa at low levels in plasma. Upon administration of such a Factor VII activation mutant, the mutant would generally localize within the vasculature of a vascularized tumor, as would any TF construct of the invention. Prior to localization, the TF mutant would be generally unable to promote coagulation in any other body sites, on the basis of its inability to convert Factor VII to Factor VIIa. However, upon localization and accumulation within the tumor region, the mutant will then encounter sufficient Factor VIIa from the plasma in order to initiate the extrinsic coagulation pathway, leading to tumor-specific thrombosis.

As is developed more fully below, a preferred use of the Factor VII activation mutants is in combination with the co-administration of Factor VIIa. Although useful in and of themselves, as described above, such mutants will generally have less than optimal activity given that Factor VIIa is known to be present in plasma only at low levels (about 1 ng/ml, in contrast to about 500 ng/ml of Factor VII in plasma. U.S. Pat. Nos. 5,374,617; 5,504,064; and 5,504,067). Therefore, the co-administration of exogenous Factor VIIa along with the Factor VII activation mutant is preferred over the administration of the mutants alone. In that these mutants are expected to have almost no side effects, their combined use with simultaneous, preceding or subsequent administration of Factor VIIa is an advantageous aspect of the present invention.

Any one or more of a variety of Factor VII activation mutants may be prepared and used in connection with either aspect of the present invention. There is a significant amount of scientific knowledge concerning the recognition sites on the TF molecule for Factor VII/VIIa. By way of example only, one may refer to the articles by Ruf and Edgington (1991 a), Ruf et al. (1992c), and to WO 94/07515 and WO 94/28017, each specifically incorporated herein by reference for further guidance on these matters. It will thus be understood that the Factor VII activation region generally lies between about amino acid 157 and about amino acid 167 of the TF molecule. However, it is contemplated that residues outside this region may also prove to be relevant to the Factor VII activating activity, and one may therefore consider introducing mutations into any one or more of the residues generally located between about amino acid 106 and about amino acid 209 of the TF sequence (WO 94/07515).

In terms of the preferred region, one may generally consider mutating any one or more of amino acids 147, 152, 154, 156, 157, 158, 159, 160, 161, 162, 163, 164, 165, 166 and/or 167. With reference to the generally preferred candidate mutations outside this region, one may refer to the following amino acid substitutions: S16, T17, S39, T30, S32, D34, V67, L104, B105, T106, R131, R136, V145, V146, F147, V198, N199, R200 and K201, with amino acids A34, E34 and R34 also being considered (WO 94/28017).

As mentioned, preferably the Tissue Factors are rendered deficient in the ability to activate Factor VII by altering one or more amino acids from the region generally between about position 157 and about position 167 in the amino acid sequence, Exemplary mutants are those wherein Trp at position 158 is changed to Arg; wherein Ser at position 162 is changed to Ala; wherein Gly at position 164 is changed to Ala; and the double mutant wherein Trp at position 158 is changed to Arg and Ser at position 162 is changed to Ala. Of course these are exemplary mutations and it is envisioned that any Tissue Factor mutant having an altered amino acid composition that has the desirable characteristic of binding to Factor VII/VIIa but not activating the coagulation cascade will be useful in the context of the present invention.

B6. Quantitative Assessment of Coagulant Deficiency

The coagulation-deficient Tissue Factor constructs, whether they are truncated. mutated, truncated and mutated, dimeric, multimeric, conjugated to inert carriers to increase their half-life, or any combination of the foregoing, are each coagulation-deficient as compared to native, wild-type Tissue Factor. By the term “coagulation-deficient”, as used herein, is meant that the TF constructs have an impaired ability to promote coagulation such that their administration into the systemic circulation of an animal or human patient does not lead to significant side effects or limiting toxicity. A TF construct can be readily analyzed in order to determine whether it meets this definition, simply by conducting a test in an experimental animal. However, the following detailed guidance is provided to assist those of skill in the art in the prior characterization and selection of appropriate candidates coagulation-deficient TF constructs, in order that any experimental animal studies may be conducted efficiently and cost-efficiently.

In quantitative terms, the coagulation-deficient TFs will be 100-fold or more less active than full length, native TF, that is, they will be 100-fold or more less able to induce coagulation of plasma than is full length, native TF when tested in an appropriate phospholipid environment.

More preferably, the impaired TFs should be 1,000-fold or more less able to induce coagulation of plasma than is full length, wild type TF in an appropriate phospholipid environment; even more preferably, the TFs should be 10,000-fold or more less able to induce coagulation of plasma than full length, wild type TF in such an environment; and most preferably, the impaired TFs should be 100,000-fold or more less able to induce coagulation of plasma than is full length, native TF in an appropriate phospholipid environment. It will be appreciated that this “100,000-fold” generally corresponds to one of the currently preferred constructs, the truncated Tissue Factor of 219 amino acids in length.

Inherent within the definition of “X-fold or more less able to induce coagulation of plasma” is the concept that the subject TF undergoing investigation is still able to induce coagulation of plasma. Evidently, a TF that has been modified to render its completely unable to induce coagulation will generally not be useful in the context of the present invention. TFs that are less active than wild-type TF in the controlled, phospholipid assays by about 500,000-fold are still contemplated to have utility in connection herewith. Similarly, all TF variants and mutants that are between about 500,000-fold and about 1,000,000-fold less able to induce coagulation of plasma than is full length, native TF in an appropriate phospholipid environment are still envisioned to have utility in certain embodiments. It is generally considered that 1,000,000-fold (10⁶) impairment of activity will generally be about the least active that one would consider for use in the present invention. However, those TF constructs that are towards the less active end of the stated range still have utility in connection the present invention, given the surprising effectiveness of the combination therapies. The choice of particular TF variant and the initial therapeutic strategy will be readily determined by one of ordinary skill in the art.

Notwithstanding that there will be certain preferred and/or optimal uses and combinations of the various TF elements, the coagulation-deficient TFs for use in the present invention will generally be between about 100-fold and about 1,000,000-fold less active than wild-type TF; more preferably, will be between about 1,000-fold and about 100,000-fold less active; and may be categorized as less active by any number within the stated ranges, including by about 10,000-fold. The ranges themselves may also be varied between about 1,000-fold and 1,000,000-fold, or between about 10,000-fold and 500,000-fold, or such like.

Any one or more of a number of in vitro plasma coagulation activity assays may be employed in connection with the quantitative testing of candidate coagulation-deficient Tissue Factors, For example, suitable assays are described in U.S. Pat. Nos. 6,156,321, 6,132,729 and 6,132,730, and WO 98/31394, all specifically incorporated herein by reference. For further details regarding tTF and procoagulation assays, the skilled practitioner is referred to U.S. Pat. Nos. 5,437,864; 5,223,427; and 5,110,730 and PCT publication numbers WO 94/28017; WO 94/05328; and WO 94/07515, each of which are specifically incorporated by reference herein for the purposes of even further supplementing the present disclosure in regard to assays. Candidate TF compositions may be tested using the foregoing and similar assays to confirm that their functionality has been maintained, but that their ability to promote coagulation has been impaired by at least the required amount of about 100-fold and preferably by about 1,000-fold, more preferably by about 10,000-fold, and most preferably by about 100,000-fold.

B7. Prolonged Half-Life TF

It is demonstrated herein that the anti-tumor activity of tTF is enhanced by conjugating tTF to inert carrier molecules, such as immunoglobulins, that delay clearance of tTF from the body. For example, linking tTF to immunoglobulin enhances the anti-tumor activity by prolonging the in vivo half-life of tTF such that tTF persists for longer and has more time to induce thrombotic events in tumor vessels. The prolongation in half-life either results from the increase in size of tTF above the threshold for glomerular filtration; or from active readsorption of the conjugate within the kidney, a property of the Fc piece of immunoglobulin (Spiegelberg and Weigle, 1965). It is also possible that the immunoglobulin component changes the conformation of tTF to render it more active or stable. Other carrier molecules besides immunoglobulin are contemplated to produce similar effects and are thus encompassed within the present invention.

Given that a first interpretation of the prolonged half-life observed upon the linkage of tTF to immunoglobulin is simply that the resultant increase in size leads to prolonged plasma half-life, the inventors contemplate that other modifications that increase the size of TF constructs can be advantageously used in connection with the present invention, so long as the lengthening modification does not substantially restore membrane-binding functionality to the modified TF construct. Absent such a possibility, which can be readily tested. virtually any generally inert biologically acceptable molecule may be conjugated with a TF construct in order to prepare a modified TF with increased in vivo half-life.

Modification may also be made to the structure of TF itself to render it either more stable, or perhaps to reduce the rate of catabolism in the body. One mechanism for such modifications is the use of d-amino acids in place of l-amino acids in the TF molecule. Those of ordinary skill in the art will understand that the introduction of such modifications needs to be followed by rigorous testing of the resultant molecule to ensure that it still retains the desired biological properties. Further stabilizing modifications include the use of the addition of stabilizing moieties to either the N-terminal or the C-terminal, or both, which is generally used to prolong the half-life of biological molecules. By way of example only, one may wish to modify the termini of the TF constructs by acylation or amination. The variety of such modifications may also be employed together, and portions of the TF molecule may also be replaced by peptidomimetic chemical structures that result in the maintenance of biological function and yet improve the stability of the molecule.

Techniques useful in connection with conjugation proteins of interest to carrier proteins are widely used in the scientific community. It will be generally understood that in the preparation of such TF conjugates for use in the present invention, the protein chosen as a carrier molecule should have certain defined properties. For example, it must of course be biologically compatible and not result in any significant untoward effects upon administration to a patient. Furthermore, it is generally required that the carrier protein be relatively inert, and non-immunogenic, both of which properties will result in the maintenance of TF function and will allow the resultant construct to avoid excretion through the kidney. Exemplary proteins are albumins and globulins.

In common with the protein conjugates described above, the TF molecules of the present invention may also be conjugated to non-protein elements in order to improve their half-life in vivo. Again, the choice of non-protein molecules for use in such conjugates will be readily apparent to those of ordinary skill in the art. For example, one may use any one or more of a variety of natural or synthetic polymers, including polysaccharides and PEG.

In the context of preparing conjugates, whether proteinaceous or non-proteinaceous, one should take care that the introduced conjugate does not substantially reassociate the modified TF molecule with the plasma membrane such that it increases its coagulation ability and results in a molecule that exerts harmful side effects following administration. As a general rule, it is believed that hydrophobic additions or conjugates should largely be avoided in connection with these embodiments.

Where antibodies are used to conjugate to the tTF compositions of the present invention the choice of antibody will generally be dependent on the intended use of the TF-antibody conjugate. Where a naked TF immunoconjugate is the secondary therapeutic agent, rather than a targeted coaguligand, the immunoconjugates will not in any sense be a “targeted immunoconjugate”. In these aspects, the conjugation of the TF molecule to an antibody or portion thereof is simply performed in order to generate a construct that has improved half-life and/or bioavailability in comparison to the original TF molecule. In any event, certain advantages may be achieved through the application of particular types of antibodies. For example, while IgG based antibodies may be expected to exhibit better binding capability and slower blood clearance than their Fab′ counterparts, Fab′ fragment-based compositions will generally exhibit better tissue penetrating capability.

The inventors contemplate that the Fc portion of the immunoglobulin in the tTF-immunoglobulin construct employed in the advantageous studies disclosed herein may actually be the relevant portion of the antibody molecule, resulting in increased in vivo half-life. It is reasonable to presume that the conjugation to the Fc region results in active readsorption of a TF-Fc conjugate within the kidney, restoring the conjugate to the systemic circulation. As such, one may conjugate any of the coagulation-deficient TF constructs or variants of the invention to an Fc region in order to increase the in vivo half-life of the resultant conjugate.

Various methods are available for producing Fc regions in sufficient purity to enable their conjugation to the TF constructs. By way of example only, the chemical cleavage of antibodies to provide the defined domains or portions is well known and easily practiced, and recombinant technology can also be employed to prepare either substantial quantities of Fc regions or, indeed, to prepare the entire TF-Fc conjugate following generation of a recombinant vector that expresses the desired fusion protein.

Further manipulations of the general immunoglobulin structure may also be conducted with a view to providing second generation TF constructs with increased half-life. By way of example only, one may consider replacing the C_{H}3 domain of an IgG molecule with a truncated Tissue Factor or variant thereof. In general, the most effective mechanism for producing such a hybrid molecule will be to use molecular cloning techniques and recombinant expression. All such techniques are generally known to those of ordinary skill in the art, and are further described in detail herein.

Once a candidate TF construct has been generated with the intention of providing a construct with increased in vivo half-life, the construct should generally be tested to ensure that the desired properties have been imparted to the resultant compound. The various assays for use in determining such changes in function are routine and easily practiced by those of ordinary skill in the art.

In TF conjugates designed simply in order to increase their size, confirmation of increased size is completely routine. For example, one will simply separate the candidate composition using any methodology that is designed to separate biological components on the basis of size and one will analyze the separated products in order to determine that a TF construct of increased size has been generated. By way of example only, one may mention separation gels and separation columns, such as gel filtration columns. The use of gel filtration columns in the separation of mixtures of conjugated and non-conjugated components may also be useful in other aspects of the present invention, such as in the generation of relatively high levels of conjugates, immunotoxins or coaguligands.

As the objective of the present class of conjugates is to provide a coagulation-deficient TF molecule having an increased in vivo half-life, the candidate TF modified variants or conjugates should generally be tested in order to confirm that this property is present. Again, such assays are routine in the art. A first simple assay would be to determine the half-life of the candidate modified or conjugated TF in an in vitro assay. Such assays generally comprise mixing the candidate molecule in sera and determining whether or not the molecule persists in a relatively intact form for a longer period of time, as compared to the initial sample of coagulation-deficient Tissue Factor. One would again sample aliquots from the admixture and determine their size, and preferably, their biological function.

In vivo assays of biological half-life or “clearance” can also be easily conducted. In these systems, it is generally preferred to label the test candidate TF constructs with a detectable marker and to follow the presence of the marker after administration to the animal. preferably via the route intended in the ultimate therapeutic treatment strategy. As part of this process, one would take samples of body fluids, particularly serum and/or urine samples, and one would analyze the samples for the presence of the marker associated with the TF construct, which will indicate the longevity of the construct in the natural environment in the body.

C. Coaguligands

Irrespective of the sensitizing agent employed in the combination treatment methods of the present invention, the “coagulative tumor therapy” may be achieved using a “coaguligand”. i.e., a coagulant that is operatively attached to a targeting agent. Preferably, the targeting agent binds to a targetable component of tumor vasculature or stroma. However, targeting tumor cells and/or tumor cell components with a coaguligand can also be effective. The targeting agents also preferably bind to a surface-expressed, surface-accessible or surface-localized component of a tumor cell, tumor vasculature or tumor stroma. However, once tumor vasculature and tumor cell destruction begins, internal components will be released, allowing additional targeting of virtually any tumor component.

U.S. Pat. Nos. 5,877,289, 6,004,555 and 6,093,399 exemplify the preparation and use of a range of tumor-targeted coaguligands, which have been employed to specifically induce coagulation in the tumor's blood supply, resulting in tumor necrosis. These coaguligands exemplify the types of tumor-targeted coagulative therapeutic agents for use in the non-sensitizing or treatment aspects of the combination therapies of the present invention.

C1. Tumor Cell Targeting Agents

Those aspects of the present invention that involve targeting tumor cells and tumor cell components are still effective anti-vascular strategies as they function to block or destroy the tumor vessels, and are not aimed at killing the tumor cells directly. In binding to a tumor cell component or to a component associated with a tumor cell, the binding ligands cause the attached coagulant to concentrate on those perivascular tumor cells nearest to the blood vessel and thus exert anti-vascular effects.

Suitable targeting agents and binding regions are therefore components, such as antibodies and other agents, which bind to a tumor cell. Agents that “bind to a tumor cell” are defined herein as targeting agents that bind to any accessible component or components of a tumor cell, or that bind to a component that is itself bound to, or otherwise associated with, a tumor cell, as further described herein.

The majority of such tumor cell-targeting agents and binding ligands are contemplated to be agents, particularly antibodies, that bind to a cell surface tumor antigen or marker. Many such antigens are known, as are a variety of antibodies for use in antigen binding and tumor targeting. The invention thus includes first targeting agents and binding regions, such as antigen binding regions of antibodies, that bind to an identified tumor cell surface antigen and/or that bind to an intact tumor cell. The identified tumor cell surface antigens and intact tumor cells of Table I and Table II of U.S. Pat. Nos. 5,877,289; 6,004,555; 6,036,955; 6,093,399 are specifically incorporated herein by reference for the purpose of exemplifying suitable tumor cell surface antigens.

Currently preferred examples of tumor cell binding regions are those that comprise an antigen binding region of an antibody that binds to the cell surface tumor antigen p185^{HER2}, milk mucin core protein, TAG-72, Lewis a or carcinoembryonic antigen (CEA). Another group of currently preferred tumor cell binding regions are those that comprise an antigen binding region of an antibody that binds to a tumor-associated antigen that binds to the antibody 9.2.27, OV-TL3, MOv18, B3 (ATCC HB 10573), KS1/4 (obtained from a cell comprising the vector pGKC2310 (NRRL B-18356) or the vector pG2A52 (NRRL B-18357), 260F9 (ATCC HB 8488) or D612 (ATCC HB 9796).

The antibody 9.2.27 binds to high Mᵣ melanoma antigens, OV-TL3 and MOv18 both bind to ovarian-associated antigens, B3 and KS1/4 bind to carcinoma antigens, 260F9 binds to breast carcinoma and D612 binds to colorectal carcinoma. Antigen binding moieties that bind to the same antigen as D612, B3 or KS1/4 are particularly preferred. D612 is described in U.S. Pat. No. 5,183,756, and has ATCC Accession No. HB 9796; B3 is described in U.S. Pat. No. 5,242,813, and has ATCC Accession No. HB 10573; and recombinant and chimeric KS1/4 antibodies are described in U.S. Pat. No. 4,975,369; each incorporated herein by reference.

In tumor cell targeting, where the tumor marker is a component, such as a receptor, for which a biological ligand has been identified, the ligand itself may also be employed as the targeting agent, rather than an antibody. Active fragments or binding regions of such ligands may also be employed.

Targeting agents and binding regions for use in the invention may also be components that bind to a ligand that is associated with a tumor cell marker. For example, where the tumor antigen in question is a cell-surface receptor, tumor cells in vivo will have the corresponding biological ligand. e.g., hormone, cytokine or growth factor, bound to their surface and available as a target. This includes both circulating ligands and “paracrine-type” ligands that may be generated by the tumor cell and then bound to the cell surface.

The present invention thus further includes first binding regions, such as antibodies and fragments thereof, that bind to a ligand that binds to an identified tumor cell surface antigen, or that preferentially or specifically binds to one or more intact tumor cells. Additionally, the receptor itself, or preferably an engineered or otherwise soluble form of the receptor or receptor binding domain, could also be employed as the binding region.

Targetable components of tumor cells further include components released from necrotic or otherwise damaged tumor cells, including cytosolic and/or nuclear tumor cell antigens. These are preferably insoluble intracellular antigen(s) present in cells that may be induced to be permeable, or in cell ghosts of substantially all neoplastic and normal cells, that are not present or accessible on the exterior of normal living cells of a mammal.

U.S. Pat. Nos. 5,019,368, 4,861,581 and 5,882,626, each issued to Alan Epstein and colleagues, are each specifically incorporated herein by reference for purposes of even further describing and teaching how to make and use antibodies specific for intracellular antigens that become accessible from malignant cells in vivo. The antibodies described are sufficiently specific to internal cellular components of mammalian malignant cells, but not to external cellular components. Exemplary targets include histones, but all intracellular components specifically released from necrotic tumor cells are encompassed.

Upon administration to an animal or patient with a vascularized tumor, such antibodies localize to the malignant cells by virtue of the fact that vascularized tumors naturally contain necrotic tumor cells, due to the process(es) of tumor re-modeling that occur in vivo and cause at least a proportion of malignant cells to become necrotic. In addition, the use of such antibodies in combination with other therapies that enhance tumor necrosis serves to enhance the effectiveness of targeting and subsequent therapy.

These types of antibodies may thus be used to directly or indirectly associate with coagulants and to administer the coagulants to necrotic malignant cells within vascularized tumors, as generically disclosed herein.

As also disclosed in U.S. Pat. Nos. 5,019,368, 4,861,581 and 5,882,626, each incorporated herein by reference, these antibodies may be used in combined diagnostic methods and in methods for measuring the effectiveness of anti-tumor therapies. Such methods generally involve the preparation and administration of a labeled version of the antibodies and measuring the binding of the labeled antibody to the internal cellular component target preferentially bound within necrotic tissue. The methods thereby image the necrotic tissue, wherein a localized concentration of the antibody is indicative of the presence of a tumor and indicate ghosts of cells that have been killed by the anti-tumor therapy.

C2. Tumor Vascular Targeting Agents

A range of suitable targeting agents are available that bind to markers present on tumor endothelium and stroma, but largely absent from normal cells, endothelium and stroma. Generally speaking, the antibodies, ligands and conjugates thereof will preferably exhibit properties of high affinity and will not exert significant in vivo side effects against life-sustaining normal tissues, such as one or more tissues selected from heart, kidney, brain, liver, bone marrow, colon, breast, prostate, thyroid, gall bladder, lung, adrenals, muscle, nerve fibers, pancreas, skin, or other life-sustaining organ or tissue in the human body. The term “significant side effects”, as used herein, refers to an antibody, ligand or antibody conjugate that, when administered in vivo, will produce only negligible or clinically manageable side effects, such as those normally encountered during chemotherapy.

For tumor vasculature targeting, the targeting antibody or ligand will often bind to a marker expressed by, adsorbed to, induced on or otherwise localized to the intratumoral blood vessels of a vascularized tumor. “Components of tumor vasculature” thus include both tumor vasculature endothelial cell surface molecules and any components, such as growth factors, that may be bound to these cell surface receptors or molecules.

The following patents are specifically incorporated herein by reference for the purposes of even further supplementing the present teachings regarding the preparation and use of immunotoxins directed against expressed, adsorbed, induced or localized markers of tumor vasculature: U.S. Pat. Nos. 5,855,866; 5,776,427; 5,863,538; 5,660,827; 5,855,866; 5,877,289; 6,004,554; 5,965,132; 6,036,955; 6,093,399; 6,004,555,

Particular examples of surface-expressed targets of tumor and intratumoral blood vessels include vascular cell surface receptors and cell adhesion molecules, such as those listed in Table 1 of Thorpe and Ran (2000; specifically incorporated herein by reference). All references identified in the last column of Table 1 of Thorpe and Ran (2000) are also specifically incorporated herein by reference for purposes including describing and enabling a range of selective markers of tumor vasculature known to those of ordinary skill in the art. As described in Thorpe and Ran (2000), particular suitable examples include endoglin, targeted by, e g., TEC-4, TEC-11, E-9 and Snef antibodies; E-selectin, targeted by. e.g. H4/18 antibodies; VCAM-1, targeted by, e.g, E1/6 and 1.4c3 antibodies; endosialin, targeted by, e.g. FB5 antibodies; αᵥβ₃ integrin, targeted by, e.g., LM609 and peptide targeting agents; the VEGF receptor VEGFR1, targeted by a number of antibodies, and particularly by VEGF; the VEGF receptor complex, also targeted by a number of antibodies, such as 3E7 and GV39; and PSMA, targeted by antibodies such as J591.

Examples such as endoglin, TGFβ receptors, E-selectin, P-selectin, VCAM-1, ICAM-1, a ligand reactive with LAM-1, a VEGF/VPF receptor, an FGF receptor, αᵥβ₃ integrin, pleiotropin, endosialin are further described and enabled in U.S. Pat. Nos. 5,855,866; 5,877,289; 6,004,555; 6,093,399; Burrows et al., 1992; Burrows and Thorpe, 1993; Huang et al., 1997; Liu et al., 1997; Ohizumi et al., 1997; each incorporated herein by reference.

As described in Thorpe and Ran (2000), further particular suitable examples include proteoglycans, such as NG2. and matrix metalloproteinases (MMPs), such as MMP2 and MMP9, each targeted by particular peptide targeting agents. These are examples of remodeling enzymes that are expressed as targetable entities in the tumor, which is a site of vascular remodeling. Further suitable targets are thrombomodulin, Thy-1 and cystatin. Studies identifying sequences elevated in tumor endothelium have also identified thrombomodulin, MMP 11 (stromelysin). MMP 2 (gelatinase) and various collagens as targetable tumor vascular markers, which is also in accordance with U.S. Pat. Nos. 6,004,555 and 6,093,399, specifically incorporated herein by reference.

Antibodies and fragments that bind to endoglin are exemplified by antibodies and fragments that bind to the same epitope as the monoclonal antibody TEC-4 or the monoclonal antibody TEC-11 (U.S. Pat. No. 5,660,827). An extensive range of antibodies are available that bind to the VFGF receptor, as exemplified by monoclonal antibodies 3E11, 3E7, 5G6, 4D8, 10B10, TEC-110, 1B4, 4B7, 1B8, 2C9, 7D9, 12D2, 12D7, 12E10, 5E5, 8E5, 5E11, 7E11, 3F5, 10F3, 1F4, 2F8, 2F9, 2F10, 1G6, 1G11, 3G9, 9G11, 10G9, GV97, GV39, GV97γ, GV39γ, GV59, GV14, A4.6.1, A3.13.1, A4.3.1, B2.6.2, SBS94.1, G143-264, G143-856.

One suitable target for clinical applications is vascular endothelial adhesion molecule-1 (VCAM-1) (U.S. Pat. Nos. 5,855,866, 5,877,289, 6,004,555 and 6,093,399: each incorporated herein by reference). VCAM-1 is a cell adhesion molecule that is induced by inflammatory cytokines IL-1α, IL-4 (Thornhill et al., 1990) and TNFα (Munro, 1993) and whose role in vivo is to recruit leukocytes to sites of acute inflammation (Bevilacqua. 1993).

VCAM-1 is present on vascular endothelial cells in a number of human malignant tumors including neuroblastoma (Patey et al., 1996), renal carcinoma (Droz et al., 1994), non-small lung carcinoma (Staal-van den Brekel et al., 1996), Hodgkin's disease (Patey et al., 1996), and angiosarcoma (Kuzu et al., 1993), as well as in benign tumors, such as angioma (Patey et al., 1996) and hemangioma (Kuzu et al., 1993). Constitutive expression of VCAM-1 in man is confined to a few vessels in the thyroid, thymus and kidney (Kuzu et al., 1993; Bruijn and Dinklo, 1993), and in the mouse to vessels in the heart and lung (Fries et al., 1993).

Data from the inventor shows the selective induction of thrombosis and tumor infarction resulting from administration of an anti-VCAM-1●-tTF coaguligand. Using a covalently-linked anti-VCAM-1●-tTF coaguligand, in which tTF was directly linked to the anti-VCAM-1 antibody, it was shown that the coaguligand localizes selectively to tumor vessels, induces thrombosis of those vessels, causes necrosis to develop throughout the tumor and retards tumor growth in mice bearing solid L540 Hodgkin tumors. The thrombin generation caused by the initial administration of the coaguligand likely leads to further VCAM-1 induction on central vessels (Sluiter et al., 1993), resulting in an amplified signal and evident destruction of the intratumoral region. This type of coagulant-induced expression of further targetable markers, and hence signal amplification, is also disclosed in U.S. Pat. No. 6,036,955, incorporated herein by reference.

The failure of anti-VCAM-1 coaguligands to cause thrombosis in vessels of normal tissues, despite localization to vessels in certain normal tissues, shows the safety of anti-vascular strategies even in the absence of totally stringent targeting. Such beneficial safety issues are an important aspect of the present invention as, even with some potential misdirection, the attached coagulants of the presently claimed invention will not exert adverse side-effects in healthy tissues.

Another suitable target listed in Table 1 of Thorpe and Ran (2000) is PSMA (prostate-specific membrane antigen). PSMA, initially defined by monoclonal antibody 7E11, was originally identified as a marker of prostate cancer and is known to be a type 2 integral membrane glycoprotein. The 7E11 antibody binds to an intracellular epitope of PSMA that, in viable cells, is not available for binding. In the context of the present invention, PSMA is thus targeted using antibodies to the extracellular domain. Such antibodies react with tumor vascular endothelium in a variety of carcinomas, including lung, colon and breast, but not with normal vascular endothelium (Liu et al., 1997; Silver et al., 1997).

Many antibodies that bind to the external domain of PSMA are readily available and may be used in the present invention. Monoclonal antibodies 3E11, 3C2, 4E10-1.14, 3C9 and 1G3 display specificities for differing regions of the extracellular domain of the PSMA protein and are suitable for use herein (Murphy et al., 1998, specifically incorporated herein by reference). Chang et al. (1999, specifically incorporated herein by reference) describe three additional antibodies to the extracellular domain of PSMA, J591, J415 and PEQ226.5, which confirm PSMA expression in tumor-associated vasculature and may used in the invention. As the nucleic acids encoding PSMA and variants thereof are also readily available, U.S. Pat. Nos. 5,935,818 and 5,538,866, additional antibodies can be generated if desired.

U.S. Pat. No. 6,150,508, specifically incorporated herein by reference, describes various other monoclonal antibodies that bind to the extracellular domain of PSMA, which may be used in the present invention. Any one or more of the thirty-five exemplary monoclonal antibodies reactive with PSMA expressed on the cell surface may be used. These include, 3F5.4G6 (ATCC HB12060); 3D7-1.I. (ATCC HB12309); 4E10-1.14 (ATCC HB12310); 3E11 (ATCC HB12488); 4D8 (ATCC HB12487); 3E6 (ATCC HB12486); 3C9 (ATCC HB 12484); 2C7 (ATCC HB 12490); 1G3 (ATCC HB 12489); 3C4 (ATCC HB 12494); 3C6 (ATCC HB12491); 4D4 (ATCC HB12493); 1G9 (ATCC HB12495); 5C8B9 (ATCC HB12492); 3G6 (ATCC HB12485); and 4C8B9 (ATCC HB12492).

Further antibodies, or binding portions thereof, that recognize an extracellular domain of PSMA are described in U.S. Pat. Nos. 6,107,090 and 6,136,311. each specifically incorporated herein by reference. Four hybridoma cell lines in particular are described, being E99, J415, J533, and J591 (ATCC HB-12101, HB-12109, HB-12127, and HB-12126), any one or more of which may thus be used as a targeting agent in accordance with the claimed invention.

Targeting agents that bind to “adsorbed” targets are another suitable group, such as those that bind to ligands or growth factors that bind to tumor or intratumoral vasculature cell surface receptors. Such antibodies include those that bind to VEGF, FGF, TGFβ, HGF, PF4, PDGF, TIMP or a tumor-associated fibronectin isoform (U.S. Pat. Nos. 5,877,289; 5,965,132; 6,093,399 and 6,004,555; each incorporated herein by reference).

Other suitable targeting antibodies, or fragments thereof, are those that bind to epitopes that are present on ligand-receptor complexes or growth factor-receptor complexes, but absent from both the individual ligand or growth factor and the receptor. Such antibodies will recognize and bind to a ligand-receptor or growth factor-receptor complex, as presented at the cell surface, but will not bind to the free ligand or growth factor or the uncomplexed receptor. A “bound receptor complex”, as used herein, therefore refers to the resultant complex produced when a ligand or growth factor specifically binds to its receptor, such as a growth factor receptor.

These aspects are exemplified by the VEGF/VEGF receptor complex. Such ligand-receptor complexes will be present in a significantly higher number on tumor-associated endothelial cells than on non-tumor associated endothelial cells, and may thus be targeted by anti-complex antibodies. Anti-complex antibodies include the monoclonal antibodies 2E5, 3E5 and 4E5 and fragments thereof.

Antigens naturally and artificially inducible by cytokines and coagulants may also be targeted. Exemplary cytokine-inducible antigens are E-selectin, VCAM-1, ICAM-1, endoglin, a ligand reactive with LAM-1, and even MHC Class II antigens,, which are induced by, e.g., IL-1, IL-4, TNF-α, TNF-β or IFN-γ, as may be released by monocytes, macrophages, mast cells, helper T cells, CD8-positive T-cells, NK cells or even tumor cells.

Further inducible antigens include those inducible by a coagulant, such as by thrombin, Factor IX/IXa, Factor X/Xa, plasmin or a metalloproteinase (matrix metalloproteinase, MMP). Generally, antigens inducible by thrombin will be used. This group of antigens includes P-selectin, E-selectin, PDGF and ICAM-1. with the induction and targeting of P-selectin and/or E-selectin being generally preferred.

Other targets inducible by the natural tumor environment or following intervention by man are also targetable entities, as described in U.S. Pat. Nos. 5,776,427, 5,863,538, 6,004,554 and 6,036,955. When used in conjunction with prior suppression in normal tissues and tumor vascular induction, MHC Class II antigens may also be employed as targets (U.S. Pat. Nos. 5,776,427; 5,863,538; 6,004,554 and 6,036,955; each incorporated herein by reference). The suppression of MHC Class II in normal tissues may be achieved using a cyclosporin, such as Cyclosporin A (CsA), or a functionally equivalent agent.

In other embodiments, the vasculature and stroma targeting agents (see below) of the invention will be targeting agents that are themselves biological ligands, or portions thereof, rather than an antibodies. “Biological ligands” in this sense will be those molecules that bind to or associate with cell surface molecules, such as receptors, that are accessible in the stroma or on vascular cells; as exemplified by cytokines, hormones, growth factors, and the like. Any such growth factor or ligand may be used so long as it binds to the disease-associated stroma or vasculature, e.g, to a specific biological receptor present on the surface of a tumor vasculature endothelial cell.

Suitable growth factors for use in these aspects of the invention include, for example, VEGF/VPF (vascular endothelial growth factor/vascular permeability factor). FGF (the fibroblast growth factor family of proteins), TGFβ (transforming growth factor B), a tumor-associated fibronectin isoform, scatter factor/hepatocyte growth factor (HGF), platelet factor 4 (PF4), PDGF (platelet derived growth factor), TIMP or even IL-8, IL-6 or Factor XIIIa. VEGF/VPF and FGF will often be preferred.

Targeting an endothelial cell-bound component, e.g., a cytokine or growth factor, with a binding ligand construct based on a known receptor is also contemplated. Generally, where a receptor is used as a targeting component, a truncated or soluble form of the receptor will be employed. In such embodiments, it is particularly preferred that the targeted endothelial cell-bound component be a dimeric ligand, such as VEGF. This is preferred, as one component of the dimer will already be bound to the cell surface receptor in situ, leaving the other component of the dimer available for binding the soluble receptor portion of the bispecific coagulating ligand.

C3. Tumor Stromal Targeting Agents

Further suitable targeting agents are those that bind to stromal components associated with angiogenic diseases, notably components of tumor-associated stroma. During tumor progression, the extracellular matrix of the surrounding tissue is remodeled through two main processes: the proteolytic degradation of extracellular matrix components of normal tissue; and the de novo synthesis of extracellular matrix components by tumor cells and stromal cells activated by tumor-induced cytokines. These two processes generate a “tumor extracellular matrix” or “tumor stroma”, which is permissive for tumor progression and is qualitatively and quantitatively distinct from the extracellular matrices or stroma of normal tissues.

The “tumor stroma” thus has targetable components that are not present in formal tissues. Certain preferred tumor stromal targeting agents for use in the invention are those that bind to basement membrane markers, type IV collagen, laminin, heparan sulfate, proteoglycan. fibronectins, activated platelets, LIBS, RIBS and tenascin. The following patents are specifically incorporated herein by reference for the purposes of even further supplementing the present teachings regarding the preparation and use of tumor stromal targeting agents: U.S. Pat. No. 5,877,289; 6,093,399; 6,004,555; and 6,036,955.

“Components of disease- and tumor-associated stroma” include structural and functional components of the stroma, extracellular matrix and connective tissues. Tumor stroma targeting agents thus include those that bind to components such as basement membrane markers, type IV collagens, laminin, fibrin, heparan sulfate, proteoglycans, glycoproteins, anionic polysaccharides such as heparin and heparin-like compounds and fibronectins.

Exemplary useful antibodies are those that bind to tenascin, a large molecular weight extracellular glycoprotein expressed in the stroma of various benign and malignant tumors. Anti-tenascin antibodies may thus be used as the binding portions of the coaguligands (U.S. Pat. Nos. 6,093,399 and 6,004,555. specifically incorporated herein by reference).

“Components of disease- and tumor-associated stroma” further include components bound within the extracellular matrix or stroma, including various cell types located therein. “Components of disease- and tumor-associated stroma” thus include cells, matrix components, effectors and other molecules that may be considered, by some, to be outside the narrowest definition of “stroma”, but are nevertheless “targetable entities” that are preferentially associated with a disease region, such as a tumor.

Accordingly, the targeting agents of the invention include antibodies and ligands that bind to a smooth muscle cell, a pericyte, a fibroblast, a macrophage, and an infiltrating lymphocyte or leucocyte. “Activated platelets” are further components of tumor stroma, as platelets bind to the stroma when activated, and such platelets may thus be targeted by the invention.

Further suitable stromal targeting agents, antibodies and antigen binding regions thereof bind to “inducible” tumor stroma components, such as those inducible by cytokines, and especially those inducible by coagulants, such as thrombin. A group of preferred anti-stromal antibodies are those that bind to RIBS, the receptor-induced binding site, on fibrinogen. “RIBS” is thus a targetable antigen, the expression of which in stroma is dictated by activated platelets. Antibodies that bind to LIBS, the ligand-induced binding site, on activated platelets are also useful.

Preferred targetable elements of tumor-associated stroma are currently the tumor-associated fibronectin (FN) isoforms. Fibronectins are multifunctional, high molecular weight glycoprotein constituents of both extracellular matrices and body fluids. They are involved in many different biological processes, such as the establishment and maintenance of normal cell morphology, cell migration, haemostasis and thrombosis, wound healing and oncogenic transformation.

Fibronectin isoforms are ligands that bind to the integrin family of receptors. Although the terminology is not particularly important. “tumor-associated fibronectin isoforms” may thus be considered to be part of the tumor vasculature and/or the tumor stroma. Fibronectin isoforms have extensive structural heterogeneity, which is brought about at the transcriptional. post-transcriptional and post-translational levels.

Structural diversity in fibronectins is first brought about by alternative splicing of three regions (ED-A. Ed-B and IIICS) of the primary fibronectin transcript to generate at least 20 different isoforms. As well as being regulated in a tissue- and developmentally-specific manner, it is known that the splicing pattern of fibronectin-pre-mRNA is deregulated in transformed cells and in malignancies. In fact, the fibronectin isoforms containing the ED-A. ED-B and IIICS sequences are expressed to a greater extent in transformed and malignant tumor cells than in normal cells.

In particular, the fibronectin isoform containing the ED-B sequence (B+ isoform), is highly expressed in foetal and tumor tissues as well as during wound healing, but restricted in expression in normal adult tissues. B+ fibronectin molecules are undetectable in mature vessels, but upregulated in angiogenic blood vessels in normal situations (e.g., development of the endometrium), pathological angiogenesis (e.g., in diabetic retinopathy) and in tumor development. The so-called B+ isoform of fibronectin (B-FN) is thus particularly suitable for use with the present invention.

The ED-B sequence is a complete type III-homology repeat encoded by a single exon and comprising 91 amino acids. The presence of B+ isoform itself constitutes a tumor-induced neoantigen, but in addition, ED- expression exposes a normally cryptic antigen within the type III repeat 7 (preceding ED-B); since this epitope is not exposed in fibronectin molecules lacking ED-B, it follows that ED-B expression induces the expression of neoantigens both directly and indirectly. This cryptic antigenic site forms the target of the monoclonal antibody, BC-1 (European Collection of Animal Cell Cultures, Porton Down, Salisbury, UK, number 88042101). The BC1 antibody may be used as a vascular targeting component of the present invention.

Improved antibodies with specificity for the ED-B isoform are described in WO 97/45544. specifically incorporated herein by reference. Such antibodies have been obtained as single chain Fvs (scFvs) from libraries of human antibody variable regions displayed on the surface of filamentous bacteriophage (see also WO 92/01047, WO 92/20791, WO 93/06213, WO 93/11236 and WO 93/19172).

Using an antibody phage library, specific scFvs can be isolated both by direct selection on recombinant fibronectin-fragments containing the ED-B domain and on recombinant ED-B itself when these antigens are coated onto a solid surface (“panning”). These same sources of antigen have also been successfully used to produce “second generation” scFvs with improved properties relative to the parent clones in a process of “affinity maturation”. The isolated scFvs react strongly and specifically with the B+ isoform of human fibronectin, preferably without prior treatment with N-glycanase.

The antibodies of WO 97/45544 are thus particularly contemplated for use herewith. In anti-tumor applications, these human antibody antigen-binding domains are advantageous as they have less side-effects upon human administration. The referenced antibodies bind the ED-B domain directly. Preferably, the antibodies bind both human fibronectin ED-B and a non-human fibronectin ED-B, such as that of a mouse, allowing for testing and analysis in animal models. The antibody fragments extend to single chain Fv (scFv), Fab, Fab′, F(ab′)2. Fabc, Facb and diabodies.

Even further improved antibodies specific for the ED-domain of fibronectin have been produced with sub-nanomolar dissociation constants, as described in WO 99/58570, and are thus even more preferred for use herewith. These targeting agents are exemplified by the L19 antibody, described in WO 99/58570, specifically incorporated herein by reference for the purpose of teaching how to make and use this and related antibodies. These antibodies have specific affinity for a characteristic epitope of the ED-B domain of fibronectin and have improved affinity to the ED-B epitope.

Such improved recombinant antibodies are available in scFv format, from an antibody phage display library. In addition to H10 and L19. the latter of which has a dissociation constant for the ED-B domain of fibronectin in the sub-nanomolar concentration range, the techniques of WO 99/58570. specifically incorporated herein by reference, may be used to prepare like antibodies. The isolation of human scFv antibody fragments specific for the ED-B domain of fibronectin from antibody phase-display libraries and the isolation of a human scfv antibody fragment binding to the ED-B with sub-nanomolar affinity are particularly described in Examples 1 and 2 of WO 99/58570.

Preferred antibodies thus include those with specific affinity for a characteristic epitope of the ED-B domain of fibronectin, wherein the antibody has improved affinity for the ED-B epitope, wherein the affinity is in the subnanomolar range, and wherein the antibody recognizes ED-B(+) fibronectin. Other preferred formats are wherein the antibody is a scFv or recombinant antibody and wherein the affinity is improved by introduction of a limited number of mutations in its CDR residues. Exemplary residues to be mutated include 31-33, 50, 52 and 54 of the VH domain and residues 32 and 50 of its VL domain. Such antibodies are able to bind the ED-B domain of fibronectin with a Kd of 27 to 54 pM; as exemplifed by the L19 antibody or functionally equivalent variants form of L19.

C4. Targeted Coagulants

Aside from the particular tumor-targeting agent employed in the non-sensitizing or treatment aspect of the combined therapy, any one or more of a variety of coagulants may be used in the coaguligands. The targeting antibody or ligand may be directly or indirectly, e.g, via another antibody, linked to any factor that directly or indirectly stimulates coagulation. As used herein, the terms “coagulant” and “coagulation factor” are each used to refer to a component that is capable of directly or indirectly stimulating coagulation under appropriate conditions, preferably when provided to a specific in vivo environment, such as the tumor vasculature.

Preferred coagulation factors are Tissue Factor compositions, such as the truncated, dimeric, multimeric and mutant TF molecules described in detail above in connection with the naked TF combinations, U.S. Pat. No. 5,504,067 is specifically incorporated herein by reference for the purposes of further describing such truncated Tissue Factor proteins. Preferably, the Tissue Factors for use in these aspects of the present invention will generally lack the transmembrane and cytosolic regions (amino acids 220-263) of the protein. However, there is no need for the truncated TF molecules to be limited to molecules of the exact length of 219 amino acids.

Tissue Factor compositions may also be useful as dimers. Any of the truncated. mutated or other Tissue Factor constructs may be prepared in a dimeric form for use in the present invention. As will be known to those of ordinary skill in the art, such TF dimers may be prepared by employing the standard techniques of molecular biology and recombinant expression, in which two coding regions are prepared in-frame and expressed from an expression vector, Equally, various chemical conjugation technologies may be employed in connection with the preparation of TF dimers. The individual TF monomers may be derivatized prior to conjugation. All such techniques would be readily known to those of skill in the art.

If desired, the Tissue Factor dimers or multimers may be joined via a biologically-releasable bond, such as a selectively-cleavable linker or amino acid sequence. For example, peptide linkers that include a cleavage site for an enzyme preferentially located or active within a tumor environment are contemplated. Exemplary forms of such peptide linkers are those that are cleaved by urokinase, plasmin, thrombin, Factor IXa, Factor Xa, or a metalloproteinase, such as collagenase, gelatinase or stromelysin.

In certain embodiments, the Tissue Factor dimers may further comprise a hindered hydrophobic membrane insertion moiety, to later encourage the functional association of the Tissue Factor with the phospholipid membrane, but only under certain defined conditions. As described in the context of the truncated Tissue Factors, hydrophobic membrane-association sequences are generally stretches of amino acids that promote association with the phospholipid environment due to their hydrophobic nature. Equally, fatty acids may be used to provide the potential membrane insertion moiety.

Such membrane insertion sequences may be located either at the N-terminus or the C-terminus of the TF molecule, or generally appended at any other point of the molecule so long as their attachment thereto does not hinder the functional properties of the TF construct. The intent of the hindered insertion moiety is that it remains non-functional until the TF construct localizes within the tumor environment, and allows the hydrophobic appendage to become accessible and even further promote physical association with the membrane. Again, it is contemplated that biologically-releasable bonds and selectively-cleavable sequences will be particularly useful in this regard, with the bond or sequence only being cleaved or otherwise modified upon localization within the tumor environment and exposure to particular enzymes or other bioactive molecules.

In other embodiments, the tTF constructs may be multimeric or polymeric. In this context a “polymeric construct” contains 3 or more Tissue Factor constructs. A “multimeric or polymeric TF construct” is a construct that comprises a first TF molecule or derivative operatively attached to at least a second and a third TF molecule or derivative. The multimers may comprise between about 3 and about 20 such TF molecules. The individual TF units within the multimers or polymers may also be linked by selectively-cleavable peptide linkers or other biological-releasable bonds as desired. Again, as with the TF dimers discussed above, the constructs may be readily made using either recombinant manipulation and expression or using standard synthetic chemistry.

Even further TF constructs useful in combination with the present invention are those mutants deficient in the ability to activate Factor VII. Such “Factor VII activation mutants” are generally defined herein as TF mutants that bind functional Factor VII/VIIa, proteolytically activate Factor X, but are substantially free from the ability to proteolytically activate Factor VII. Accordingly, such constructs are TF mutants that lack Factor VII activation activity.

The ability of such Factor VII activation mutants to function in promoting tumor-specific coagulation is based upon their specific delivery to the tumor vasculature, and the presence of Factor VIIa at low levels in plasma. Upon administration of such a Factor VII activation mutant-targeting agent conjugate, the mutant will be localized within the vasculature of a vascularized tumor. Prior to localization, the TF mutant would be generally unable to promote coagulation in any other body sites, on the basis of its inability to convert Factor VII to Factor VIIa. However, upon localization and accumulation within the tumor region, the mutant will then encounter sufficient Factor VIIa from the plasma in order to initiate the extrinsic coagulation pathway, leading to tumor-specific thrombosis. Exogenous Factor VIIa could also be administered to the patient.

Any one or more of a variety of Factor VII activation mutants may be prepared and used in combination with the present invention. There is a significant amount of scientific knowledge concerning the recognition sites on the TF molecule for Factor VII/VIIa. It will thus be understood that the Factor VII activation region generally lies between about amino acid 157 and about amino acid 167 of the TF molecule. However, it is contemplated that residues outside this region may also prove to be relevant to the Factor VII activating activity, and one may therefore consider introducing mutations into any one or more of the residues generally located between about amino acid 106 and about amino acid 209 of the TF sequence (WO 94/07515; WO 94/28017; each incorporated herein by reference).

A variety of other coagulation factors may be used in combination with the present invention, as exemplified by the agents set forth below. Thrombin, Factor V/Va and derivatives, Factor VIII/VIIIa and derivatives, Factor IX/IXa and derivatives, Factor X/Xa and derivatives, Factor XI/XIa and derivatives, Factor XII/XIIa and derivatives, Factor XIII/XIIIa and derivatives, Factor X activator and Factor V activator may be used in the present invention.

Russell's viper venom Factor X activator is contemplated for combined use with this invention. Monoclonal antibodies specific for the Factor X activator present in Russell's viper venom have also been produced, and could be used to specifically deliver the agent as part of a bispecific binding ligand.

Thromboxane A₂ is formed from endoperoxides by the sequential actions of the enzymes cyclooxygenase and thromboxane synthetase in platelet microsomes. Thromboxane A₂ is readily generated by platelets and is a potent vasoconstrictor, by virtue of its capacity to produce platelet aggregation. Both thromboxane A₂ and active analogues thereof are contemplated for combined use with the present invention.

Thromboxane synthase, and other enzymes that synthesize platelet-activating prostaglandins, may also be used as “coagulants” in the present context. Monoclonal antibodies to, and immunoaffinity purification of, thromboxane synthase are known; as is the cDNA for human thromboxane synthase.

α2-antiplasmin, or α2-plasmin inhibitor, is a proteinase inhibitor naturally present in human plasma that functions to efficiently inhibit the lysis of fibrin clots induced by plasminogen activator. α2-antiplasmin is a particularly potent inhibitor, and is contemplated for combined use with the present invention.

As the cDNA sequence for α2-antiplasmin is available, recombinant expression and/or fusion proteins are preferred. Monoclonal antibodies against α2-antiplasmin are also available that may be used along with this invention. These antibodies could both be used to deliver exogenous α2-antiplasmin to the target site or to garner endogenous α2-antiplasmin and concentrate it within the targeted region.

D. Antibodies

D1. Polyclonal Antibodies

Means for preparing and characterizing antibodies are well known in the art (see, e.g., Antibodies: A Laboratory Manual, Cold Spring Harbor Laboratory, 1988; incorporated herein by reference). To prepare polyclonal antisera an animal is immunized with an immunogenic composition, and antisera collected from that immunized animal. A wide range of animal species can be used for the production of antisera. Typically the animal used for production of anti-antisera is a rabbit, mouse, rat, hamster, guinea pig or goat. Because of the relatively large blood volume of rabbits, a rabbit is a preferred choice for production of polyclonal antibodies.

The amount of immunogen composition used in the production of polyclonal antibodies varies upon the nature of the immunogen as well as the animal used for immunization. A variety of routes can be used to administer an immunogen: subcutaneous, intramuscular, intradermal, intravenous, intraperitoneal and intrasplenic. The production of polyclonal antibodies may be monitored by sampling blood of the immunized animal at various points following immunization. A second, booster injection, may also be given. The process of boosting and titering is repeated until a suitable titer is achieved. When a desired titer level is obtained, the immunized animal can be bled and the serum isolated and stored. The animal can also be used to generate monoclonal antibodies.

As is well known in the art, the immunogenicity of a particular composition can be enhanced by the use of non-specific stimulators of the immune response, known as adjuvants. Exemplary adjuvants include complete Freund's adjuvant, a non-specific stimulator of the immune response containing killed Mycobacterium tuberculosis; incomplete Freund's adjuvant; and aluminum hydroxide adjuvant.

It may also be desired to boost the host immune system, as may be achieved by associating the immunogens with, or coupling to, a carrier. Exemplary carriers are keyhole limpet hemocyanin (KLH) and bovine serum albumin (BSA). Other albumins such as ovalbumin, mouse serum albumin or rabbit serum albumin can also be used as carriers.

D2. Monoclonal Antibodies

Various methods for generating monoclonal antibodies (MAbs) are also now very well known in the art. The most standard monoclonal antibody generation techniques generally begin along the same lines as those for preparing polyclonal antibodies (Antibodies: A Laboratory Manual, Cold Spring Harbor Laboratory, 1988; incorporated herein by reference). A polyclonal antibody response is initiated by immunizing an animal with an immunogenic composition and, when a desired titer level is obtained, the immunized animal can be used to generate MAbs.

MAbs may be readily prepared through use of well-known techniques, which typically involve immunizing a suitable animal with a selected immunogen composition. The immunizing composition is administered in a manner effective to stimulate antibody producing cells. Rodents such as mice and rats are preferred animals, however, the use of rabbit, sheep and frog cells is also possible. The use of rats may provide certain advantages (Goding, 1986, pp. 60-61; incorporated herein by reference), but mice are preferred, with the BALB/c mouse being most preferred as this is most routinely used and generally gives a higher percentage of stable fusions.

Following immunization, somatic cells with the potential for producing antibodies, specifically B lymphocytes (B cells), are selected for use in the MAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, pp. 65-66, 1986; Campbell, pp. 75-83. 1984; each incorporated herein by reference). For example, where the immunized animal is a mouse, one may use P3-X63/Ag8, X63-Ag8.653, NS1/1.Ag 4 1. Sp210-Ag14, FO, NSO/U, MPC-11, MPC11-X45-GTG 1.7 and S194/5XX0 Bul; for rats, one may use R210.RCY3, Y3-Ag 1.2.3, IR983F, 4B210 or one of the above listed mouse cell lines; and U-266, GM1500-GRG2, LICR-LON-HMy2 and UC729-6, are all useful in connection with human cell fusions.

Methods for generating hybrids of antibody-producing spleen or lymph node cells and myeloma cells usually comprise mixing somatic cells with myeloma cells in a 4:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (1975; 1976; each incorporated herein by reference), and those using polyethylene glycol (PEG), such as 37% (v/v) PEG, by Gefter et al. (1977; incorporated herein by reference). The use of electrically induced fusion methods is also appropriate (Goding pp. 71-74. 1986; incorporated herein by reference).

Fusion procedures usually produce viable hybrids at low frequencies, about 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide MAbs. The cell lines may be exploited for MAb production in two basic ways. A sample of the hybridoma can be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they can be readily obtained in high concentrations.

MAbs produced by either means will generally be further purified, e.g, using filtration. centrifugation and various chromatographic methods, such as HPLC or affinity chromatography, all of which purification techniques are well known to those of skill in the art. These purification techniques each involve fractionation to separate the desired antibody from other components of a mixture. Analytical methods particularly suited to the preparation of antibodies include, for example, protein A-Sepharose and/or protein G-Sepharose chromatography.

D3. Antibodies from Phagemid Libraries

Recombinant technology now allows the preparation of antibodies having the desired specificity from recombinant genes encoding a range of antibodies (Van Dijk et al., 1989; incorporated herein by reference). Certain recombinant techniques involve the isolation of the antibody genes by immunological screening of combinatorial immunoglobulin phage expression libraries prepared from RNA isolated from the spleen of an immunized animal (Morrison et al., 1986; Winter and Milstein, 1991; each incorporated herein by reference).

For such methods, combinatorial immunoglobulin phagemid libraries are prepared from RNA isolated from the spleen of the immunized animal, and phagemids expressing appropriate antibodies are selected by panning using cells expressing the antigen and control cells. The advantages of this approach over conventional hybridoma techniques are that approximately 10⁴ times as many antibodies can be produced and screened in a single round, and that new specificities are generated by H and L chain combination, which further increases the percentage of appropriate antibodies generated.

One method for the generation of a large repertoire of diverse antibody molecules in bacteria utilizes the bacteriophage lambda as the vector (Huse et al., 1989; incorporated herein by reference). Production of antibodies using the lambda vector involves the cloning of heavy and light chain populations of DNA sequences into separate starting vectors. The vectors are subsequently combined randomly to form a single vector that directs the co-expression of heavy and light chains to form antibody fragments. The heavy and light chain DNA sequences are obtained by amplification, preferably by PCR™ or a related amplification technique, of mRNA isolated from spleen cells (or hybridomas thereof) from an animal that has been immunized with a selected antigen. The heavy and light chain sequences are typically amplified using primers that incorporate restriction sites into the ends of the amplified DNA segment to facilitate cloning of the heavy and light chain segments into the starting vectors.

Another method for the generation and screening of large libraries of wholly or partially synthetic antibody combining sites, or paratopes, utilizes display vectors derived from filamentous phage such as M13, fl or fd. These filamentous phage display vectors, referred to as “phagemids”, yield large libraries of monoclonal antibodies having diverse and novel immunospecificities. The technology uses a filamentous phage coat protein membrane anchor domain as a means for linking gene-product and gene during the assembly stage of filamentous phage replication, and has been used for the cloning and expression of antibodies from combinatorial libraries (Kang et al., 1991; Barbas et al., 1991; each incorporated herein by reference).

This general technique for filamentous phage display is described in U.S. Pat. No. 5,658,727, incorporated herein by reference. In a most general sense, the method provides a system for the simultaneous cloning and screening of pre-selected ligand-binding specificities from antibody gene repertoires using a single vector system. Screening of isolated members of the library for a pre-selected ligand-binding capacity allows the correlation of the binding capacity of an expressed antibody molecule with a convenient means to isolate the gene that encodes the member from the library.

Linkage of expression and screening is accomplished by the combination of targeting of a fusion polypeptide into the periplasm of a bacterial cell to allow assembly of a functional antibody, and the targeting of a fusion polypeptide onto the coat of a filamentous phage particle during phage assembly to allow for convenient screening of the library member of interest. Periplasmic targeting is provided by the presence of a secretion signal domain in a fusion polypeptide. Targeting to a phage particle is provided by the presence of a filamentous phage coat protein membrane anchor domain (i.e., a cpIII- or cpVIII-derived membrane anchor domain) in a fusion polypeptide.

The diversity of a filamentous phage-based combinatorial antibody library can be increased by shuffling of the heavy and light chain genes, by altering one or more of the complementarity determining regions of the cloned heavy chain genes of the library, or by introducing random mutations into the library by error-prone polymerase chain reactions. Additional methods for screening phagemid libraries are described in U.S. Pat. Nos. 5,580,717; 5,427,908; 5,403,484; and 5,223,409, each incorporated herein by reference.

Another method for the screening of large combinatorial antibody libraries has been developed, utilizing expression of populations of diverse heavy and light chain sequences on the surface of a filamentous bacteriophage, such as M13, fl or fd (U.S. Pat. No. 5,698,426; incorporated herein by reference). Two populations of diverse heavy (Hc) and light (Lc) chain sequences are synthesized by polymerase chain reaction (PCR™). These populations are cloned into separate M13-based vector containing elements necessary for expression. The heavy chain vector contains a gene VIII (gVIII) coat protein sequence so that translation of the heavy chain sequences produces gVIII-Hc fusion proteins. The populations of two vectors are randomly combined such that only the vector portions containing the He and Lc sequences are joined into a single circular vector.

The combined vector directs the co-expression of both He and Lc sequences for assembly of the two polypeptides and surface expression on M13 (U.S. Pat. No.5,698,426; incorporated herein by reference). The combining step randomly brings together different He and Lc encoding sequences within two diverse populations into a single vector. The vector sequences donated from each independent vector are necessary for production of viable phage. Also, since the pseudo gVIII sequences are contained in only one of the two starting vectors, co-expression of functional antibody fragments as Lc associated gVIII-Hc fusion proteins cannot be accomplished on the phage surface until the vector sequences are linked in the single vector.

Surface expression of the antibody library is performed in an amber suppressor strain. An amber stop codon between the He sequence and the gVIII sequence unlinks the two components in a non-suppressor strain. Isolating the phage produced from the non-suppressor strain and infecting a suppressor strain will link the He sequences to the gVIII sequence during expression. Culturing the suppressor strain after infection allows the coexpression on the surface of M13 of all antibody species within the library as gVIIl fusion proteins (gVIII-Fab fusion proteins). Alternatively, the DNA can be isolated from the non-suppressor strain and then introduced into a suppressor strain to accomplish the same effect.

The surface expression library is screened for specific Fab fragments that bind preselected molecules by standard affinity isolation procedures. Such methods include, for example, panning (Parmley and Smith, 1988; incorporated herein by reference), affinity chromatography and solid phase blotting procedures, Panning is preferred, because high titers of phage can be screened easily, quickly and in small volumes. Furthermore, this procedure can select minor Fab fragments species within the population, which otherwise would have been undetectable, and amplified to substantially homogenous populations. The selected Fab fragments can be characterized by sequencing the nucleic acids encoding the polypeptides after amplification of the phage population.

Another method for producing diverse libraries of antibodies and screening for desirable binding specificities is described in U.S. Pat. Nos. 5,667,988 and 5,759,817, each incorporated herein by reference. The method involves the preparation of libraries of heterodimeric immunoglobulin molecules in the form of phagemid libraries using degenerate oligonucleotides and primer extension reactions to incorporate the degeneracies into the CDR regions of the immunoglobulin variable heavy and light chain variable domains, and display of the mutagenized polypeptides on the surface of the phagemid. Thereafter, the display protein is screened for the ability to bind to a preselected antigen.

The method for producing a heterodimeric immunoglobulin molecule generally involves (1) introducing a heavy or light chain V region-coding gene of interest into the phagemid display vector; (2) introducing a randomized binding site into the phagemid display protein vector by primer extension with an oligonucleotide containing regions of homology to a CDR of the antibody V region gene and containing regions of degeneracy for producing randomized coding sequences to form a large population of display vectors each capable of expressing different putative binding sites displayed on a phagemid surface display protein; (3) expressing the display protein and binding site on the surface of a filamentous phage particle; and (4) isolating (screening) the surface-expressed phage particle using affinity techniques such as panning of phage particles against a preselected antigen, thereby isolating one or more species of phagemid containing a display protein containing a binding site that binds a preselected antigen.

A further variation of this method for producing diverse libraries of antibodies and screening for desirable binding specificities is described in U.S. Pat. No. 5,702,892, incorporated herein by reference. In this method, only heavy chain sequences are employed, the heavy chain sequences are randomized at all nucleotide positions which encode either the CDRI or CDRIII hypervariable region, and the genetic variability in the CDRs is generated independent of any biological process.

In the method, two libraries are engineered to genetically shuffle oligonucleotide motifs within the framework of the heavy chain gene structure. Through random mutation of either CDRI or CDRIII, the hypervariable regions of the heavy chain gene were reconstructed to result in a collection of highly diverse sequences. The heavy chain proteins encoded by the collection of mutated gene sequences possessed the potential to have all of the binding characteristics of an immunoglobulin while requiring only one of the two immunoglobulin chains.

Specifically, the method is practiced in the absence of the immunoglobulin light chain protein. A library of phage displaying modified heavy chain proteins is incubated with an immobilized ligand to select clones encoding recombinant proteins that specifically bind the immobilized ligand. The bound phage are then dissociated from the immobilized ligand and amplified by growth in bacterial host cells. Individual viral plaques, each expressing a different recombinant protein, are expanded, and individual clones can then be assayed for binding activity.

D4. Antibodies from Human Patients

Antibodies against tumor components occur in the human population. These antibodies would thus be appropriate as starting materials for generating an antibody for use in the coaguligand combination aspects of the present invention.

To prepare an antibody from a human patient, one would simply obtain human lymphocytes from an individual having anti-tumor antibodies, for example from human peripheral blood, spleen, lymph nodes, tonsils or the like, utilizing techniques that are well known to those of skill in the art. The use of peripheral blood lymphocytes will often be preferred.

Human monoclonal antibodies may be obtained from the human lymphocytes producing the desired anti-tumor antibodies by immortalizing the human lymphocytes, generally in the same manner as described above for generating any monoclonal antibody. The reactivities of the antibodies in the culture supernatants are generally first checked, employing one or more selected tumor antigen(s), and the lymphocytes that exhibit high reactivity are grown. The resulting lymphocytes are then fused with a parent line of human or mouse origin, and further selection gives the optimal clones.

The recovery of monoclonal antibodies from the immortalized cells may be achieved by any method generally employed in the production of monoclonal antibodies. For instance, the desired monoclonal antibody may be obtained by cloning the immortalized lymphocyte by the limiting dilution method or the like, selecting the cell producing the desired antibody, growing the selected cells in a medium or the abdominal cavity of an animal, and recovering the desired monoclonal antibody from the culture supernatant or ascites.

Such techniques have been used, for example, to isolate human monoclonal antibodies to Pseudomonas aeruginosa epitopes (U.S. Pat. Nos. 5,196,337 and 5,252,480, each incorporated herein by reference); polyribosylribitol phosphate capsular polysaccharides (U.S. Pat. No. 4,954,449, incorporated herein by reference); the Rh(D) antigen (U.S. Patent No. 5,665,356, incorporated herein by reference); and viruses, such as human immunodeficiency virus, respiratory syncytial virus, herpes simplex virus, varicella zoster virus and cytomegalovirus (U.S. Pat. Nos. 5,652,138; 5,762,905; and 4,950,595, each incorporated herein by reference). The applicability of the foregoing techniques to the generation of human anti-tumor antibodies is thus clear.

Additionally, the methods described in U.S. Pat. No. 5,648,077 (incorporated herein by reference) can be used to form a trioma or a quadroma that produces a human antibody against a selected tumor antigen. In a general sense, a hybridoma cell line comprising a parent rodent immortalizing cell, such as a murine myeloma cell, e.g, SP-2, is fused to a human partner cell, resulting in an immortalizing xenogeneic hybridoma cell. This xenogeneic hybridoma cell is fused to a cell capable of producing an anti-tumor human antibody, resulting in a trioma cell line capable of generating human antibody effective against such antigen in a human. Alternately, when greater stability is desired, a trioma cell line that preferably no longer has the capability of producing its own antibody is made, and this trioma is then fused with a further cell capable of producing an antibody useful against the tumor antigen to obtain a still more stable hybridoma (quadroma) that produces antibody against the antigen.

D5. Antibodies from Human Lymphocytes

In vitro immunization, or antigen stimulation, may also be used to generate a human anti-tumor antibody. Such techniques can be used to stimulate peripheral blood lymphocytes from both anti-tumor antibody-producing human patients, and also from normal, healthy subjects. Anti-tumor antibodies can be prepared from healthy human subjects simply by stimulating antibody-producing cells in vitro.

Such “in vitro immunization” involves antigen-specific activation of non-immunized B lymphocytes, generally within a mixed population of lymphocytes (mixed lymphocyte cultures, MLC). In vitro immunizations may also be supported by B cell growth and differentiation factors and lymphokines. The antibodies produced by these methods are often IgM antibodies.

Another method has been described (U.S. Pat. No. 5,681,729, incorporated herein by reference) wherein human lymphocytes that mainly produce IgG (or IgA) antibodies can be obtained. The method involves, in a general sense, transplanting human lymphocytes to an immunodeficient animal so that the human lymphocytes “take” in the animal body; immunizing the animal with a desired antigen, so as to generate human lymphocytes producing an antibody specific to the antigen; and recovering the human lymphocytes producing the antibody from the animal. The human lymphocytes thus produced can be used to produce a monoclonal antibody by immortalizing the human lymphocytes producing the antibody, cloning the obtained immortalized human-originated lymphocytes producing the antibody, and recovering a monoclonal antibody specific to the desired antigen from the cloned immortalized human-originated lymphocytes.

The immunodeficient animals that may be employed in this technique are those that do not exhibit rejection when human lymphocytes are transplanted to the animals. Such animals may be artificially prepared by physical, chemical or biological treatments. Any immunodeficient animal may be employed. The human lymphocytes may be obtained from human peripheral blood, spleen, lymph nodes, tonsils or the like.

The “taking” of the transplanted human lymphocytes in the animals can be attained by merely administering the human lymphocytes to the animals. The administration route is not restricted and may be, for example, subcutaneous, intravenous or intraperitoneal. The dose of the human lymphocytes is not restricted, and can usually be 10⁶ to 10⁸ lymphocytes per animal. The immunodeficient animal is then immunized with the desired tumor antigen.

After the immunization, human lymphocytes are recovered from the blood, spleen, lymph nodes or other lymphatic tissues by any conventional method. For example, mononuclear cells can be separated by the Ficoll-Hypaque (specific gravity: 1.077) centrifugation method, and the monocytes removed by the plastic dish adsorption method. The contaminating cells originating from the immunodeficient animal may be removed by using an antiserum specific to the animal cells. The antiserum may be obtained by, for example, immunizing a second, distinct animal with the spleen cells of the immunodeficient animal, and recovering serum from the distinct immunized animal. The treatment with the antiserum may be carried out at any stage. The human lymphocytes may also be recovered by an immunological method employing a human immunoglobulin expressed on the cell surface as a marker.

By these methods, human lymphocytes mainly producing IgG and IgA antibodies specific to one or more selected tumor antigens can be obtained. Monoclonal antibodies are then obtained from the human lymphocytes by immortalization, selection, cell growth and antibody production.

D6. Transgenic Mice Containing Human Antibody Libraries

Recombinant technology is now available for the preparation of antibodies. In addition to the combinatorial immunoglobulin phage expression libraries disclosed above, another molecular cloning approach is to prepare antibodies from transgenic mice containing human antibody libraries. Such techniques are described in U.S. Pat. No. 5,545.807, incorporated herein by reference.

In a most general sense, these methods involve the production of a transgenic animal that has inserted into its germline genetic material that encodes for at least part of an immunoglobulin of human origin or that can rearrange to encode a repertoire of immunoglobulins. The inserted genetic material may be produced from a human source, or may be produced synthetically. The material may code for at least part of a known immunoglobulin or may be modified to code for at least part of an altered immunoglobulin.

The inserted genetic material is expressed in the transgenic animal, resulting in production of an immunoglobulin derived at least in part from the inserted human immunoglobulin genetic material. It is found the genetic material is rearranged in the transgenic animal, so that a repertoire of immunoglobulins with part or parts derived from inserted genetic material may be produced, even if the inserted genetic material is incorporated in the germline in the wrong position or with the wrong geometry.

The inserted genetic material may be in the form of DNA cloned into prokaryotic vectors such as plasmids and/or cosmids. Larger DNA fragments are inserted using yeast artificial chromosome vectors (Burke et al., 1987; incorporated herein by reference), or by introduction of chromosome fragments (Richer and Lo, 1989; incorporated herein by reference). The inserted genetic material may be introduced to the host in conventional manner, for example by injection or other procedures into fertilized eggs or embryonic stem cells.

In preferred aspects, a host animal that initially does not carry genetic material encoding immunoglobulin constant regions is utilized, so that the resulting transgenic animal will use only the inserted human genetic material when producing immunoglobulins. This can be achieved either by using a naturally occurring mutant host lacking the relevant genetic material, or by artificially making mutants e.g., in cell lines ultimately to create a host from which the relevant genetic material has been removed.

Where the host animal carries genetic material encoding immunoglobulin constant regions, the transgenic animal will carry the naturally occurring genetic material and the inserted genetic material and will produce immunoglobulins derived from the naturally occurring genetic material, the inserted genetic material, and mixtures of both types of genetic material. In this case the desired immunoglobulin can be obtained by screening hybridomas derived from the transgenic animal, e.g., by exploiting the phenomenon of allelic exclusion of antibody gene expression or differential chromosome loss.

Once a suitable transgenic animal has been prepared, the animal is simply immunized with the desired immunogen. Depending on the nature of the inserted material the animal may produce a chimeric immunoglobulin, e.g, of mixed mouse/human origin, where the genetic material of foreign origin encodes only part of the immunoglobulin; or the animal may produce an entirely foreign immunoglobulin, e.g. of wholly human origin, where the genetic material of foreign origin encodes an entire immunoglobulin.

Polyclonal antisera may be produced from the transgenic animal following immunization. Immunoglobulin-producing cells may be removed from the animal to produce the immunoglobulin of interest. Preferably, monoclonal antibodies are produced from the transgenic animal, e.g., by fusing spleen cells from the animal with myeloma cells and screening the resulting hybridomas to select those producing the desired antibody. Suitable techniques for such processes are described herein.

In an alternative approach, the genetic material may be incorporated in the animal in such a way that the desired antibody is produced in body fluids such as serum or external secretions of the animal, such as milk, colostrum or saliva. For example, by inserting in vitro genetic material encoding for at least part of a human immunoglobulin into a gene of a mammal coding for a milk protein and then introducing the gene to a fertilized egg of the mammal, e.g., by injection, the egg may develop into an adult female mammal producing milk containing immunoglobulin derived at least in part from the inserted human immunoglobulin genetic material. The desired antibody can then be harvested from the milk. Suitable techniques for carrying out such processes are known to those skilled in the art.

The foregoing transgenic animals are usually employed to produce human antibodies of a single isotype, more specifically an isotype that is essential for B cell maturation, such as IgM and possibly IgD. Another preferred method for producing human anti-tumor antibodies is to use the technology described in U.S. Pat. Nos. 5,545,806; 5,569,825; 5,625,126; 5,633,425; 5,661,016; and 5,770,429; each incorporated by reference, wherein transgenic animals are described that are capable of switching from an isotype needed for B cell development to other isotypes.

In the development of a B lymphocyte, the cell initially produces IgM with a binding specificity determined by the productively rearranged V_{H} and V_{L} regions. Subsequently, each B cell and its progeny cells synthesize antibodies with the same L and H chain V regions, but they may switch the isotype of the H chain. The use of mu or delta constant regions is largely determined by alternate splicing, permitting IgM and IgD to be coexpressed in a single cell. The other heavy chain isotypes (gamma, alpha, and epsilon) are only expressed natively after a gene rearrangement event deletes the C mu and C delta exons. This gene rearrangement process, termed isotype switching, typically occurs by recombination between so called switch segments located immediately upstream of each heavy chain gene (except delta). The individual switch segments are between 2 and 10 kb in length, and consist primarily of short repeated sequences.

For these reasons, it is preferable that transgenes incorporate transcriptional regulatory sequences within about 1-2 kb upstream of each switch region that is to be utilized for isotype switching. These transcriptional regulatory sequences preferably include a promoter and an enhancer element, and more preferably include the 5′ flanking (i.e., upstream) region that is naturally associated (i.e., occurs in germline configuration) with a switch region. Although a 5′ flanking sequence from one switch region can be operably linked to a different switch region for transgene construction, in some embodiments it is preferred that each switch region incorporated in the transgene construct have the 5′ flanking region that occurs immediately upstream in the naturally occurring germline configuration. Sequence information relating to immunoglobulin switch region sequences is known (Mills et al., 1990; Sideras et al., 1989; each incorporated herein by reference).

In the method described in U.S. Pat. Nos. 5,545,806; 5,569,825; 5,625,126; 5,633,425; 5,661,016; and 5,770,429, the human immunoglobulin transgenes contained within the transgenic animal function correctly throughout the pathway of B-cell development. leading to isotype switching. Accordingly, in this method, these transgenes are constructed so as to produce isotype switching and one or more of the following: (1) high level and cell-type specific expression. (2) functional gene rearrangement, (3) activation of and response to allelic exclusion, (4) expression of a sufficient primary repertoire, (5) signal transduction, (6) somatic hypermutation, and (7) domination of the transgene antibody locus during the immune response.

An important requirement for transgene function is the generation of a primary antibody repertoire that is diverse enough to trigger a secondary immune response for a wide range of antigens. The rearranged heavy chain gene consists of a signal peptide exon, a variable region exon and a tandem array of multi-domain constant region regions, each of which is encoded by several exons. Each of the constant region genes encode the constant portion of a different class of immunoglobulins. During B-cell development, V region proximal constant regions are deleted leading to the expression of new heavy chain classes. For each heavy chain class, alternative patterns of RNA splicing give rise to both transmembrane and secreted immunoglobulins.

The human heavy chain locus consists of approximately 200 V gene segments spanning 2 Mb, approximately 30 D gene segments spanning about 40 kb, six J segments clustered within a 3 kb span, and nine constant region gene segments spread out over approximately 300 kb. The entire locus spans approximately 2.5 Mb of the distal portion of the long arm of chromosome 14. Heavy chain transgene fragments containing members of all six of the known V_{H} families, the D and J gene segments, as well as the mu, delta, gamma 3, gamma 1 and alpha 1 constant regions are known (Berman et al., 1988; incorporated herein by reference). Genomic fragments containing all of the necessary gene segments and regulatory sequences from a human light chain locus is similarly constructed.

The expression of successfully rearranged immunoglobulin heavy and light transgenes usually has a dominant effect by suppressing the rearrangement of the endogenous immunoglobulin genes in the transgenic nonhuman animal. However, in certain embodiments, it is desirable to effect complete inactivation of the endogenous Ig loci so that hybrid immunoglobulin chains comprising a human variable region and a non-human (e.g., murine) constant region cannot be formed, for example by trans-switching between the transgene and endogenous Ig sequences. Using embryonic stem cell technology and homologous recombination, the endogenous immunoglobulin repertoire can be readily eliminated. In addition, suppression of endogenous Ig genes may be accomplished using a variety of techniques, such as antisense technology.

In other aspects of the invention, it may be desirable to produce a trans-switched immunoglobulin. Antibodies comprising such chimeric trans-switched immunoglobulins can be used for a variety of applications where it is desirable to have a non-human (e.g., murine) constant region, e.g., for retention of effector functions in the host. The presence of a murine constant region can afford advantages over a human constant region, for example, to provide murine effector functions (e.g., ADCC, murine complement fixation) so that such a chimeric antibody may be tested in a mouse disease model. Subsequent to the animal testing, the human variable region encoding sequence may be isolated, e.g., by PCR™ amplification or cDNA cloning from the source (hybridoma clone), and spliced to a sequence encoding a desired human constant region to encode a human sequence antibody more suitable for human therapeutic use.

D7. Humanized Antibodies

Human antibodies generally have at least three potential advantages for use in human therapy. First, because the effector portion is human, it may interact better with the other parts of the human immune system, e.g., to destroy target cells more efficiently by complement-dependent cytotoxicity (CDC) or antibody-dependent cellular cytotoxicity (ADCC). Second, the human immune system should not recognize the antibody as foreign. Third, the half-life in the human circulation will be similar to naturally occurring human antibodies, allowing smaller and less frequent doses to be given.

Various methods for preparing human anti-tumor antibodies are provided herein. In addition to human antibodies. “humanized” antibodies have many advantages. “Humanized” antibodies are generally chimeric or mutant monoclonal antibodies from mouse, rat, hamster, rabbit or other species, bearing human constant and/or variable region domains or specific changes. Techniques for generating a so-called “humanized” anti-tumor antibodies are well known to those of skill in the art.

Humanized antibodies also share the foregoing advantages. First, the effector portion is still human. Second, the human immune system should not recognize the framework or constant region as foreign, and therefore the antibody response against such an injected antibody should be less than against a totally foreign mouse antibody. Third, injected humanized antibodies, as opposed to injected mouse antibodies, will presumably have a half-life more similar to naturally occurring human antibodies, also allowing smaller and less frequent doses.

A number of methods have been described to produce humanized antibodies. Controlled rearrangement of antibody domains joined through protein disulfide bonds to form new, artificial protein molecules or “chimeric” antibodies can be utilized (Konieczny et al., 1981; incorporated herein by reference). Recombinant DNA technology can also be used to construct gene fusions between DNA sequences encoding mouse antibody variable light and heavy chain domains and human antibody light and heavy chain constant domains (Morrison et al., 1984; incorporated herein by reference).

DNA sequences encoding the antigen binding portions or complementarity determining regions (CDR's) of murine monoclonal antibodies can be grafted by molecular means into the DNA sequences encoding the frameworks of human antibody heavy and light chains (Jones et al., 1986; Riechmann et al., 1988; each incorporated herein by reference). The expressed recombinant products are called “reshaped” or humanized antibodies, and comprise the framework of a human antibody light or heavy chain and the antigen recognition portions, CDR's, of a murine monoclonal antibody.

Another method for producing humanized antibodies is described in U.S. Pat. No. 5,639,641, incorporated herein by reference. The method provides, via resurfacing, humanized rodent antibodies that have improved therapeutic efficacy due to the presentation of a human surface in the variable region. In the method: (1) position alignments of a pool of antibody heavy and light chain variable regions is generated to give a set of heavy and light chain variable region framework surface exposed positions, wherein the alignment positions for all variable regions are at least about 98% identical; (2) a set of heavy and light chain variable region framework surface exposed amino acid residues is defined for a rodent antibody (or fragment thereof); (3) a set of heavy and light chain variable region framework surface exposed amino acid residues that is most closely identical to the set of rodent surface exposed amino acid residues is identified; (4) the set of heavy and light chain variable region framework surface exposed amino acid residues defined in step (2) is substituted with the set of heavy and light chain variable region framework surface exposed amino acid residues identified in step (3), except for those amino acid residues that are within 5 Å of any atom of any residue of the complementarity determining regions of the rodent antibody; and (5) the humanized rodent antibody having binding specificity is produced.

A similar method for the production of humanized antibodies is described in U.S. Pat. Nos. 5,693,762; 5,693,761; 5,585,089; and 5,530,101, each incorporated herein by reference. These methods involve producing humanized immunoglobulins having one or more complementarity determining regions (CDR's) and possible additional amino acids from a donor immunoglobulin and a framework region from an accepting human immunoglobulin. Each humanized immunoglobulin chain usually comprises, in addition to the CDR's, amino acids from the donor immunoglobulin framework that are capable of interacting with the CDR's to effect binding affinity, such as one or more amino acids that are immediately adjacent to a CDR in the donor immunoglobulin or those within about 3 Å as predicted by molecular modeling. The heavy and light chains may each be designed by using any one, any combination, or all of the various position criteria described in U.S. Pat. Nos. 5,693,762; 5,693,761; 5,585,089; and 5,530,101, each incorporated herein by reference. When combined into an intact antibody, the humanized immunoglobulins are substantially non-immunogenic in humans and retain substantially the same affinity as the donor immunoglobulin to the original antigen.

An additional method for producing humanized antibodies is described in U.S. Pat. Nos. 5,565,332 and 5,733,743, each incorporated herein by reference. This method combines the concept of humanizing antibodies with the phagemid libraries also described in detail herein. In a general sense, the method utilizes sequences from the antigen binding site of an antibody or population of antibodies directed against an antigen of interest. Thus for a single rodent antibody, sequences comprising part of the antigen binding site of the antibody may be combined with diverse repertoires of sequences of human antibodies that can, in combination, create a complete antigen binding site.

The antigen binding sites created by this process differ from those created by CDR grafting, in that only the portion of sequence of the original rodent antibody is likely to make contacts with antigen in a similar manner. The selected human sequences are likely to differ in sequence and make alternative contacts with the antigen from those of the original binding site. However, the constraints imposed by binding of the portion of original sequence to antigen and the shapes of the antigen and its antigen binding sites, are likely to drive the new contacts of the human sequences to the same region or epitope of the antigen. This process has therefore been termed “epitope imprinted selection” (EIS).

Starting with an animal antibody, one process results in the selection of antibodies that are partly human antibodies. Such antibodies may be sufficiently similar in sequence to human antibodies to be used directly in therapy or after alteration of a few key residues. Sequence differences between the rodent component of the selected antibody with human sequences could be minimized by replacing those residues that differ with the residues of human sequences, for example, by site directed mutagenesis of individual residues, or by CDR grafting of entire loops. However, antibodies with entirely human sequences can also be created. EIS therefore offers a method for making partly human or entirely human antibodies that bind to the same epitope as animal or partly human antibodies respectively. In EIS, repertoires of antibody fragments can be displayed on the surface of filamentous phase and the genes encoding fragments with antigen binding activities selected by binding of the phage to antigen.

Additional methods for humanizing antibodies contemplated for use in the present invention are described in U.S. Pat. Nos. 5,750,078; 5,502,167; 5,705,154; 5,770,403; 5,698,417; 5,693,493; 5,558,864; 4,935,496; and 4,816,567, each incorporated herein by reference.

D8. Antibody Fragments and Derivatives

Irrespective of the source of the original anti-tumor antibody, either the intact antibody. antibody multimers, or any one of a variety of functional, antigen-binding regions of the antibody may be used in the present invention. Exemplary functional regions include scFv, Fv, Fab′, Fab and F(ab′)₂ fragments of the anti-tumor antibodies. Techniques for preparing such constructs are well known to those in the art and are further exemplified herein.

The choice of antibody construct may be influenced by various factors. For example. prolonged half-life can result from the active readsorption of intact antibodies within the kidney, a property of the Fc piece of immunoglobulin, IgG based antibodies, therefore, are expected to exhibit slower blood clearance than their Fab′ counterparts. However, Fab′ fragment-based compositions will generally exhibit better tissue penetrating capability.

Antibody fragments can be obtained by proteolysis of the whole immunoglobulin by the non-specific thiol protease, papain. Papain digestion yields two identical antigen-binding fragments, termed “Fab fragments”, each with a single antigen-binding site, and a residual “Fc fragment”.

Papain should first be activated by reducing the sulphydryl group in the active site with cysteine, 2-mercaptoethanol or dithiothreitol. Heavy metals in the stock enzyme should be removed by chelation with EDTA (2 mM) to ensure maximum enzyme activity. Enzyme and substrate are normally mixed together in the ratio of 1:100 by weight. After incubation, the reaction can be stopped by irreversible alkylation of the thiol group with iodoacetamide or simply by dialysis. The completeness of the digestion should be monitored by SDS-PAGE and the various fractions separated by protein A-Sepharose or ion exchange chromatography.

The usual procedure for preparation of F(ab′)₂ fragments from IgG of rabbit and human origin is limited proteolysis by the enzyme pepsin. The conditions, 100× antibody excess w/w in acetate buffer at pH 4.5, 37° C. suggest that antibody is cleaved at the C-terminal side of the inter-heavy-chain disulfide bond. Rates of digestion of mouse IgG may vary with subclass and it may be difficult to obtain high yields of active F(ab′)₂ fragments without some undigested or completely degraded IgG. In particular. IgG2b is highly susceptible to complete degradation. The other subclasses require different incubation conditions to produce optimal results, all of which is known in the art.

Pepsin treatment of intact antibodies yields an F(ab′)₂ fragment that has two antigen-combining sites and is still capable of cross-linking antigen. Digestion of rat IgG by pepsin requires conditions including dialysis in 0.1 M acetate buffer, pH 4.5, and then incubation for four hours with 1% w/w pepsin, IgG₁ and IgG₂ₐ digestion is improved if first dialyzed against 0.1 M formate buffer. pH 2.8, at 4° C., for 16 hours followed by acetate buffer. IgG_{2b} gives more consistent results with incubation in staphylococcal V8 protease (3% w/w) in 0.1 M sodium phosphate buffer, pH 7.8, for four hours at 37° C.

An Fab fragment also contains the constant domain of the light chain and the first constant domain (CH1) of the heavy chain, Fab′ fragments differ from Fab fragments by the addition of a few residues at the carboxyl terminus of the heavy chain CH1 domain including one or more cysteine(s) from the antibody hinge region, F(ab′)₂ antibody fragments were originally produced as pairs of Fab′ fragments that have hinge cysteines between them. Other chemical couplings of antibody fragments are also known.

The term “variable”, as used herein in reference to antibodies, means that certain portions of the variable domains differ extensively in sequence among antibodies, and are used in the binding and specificity of each particular antibody to its particular antigen. However, the variability is not evenly distributed throughout the variable domains of antibodies. It is concentrated in three segments termed “hypervariable regions”, both in the light chain and the heavy chain variable domains.

The more highly conserved portions of variable domains are called the framework region (FR). The variable domains of native heavy and light chains each comprise four FRs (FR1, FR2, FR3 and FR4, respectively), largely adopting a β-sheet configuration, connected by three hypervariable regions, which form loops connecting, and in some cases, forming part of, the β-sheet structure.

The hypervariable regions in each chain are held together in close proximity by the FRs and, with the hypervariable regions from the other chain, contribute to the formation of the antigen-binding site of antibodies (Kabat et al., 1991, specifically incorporated herein by reference). The constant domains are not involved directly in binding an antibody to an antigen, but exhibit various effector functions, such as participation of the antibody in antibody-dependent cellular toxicity.

The term “hypervariable region”, as used herein, refers to the amino acid residues of an antibody that are responsible for antigen-binding. The hypervariable region comprises amino acid residues from a “complementarity determining region” or “CDR” (i.e. residues 24-34 (L1). 50-56 (L2) and 89-97 (L3) in the light chain variable domain and 31-35 (H1). 50-56 (H2) and 95-102 (H3) in the heavy chain variable domain (Kabat et al., 1991, specifically incorporated herein by reference) and/or those residues from a “hypervariable loop” (i.e. residues 26-32 (L1), 50-52(L2) and 91-96 (L3) in the light chain variable domain and 26-32 (H1), 53-55 (H2) and 96-101 (H3) in the heavy chain variable domain). “Framework” or “FR” residues are those variable domain residues other than the hypervariable region residues as herein defined.

An “Fv” fragment is the minimum antibody fragment that contains a complete antigen-recognition and binding site. This region consists of a dimer of one heavy chain and one light chain variable domain in tight, con-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

“Single-chain Fv” or “sFv” antibody fragments comprise the V_{H} and V_{L} domains of antibody, wherein these domains are present in a single polypeptide chain. Generally, the Fv polypeptide further comprises a polypeptide linker between the V_{H} and V_{L} domains that enables the sFv to form the desired structure for antigen binding.

The following patents are specifically incorporated herein by reference for the purposes of even further supplementing the present teachings regarding the preparation and use of functional, antigen-binding regions of antibodies, including scFv, Fv, Fab′, Fab and F(ab′)₂ fragments of the anti-tumor antibodies: U.S. Pat. Nos. 5,855.866; 5,877,289; 5,965,132; 6,093.399; and 6,004,555. WO 98/45331 is also incorporated herein by reference for purposes including even further describing and teaching the preparation of variable, hypervariable and complementarity determining (CDR) regions of antibodies.

“Diabodies” are small antibody fragments with two antigen-binding sites, which fragments comprise a heavy chain variable domain (V_{H}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described in EP 404,097 and WO 93/11161. each specifically incorporated herein by reference. “Linear antibodies”, which can be bispecific or monospecific, comprise a pair of tandem Fd segments (V_{H}-C_{H}1-V_{H}-C_{H}1) that form a pair of antigen binding regions, as described in Zapata et al. (1995), specifically incorporated herein by reference.

Other types of variants are antibodies with improved biological properties relative to the parent antibody from which they are generated. Such variants, or second generation compounds, are typically substitutional variants involving one or more substituted hypervariable region residues of a parent antibody. A convenient way for generating such substitutional variants is affinity maturation using phage display.

In affinity maturation using phage display, several hypervariable region sites (e.g. 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine scanning mutagenesis can be performed to identified hypervariable region residues contributing significantly to antigen binding.

Alternatively, or in addition, the crystal structure of the antigen-antibody complex be delineated and analyzed to identify contact points between the antibody and target. Such contact residues and neighboring residues are candidates for substitution. Once such variants are generated, the panel of variants is subjected to screening, and antibodies with analogues but different or even superior properties in one or more relevant assays are selected for further development.

In using a Fab′ or antigen binding fragment of an antibody, with the attendant benefits on tissue penetration, one may derive additional advantages from modifying the fragment to increase its half-life. A variety of techniques may be employed, such as manipulation or modification of the antibody molecule itself, and also conjugation to inert carriers. Any conjugation for the sole purpose of increasing half-life, rather than to deliver an agent to a target, should be approached carefully in that Fab′ and other fragments are chosen to penetrate tissues. Nonetheless, conjugation to non-protein polymers, such PEG and the like, is contemplated.

Modifications other than conjugation are therefore based upon modifying the structure of the antibody fragment to render it more stable, and/or to reduce the rate of catabolism in the body. One mechanism for such modifications is the use of D-amino acids in place of L-amino acids. Those of ordinary skill in the art will understand that the introduction of such modifications needs to be followed by rigorous testing of the resultant molecule to ensure that it still retains the desired biological properties. Further stabilizing modifications include the use of the addition of stabilizing moieties to either the N-terminal or the C-terminal, or both, which is generally used to prolong the half-life of biological molecules. By way of example only, one may wish to modify the termini by acylation or amination.

Moderate conjugation-type modifications for use with the present invention include incorporating a salvage receptor binding epitope into the antibody fragment. Techniques for achieving this include mutation of the appropriate region of the antibody fragment or incorporating the epitope as a peptide tag that is attached to the antibody fragment. WO 96/32478 is specifically incorporated herein by reference for the purposes of further exemplifying such technology. Salvage receptor binding epitopes are typically regions of three or more amino acids from one or two lops of the Fc domain that are transferred to the analogous position on the antibody fragment. The salvage receptor binding epitopes of WO 98/45331 are incorporated herein by reference for use with the present invention.

E. Biologically Functional Equivalents

Equivalents, or even improvements, of anti-tumor antibodies and tumor binding proteins can now be made, generally using the materials provided above as a starting point. This discussion of equivalents also applies to equivalents and/or improvements of naked Tissue Factor and other coagulants generally using the materials provided above as a starting point. Modifications and changes may be made in the structure of an antibody, binding protein or coagulant and still obtain a molecule having like or otherwise desirable characteristics. For example, certain amino acids may substituted for other amino acids in a protein structure without appreciable loss of interactive binding capacity, such as, binding to tumor targets.

Since it is the interactive capacity and nature of a protein that defines that protein's biological functional activity, certain amino acid sequence substitutions can be made in a protein sequence (or of course, the underlying DNA sequence) and nevertheless obtain a protein with like (agonistic) properties. It is thus contemplated that various changes may be made in the sequence of known antibodies, binding proteins or peptides (or underlying DNA sequences) without appreciable loss of their biological utility or activity. Biological functional equivalents made from mutating an underlying DNA sequence can be generated using the supporting technical details on site-specific mutagenesis (see below) and the codon information provided in Table B.

TABLE B

| Amino Acids | Codons | | | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutamic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

It also is well understood by the skilled artisan that, inherent in the definition of a “biologically functional equivalent” protein or peptide, is the concept that there is a limit to the number of changes that may be made within a defined portion of the molecule and still result in a molecule with an acceptable level of equivalent biological activity. Biologically functional equivalent antibodies, proteins and peptides are thus defined herein as those antibodies, proteins and peptides in which certain, not most or all, of the amino acids may be substituted. Of course, a plurality of distinct antibodies, proteins/peptides with different substitutions may easily be made and used in accordance with the invention.

Amino acid substitutions are generally based on the relative similarity of the amino acid side-chain substituents, for example, their hydrophobicity, hydrophilicity, charge, size, and the like, An analysis of the size, shape and type of the amino acid side-chain substituents reveals that arginine, lysine and histidine are all positively charged residues; that alanine, glycine and serine are all a similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents.

In making more quantitative changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte and Doolittle, 1982, incorporated herein by reference). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those which are within ±1 are particularly preferred, and those within ±0.5 are even more particularly preferred.

It is thus understood that an amino acid can be substituted for another having a similar hydrophilicity value and still obtain a biologically equivalent protein. As detailed in U.S. Pat. No. 4,554,101 (incorporated herein by reference), the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon hydrophilicity values, the substitution of amino acids whose hydrophilicity values are within ±2 is preferred, those which are within ±1 are particularly preferred, and those within ±0.5 are even more particularly preferred.

F. Antibody Conjugation

According to these aspects of the present invention, anti-tumor targeting agents, antibodies, growth factors and such like are conjugated to, or operatively associated with, coagulants, either directly or indirectly, to prepare “coaguligands”. The operative linkages are the same type as those used with anti-cellular and cytotoxic agents to prepare “immunotoxins”. The targeting agents may thus be directly linked to a coagulant, or may be linked to a second binding region that binds and then releases a coagulant. The “second binding region” can result in a bispecific antibody construct. The preparation and use of bispecific antibodies in general is well known in the art, and is further disclosed herein.

In using immunoconjugate technology, the preparation of coaguligands is now generally known in the art. However, certain advantages may be achieved through the application of certain preferred technology, both in the preparation and purification for subsequent clinical administration. For example, while IgG based coaguligands will typically exhibit better binding capability and slower blood clearance than their Fab′ counterparts, Fab′ fragment-based coaguligands will generally exhibit better tissue penetrating capability as compared to IgG based coaguligands.

Additionally, while numerous types of disulfide-bond containing linkers are known that can be successfully employed to conjugate the coagulant to the targeting agent, certain linkers will generally be preferred over other linkers, based on differing pharmacological characteristics and capabilities. For example, linkers that contain a disulfide bond that is sterically “hindered” are to be preferred, due to their greater stability in vivo, thus preventing release of the coagulant prior to binding at the site of action.

Each type of cross-linker, as well as how the cross-linking is performed, will tend to vary the pharmacodynamics of the resultant conjugate. One may desire to have a conjugate that will remain intact under conditions found everywhere in the body except the intended site of action, at which point it is desirable that the conjugate have good “release” characteristics. Therefore, the particular cross-linking scheme, including in particular the particular cross-linking reagent used and the structures that are cross-linked, will be of some significance.

Depending on the specific coagulant used as part of the fusion protein, it may be necessary to provide a peptide spacer operatively attaching the targeting agent and the coagulant, which is capable of folding into a disulfide-bonded loop structure. Proteolytic cleavage within the loop would then yield a heterodimeric polypeptide wherein the targeting agent and the coagulant are linked by only a single disulfide bond. Non-cleavable peptide spacers may also be provided to operatively attach the targeting agent and the coagulant of the fusion protein.

A variety of chemotherapeutic and other pharmacological agents have now been successfully conjugated to antibodies and shown to function pharmacologically. Exemplary antineoplastic agents that have been investigated include doxorubicin, daunomycin, methotrexate, vinblastine, and various others. Moreover, the attachment of other agents such as neocarzinostatin, macromycin, trenimon and α-amanitin has been described. These attachment methods can be adapted fur use herewith.

Any covalent linkage to the antibody or targeting agent should ideally be made at a site distinct from the functional site of the coagulant. The compositions are thus “linked” in any operative manner that allows each region to perform its intended function without significant impairment. Thus, the targeting agents bind to tumor antigens, and the coagulant directly or indirectly causes coagulation.

F1. Biochemical Cross-linkers

In additional to the general information provided above, anti-tumor antibodies may be conjugated to coagulants using certain preferred biochemical cross-linkers. Cross-linking reagents are used to form molecular bridges that tie together functional groups of two different molecules. To link two different proteins in a step-wise manner, hetero-bifunctional cross-linkers can be used that eliminate unwanted homopolymer formation. Exemplary hetero-bifunctional cross-linkers are referenced in Table C.

TABLE C
HETERO-BIFUNCTIONAL CROSS-LINKERS

| | | | Spacer Arm |
|---|---|---|---|
| | | | Length after |
| Linker | Reactive Toward | Advantages and Applications | cross-linking |
| SMPT | Primary amines Sulfhydryls | Greater stability | 11.2 A |
| SPDP | Primary amines Sulfhydryls | Thiolation | 6.8 A |
| | | Cleavable cross-linking | |
| LC-SPDP | Primary amines Sulthydryls | Extended spacer arm | 15.6 A |
| Sulfo-LC-SPDP | Primary amines Sulfhydryls | Extended spacer arm | 15.6 A |
| | | Water-soluble | |
| SMCC | Primary amines Sulfhydryls | Stable maleimide reactive group | 11.6 A |
| | | Enzyme-antibody conjugation | |
| | | Hapten-carrier protein conjugation | |
| Sulfo-SMCC | Primary amines Sulfhydryls | Stable maleimide reactive group | 11.6 A |
| | | Water-soluble | |
| | | Enzyme-antibody conjugation | |
| MBS | Primary amines Sulfhydryls | Enzyme-antibody conjugation | 9.9 A |
| | | Hapten-carrier protein conjugation | |
| Sulfo-MBS | Primary amines Sulfhydryls | Water-soluble | 9.9 A |
| SIAB | Primary amines Sulfhydryls | Enzyme-antibody conjugation | 10.6 A |
| Sulfo-SIAB | Primary amines Sulthydryls | Water-soluble | 10.6 A |
| SMPB | Primary amines Sulfhydryls | Extended spacer arm | 14.5 A |
| | | Enzyme-antibody conjugation | |
| Sulfo-SMPB | Primary amines Sulfhydryls | Extended spacer arm | 14.5 A |
| | | Water-soluble | |
| EDC/Sulfo-NHS | Primary amines Carboxyl | Hapten-Carrier conjugation | 0 |
| | groups | | |
| ABH | Carbohydrates Nonselective | Reacts with sugar groups | 11.9 A |

Hetero-bifunctional cross-linkers contain two reactive groups: one generally reacting with primary amine group (e.g.. N-hydroxy succinimide) and the other generally reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein.

Compositions therefore generally have, or are derivatized to have, a functional group available for cross-linking purposes. This requirement is not considered to be limiting in that a wide variety of groups can be used in this manner. For example, primary or secondary amine groups, hydrazide or hydrazine groups, carboxyl alcohol, phosphate, or alkylating groups may be used for binding or cross-linking.

The spacer arm between the two reactive groups of a cross-linkers may have various length and chemical compositions. A longer spacer arm allows a better flexibility of the conjugate components while some particular components in the bridge (e.g., benzene group) may lend extra stability to the reactive group or an increased resistance of the chemical link to the action of various aspects (e g., disulfide bond resistant to reducing agents). The use of peptide spacers, such as L-Leu-L-Ala-L-Leu-L-Ala, is also contemplated.

It is preferred that a cross-linker having reasonable stability in blood will be employed. Numerous types of disulfide-bond containing linkers are known that can be successfully employed to conjugate coagulants. Linkers that contain a disulfide bond that is sterically hindered may prove to give greater stability in vivo, preventing release of the agent prior to binding at the site of action. These linkers are thus one preferred group of linking agents.

One of the most preferred cross-linking reagents is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the tumor site. It is contemplated that the SMPT agent may also be used in connection with the bispecific ligands of this invention.

The SMPT cross-linking reagent, as with many other known cross-linking reagents. lends the ability to cross-link functional groups such as the SH of cysteine or primary amines (e.g. the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1 ,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers can also be employed in accordance herewith. Other useful cross-linkers, not considered to contain or generate a protected disulfide, include SATA, SPDP and 2-iminothiolane. The use of such cross-linkers is well understood in the art.

Once conjugated, the conjugate is separated from unconjugated targeting agents and coagulants and from other contaminants. A large a number of purification techniques are available for use in providing conjugates of a sufficient degree of purity to render them clinically useful. Purification methods based upon size separation, such as gel filtration, gel permeation or high performance liquid chromatography, will generally be of most use. Other chromatographic techniques, such as Blue-Sepharose separation, may also be used.

F2. Biologically Releasable Linkers

Although it is preferred that any linking moiety will have reasonable stability in blood, to prevent substantial release of the attached coagulant before targeting to the disease or tumor site, in certain aspects, the use of biologically-releasable bonds and/or selectively cleavable spacers or linkers is contemplated. “Biologically-releasable bonds” and “selectively cleavable spacers or linkers” still have reasonable stability in the circulation.

The targeting agents and/or antibodies in accordance with the invention may thus be linked to one or more coagulants via a biologically-releasable bond. Any form of targeting agent or antibody may be employed, including intact antibodies, although ScFv fragments will be preferred in certain embodiments.

“Biologically-releasable bonds” or “selectively hydrolyzable bonds” include all linkages that are releasable, cleavable or hydrolyzable only or preferentially under certain conditions. This includes disulfide and trisulfide bonds and acid-labile bonds, as described in U.S. Pat. Nos. 5,474,765 and 5,762,918, each specifically incorporated herein by reference.

The use of an acid sensitive spacer for attachment of a coagulant to an antibody of the invention is particularly contemplated. In such embodiments, the coagulants are released within the acidic compartments inside a cell. It is contemplated that acid-sensitive release may occur extracellularly, but still after specific targeting, preferably to the tumor site. Attachment via carbohydrate moieties of antibodies is also contemplated. In such embodiments, the coagulants are released within the acidic compartments inside a cell.

The targeting agent or antibody may also be derivatized to introduce functional groups permitting the attachment of the coagulants through a biologically releasable bond. The targeting agent or antibody may thus be derivatized to introduce side chains terminating in hydrazide, hydrazine, primary amine or secondary amine groups. Coagulants may be conjugated through a Schiff's base linkage, a hydrazone or acyl hydrazone bond or a hydrazide linker (U.S. Pat. Nos. 5,474,765 and 5,762,918, each specifically incorporated herein by reference).

Also as described in U.S. Pat. Nos. 5,474,765 and 5,762,918, each specifically incorporated herein by reference, the targeting agent or antibody may be operatively attached to the coagulant through one or more biologically releasable bonds that are enzyme-sensitive bonds, including peptide bonds, esters, amides, phosphodiesters and glycosides.

Certain preferred aspects of the invention concern the use of peptide linkers that include at least a first cleavage site for a peptidase and/or proteinase that is preferentially located within a disease site, particularly within the tumor environment. The antibody-mediated delivery of the attached coagulant thus results in cleavage specifically within the disease site or tumor environment, resulting in the specific release of the active coagulant. Certain peptide linkers will include a cleavage site that is recognized by one or more enzymes involved in remodeling.

Peptide linkers that include a cleavage site for urokinase, pro-urokinase, plasmin. plasminogen, TGFβ, staphylokinase, Thrombin, Factor IXa, Factor Xa or a metalloproteinase, such as an interstitial collagenase, a gelatinase or a stromelysin, are particularly preferred. U.S. Pat. Nos. 6,004,555, 5,877,289, and 6,093,399 are specifically incorporated herein by reference for the purpose of further describing and enabling how to make and use coaguligands comprising biologically-releasable bonds and selectively-cleavable linkers and peptides. U.S. Pat. No. 5,877,289 is particularly incorporated herein by reference for the purpose of further describing and enabling how to make and use coaguligands that comprise a selectively-cleavable peptide linker that is cleaved by urokinase, plasmin, Thrombin, Factor IXa, Factor Xa or a metalloproteinase, such as an interstitial collagenase, a gelatinase or a stromelysin, within a tumor environment.

Currently preferred selectively-cleavable peptide linkers are those that include a cleavage site for plasmin or a metalloproteinase (also known as “matrix metalloproteases” or “MMPs”), such as an interstitial collagenase, a gelatinase or a stromelysin. Additional peptide linkers that may be advantageously used in connection with the present invention include, for example, plasmin cleavable sequences, such as those cleavable by pro-urokinase, TGFβ, plasminogen and staphylokinase; Factor Xa cleavable sequences; MMP cleavable sequences, such as those cleavable by gelatinase A; collagenase cleavable sequences, such as those cleavable by calf skin collagen (α1(I) chain), calf skin collagen (α2(l) chain), bovine cartilage collagen (α1(II)chain), human liver collagen (α1(III) chain), human α₂M, human PZP, rat α₁M, rat α₂M, rat α₁I₃(2J), rat α₁I₃(27J), and the human fibroblast collagenase autolytic cleavage sites. In addition to the knowledge available to those of ordinary skill in the art, the text and sequences from Table B2 in U.S. Pat. Nos. 6,342,219, 6,342,221 and 6,416,758 are specifically incorporated herein by reference without disclaimer for purposes of even further describing and enabling the use of such cleavable sequences.

F3. Bispecific Antibodies

Bispecific antibodies in general may be employed, so long as one arm binds to a tumor antigen and the bispecific antibody is attached to a coagulant. The bispecific antibody may be attached to a coagulant at a site distant from the antigen-binding region, or a coagulant-binding arm may be used.

In general, the preparation of bispecific antibodies is also well known in the art. One method involves the separate preparation of antibodies having specificity for the targeted antigen, on the one hand, and (as herein) a coagulant on the other. Peptic F(ab′γ)₂ fragments are prepared from the two chosen antibodies, followed by reduction of each to provide separate Fab′_{γSH} fragments. The SH groups on one of the two partners to be coupled are then alkylated with a cross-linking reagent such as o-phenylenedimaleimide to provide free maleimide groups on one partner. This partner may then be conjugated to the other by means of a thioether linkage, to give the desired F(ab′γ)₂ heteroconjugate. Other techniques are known wherein cross-linking with SPDP or protein A is carried out, or a trispecific construct is prepared.

Another method for producing bispecific antibodies is by the fusion of two hybridomas to form a quadroma. As used herein, the term “quadroma” is used to describe the productive fusion of two B cell hybridomas. Using now standard techniques, two antibody producing hybridomas are fused to give daughter cells, and those cells that have maintained the expression of both sets of clonotype immunoglobulin genes are then selected.

A preferred method of generating a quadroma involves the selection of an enzyme deficient mutant of at least one of the parental hybridomas. This first mutant hybridoma cell line is then fused to cells of a second hybridoma that had been lethally exposed, e.g., to iodoacetamide, precluding its continued survival. Cell fusion allows for the rescue of the first hybridoma by acquiring the gene for its enzyme deficiency from the lethally treated hybridoma, and the rescue of the second hybridoma through fusion to the first hybridoma. Preferred, but not required, is the fusion of immunoglobulins of the same isotype, but of a different subclass. A mixed subclass antibody permits the use if an alternative assay for the isolation of a preferred quadroma.

In more detail, one method of quadroma development and screening involves obtaining a hybridoma line that secretes the first chosen MAb and making this deficient for the essential metabolic enzyme, hypoxanthine-guanine phosphoribosyltransferase (HGPRT). To obtain deficient mutants of the hybridoma, cells are grown in the presence of increasing concentrations of 8-azaguanine (1×10⁻⁷M to 1×10⁻⁵M). The mutants are subcloned by limiting dilution and tested for their hypoxanthine/aminopterin/thymidine (HAT) sensitivity. The culture medium may consist of, for example, DMEM supplemented with 10% FCS. 2 mM L-Glutamine and 1 mM penicillin-streptomycin.

A complementary hybridoma cell line that produces the second desired MAb is used to generate the quadromas by standard cell fusion techniques. Briefly, 4.5×10⁷ HAT-sensitive first cells are mixed with 2.8×10⁷ HAT-resistant second cells that have been pre-treated with a lethal dose of the irreversible biochemical inhibitor iodoacetamide (5 mM in phosphate buffered saline) for 30 minutes on ice before fusion. Cell fusion is induced using polyethylene glycol (PEG) and the cells are plated out in 96 well microculture plates. Quadromas are selected using HAT-containing medium. Bispecific antibody-containing cultures are identified using, for example, a solid phase isotype-specific ELISA and isotype-specific immunofluorescence staining.

In one identification embodiment to identify the bispecific antibody, the wells of microtiter plates (Falcon, Becton Dickinson Labware) are coated with a reagent that specifically interacts with one of the parent hybridoma antibodies and that lacks cross-reactivity with both antibodies. The plates are washed, blocked, and the supernatants (SNs) to be tested are added to each well. Plates are incubated at room temperature for 2 hours, the supernatants discarded, the plates washed, and diluted alkaline phosphatase-anti-antibody conjugate added for 2 hours at room temperature. The plates are washed and a phosphatase substrate, e.g, P-Nitrophenyl phosphate (Sigma, St. Louis) is added to each well. Plates are incubated. 3N NaOH is added to each well to stop the reaction, and the OD₄₁₀ values determined using an ELISA reader.

In another identification embodiment, microtiter plates pre-treated with poly-L-lysine are used to bind one of the target cells to each well, the cells are then fixed, e.g. using 1% glutaraldehyde, and the bispecific antibodies are tested for their ability to bind to the intact cell. In addition, FACS, immunofluorescence staining, idiotype specific antibodies, antigen binding competition assays, and other methods common in the art of antibody characterization may be used in conjunction with the present invention to identify preferred quadromas.

Following the isolation of the quadroma, the bispecific antibodies are purified away from other cell products. This may be accomplished by a variety of protein isolation procedures, known to those skilled in the art of immunoglobulin purification. Means for preparing and characterizing antibodies are well known in the art (See, e.g.. Antibodies: A Laboratory Manual, 1988).

For example, supernatants from selected quadromas are passed over protein A or protein G sepharose columns to bind IgG (depending on the isotype). The bound antibodies are then eluted with, e.g. a pH 5.0 citrate buffer. The elute fractions containing the BsAbs, are dialyzed against an isotonic buffer. Alternatively, the eluate is also passed over an anti-immunoglobulin-sepharose column. The BsAb is then eluted with 3.5 M magnesium chloride. BsAbs purified in this way are then tested for binding activity by, e.g., an isotype-specific ELISA and immunofluorescence staining assay of the target cells, as described above.

Purified BsAbs and parental antibodies may also be characterized and isolated by SDS-PAGE electrophoresis, followed by staining with silver or Coomassie. This is possible when one of the parental antibodies has a higher molecular weight than the other, wherein the band of the BsAbs migrates midway between that of the two parental antibodies. Reduction of the samples verifies the presence of heavy chains with two different apparent molecular weights.

G. Fusion Proteins and Recombinant Expression

Certain aspects of the present invention are directed to the combined use of tumor-targeting agents in the delivery of coagulants. In the preparation of such constructs, recombinant expression may be employed to create a fusion protein, as is known to those of skill in the art and further disclosed herein. Equally, coagulant-containing constructs may be generated using avidin:biotin bridges or any of the foregoing chemical conjugation and cross-linker technologies, mostly developed in reference to antibody conjugates. Therefore, any suitable binding protein, ligand or peptide may be conjugated to a coagulant in the same manner as used for antibody conjugates, described herein.

In using recombinant expression to prepare tumor-targeted coagulants, the nucleic acid sequences encoding the chosen targeting agent are attached, in-frame, to nucleic acid sequences encoding the chosen coagulant or second binding region to create an expression unit or vector. Recombinant expression results in translation of the new nucleic acid, to yield the desired protein product. The recombinant approach is essentially the same whether nucleic acids encoding antibodies or protein binding ligands are employed.

The coaguligands of the present invention may be readily prepared as fusion proteins using molecular biological techniques. The use of recombinant DNA techniques to achieve such ends is now standard practice to those of skill in the art. These methods include, for example, in vitro recombinant DNA techniques, synthetic techniques and in vivo recombination/genetic recombination. DNA and RNA synthesis may, additionally, be performed using an automated synthesizers (see, for example, the techniques described in Sambrook et al., 1989).

The preparation of such a fusion protein generally entails the preparation of a first and second DNA coding region and the functional ligation or joining of such regions, in frame, to prepare a single coding region that encodes the desired fusion protein. In the present context, the targeting agent DNA sequence will be joined in frame with a DNA sequence encoding a coagulant. It is not generally believed to be particularly relevant which portion of the coaguligand is prepared as the N-terminal region or as the C-terminal region.

Once the desired coding region has been produced, an expression vector is created. Expression vectors contain one or more promoters upstream of the inserted DNA regions that act to promote transcription of the DNA and to thus promote expression of the encoded recombinant protein. This is the meaning of “recombinant expression”.

To obtain a so-called “recombinant” version of the coaguligand, the vector is expressed in a recombinant cell. The engineering of DNA segment(s) for expression in a prokaryotic or eukaryotic system may be performed by techniques generally known to those of skill in recombinant expression. It is believed that virtually any expression system may be employed in the expression of the coaguligands.

Such proteins may be successfully expressed in eukaryotic expression systems. eg, CHO cells, however, it is envisioned that bacterial expression systems, such as E coli pQE-60 will be particularly useful for the large-scale preparation and subsequent purification of the coaguligands. cDNAs may also be expressed in bacterial systems, with the encoded proteins being expressed as fusions with β-galactosidase, ubiquitin. Schistosoma japonicum glutathione S-transferase, and the like. It is believed that bacterial expression will have advantages over eukaryotic expression in terms of ease of use and quantity of materials obtained thereby.

In terms of microbial expression, U.S. Pat. Nos. 5,583,013; 5,221,619; 4,785,420; 4,704,362; and 4,366,246 are incorporated herein by reference for the purposes of even further supplementing the present disclosure in connection with the expression of genes in recombinant host cells.

Recombinantly produced coaguligands may be purified and formulated for human administration. Alternatively, nucleic acids encoding the coaguligands may be delivered via gene therapy. Although naked recombinant DNA or plasmids may be employed, the use of liposomes or vectors is preferred. The ability of certain viruses to enter cells via receptor-mediated endocytosis and to integrate into the host cell genome and express viral genes stably and efficiently have made them attractive candidates for the transfer of foreign genes into mammalian cells. Preferred gene therapy vectors for use in the present invention will generally be viral vectors.

Retroviruses have promise as gene delivery vectors due to their ability to integrate their genes into the host genome, transferring a large amount of foreign genetic material, infecting a broad spectrum of species and cell types and of being packaged in special cell-lines. Other viruses, such as adenovirus, herpes simplex viruses (HSV), cytomegalovirus (CMV), and adeno-associated virus (AAV), such as those described by U.S. Pat. No. 5,139,941 (incorporated herein by reference), may also be engineered to serve as vectors for gene transfer.

Although some viruses that can accept foreign genetic material are limited in the number of nucleotides they can accommodate and in the range of cells they infect, these viruses have been demonstrated to successfully effect gene expression. However, adenoviruses do not integrate their genetic material into the host genome and therefore do not require host replication for gene expression, making them ideally suited for rapid, efficient, heterologous gene expression. Techniques for preparing replication-defective infective viruses are well known in the art.

In certain further embodiments, the gene therapy vector will be HSV. A factor that makes HSV an attractive vector is the size and organization of the genome. Because HSV is large, incorporation of multiple genes or expression cassettes is less problematic than in other smaller viral systems. In addition, the availability of different viral control sequences with varying performance (e.g., temporal, strength) makes it possible to control expression to a greater extent than in other systems. It also is an advantage that the virus has relatively few spliced messages, further easing genetic manipulations. HSV also is relatively easy to manipulate and can be grown to high titers.

Of course, in using viral delivery systems, one will desire to purify the virion sufficiently to render it essentially free of undesirable contaminants, such as defective interfering viral particles or endotoxins and other pyrogens such that it will not cause any untoward reactions in the cell, animal or individual receiving the vector construct. A preferred means of purifying the vector involves the use of buoyant density gradients, such as cesium chloride gradient centrifugation.

H. Anti-Aminophospholipid Antibodies and Immunoconjugates

In certain aspects of the invention, implementing the sensitizing step of the combination treatment methods will result in increased expression of aminophospholipids, such as phosphatidylserine or phosphatidylethanolamine, or certain other asymmetrically distributed phospholipids, such as phosphatidylinositol (PI), which may be targeted using naked antibodies or immunoconjugates directed to such phospholipid markers. Therefore, in these defined treatment steps, the additional therapeutic agents are not limited to agents for coagulative tumor therapy, although aminophospholipid- and phospholipid-targeted coagulants may certainly be used.

In the sensitizing, typically the first, steps of such methods, the initial administration of one or more agents is designed to increase aminophospholipid expression. This may be achieved by using TNF and platelet activating factor (PAF) inducers and/or mimetics. Other preferred first steps include the use of Reactive Oxygen Species (ROS) generators, such as H₂O₂, peroxides, thrombin, IL-1 and also TNF. Of these, agents that increase H₂O₂ or thrombin in the tumor vasculature are particularly preferred.

Other mechanisms for increasing aminophospholipid expression include the use of hypoxia, low pH and inducers thereof. Exemplary suitable agents are NFκB activators, which function as inflammatory mediators and apoptosis inducers. Signaling mediators form another group of agents for use in increase aminophospholipid expression in tumor vasculature. These include, e.g., thapsigargin, phorbol esters and calcium ionophores, such as A23187.

It will be seen that various of the foregoing agents injure, or induce apoptosis in, the tumor endothelium. In addition to agents such as calcium ionophores, cyclophosphamide, mitomycin C and vinca alkaloids, a further exemplary agent is bleomycin.

Phosphatidylserine-binding molecules may themselves be used to induce further PS expression, which may then be used as the basis for the second or treatment step of the therapy. Anti-PS antibodies, coagulation factors II, Ia, IX, IXa, X, Xa, XI, XIa, XII, XIIa, β₂-glycoprotein and one or more of the annexins may be used in this regard.

A further means for increasing aminophospholipid expression is the use of agents that block survival factors. Particularly preferred examples of “blockers of survival factors” are anti-VEGF agents, such as anti-VEGF antibodies, VEGF RTK inhibitors, sFlk-1/sFLK-1, and anti-angiopoietin-1 agents, such as anti-Ang-1 antibodies and soluble Tie2 receptors capable of blocking Tie2 activation.

After administration of agents to induce PS, PE or other phospholipid expression, including PI, the second step of the methods may therefore involve the administration of naked antibodies targeting the over-expressed or induced aminophospholipids or phospholipids. These aspects of the overall invention are based on the surprising discovery that administration of naked anti-aminophospholipid antibodies alone is sufficient to induce thrombosis and tumor regression.

In using unconjugated, anti-phosphatidylserine and/or phosphatidylethanolamine antibodies in these second method steps, U.S. Pat. No. 6,406,693 is specifically incorporated herein by reference for the purposes of even further supplementing the present teachings regarding the preparation and use of such antibodies.

In targeting aminophospholipids, an “aminophospholipid”, as used herein, means a phospholipid that includes within its structure at least a first primary amino group. Preferably, the term “aminophospholipid” is used to refer to a primary amino group-containing phospholipid that occurs naturally in mammalian cell membranes. However, this is not a limitation on the meaning of the term “aminophospholipid”, as this term also extends to non-naturally occurring or synthetic aminophospholipids that nonetheless have uses in the invention, e g., as an immunogen in the generation of anti-aminophospholipid antibodies (“cross-reactive antibodies”) that do bind to aminophospholipids of mammalian plasma membranes. The aminophospholipids of U.S. Pat. No. 5,767,298, incorporated herein by reference, are appropriate examples.

The prominent aminophospholipids found in mammalian biological systems are the negatively-charged phosphatidylserine (“PS”) and the neutral or zwitterionic phosphatidylethanolamine (“PE”), which are therefore preferred aminophospholipids for targeting by the present invention. However, these aspects of the invention are by no means limited to the targeting of phosphatidylserines and phosphatidylethanolamines, and any other aminophospholipid target may be employed so long as it is expressed, accessible or complexed on the luminal surface of tumor vascular endothelial cells.

All aminophospholipid-, phosphatidylserine- and phosphatidylethanolamine-based components are encompassed as targets of these aspects of the invention, irrespective of the type of fatty acid chains involved, including those with short, intermediate or long chain fatty acids, and those with saturated, unsaturated and polyunsaturated fatty acids. Preferred compositions for raising antibodies for use in the present invention may be aminophospholipids with fatty acids of C18. with C18:1 being more preferred. To the extent that they are accessible on tumor vascular endothelial cells, aminophospholipid degradation products having only one fatty acid (lyso derivatives), rather than two, may also be targeted.

Another group of potential aminophospholipid targets include, for example. phosphatidal derivatives (plasmalogens), such as phosphatidalserine and phosphatidalethanolamine (having an ether linkage giving an alkenyl group, rather than an ester linkage giving an acyl group). Indeed, the targets for therapeutic intervention by these aspects of the invention include any substantially lipid-based component that comprises a nitrogenous base and that is present, expressed, translocated, presented or otherwise complexed in a targetable form on the luminal surface of tumor vascular endothelial cells, not excluding phosphatidylcholine (“PC”). Lipids not containing glycerol may also form appropriate targets, such as the sphingolipids based upon sphingosine and derivatives.

The biological basis for including a range of lipids in the group of targetable components lies, in part, with the observed biological phenomena of lipids and proteins combining in membranous environments to form unique lipid-protein complexes. Such lipid-protein complexes extend to antigenic and immunogenic forms of lipids such as phosphatidylserine, phosphatidylethanolamine and phosphatidylcholine with, e.g., proteins such as β₂-glycoprotein I, prothrombin, kininogens and prekallikrein. Therefore, as proteins and polypeptides can have one or more free primary amino groups, it is contemplated that a range of effective “aminophospholipid targets” may be formed in vivo from lipid components that are not aminophospholipids in the strictest sense. Nonetheless, all such targetable complexes that comprise lipids and primary amino groups constitute an “aminophospholipid” within the scope of these aspects of the invention.

The terms “naked” and “unconjugated” antibody, as used herein, are intended to refer to an antibody that is not conjugated, operatively linked or otherwise physically or functionally associated with an effector moiety, such as a cytotoxic or coagulative agent. It will be understood that the terms “naked” and “unconjugated” antibody do not exclude antibody constructs that have been stabilized, multimerized, humanized or in any other way manipulated, other than by the attachment of an effector moiety.

Accordingly, all post-translationally modified naked and unconjugated antibodies are included herewith, including where the modifications are made in the natural antibody-producing cell environment, by a recombinant antibody-producing cell, and are introduced by the hand of man after initial antibody preparation. Of course, the term “naked” antibody does not exclude the ability of the antibody to form functional associations with effector cells and/or molecules after administration to the body, as some such interactions are necessary in order to exert a biological effect. The lack of associated effector group is therefore applied in definition to the naked antibody in vitro, not in vivo.

Where the first steps of the combination treatment methods result in increased expression of targetable phospholipids and/or aminophospholipids, the second steps may utilize conjugated, anti-phosphatidylserine and/or anti-phosphatidylethanolamine antibodies or immunoconjugates based upon phospholipid or aminophospholipid binding proteins, U.S. Pat. No. 6,312,694 is specifically incorporated herein by reference for the purposes of even further supplementing the present teachings regarding the preparation and use of such immunoconjugates. In certain particular embodiments, the second step of the overall methods may involve the administration of an anti-aminophospholipid antibody conjugate, or an aminophospholipid binding protein conjugate, such as annexin conjugate, operatively attached to a coagulant. Where such aspects are intended, they will be particularly stated.

In the use of anti-phosphatidylserine and/or anti-phosphatidylethanolamine immunoconjugates, any one or more of the foregoing antibodies may be employed. However. phospholipid and aminophospholipid binding proteins may also be used in such constructs. These binding proteins or “ligands” may bind phosphatidylserine or phosphatidylethanolamine.

In terms of binding proteins that bind phosphatidylserine, preferred amongst these are annexins (sometimes spelt “annexines”), a group of calcium-dependent phospholipid binding proteins. At least nine members of the annexin family have been identified in mammalian tissues (Annexin I through Annexin IX). Most preferred amongst these is annexin V (also known as PAP-I).

U.S. Pat. No. 5,658,877, incorporated herein by reference, describes Annexin I, effective amounts of Annexin I and pharmaceutical compositions thereof, Annexin V contains one free sulfhydryl group and does not have any attached carbohydrate chains. The primary structure of annexin V deduced from the cDNA sequence shows that annexin V comprises four internal repeating units (U.S. Pat. No. 4,937,324; incorporated herein by reference).

U.S. Pat. No. 5,296,467 and WO 91/07187 are also each incorporated herein by reference as they provide pharmaceutical compositions comprising ‘annexine’ (annexin). WO 91/07187 provides natural, synthetic or genetically prepared derivatives and analogues of ‘annexine’ (annexin), which may now be used in the conjugates of the present invention. Particular annexins are provided of 320 amino acids, containing variant amino acids and. optionally, a disulphide bridge between the 316-Cys and the 2-Ala.

U.S. Pat. No. 5,296,467 is incorporated herein by reference in its entirety, including all figures and sequences, for purposes of even further describing annexins and pharmaceutical compositions thereof. U.S. Pat. No. 5,296,467 describes annexin cloning, recombinant expression and preparation. Aggregates of two or more annexines, e.g., linked by disulfide bonds between one or more cysteine groups on the respective annexine, are also disclosed. Yet a further example of suitable annexin starting materials is provided by WO 95/27903 (incorporated herein by reference), which provides annexins for use in detecting apoptotic cells.

To the extent that they clearly describe appropriate annexin starting materials for preparing therapeutic constructs of the present invention, each of the diagnostic approaches of U.S. Pat. No. 5,627,036; WO 95/19791; WO 95/27903; WO 95/34315; WO 96/17618; and WO 98/04294; are also specifically incorporated herein by reference. Various of these documents also concern recombinant expression vectors useful for adaptation into the present invention.

U.S. Pat. No. 5,632,986 is also specifically incorporated herein by reference for purposes of further describing mutants and variants of the annexin molecule that are subdivided or altered at one or more amino acid residues so long as the phospholipid binding capability is not reduced substantially. Appropriate annexins for use in the present invention can thus be truncated, for example, to include one or more domains or contain fewer amino acid residues than the native protein, or can contain substituted amino acids. Any changes are acceptable within the scope of the invention so long as the mutein or second generation annexin molecule does not contain substantially lower affinity for aminophospholipid. Such guidance can also be applied to phosphatidylethanolamine binding proteins.

The chemical cross-linking of annexins and other agents is also described in U.S. Pat. No. 5,632,986, incorporated herein by reference. All such techniques can be adapted for use herewith simply by substituting the thrombolytic agents for those described herein. Aliphatic diamines; succinimide esters; hetero-bifunctional coupling reagents, such as SPDP; maleimide compounds; linkers with spacers; and the like, may thus be used, U.S. Pat. No. 5,632,986 is yet further specifically incorporated herein by reference for purposes of describing the recombinant production of annexin-containing conjugates.

As to binding proteins that bind phosphatidylethanolamine, preferred amongst these are kininogens, which are naturally occurring proteins that normally have anti-thrombotic effects. Low or high molecular weight kininogens may now be attached to therapeutic agents and used in the delivery of therapeutics to phosphatidylethanolamine, a marker of tumor vasculature.

Various mammalian and human kininogen genes have now been cloned, and such genes and proteins can be used in the various recombinant and/or chemical embodiments of the present invention. For example, the complete nucleotide and amino acid sequences of the genes and proteins described in Nakanishi et al., 1983, are incorporated herein by reference for such purposes.

cDNA, gene and protein sequences for bovine low molecular weight kininogens are known Kitamura et al. (1983; incorporated herein by reference). Kitamura et al. (1983) reported that a single gene encodes the bovine high molecular weight and low molecular weight kininogens. Kitamura et al. (1987) is also specifically incorporated herein by reference for purposes of providing further information concerning the bovine, rat and human kininogens, including low molecular weight, high molecular weight and T-kininogens.

Preferred high and low molecular weight kininogens for use in these aspects of the invention will be the human genes and proteins, as described by Kitamura et al. (1985) and Kellermann et al. (1986), each incorporated herein by reference. The complete nucleotide and amino acid sequences of human low and high molecular weight prekininogens are known.

Kitamura et al (1985) is also specifically incorporated herein by reference for purposes of providing further information regarding the structural organization of the human kininogen gene, as may be used, e g., to design particular expression constructs for use herewith. Kitamura et al (1988) is further incorporated by reference for purposes of providing detailed information regarding the cloning of cDNAs and genomic kininogens, such that any desired kininogen may be cloned.

In addition to the T-kininogens described by Kitamura et al. (1987; incorporated herein by reference), Anderson et al. (1989) is also specifically incorporated herein by reference for purposes of providing the gene and protein sequences of T-kininogen.

Other phosphatidylethanolamine binding proteins are known that can be used in such embodiments. A number of studies, particularly by Jones and Hall, and Bernier and Jolles, have concerned the purification, characterization and cloning of phosphatidylethanolamine binding proteins. For example, Bernier and Jolles (1984; incorporated herein by reference) first reported the purification and characterization of a basic ˜23 kDa cytosolic protein from bovine brain that was later characterized as a phosphatidylethanolamine-binding protein (Bernier et al., 1986; incorporated herein by reference). Schoentgen et al. (1987; incorporated herein by reference) reported the complete amino acid sequence of this bovine protein, then shown to be 21 kDa.

Jones and Hall (1991; incorporated herein by reference) later purified and partially sequenced a ˜23 kDa protein from rat sperm plasma membranes that showed sequence similarity and phospholipid binding properties similar to the bovine brain cytosolic protein of Bernier and Jolles (Bernier and Jolles, 1984; Bernier et al., 1986; Schoentgen et al, 1987). The rat 23 kDa protein of Jones and Hall (1991; incorporated herein by reference) also showed selective affinity for phosphatidylethanolamine (Kd=1.6×10⁻⁵ M).

Perry et al. (1994; incorporated herein by reference) then cloned and sequenced rat and monkey versions of the phosphatidylethanolamine binding protein of Jones and Hall (1991). FIGS. 4, 5 and 6 of Perry et al. (1994) are specifically incorporated herein by reference for purposes of providing the complete DNA and amino acid sequences of the rat and monkey phosphatidylethanolamine binding proteins, and comparison to the bovine protein sequence. Any of the foregoing mammalian phosphatidylethanolamine binding proteins, or their human counterparts, may be attached to therapeutic agents and used in the present invention. These mammalian sequences have EMBL Nucleotide Sequence Database Accession Numbers X71873 (rat) and X73137 (monkey), and are each incorporated herein by reference.

To counterpart human phosphatidylethanolamine binding protein has also been cloned (Horn et al., 1994; incorporated herein by reference). GenBank, EMBL and DDBJ Accession Number D16111 are also incorporated herein by reference for purposes of providing the complete DNA and amino acid sequences of the human phosphatidylethanolamine binding proteins. The mammalian and human sequences, as incorporated herein, may be employed in well-known expression techniques, either to express the proteins themselves or therapeutic agent-fusions thereof. Phosphatidylethanolamine binding proteins and genes from other sources, such as yeast, Drosophila, simian, T. canis and O. volvulus may also be employed in these embodiments (Gems et al., 1995; incorporated herein by reference).

Variant, mutant or second generation phosphatidylethanolamine binding protein nucleic acids may also be readily prepared by standard molecular biological techniques, and may optionally be characterized as hybridizing to any of the phosphatidylethanolamine binding protein nucleotide sequences set forth in any one or more of Nakanishi et al. (1983); Kitamura et al. (1983; 1985; 1987; 1988); Kellermann et al. (1986); Anderson et al. (1989): Bernier and Jolles (1984); Bernier et al. (1986); Schoentgen et al. (1987); Jones and Hall (1991); Perry et al. (1994); and Hori et al. (1994); each incorporated herein by reference. Exemplary suitable hybridization conditions include hybridization in about 7% sodium dodecyl sulfate (SDS). about 0.5 M NaPO₄, about 1 mM EDTA at about 50° C.; and washing with about 1% SDS at about 42° C.

I. Imaging

The present invention may also be used in combined treatment and imaging methods. preferably tumor treatment and imaging methods, based upon diagnostic and therapeutic binding ligands. Such methods are applicable for use in generating diagnostic, prognostic or imaging information for any angiogenic disease, as exemplified by arthritis, psoriasis and solid tumors, but including all the angiogenic diseases disclosed herein. Targeting agents and tumor binding proteins and antibodies that are linked to one or more detectable agents are thus used in pre-imaging angiogenic sites and tumors, forming a reliable image prior to the combined treatment of the invention.

Antibody and binding protein conjugates for use as diagnostic agents generally fall into two classes, those for use in in vitro diagnostics, such as in a variety of immunoassays, and those for use in vivo diagnostic protocols. Although preferred for use in in vivo diagnostic and imaging methods, the present invention may also be used in in vitro diagnostic tests, preferably either where samples can be obtained non-invasively and tested in high throughput assays and/or where the clinical diagnosis in ambiguous and confirmation is desired prior to combined coagulant treatment. In addition to the routine knowledge in the art, further description and enabling teaching concerning the use of immunodetection methods and kits to detect, and then treat, angiogenic diseases is specifically incorporated herein by reference from U.S. Pat. Nos. 6,342,219, 6,342,221 and 6,416,758.

The in vivo imaging aspects of the invention are intended for use in combined treatment and imaging methods wherein a targeting agent is linked to one or more detectable agents and used to form a reliable image of an angiogenic disease site or tumor prior to treatment, preferably using the same targeting agent linked to one or more coagulants. Such compositions and methods can be applied to the imaging and diagnosis of any angiogenic disease or condition, particularly malignant and non-malignant tumors, atherosclerosis and conditions in which an internal image is desired for diagnostic or prognostic purposes or to design treatment.

The angiogenic and/or anti-tumor imaging ligands or antibodies, or conjugates thereof, will generally comprise an anti-tumor antibody or binding ligand operatively attached, or conjugated to, a detectable label. “Detectable labels” are compounds or elements that can be detected due to their specific functional properties, or chemical characteristics, the use of which allows the component to which they are attached to be detected, and further quantified if desired. Preferably, the detectable labels are those detectable in vivo using non-invasive methods.

Many appropriate imaging agents are known in the art, as are methods for their attachment to antibodies and binding ligands (see, e g., U.S. Pat. Nos. 5,021,236 and 4,472,509, both incorporated herein by reference). Certain attachment methods involve the use of a metal chelate complex employing, for example, an organic chelating agent such a DTPA attached to the antibody (U.S. Pat. No. 4.472.509). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate.

An example of detectable labels are the paramagnetic ions. In this case. suitable ions include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), with gadolinium being particularly preferred.

Ions useful in other contexts, such as X-ray imaging, include but are not limited to lanthanum (III), gold (III), lead (II), and especially bismuth (III). Fluorescent labels include rhodamine, fluorescein and renographin. Rhodamine and fluorescein are often linked via an isothiocyanate intermediate.

In the case of radioactive isotopes for diagnostic applications, suitable examples include ¹⁴carbon, ⁵¹chromium, ³⁶chlorine, ⁵⁷cobalt, ⁵⁸cobalt, copper⁶⁷, ¹⁵²Eu, gallium⁶⁷. ³hydrogen, iodine¹²³, iodine¹²⁵, iodine¹³¹, indium¹¹¹, ⁵⁹iron, ³²phosphorus, rhenium¹⁸⁶, rhenium^{188,} ⁷⁵selenium, ³⁵sulphur, technetium^{99m} and yttrium⁹⁰. ¹²⁵I is often being preferred for use in certain embodiments, and technicium^{99m} and indium¹¹¹ are also often preferred due to their low energy and suitability for long range detection.

Radioactively labeled anti-tumor antibodies and binding ligands for use in the present invention may be produced according to well-known methods in the art. For instance, intermediary functional groups that are often used to bind radioisotopic metallic ions to antibodies are diethylenetriaminepentaacetic acid (DTPA) and ethylene diaminetetracetic acid (EDTA).

Monoclonal antibodies can also be iodinated by contact with sodium or potassium iodide and a chemical oxidizing agent such as sodium hypochlorite, or an enzymatic oxidizing agent, such as lactoperoxidase. Anti-tumor antibodies according to the invention may be labeled with technetium-⁹⁹m by a ligand exchange process, for example, by reducing pertechnate with stannous solution, chelating the reduced technetium onto a Sephadex column and applying the antibody to this column. Direct labeling techniques are also suitable, e g, by incubating pertechnate, a reducing agent such as SNCl₂, a buffer solution such as sodium-potassium phthalate solution, and the antibody.

Any of the foregoing type of detectably labeled antibodies and binding ligands may be used in the imaging aspects of the present invention. Although suitable for use in in vitro diagnostics, the present detection methods are more intended for forming an image of an angiogenic disease site or tumor of a patient prior to combined treatment involving coagulants. The in vivo diagnostic or imaging methods generally comprise administering to a patient a diagnostically effective amount of an antibody or binding ligand that is conjugated to a marker that is detectable by non-invasive methods. The antibody- or binding ligand-marker conjugate is allowed sufficient time to localize and bind to the angiogenic disease site or tumor. The patient is then exposed to a detection device to identify the detectable marker, thus forming an image of the angiogenic disease site or tumor.

The nuclear magnetic spin-resonance isotopes, such as gadolinium, are detected using a nuclear magnetic imaging device; and radioactive substances, such as technicium^{99m} or indium¹¹¹, are detected using a gamma scintillation camera or detector. U.S. Pat. No. 5,627,036 is also specifically incorporated herein by reference for purposes of providing even further guidance regarding the safe and effective introduction of such detectably labeled constructs into the blood of an individual, and means for determining the distribution of the detectably labeled annexin extracorporally, e.g., using a gamma scintillation camera or by magnetic resonance measurement.

Dosages for imaging embodiments are generally less than for therapy, but are also dependent upon the age and weight of a patient. A one time dose of between about 0.1, 0.5 or about 1 mg and about 9 or 10 mgs, and more preferably, of between about 1 mg and about 5-10 mgs of antibody- or binding ligand-conjugate per patient is contemplated to be useful.

J. Pharmaceutical Compositions

The therapeutic agents for use in the present invention will generally be formulated as pharmaceutical compositions. The pharmaceutical compositions of the invention will thus generally comprise an effective amount of any of the agents of the invention, whether intended for the first, second or concurrent treatment steps, dissolved or dispersed in a pharmaceutically acceptable carrier or aqueous medium. Certain types of combined therapeutics are also contemplated, and the same type of underlying pharmaceutical compositions may be employed for both single and combined medicaments.

The phrases “pharmaceutically or pharmacologically acceptable” refer to molecular entities and compositions that do not produce an adverse, allergic or other untoward reaction when administered to an animal, or a human, as appropriate. Veterinary uses are equally included within the invention and “pharmaceutically acceptable” formulations include formulations for both clinical and/or veterinary use.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. For human administration, preparations should meet sterility, pyrogenicity, general safety and purity standards as required by FDA Office of Biologics standards. Supplementary active ingredients can also be incorporated into the compositions.

“Unit dosage” formulations are those containing a dose or sub-dose of the administered ingredient adapted for a particular timed delivery. For example, exemplary “unit dosage” formulations are those containing a daily dose or unit or daily sub-dose or a weekly dose or unit or weekly sub-dose and the like.

J1. Injectable Formulations

The therapeutic agents for use in the present invention will most often be formulated for parenteral administration. e g., formulated for injection via the intravenous, intramuscular, sub-cutaneous. transdermal, or other such routes, including peristaltic administration and direct instillation into a tumor or disease site (intracavity administration). The preparation of an aqueous composition that contains such an antibody or immunoconjugate as an active ingredient will be known to those of skill in the art in light of the present disclosure. Typically, such compositions can be prepared as injectables, either as liquid solutions or suspensions: solid forms suitable for using to prepare solutions or suspensions upon the addition of a liquid prior to injection can also be prepared; and the preparations can also be emulsified.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form should be sterile and fluid to the extent that syringability exists. It should be stable under the conditions of manufacture and storage and should be preserved against the contaminating action of microorganisms, such as bacteria and fungi.

The therapeutic agents can be formulated into a sterile aqueous composition in a neutral or salt form. Solutions of therapeutic agents as free base or pharmacologically acceptable salts can be prepared in water suitably mixed with a surfactant, such as hydroxypropylcellulose. Pharmaceutically acceptable salts, include the acid addition salts (formed with the free amino groups of the protein), and those that are formed with inorganic acids such as, for example, hydrochloric or phosphoric acids, or such organic acids as acetic. trifluoroacetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Suitable carriers include solvents and dispersion media containing, for example, water, ethanol, polyol (for example, glycerol, propylene glycol, and liquid polyethylene glycol, and the like), suitable mixtures thereof, and vegetable oils. In many cases, it will be preferable to include isotonic agents, for example, sugars or sodium chloride. The proper fluidity can be maintained, for example, by the use of a coating, such as lecithin, by the maintenance of the required particle size in the case of dispersion and/or by the use of surfactants.

Under ordinary conditions of storage and use, all such preparations should contain a preservative to prevent the growth of microorganisms. The prevention of the action of microorganisms can be brought about by various antibacterial and antifungal agents, for example, parabens, chlorobutanol, phenol, sorbic acid, thimerosal, and the like. Prolonged absorption of the injectable compositions can be brought about by the use in the compositions of agents delaying absorption, for example, aluminum monostearate and gelatin.

Prior to or upon formulation, the therapeutic agents should be extensively dialyzed to remove undesired small molecular weight molecules, and/or lyophilized for more ready formulation into a desired vehicle, where appropriate. Sterile injectable solutions are prepared by incorporating the active agents in the required amount in the appropriate solvent with various of the other ingredients enumerated above, as desired, followed by filtered sterilization. Generally, dispersions are prepared by incorporating the various sterilized active ingredients into a sterile vehicle that contains the basic dispersion medium and the required other ingredients from those enumerated above.

In the case of sterile powders for the preparation of sterile injectable solutions, the preferred methods of preparation are vacuum-drying and freeze-drying techniques that yield a powder of the active ingredient, plus any additional desired ingredient from a previously sterile-filtered solution thereof.

Suitable pharmaceutical compositions in accordance with the invention will generally include an amount of the therapeutic agent admixed with an acceptable pharmaceutical diluent or excipient, such as a sterile aqueous solution, to give a range of final concentrations. depending on the intended use. The techniques of preparation are generally well known in the art as exemplified by Remington's Pharmaceutical Sciences, 16th Ed. Mack Publishing Company. 1980, incorporated herein by reference. For human administration, preparations should meet sterility, pyrogenicity, general safety and purity standards as required by FDA Office of Biological Standards. Upon formulation, the therapeutic agents will be administered in a manner compatible with the dosage formulation and in such amount as is therapeutically effective.

J2. Sustained Release Formulations

Formulations are easily administered in a variety of dosage forms, such as the type of injectable solutions described above, but other pharmaceutically acceptable forms are also contemplated. e.g., tablets, pills, capsules or other solids for oral administration, suppositories. pessaries, nasal solutions or sprays, aerosols, inhalants, liposomal forms and the like. Pharmaceutical “slow release” capsules or compositions may also be used. Slow release formulations are generally designed to give a constant drug level over an extended period and may be used to deliver therapeutic agents in accordance with the present invention.

Pharmaceutical “slow release” capsules or “sustained release” compositions or preparations may also be used. Slow release formulations are generally designed to give a constant drug level over an extended period and may be used to deliver therapeutic agents in accordance with the present invention. The slow release formulations are typically implanted in the vicinity of the disease site, for example, at the site of a tumor.

Suitable examples of sustained-release preparations include semipermeable matrices of solid hydrophobic polymers containing therapeutic agents, which matrices are in the form of shaped articles, e.g., films or microcapsule. Examples of sustained-release matrices include polyesters; hydrogels, for example, poly(2-hydroxyethyl-methacrylate) or poly(vinylalcohol); polylactides, e.g, U.S. Pat. No. 3,773,919; copolymers of L-glutamic acid and γ ethyl-L-glutamate; non-degradable ethylene-vinyl acetate; degradable lactic acid-glycolic acid copolymers, such as the Lupron Depot™ (injectable microspheres composed of lactic acid-glycolic acid copolymer and leuprolide acetate); and poly-D-(−)-3-hydroxybutyric acid.

While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., thus reducing biological activity and/or changing immunogenicity. Rational strategies are available for stabilization depending on the mechanism involved. For example, if the aggregation mechanism involves intermolecular S—S bond formation through thio-disulfide interchange, stabilization is achieved by modifying sulfhydryl residues, lyophilizing from acidic solutions, controlling moisture content, using appropriate additives, developing specific polymer matrix compositions, and the like.

J3. Liposomes and Nanocapsules

In certain embodiments, liposomes and/or nanoparticles may also be employed with the therapeutic agents. The formation and use of liposomes is generally known to those of skill in the art, as summarized below.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Phospholipids can form a variety of structures other than liposomes when dispersed in water, depending on the molar ratio of lipid to water. At low ratios the liposome is the preferred structure. The physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

Liposomes interact with cells via four different mechanisms: Endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. Varying the liposome formulation can alter which mechanism is operative, although more than one may operate at the same time.

Nanocapsules can generally entrap compounds in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention, and such particles may be are easily made.

J4. Ophthalmic Formulations

Many diseases with an angiogenic component are associated with the eye and can be treated by the present invention. In selecting targeting agents for use in treating angiogenic diseases associated with the eye, a targeting agent that binds to a prominent angiogenic marker may be preferred, such as, e.g., a targeting agent that binds to VEGF. As such therapeutics can be readily administered to the eye, localization will not be a problem. In any event, as the sensitizing or pre-treatment aspects of the invention enable lower doses of the treatment or second agents to be employed, and coagulants exert little if any adverse effects even if mis-targeted, there are minimal safety concerns in treating eye diseases according to the invention.

Exemplary diseases associated with corneal neovascularization that can be treated according to the present invention include, but are not limited to, diabetic retinopathy, retinopathy of prematurity, corneal graft rejection, neovascular glaucoma and retrolental fibroplasia, epidemic keratoconjunctivitis, Vitamin A deficiency, contact lens overwear, atopic keratitis, superior limbic keratitis, pterygium keratitis sicca, sjogrens, acne rosacea, phylectenulosis, syphilis, Mycobacteria infections, lipid degeneration, chemical burns. bacterial ulcers, fungal ulcers, Herpes simplex infections, Herpes zoster infections, protozoan infections, Kaposi sarcoma, Mooren ulcer, Terrien's marginal degeneration, mariginal keratolysis, trauma, rheumatoid arthritis, systemic lupus, polyarteritis, Wegeners sarcoidosis, Scleritis, Steven's Johnson disease, periphigoid radial keratotomy, and corneal graph rejection.

Diseases associated with retinal/choroidal neovascularization that can be treated according to the present invention include, but are not limited to, diabetic retinopathy, macular degeneration, sickle cell anemia, sarcoid, syphilis, pseudoxanthoma elasticum, Pagets disease, vein occlusion, artery occlusion, carotid obstructive disease, chronic uveitis/vitritis, mvcobacterial infections, Lyme's disease, systemic lupus erythematosis, retinopathy of prematurity, Eales disease, Bechets disease, infections causing a retinitis or choroiditis, presumed ocular histoplasmosis, Bests disease, myopia, optic pits, Stargarts disease, pars planitis, chronic retinal detachment, hyperviscosity syndromes, toxoplasmosis, trauma and post-laser complications.

Other diseases that can be treated according to the present invention include, but are not limited to, diseases associated with rubeosis (neovascularization of the angle) and diseases caused by the abnormal proliferation of fibrovascular or fibrous tissue including all forms of proliferative vitreoretinopathy, whether or not associated with diabetes.

The therapeutic agents of the present invention may thus be advantageously employed in the preparation of pharmaceutical compositions suitable for use as ophthalmic solutions, including those for intravitreal and/or intracameral administration. For the treatment of any of the foregoing or other disorders the therapeutic agents are administered to the eye or eyes of the subject in need of treatment in the form of an ophthalmic preparation prepared in accordance with conventional pharmaceutical practice, see for example “Remington's Pharmaceutical Sciences” (Mack Publishing Co., Easton, Pa.).

The ophthalmic preparations will contain a therapeutic agent in a concentration from about 0.01 to about 1% by weight, preferably from about 0.05 to about 0.5% in a pharmaceutically acceptable solution. suspension or ointment. Some variation in concentration will necessarily occur, depending on the particular compound employed, the condition of the subject to be treated and the like, and the person responsible for treatment will determine the most suitable concentration for the individual subject. The ophthalmic preparation will preferably be in the form of a sterile aqueous solution containing, if desired, additional ingredients, for example preservatives, buffers, tonicity agents, antioxidants and stabilizers, nonionic wetting or clarifying agents, viscosity-increasing agents and the like.

Suitable preservatives for use in such a solution include benzalkonium chloride. benzethonium chloride, chlorobutanol, thimerosal and the like. Suitable buffers include boric acid, sodium and potassium bicarbonate, sodium and potassium borates, sodium and potassium carbonate, sodium acetate, sodium biphosphate and the like, in amounts sufficient to maintain the pH at between about pH 6 and pH 8, and preferably, between about pH 7 and pH 7.5. Suitable tonicity agents are dextran 40. dextran 70, dextrose, glycerin, potassium chloride, propylene glycol, sodium chloride, and the like, such that the sodium chloride equivalent of the ophthalmic solution is in the range 0.9 plus or minus 0.2%.

Suitable antioxidants and stabilizers include sodium bisulfite, sodium metabisulfite, sodium thiosulfite, thiourea and the like. Suitable wetting and clarifying agents include polysorbate 80, polysorbate 20, poloxamer 282 and tyloxapol. Suitable viscosity-increasing agents include dextran 40, dextran 70, gelatin, glycerin, hydroxyethylcellulose, hydroxmethylpropylcellulose, lanolin, methylcellulose, petrolatum, polyethylene glycol, polyvinyl alcohol, polyvinylpyrrolidone, carboxymethylcellulose and the like. The ophthalmic preparation will be administered topically to the eye of the subject in need of treatment by conventional methods, for example in the form of drops or by bathing the eye in the ophthalmic solution.

J5. Topical Formulations

In the broadest sense, formulations for topical administration include those for delivery via the mouth (buccal) and through the skin. “Topical delivery systems” also include transdermal patches containing the ingredient to be administered. Delivery through the skin can further be achieved by iontophoresis or electrotransport, if desired.

Formulations suitable for topical administration in the mouth include lozenges comprising the ingredients in a flavored basis, usually sucrose and acacia or tragacanth; pastiles comprising the active ingredient in an inert basis such as gelatin and glycerin, or sucrose and acacia; and mouthwashes comprising the ingredient to be administered in a suitable liquid carrier.

Formulations suitable for topical administration to the skin include ointments, creams, gels and pastes comprising the ingredient to be administered in a pharmaceutical acceptable carrier. The formulation of therapeutic agents for topical use, such as in creams, ointments and gels, includes the preparation of oleaginous or water-soluble ointment bases, will be well known to those in the art in light of the present disclosure. For example, these compositions may include vegetable oils, animal fats, and more preferably, semisolid hydrocarbons obtained from petroleum. Particular components used may include white ointment, yellow ointment, cetyl esters wax, oleic acid, olive oil, paraffin, petrolatum, white petrolatum, spermaceti, starch glycerite, white wax, yellow wax, lanolin, anhydrous lanolin and glyceryl monostearate. Various water-soluble ointment bases may also be used, including glycol ethers and derivatives, polyethylene glycols, polyoxyl 40 stearate and polysorbates.

Formulations for rectal administration may be presented as a suppository with a suitable base comprising, for example, cocoa butter or a salicylate. Formulations suitable for vaginal administration may be presented as pessaries, tampons, creams, gels, pastes, foams or spray formulations containing in addition to the active ingredient such carriers as are known in the art to be appropriate.

J6. Nasal Formulations

Local delivery via the nasal and respiratory routes is contemplated for treating various conditions. These delivery routes are also suitable for delivering agents into the systemic circulation. Formulations of active ingredients in carriers suitable for nasal administration are therefore also included within the invention, for example, nasal solutions, sprays, aerosols and inhalants. Where the carrier is a solid, the formulations include a coarse powder having a particle size, for example, in the range of 20 to 500 microns, which is administered. e.g., by rapid inhalation through the nasal passage from a container of the powder held close up to the nose.

Suitable formulations wherein the carrier is a liquid are useful in nasal administration. Nasal solutions are usually aqueous solutions designed to be administered to the nasal passages in drops or sprays and are prepared so that they are similar in many respects to nasal secretions, so that normal ciliary action is maintained. Thus, the aqueous nasal solutions usually are isotonic and slightly buffered to maintain a pH of 5.5 to 6.5. In addition, antimicrobial preservatives, similar to those used in ophthalmic preparations, and appropriate drug stabilizers, if required, may be included in the formulation. Various commercial nasal preparations are known and include, for example, antibiotics and antihistamines and are used for asthma prophylaxis.

Inhalations and inhalants are pharmaceutical preparations designed for delivering a drug or compound into the respiratory tree of a patient. A vapor or mist is administered and reaches the affected area. This route can also be employed to deliver agents into the systemic circulation. Inhalations may be administered by the nasal or oral respiratory routes. The administration of inhalation solutions is only effective if the droplets are sufficiently fine and uniform in size so that the mist reaches the bronchioles.

Another group of products, also known as inhalations, and sometimes called insufflations, comprises finely powdered or liquid drugs that are carried into the respiratory passages by the use of special delivery systems, such as pharmaceutical aerosols, that hold a solution or suspension of the drug in a liquefied gas propellant. When released through a suitable valve and oral adapter, a metered does of the inhalation is propelled into the respiratory tract of the patient. Particle size is of major importance in the administration of this type of preparation. It has been reported that the optimum particle size for penetration into the pulmonary cavity is of the order of 0.5 to 7 μm. Fine mists are produced by pressurized aerosols and hence their use in considered advantageous.

K. Diagnostic and Therapeutic Kits

This invention also provides diagnostic and therapeutic kits comprising therapeutic and coagulant-based agents for use in the combined treatment methods, or in imaging and treatment embodiments. Such kits will generally contain, in suitable container means, a pharmaceutically acceptable formulation of at least one therapeutic agent for use in the sensitizing aspect of the method and at least one coagulant-based agent for use in the treatment step of the method. The kits may also contain other pharmaceutically acceptable formulations, either for diagnosis/imaging or additional combination therapy. For example, such kits may contain any one or more of a range of chemotherapeutic or radiotherapeutic drugs; non-targeted or differently-targeted coagulants, anti-angiogenic agents; anti-tumor cell antibodies; and/or anti-tumor vasculature or anti-tumor stroma immunotoxins or coaguligands.

Although the kits may have a single container (container means) that contains a first or sensitizing therapeutic agent and a second coagulant-based agent, distinct containers are preferred for each desired agent. The agents for the sensitizing and treatment steps are thus maintained separately within distinct containers in the kit prior to administration to a patient. Where combined therapeutics are provided for either the sensitizing and treatment steps, a single solution may be pre-mixed, either in a molar equivalent combination, or with one component in excess of the other.

When the components of the kit are provided in one or more liquid solutions, the liquid solution is preferably an aqueous solution, with a sterile aqueous solution being particularly preferred. However, the components of the kit may be provided as dried powder(s). When reagents or components are provided as a dry powder, the powder can be reconstituted by the addition of a suitable solvent. It is envisioned that the solvent may also be provided in another container.

In the diagnostic kits, including both immunodetection and imaging kits, labeled targeting agents or antibodies are included, in addition to the same targeting agents or antibodies linked to one or more coagulants. For immunodetection, the antibodies may be bound to a solid support, such as a well of a microtitre plate, although antibody solutions or powders for reconstitution are preferred. The immunodetection kits preferably comprise at least a first immunodetection reagent. The immunodetection reagents of the kit may take any one of a variety of forms, including those detectable labels that are associated with or linked to the given antibody. Detectable labels that are associated with or attached to a secondary binding ligand are also contemplated. Exemplary secondary ligands are those secondary antibodies that have binding affinity for the first antibody.

Further suitable immunodetection reagents for use in the present kits include the two-component reagent that comprises a secondary antibody that has binding affinity for the first antibody, along with a third antibody that has binding affinity for the second antibody, the third antibody being linked to a detectable label. A number of exemplary labels are known in the art and all such labels may be employed in connection with the present invention. These kits may contain antibody-label conjugates either in fully conjugated form, in the form of intermediates, or as separate moieties to be conjugated by the user of the kit. The imaging kits will preferably comprise a targeting agent or antibody that is already attached to an in vivo detectable label.

However, the label and attachment means could be separately supplied. Either form of diagnostic kit may further comprise control agents, such as suitably aliquoted biological compositions, whether labeled or unlabeled, as may be used to prepare a standard curve for a detection assay. The components of the kits may be packaged either in aqueous media or in lyophilized form.

The containers of the therapeutic and diagnostic kits will generally include at least one vial, test tube, flask, bottle, syringe or other container means, into which the therapeutic and coagulant-based agents, and any other desired agent, are placed and, preferably. suitably aliquoted. As at least two separate components are preferred, the kits will preferably include at least two such container means. The kits may also comprise a third container means for containing a sterile, pharmaceutically acceptable buffer or other diluent.

The kits may also contain a means by which to administer the therapeutic agents to an animal or patient, e.g., one or more needles or syringes, or even an eye dropper, pipette, or other such like apparatus, from which the formulations may be injected into the animal or applied to a diseased area of the body. The kits of the present invention will also typically include a means for containing the vials, or such like, and other component, in close confinement for commercial sale, such as, e.g., injection or blow-molded plastic containers into which the desired vials and other apparatus are placed and retained.

L. Anti-Angiogenic Therapy

The present invention may be used to treat animals and patients with aberrant angiogenesis, such as that contributing to a variety of diseases and disorders. In light of the mechanisms discovered to operate in the tumor treatment aspects of the invention, including the upregulation of tissue factor on endothelial cells by VEGF, the invention is particularly contemplated for use in treating the many angiogenic diseases and disorders where VEGF plays a prominent role. Where coaguligands are used as part of the combined therapy, a targeting agent or antibody chosen for use in treating a non-life threatening angiogenic disease will preferably bind to a prominent angiogenic marker, such as, e.g., a targeting agent that binds to VEGF. However, the enhanced safety provided by the sensitizing step of the present methods allows lower doses of such treatment agents to be employed, meaning that potential mis-targeting is even less of a concern.

The most prevalent and/or clinically important angiogenic diseases, outside the field of cancer treatment, include arthritis, rheumatoid arthritis, psoriasis, atherosclerosis, diabetic retinopathy, age-related macular degeneration, Grave's disease, vascular restenosis, including restenosis following angioplasty, arteriovenous malformations (AVM), meningioma, hemangioma and neovascular glaucoma. Other targets for intervention include angiofibroma. atherosclerotic plaques, corneal graft neovascularization, hemophilic joints, hypertrophic scars, osler-weber syndrome, pyogenic granuloma retrolental fibroplasia, scleroderma, trachoma, vascular adhesions, synovitis, dermatitis, various other inflammatory diseases and disorders, and even endometriosis. Further diseases and disorders that are treatable by the invention, and the unifying basis of such angiogenic disorders, are set forth below.

One prominent disease in which angiogenesis is involved is rheumatoid arthritis, wherein the blood vessels in the synovial lining of the joints undergo angiogenesis. In addition to forming new vascular networks, the endothelial cells release factors and reactive oxygen species that lead to pannus growth and cartilage destruction. The factors involved in angiogenesis may actively contribute to, and help maintain, the chronically inflamed state of rheumatoid arthritis. Factors associated with angiogenesis also have a role in osteoarthritis, contributing to the destruction of the joint. Various targetable entities, including VEGF, have been shown to be involved in the pathogenesis of rheumatoid arthritis and osteoarthritis. Such markers can be targeted using a coagulant-targeting agent construct of the present invention.

Another important example of a disease mediated by angiogenesis is ocular neovascular disease. This disease is characterized by invasion of new blood vessels into the structures of the eye, such as the retina or cornea. It is the most common cause of blindness and is involved in approximately twenty eye diseases. In age-related macular degeneration, the associated visual problems are caused by an ingrowth of chorloidal capillaries through defects in Bruch's membrane with proliferation of fibrovascular tissue beneath the retinal pigment epithelium. Angiogenic damage is also associated with diabetic retinopathy, retinopathy of prematurity, corneal graft rejection, neovascular glaucoma and retrolental fibroplasia.

Other diseases associated with corneal neovascularization include, but are not limited to, epidemic keratoconjunctivitis, Vitamin A deficiency, contact lens overwear, atopic keratitis, superior limbic keratitis, pterygium keratitis sicca, sjogrens, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi sarcoma, Mooren ulcer, Terrien's marginal degeneration, mariginal keratolysis, rheumatoid arthritis, systemic lupus, polyarteritis, trauma, Wegeners sarcoidosis, Scleritis, Steven's Johnson disease, periphigoid radial keratotomy, and corneal graph rejection.

Diseases associated with retinal/choroidal neovascularization include, but are not limited to, diabetic retinopathy, macular degeneration, sickle cell anemia, sarcoid, syphilis, pseudoxanthoma elasticum, Pagets disease, vein occlusion, artery occlusion, carotid obstructive disease, chronic uveitis/vitritis, mycobacterial infections, Lyme's disease, systemic lupus erythematosis, retinopathy of prematurity, Eales disease, Bechets disease, infections causing a retinitis or choroiditis, presumed ocular histoplasmosis, Bests disease, myopia, optic pits, Stargarts disease, pars planitis, chronic retinal detachment, hyperviscosity syndromes, toxoplasmosis, trauma and post-laser complications.

Other diseases include, but are not limited to, diseases associated with rubeosis (neovascularization of the angle) and diseases caused by the abnormal proliferation of fibrovascular or fibrous tissue including all forms of proliferative vitreoretinopathy.

Chronic inflammation also involves pathological angiogenesis. Such disease states as ulcerative colitis and Crohn's disease show histological changes with the ingrowth of new blood vessels into the inflamed tissues. Bartonellosis, a bacterial infection found in South America, can result in a chronic stage that is characterized by proliferation of vascular endothelial cells.

Another pathological role associated with angiogenesis is found in atherosclerosis. The plaques formed within the lumen of blood vessels have been shown to have angiogenic stimulatory activity. There is particular evidence of the pathophysiological significance of angiogenic markers, such as VEGF, in the progression of human coronary atherosclerosis, as well as in recanalization processes in obstructive coronary diseases. The present invention provides an effective treatment for such conditions by targeting coagulants thereto.

One of the most frequent angiogenic diseases of childhood is the hemangioma. In most cases, the tumors are benign and regress without intervention. In more severe cases, the tumors progress to large cavernous and infiltrative forms and create clinical complications. Systemic forms of hemangiomas, the hemangiomatoses, have a high mortality rate. Therapy-resistant hemangiomas exist that cannot be treated with therapeutics currently in use, but are addressed by the invention.

Angiogenesis is also responsible for damage found in hereditary diseases such as Osler-Weber-Rendu disease, or hereditary hemorrhagic telangiectasia. This is an inherited disease characterized by multiple small angiomas, tumors of blood or lymph vessels. The angiomas are found in the skin and mucous membranes, often accompanied by epistaxis (nosebleeds) or gastrointestinal bleeding and sometimes with pulmonary or hepatic arteriovenous fistula.

Angiogenesis is also involved in normal physiological processes such as reproduction and wound healing. Angiogenesis is an important step in ovulation and also in implantation of the blastula after fertilization. Prevention of angiogenesis according to the present invention could be used to induce amenorrhea, to block ovulation or to prevent implantation by the blastula. In wound healing, excessive repair or fibroplasia can be a detrimental side effect of surgical procedures and may be caused or exacerbated by angiogenesis. Adhesions are a frequent complication of surgery and lead to problems such as small bowel obstruction. This can also be treated by the invention.

Each of the foregoing diseases and disorders, along with all types of tumors, as described in the following sections, can be effectively treated by the present invention in accordance with the knowledge in the art, as disclosed in, e.g., U.S. Pat. No. 5,712,291 (specifically incorporated herein by reference), that unified benefits result from the application of anti-angiogenic strategies to the treatment of angiogenic diseases.

M. Tumor Treatment

The combined coagulant-targeted therapies of the present invention are most preferably utilized in the treatment of tumors. Tumors in which angiogenesis is important include malignant tumors, and benign tumors, such as acoustic neuroma, neurofibroma, trachoma, pyogenic granulomas and BPH. Angiogenesis is particularly prominent in solid tumor formation and metastasis. However, angiogenesis is also associated with blood-born tumors. such as leukemias, and various acute or chronic neoplastic diseases of the bone marrow in which unrestrained proliferation of white blood cells occurs, usually accompanied by anemia, impaired blood clotting, and enlargement of the lymph nodes, liver, and spleen. Angiogenesis also plays a role in the abnormalities in the bone marrow that give rise to leukemia-like tumors.

Angiogenesis is important in two stages of tumor metastasis. In the vascularization of the primary tumor, angiogenesis allows cells to enter the blood stream and to circulate throughout the body. After tumor cells have left the primary site, and have settled into the secondary, metastasis site, angiogenesis must occur before the new tumor can grow and expand. Therefore, prevention of angiogenesis can prevent metastasis of tumors and contain the neoplastic growth at the primary site, allowing treatment by other therapeutics, particularly, therapeutic agent-targeting agent constructs.

Aside from angiogenesis, the unified procoagulant tendency of tumor vasculature means that the present invention can be preferably exploited for the treatment of malignant solid tumors. The invention is thus broadly applicable to the treatment of any malignant tumor having a vascular component. Such uses may be further combined with chemotherapeutic, radiotherapeutic, apoptopic, non-targeted or differently-targeted coagulants, anti-angiogenic agents and/or immunotoxins or coaguligands.

Typical vascularized tumors for treatment are the solid tumors, particularly carcinomas, which require a vascular component for the provision of oxygen and nutrients. Exemplary solid tumors that may be treated using the invention include, but are not limited to, carcinomas of the lung, breast, ovary, stomach, pancreas, larynx, esophagus, testes, liver, parotid, biliary tract, colon, rectum, cervix, uterus, endometrium, kidney, bladder, prostate, thyroid, squamous cell carcinomas, adenocarcinomas, small cell carcinomas, melanomas, gliomas, glioblastomas, neuroblastomas, and the like. WO 98/45331 is also incorporated herein by reference to further exemplify the variety of tumor types that may be effectively treated.

Knowledge of the role of angiogenesis in the maintenance and metastasis of tumors has led to a prognostic indicator for cancers such as breast cancer. The amount of neovascularization found in the primary tumor was determined by counting the microvessel density in the area of the most intense neovascularization in invasive breast carcinoma. A high level of microvessel density was found to correlate with tumor recurrence. Control of angiogenesis by the therapies of the present invention will reduce or negate the recurrence of such tumors.

The present invention is contemplated for use in the treatment of any patient that presents with a solid tumor. In that this invention provides a range of agents and coagulants that may be directed against solid tumors, a particular coagulant may be chosen to match a tumor of small, moderate or large size, so that the patients in such categories are likely to receive more significant benefits from treatment in accordance with the methods and compositions provided herein.

Therefore, in general, the invention can be used to treat tumors of all sizes, including those about 0.3-0.5 cm and upwards, tumors of greater than 0.5 cm in size and patients presenting with tumors of between about 1.0 and about 2.0 cm in size, although tumors up to and including the largest tumors found in humans may also be treated.

The present invention can also be used as a preventative or prophylactic treatment, so use of the invention is certainly not confined to the treatment of patients having tumors of only moderate or large sizes. There are various reasons underlying this aspect of the breadth of the invention, some connected with the choice of coagulant. For example, patients with metastatic tumors considered as small in size or in the early stages of metastatic tumor seeding may be treated according to the invention, optionally with a chemotherapeutic agent. Given that the coagulants of the invention are generally administered into the systemic circulation of a patient, they will naturally have effects on the secondary, smaller and metastatic tumors, as well as any primary tumor.

The guidance provided herein regarding the most suitable patients for use in connection with the present invention is intended as teaching that certain patient's profiles may assist with the selection of patients for treatment by the present invention. The pre-selection of certain patients, or categories of patients, does not in any way negate the basic usefulness of the present invention in connection with the treatment of all patients having a vascularized tumor. A further consideration is the fact that the assault on the tumor provided by the invention may predispose the tumor to further therapeutic treatment, such that the subsequent treatment results in an overall synergistic effect or even leads to total remission or cure.

It is not believed that any particular type of tumor should be excluded from treatment using the present invention. However, the type of tumor cells may be relevant to the use of the invention in combination with tertiary therapeutic agents, particularly chemotherapeutics and anti-tumor cell immunotoxins. As the effect of the present therapy is to destroy and/or prevent regrowth of the tumor vasculature, and as the vasculature is substantially or entirely the same in all solid tumors, it will be understood that the present methodology is widely or entirely applicable to the treatment of all solid tumors, irrespective of the particular phenotype or genotype of the tumor cells themselves.

Therapeutically effective combined doses are readily determinable using data from an animal model, as shown in the studies detailed herein, and from clinical data using a range of therapeutic agents. Experimental animals bearing solid tumors are frequently used to optimize appropriate therapeutic doses prior to translating to a clinical environment. Such models are known to be very reliable in predicting effective anti-cancer strategies. For example, mice bearing solid tumors, such as used in the Examples, are widely used in pre-clinical testing. The inventors have used such art-accepted mouse models to determine working ranges of coagulant-based constructs that give beneficial anti-tumor effects with minimal toxicity.

In terms of the treatment, i.e, the coagulant step of the tumor therapy, bearing in mind the attendant safety benefits associated with the overall invention, one may refer to the scientific and patent literature on the success of using anti-vascular therapies alone. By way of example, each of U.S. Pat. Nos. 5,855,866; 5.877,289; 5.965,132; 6.051.230; 6,004,555; 5,776,427; 6,004,554; 6,036,955; and 6,093,399 are incorporated herein by reference for the purpose of further describing the use of such agents. In the present case, the combined therapies have improved safety margins due to the sensitizing step, which enhances the therapeutic use of the invention and permits lower doses of tumor-based coagulants to be used.

As is known in the art, there are realistic objectives that may be used as a guideline in connection with pre-clinical testing before proceeding to clinical treatment. However, due to the safety already demonstrated in accepted models, pre-clinical testing of the present invention will be more a matter of optimization, rather than to confirm effectiveness. Thus, pre-clinical testing may be employed to select the most advantageous agents, doses or combinations.

Any combined method or medicament that results in any consistent detectable tumor vasculature regression and/or destruction, thrombosis and anti-tumor effects will still define a useful invention. Regressive, destructive, thrombotic and necrotic effects should be observed in between about 10% and about 40-50% of the tumor blood vessels and tumor tissues. upwards to between about 50% and about 99% of such effects being observed. The present invention may also be effective against vessels downstream of the tumor, i.e., target at least a sub-set of the draining vessels, particularly as cytokines released from the tumor will be acting on these vessels, changing their antigenic profile.

It will also be understood that even in such circumstances where the anti-tumor effects of the combined therapy are towards the low end of this range, it may be that this therapy is still equally or even more effective than all other known therapies in the context of the particular tumor. It is unfortunately evident to a clinician that certain tumors cannot be effectively treated in the intermediate or long term, but that does not negate the usefulness of the present therapy, particularly where it is at least about as effective as the other strategies generally proposed.

In designing appropriate doses of combined therapeutics for the treatment of vascularized tumors, one may readily extrapolate from the animal studies described herein in order to arrive at appropriate doses for clinical administration. To achieve this conversion, one would account for the mass of the agents administered per unit mass of the experimental animal and, preferably, account for the differences in the body surface area between the experimental animal and the human patient. All such calculations are well known and routine to those of ordinary skill in the art.

Notwithstanding the dosage ranges for coaguligands and naked tissue factor, it will be understood that, given the parameters and detailed guidance presented herein, further variations in the active or optimal ranges will be encompassed within the present invention. It will thus be understood that lower doses may be more appropriate in combination with certain agents, and that high doses can still be tolerated, particularly given the enhanced safety of the present constructs. The use of human or humanized antibodies or binding proteins renders the present invention even safer for clinical use, further reducing the chances of significant toxicity or side effects in healthy tissues.

The intention of the therapeutic regimens of the present invention is generally to produce significant anti-tumor effects whilst still keeping the dose below the levels associated with unacceptable toxicity. In addition to varying the dose itself, the administration regimen can also be adapted to optimize the treatment strategy.

In administering the particular doses themselves, one would preferably provide a pharmaceutically acceptable composition (according to FDA standards of sterility, pyrogenicity, purity and general safety) to the patient systemically. Intravenous injection is generally preferred, and the most preferred method is to employ a continuous infusion over a time period of about 1 or 2 hours or so. Although it is not required to determine such parameters prior to treatment using the present invention, it should be noted that the studies detailed herein result in at least some thrombosis being observed specifically in the blood vessels of a solid tumor within about 12-24 hours of injection, and that widespread tumor necrosis is also observed in this period.

Aside from the dose reductions that may now advantageously be used in light of the sensitizing aspects of the invention, more standard doses of coaguligands may still be employed with certain sensitizing protocols. Accordingly, the coaguligand doses for use in human patients may be between about 1 mg and about 500 mgs antibody per patient; preferably, between about 7 mgs and about 140 mgs antibody per patient; more preferably, between about 10 mgs and about 10 mgs antibody per patient; and even more preferably, between about 56 mgs and about 84 mgs antibody per patient,

Accordingly, using this information, the inventors contemplate that useful low doses of coaguligands for human administration will be about 0.1, 1, 2, 3, 4, 5, 6, 7, 8, 9, 10, 15, 20, 25 or about 30 mgs or so per patient; and useful high doses of coaguligands for human administration will be about 175, 200, 225 250, 275, 300, 325, 350, 375, 400, 425, 450, 475 or about 500 mgs or so per patient. Useful intermediate doses of coaguligands for human administration are contemplated to be about 35, 40, 50, 60, 70, 80, 90, 100, 125, 140 or about 150 mgs or so per patient. Dosage ranges of between about 5-100 mgs. about 10-80 mgs. about 20-70 mgs, about 25-60 mgs. or about 30-50 mgs or so of coaguligand per patient may be used. However, any particular range using any of the foregoing recited exemplary doses or any value intermediate between the particular stated ranges is contemplated.

Turning to naked tissue factor, although reduced doses may now be used in light of the sensitizing aspects of the invention, more standard doses of naked tissue factor can again be employed with certain sensitizing protocols. In taking the successful doses of therapeutics used in the mouse studies, and applying standard calculations based upon mass and surface area, effective standard doses of naked tissue factor for use in human patients would be between about 0.2 mgs and about 200 mgs of the TF construct per patient.

Useful low doses of naked tissue factor for use in human patients would be in and around 0.05, 0.1, 0.2, 0.5, 1, 2, 3, 4 and about 5 mg up to about 10 mg. Useful intermediate doses of naked tissue factor for human administration are contemplated to be about 20, 30, 40, 50, 60, 70, 80, 90 or 100 mgs or so per patient, with useful high doses being about 110, 120, 130, 140, 150, 160, 170, 180, 190 and about 200 mgs or so per patient. Doses between about 0.2 mg and about 180 mgs; between 0.5 and about 160 mgs; between 1 and about 150 mgs; between about 5 and about 125 mgs; between about 10 and about 100 mgs; between about 15 and about 80 mgs; between about 20 and about 65 mgs; between about 30 and about 50 mgs; about 40 mgs or so per patient are also contemplated.

Naturally, before wide-spread use, clinical trials will be conducted. The various elements of conducting a clinical trial, including patient treatment and monitoring, will be known to those of skill in the art in light of the present disclosure. The following information is being presented as a general guideline for use in establishing such trials.

Patients chosen for the first treatment studies will have failed to respond to at least one course of conventional therapy, and will have objectively measurable disease as determined by physical examination, laboratory techniques, and/or radiographic procedures. Any chemotherapy should be stopped at least 2 weeks before entry into the study. Where murine monoclonal antibodies or antibody portions are employed, the patients should have no history of allergy to mouse immunoglobulin.

Certain advantages will be found in the use of an indwelling central venous catheter with a triple lumen port. The therapeutics should be filtered, for example, using a 0.22μ filter, and diluted appropriately, such as with saline, to a final volume of 100 ml. Before use, the test sample should also be filtered in a similar manner, and its concentration assessed before and after filtration by determining the A₂₈₀. The expected recovery should be within the range of 87% to 99%, and adjustments for protein loss can then be accounted for.

The constructs may be administered over a period of approximately 4-24 hours. with each patient receiving 2-4 infusions at 2-7 day intervals. Administration can also be performed by a steady rate of infusion over a 7 day period. The infusion given at any dose level should be dependent upon any toxicity observed. Hence, if Grade II toxicity was reached after any single infusion, or at a particular period of time for a steady rate infusion, further doses should be withheld or the steady rate infusion stopped unless toxicity improved. Increasing doses should be administered to groups of patients until approximately 60% of patients showed unacceptable Grade III or IV toxicity in any category. Doses that are ⅔ of this value are defined as the safe dose.

Physical examination, tumor measurements, and laboratory tests should, of course, be performed before treatment and at intervals up to 1 month later. Laboratory tests should include complete blood counts, serum creatinine, creatine kinase, electrolytes, urea, nitrogen. SGOT, bilirubin, albumin, and total serum protein. Serum samples taken up to 60 days after treatment should be evaluated by radioimmunoassay for the presence of the administered therapeutic agent-targeting agent constructs, and antibodies against any portions thereof. Immunological analyses of sera, using any standard assay such as, for example, an ELISA or RIA, will allow the pharmacokinetics and clearance of the therapeutics to be evaluated.

To evaluate the anti-tumor responses, the patients should be examined at 48 hours to 1 week and again at 30 days after the last infusion. When palpable disease was present, two perpendicular diameters of all masses should be measured daily during treatment, within 1 week after completion of therapy, and at 30 days. To measure nonpalpable disease, serial CT scans could be performed at 1-cm intervals throughout the chest, abdomen, and pelvis at 48 hours to 1 week and again at 30 days. Tissue samples should also be evaluated histologically, and/or by flow cytometry, using biopsies from the disease sites or even blood or fluid samples if appropriate.

Clinical responses may be defined by acceptable measure. For example, a complete response may be defined by the disappearance of all measurable tumor 1 month after treatment. Whereas a partial response may be defined by a 50% or greater reduction of the sum of the products of perpendicular diameters of all evaluable tumor nodules 1 month after treatment, with no tumor sites showing enlargement. Similarly, a mixed response may be defined by a reduction of the product of perpendicular diameters of all measurable lesions by 50% or greater 1 month after treatment, with progression in one or more sites.

In light of results from clinical trials, such as those described above, an even more precise treatment regimen may be formulated. Even so, some variation in dosage may later be necessary depending on the condition of the subject being treated. The physician responsible for administration will, in light of the present disclosure, be able to determine the appropriate dose for the individual subject. Such optimization and adjustment is routinely carried out in the art, and by no means reflects an undue amount of experimentation.

N. Tertiary Combination Treatments

Although the present invention is itself a combination therapy, practice of the invention is by no means limited to the execution of two steps or to the use two agents. Accordingly, whether used for treating angiogenic diseases, such as arthritis, psoriasis, atherosclerosis, diabetic retinopathy, age-related macular degeneration, Grave's disease, vascular restenosis, hemangioma and neovascular glaucoma (or other diseases described above), or solid tumors, the present invention can be combined with other therapies.

The methods of the present invention may thus be combined with any other methods generally employed in the treatment of the particular tumor, disease or disorder that the patient exhibits. So long as a particular therapeutic approach is not known to be detrimental to the patient's condition in itself, and does not significantly counteract the treatment of the invention, its combination herewith is contemplated.

In connection solid tumor treatment, the present invention may be used in combination with classical approaches, such as surgery, radiotherapy, chemotherapy, and the like. The invention therefore provides combined therapies used simultaneously with, before, or after surgery, radiation treatment and/or the administration of conventional chemotherapeutic, radiotherapeutic, anti-angiogenic agents, anti-tubulin drugs, targeted immunotoxins and the like.

In terms of surgery, any surgical intervention may be practiced in combination with the present invention. In connection with radiotherapy, any mechanism for inducing DNA damage locally within tumor cells is contemplated, such as γ-irradiation, X-rays, UV-irradiation, microwaves and even electronic emissions and the like. The directed delivery of radioisotopes to tumor cells is also contemplated, and this may be used in connection with a targeting antibody or other targeting means.

Combination therapy for other vascular diseases is also contemplated. A particular example of such is benign prostatic hyperplasia (BPH), which may be treated in combination other treatments currently practiced in the art, for example, targeting of immunotoxins to markers localized within BPH, such as PSA.

N1. Chemotherapeutics

In certain embodiments, the present invention may be used in combination with a chemotherapeutic agent. Chemotherapeutic drugs can kill proliferating tumor cells, enhancing the necrotic areas created by the overall treatment of the invention. The drugs can be rendered even more effective when the invention prevents re-vascularization.

By destroying the tumor vessels, the present invention also enhances the action of the chemotherapeutics by retaining or trapping the drugs within the tumor. The chemotherapeutics are thus retained within the tumor, while the rest of the drug is cleared from the body. Tumor cells are thus exposed to a higher concentration of drug for a longer period of time. This entrapment of drug within the tumor makes it possible to reduce the dose of drug, making the tertiary treatment even safer as well as more effective.

A variety of chemotherapeutic agents may be used in the combined treatment methods disclosed herein. As will be understood by those of ordinary skill in the art, the appropriate doses of chemotherapeutic agents will be generally around those already employed in clinical therapies wherein the chemotherapeutics are administered alone or in combination with other chemotherapeutics.

N2. Immunotoxins

The present invention may be used in combination with immunotoxins in which the targeting portion thereof, e.g., antibody or ligand, is directed to a relatively specific marker of the tumor cells. Although the combined use of more than one tumor-vasculature or tumor-stroma targeting agent is certainly included within the invention, the present description concerns the exemplary combination with anti-tumor cell immunotoxins.

In these immunotoxins, the attached agents will be cytotoxic or pharmacological agents, particularly cytotoxic, cytostatic, anti-cellular or other anti-angiogenic agents having the ability to kill or suppress the growth or cell division of tumor cells. However, other suitable anti-cellular agents also include radioisotopes. In general, these aspects of the invention contemplate the use of any pharmacological agent that can be conjugated to a targeting agent, and delivered in active form to the tumor cells.

Exemplary anti-cellular agents include chemotherapeutic agents, as well as cytotoxins. Chemotherapeutic agents that may be used include: hormones, such as steroids; anti-metabolites, such as cytosine arabinoside, fluorouracil, methotrexate or aminopterin; anthracyclines; mitomycin C; vinca alkaloids; demecolcine; etoposide; mithramycin; anti-tumor alkylating agents, such as chlorambucil or melphalan. Other embodiments may include agents such as cytokines. Basically, any anti-cellular agent may be used, so long as it can be successfully conjugated to, or associated with, a targeting agent or antibody in a manner that will allow its targeting, internalization, release and/or overall effect at the site of the targeted cells.

There may be circumstances, such as when the target antigen does not internalize by a route consistent with efficient intoxication by the toxic compound, where one will desire to target chemotherapeutic agents, such as anti-tumor drugs, cytokines, antimetabolites, alkylating agents, hormones, and the like. A variety of chemotherapeutic and other pharmacological agents have now been successfully conjugated to antibodies and shown to function pharmacologically, including doxorubicin, daunomycin, methotrexate, vinblastine, neocarzinostatin, macromycin, trenimon and α-amanitin.

In other circumstances, any potential side-effects from cytotoxin-based therapy may be eliminated by the use of DNA synthesis inhibitors, such as daunorubicin, doxorubicin, adriamycin, and the like. These agents are therefore preferred examples of anti-cellular agents for use in combination with the present invention. In terms of cytostatic agents, such compounds generally disturb the natural cell cycle of a target cell, preferably so that the cell is taken out of the cell cycle.

Any of the anti-tubulin drugs may be linked to form immunoconjugates for combined use with the present invention. These include colchicine, taxol, vinblastine, vincristine, vindescine and the combretastatins, such as combretastatin A, B and/or D, more particularly, combretastatins A-1, A-2, A-3, A-4, A-5, A-6, B-1, B-2. B-3, B-4, D-1 and combretastatin D-2.

A wide variety of cytotoxic agents are known that may be conjugated to antibodies and binding ligands. Examples include numerous useful plant-, fungus- or bacteria-derived toxins, which, by way of example, include various A chain toxins, particularly ricin A chain; ribosome inactivating proteins, such as saporin or gelonin; α-sarcin; aspergillin; restrictocin; ribonucleases, such as placental ribonuclease; diphtheria toxin; and pselidomonas exotoxin, to name just a few.

Of the toxins, ricin A chains are preferred. The most preferred toxin moiety for use herewith is toxin A chain that has been treated to modify or remove carbohydrate residues, so-called deglycosylated A chain (dgA). Deglycosylated ricin A chain is preferred because of its extreme potency, longer half-life, and because it is economically feasible to manufacture it in a clinical grade and scale.

It may be desirable from a pharmacological standpoint to employ the smallest molecule possible that nevertheless provides an appropriate biological response. One may thus desire to employ smaller A chain peptides that will provide an adequate anti-cellular response. To this end, it has been discovered that ricin A chain may be “truncated” by the removal of 30 N-terminal amino acids by Nagarase (Sigma), and still retain an adequate toxin activity. It is proposed that where desired, this truncated A chain may be employed in conjugates in accordance with the invention.

Alternatively, one may find that the application of recombinant DNA technology to the toxin A chain moiety will provide additional benefits in accordance the invention. In that the cloning and expression of biologically active ricin A chain has been achieved, it is now possible to identify and prepare smaller or otherwise variant peptides that nevertheless exhibit an appropriate toxin activity. Moreover, the fact that ricin A chain has now been cloned allows the application of site-directed mutagenesis, through which one can readily prepare and screen for A chain-derived peptides and obtain additional useful moieties for use in connection with the present invention.

N3. Naked Tissue Factor, Factor VIIa or Activators of Factor VII

In certain aspects of the invention in which the treatment step uses a non-targeted, coagulant-deficient tissue factor construct, i e., certain naked tissue factors, the therapy may also be combined with the administration of Factor VIIa or an activator of Factor VII. It is important to note that, during such combined, sensitizing treatments of the present invention, significant amounts of factor VIIa should not be made available to the systemic circulation in the presence of exogenous tTF, other than wherein the tTF is a coagulation-deficient tTF.

In combination with systemic administration of a sensitizing agent, the provision of tTF precomplexed with factor VIIa can result in thrombosis in non-tumor tissues, such as lung and heart. Although systemic administration of a sensitizing agent followed by a coaguligand or tTF alone is remarkably safe, because significant factor VIIa production is limited to local production in the tumor vessels, sensitizing treatment followed by precomplexed tTF and factor VIIa should be avoided. However, coagulation-deficient tTFs could potentially be used with care in such combined embodiments.

Studies are presented herein to demonstrate that, in treatments without pre-sensitization, the anti-tumor activity of various coagulation-deficient TF constructs is enhanced upon co-administration with Factor VIIa. Even using an experimental animal model of the HT29 tumor, which is notoriously difficult to coagulate, the co-administration of coagulation-deficient TF constructs and exogenous Factor VIIa resulted in considerable necrosis of the tumor tissue.

This data can be explained as tTF binds Factor VII but does not efficiently mediate its activation to Factor VIIa by Xa and adjacent Factor VIIa molecules. Providing a source of preformed (exogenous) Factor VIIa overcomes this block, enabling more efficient coagulation. The success of the combined coagulation-deficient TF and Factor VIIa treatment is generally based upon the surprising localization of the TF construct within the vasculature of the tumor. Absent such surprising localization and specific functional effects, the co-administration of Factor VIIa would not be meaningful in the context of tumor treatment, and may even be harmful as it may promote unwanted thrombosis in various healthy tissues. The combined use of tTF and Factor VIIa in a non-targeted manner has previously been proposed in connection with the treatment of hemophiliacs and patients with other bleeding disorders, in which there is a fundamental impairment of the coagulation cascade. In the present invention, the coagulation cascade is generally fully operative, and the therapeutic intervention concentrates this activity within a defined region of the body.

A further observation of the present invention is that the thrombotic activity of the Factor VII activation mutants of tTF (G164A) and tTF (W158R) was largely restored by Factor VIIa. These mutations lie within a region of tTF that is important for the conversion of Factor VII to Factor VIIa. As with tTF itself, the studies herein show that adding preformed Factor VIIa overcomes this block in coagulation complex formation. The invention exploits these and the aforementioned observations with a view to providing in vivo therapy of cancer.

Studies presented herein, in treatments without pre-sensitization, confirm that the co-administration of a Factor VII activation mutant variant of TF with preformed Factor VIIa results in considerable necrotic damage to the tumors, even in small tumor models that are not the most amenable to treatment with the present invention. This aspect of the invention is particularly surprising as it was not previously believed that such mutants would have any therapeutic utility in any embodiments other than, perhaps, in the competitive inhibition of TF as may be used to inhibit or reduce coagulation.

In particular tertiary embodiments, the present invention therefore involves injecting tTF (G164A), tTF (W158R) or an equivalent thereof into tumor bearing animals. The tTF mutant is then allowed to localize to tumor vessels and the residue is cleared. This is then followed by the injection of Factor VIIa, which allows the localized tTF mutants to express thrombotic activity.

Factor VII can be prepared as described by Fair (1983), and as shown in U. S. Pat. Nos. 5,374,617, 5,504,064 and 5,504,067, each of which is incorporated herein by reference. The coding portion of the human Factor VII cDNA sequence was reported by Hagen et al., (1986). The amino acid sequence from 1 to 60 corresponds to the pre-pro/leader sequence that is removed by the cell prior to secretion. The mature Factor VII polypeptide chain consists of amino acids 61 to 466. Factor VII is converted to its active form, Factor VIIa, by cleavage of a single peptide bond between arginine-212 and isoleucine-213.

Factor VII can be converted in vitro to Factor VIIa by incubation of the purified protein with Factor Xa immobilized on Affi-Gel™ 15 beads (Bio-Rad). Conversion can be monitored by SDS-polyacrylamide gel electrophoresis of reduced samples. Free Factor Xa in the Factor VIIa preparation can be detected with the chromogenic substrate methoxycarbonyl-D-cyclohexylglycyl-glycyl-arginine-p-nitroanilide acetate (Spectrozyme™ Factor Xa, American Diagnostica. Greenwich, Conn.) at 0.2 mM final concentration in the presence of 50 mM EDTA. Recombinant Factor VIIa can also be purchased from Novo Biolabs (Danbury, Conn.).

It may be desired to create a 1:1 ratio of a coagulation-deficient TF construct and Factor VIIa in a precomplex and to administer the precomplexed composition to the animal. Should this be desired, one would generally admix an amount of coagulation-deficient TF and an amount of Factor VIIa sufficient to allow the formation of an equimolar complex. To achieve this, it may be preferable to use a 2-3 molar excess of Factor VIIa in order to ensure that each of the coagulation-deficient TF molecules are adequately complexed. One would then simply separate the uncomplexed coagulation-deficient TF and Factor VIIa from the complexed mixture using any suitable technique, such as gel filtration. After formation of the TF:VIIa complex, one may simply administer the complex to a patient in need of treatment in a dose of between about not 0.2 mg and about 200 mg per patient.

As stated above, it may generally be preferred to administer the coagulation-deficient TF construct to a patient in advance, allowing the TF sufficient time to localize specifically within the tumor. Following such preadministration, one would then design an appropriate dose of Factor VIIa sufficient to coordinate and complex with the TF localized within the tumor vasculature. Again, one may design the dose of Factor VIIa in order to allow a 1:1 molar ratio of TF and Factor VIIa to form in the tumor environment. Given the differences in molecular weight of these two molecules, it will be seen that it would be advisable to add approximately twice the amount in milligrams of Factor VIIa in comparison to the milligrams of TF.

However, the foregoing analysis is merely exemplary, and any doses of Factor VIIa that generally result in an improvement in coagulation would evidently be of clinical significance. In this regard, it is notable that the studies presented herein in fact use a 16:1 excess of coagulation-deficient TF in comparison to Factor VIIa, which is generally about a 32-fold molar excess of the TF construct. Nevertheless, impressive coagulation and necrosis was specifically observed in the tumor. Therefore, it will be evident that the effective doses of Factor VIIa are quite broad. By way of example only, one may consider administering to a patient a dose of Factor VIIa between about 0.01 mg and about 500 mg per patient.

Although the detailed guidance provided above is believed to be sufficient to enable one of ordinary skill in the art how to practice these aspects of the invention, one may also refer to other quantitative analyses to assist in the optimization of the coagulation-deficient TF and Factor VIIa doses for administration. By way of example only, one may refer to U.S. Pat. Nos. 5,374,617; 5,504.064; and 5,504.067, which describe a range of therapeutically active doses and plasma levels of Factor VIIa.

Morrissey and Comp have reported that, in the context of bleeding disorders, the coagulation-deficient Tissue Factor may be administered in a dosage effective to produce in the plasma an effective level of between 100 ng/ml and 50 μg/ml, or a preferred level of between 1 μg/ml and 10 μg/ml or 60 to 600 μg/kg body weight, when administered systemically; or an effective level of between 10 μg/ml and 50 μg/ml, or a preferred level of between 10 μg/ml and 50 μg/ml, when administered topically (U.S. Pat. No. 5,504,064).

The Factor VIIa is administered in a dosage effective to produce in the plasma an effective level of between 20 ng/ml and 10 μg/ml. (1.2 to 600 μg/kg), or a preferred level of between 40 ng/ml and 700 μg/ml (2.4 to 240 μg/kg), or a level of between 1 μg Factor VIIa/ml and 10 μg Factor VIIa/ml when administered topically.

In general, one would administer coagulation-deficient Tissue Factor and Factor VII activator to produce levels of up to 10 μg coagulation-deficient Tissue Factor/ml plasma and between 40 ng and 700 μg Factor VIIa/ml plasma. While these studies were performed in the context of bleeding disorders, they have also relevance in the context of the present invention, in that levels must be effective but appropriately monitored to avoid systemic toxicity due to elevated levels of coagulation-deficient Tissue Factor and activated Factor VIIa. Therefore, the Factor VII activator is administered in a dosage effective to produce in the plasma an effective level of Factor VIIa, as defined above.

As described in U.S. Pat. No. 5,504,064, incorporated herein by reference, activators of endogenous Factor VII may also be administered in place of Factor VIIa itself. As described in the foregoing patent, Factor VIIa can also be formed in vivo, shortly before, at the time of, or preferably slightly after the administration of the coagulation-deficient Tissue Factors. In such embodiments, endogenous Factor VII is converted into Factor VIIa by infusion of an activator of Factor VIIa, such as Factor Xa (FXa) in combination with phospholipid (PCPS).

Activators of Factor VII in vivo include Factor Xa/PCPS, Factor IXa/PCPS, thrombin, Factor XIIa, and the Factor VII activator from the venom of Oxyuranus scutellatus in combination with PCPS. These have been shown to activate Factor VII to Factor VIIa in vitro. Activation of Factor VII to Factor VIIa for Xa/PCPS in vivo has also been measured directly. In general, the Factor VII activator is administered in a dosage between 1 and 10 μg/ml of carrier (U.S. Pat. No. 5.504,064).

The phospholipid can be provided in a number of forms such as phosphatidyl choline/phosphatidyl serine vesicles (PCPS). The PCPS vesicle preparations and the method of administration of Xa/PCPS is described in Giles et al., (1988), the teachings of which are specifically incorporated herein. Other phospholipid preparations can be substituted for PCPS. so long as they accelerate the activation of Factor VII by Factor Xa. Effectiveness, and therefore determination of optimal composition and dose, can be monitored as described below.

A highly effective dose of Xa/PCPS, which elevates Factor VIIa levels in vivo in the chimpanzee, has been reported to be 26 pmoles FXa+40 pmoles PCPS per kg body weight. That dose yielded an eighteen fold increase in endogenous levels of Factor VIIa (to 146 ng/ml). A marginally detectable effect was observed using a smaller dose in dogs, where the infusion of 12 pmoles Factor Xa+19 pmoles PCPS per kg body weight yielded a three fold increase in endogenous Factor VIIa levels. Accordingly, doses of Factor Xa that are at least 12 pmoles Factor Xa per kg body weight, and preferably 26 pmoles Factor Xa per kg body weight, should be useful. Doses of PCPS that are at least 19 pmoles PCPS per kg body weight, and preferably 40 pmoles PCPS per kg body weight, are similarly useful (U.S. Pat. No. 5,504,064).

The effectiveness of any infusible Factor VII activator can be monitored, following intravenous administration, by drawing citrated blood samples at varying times (at 2, 5, 10, 20, 30, 60, 90 and 120 min.) following a bolus infusion of the activator, and preparing platelet-poor plasma from the blood samples. The amount of endogenous Factor VIIa can then be measured in the citrated plasma samples by performing a coagulation-deficient Tissue Factor-based Factor VIIa clotting assay. Desired levels of endogenous Factor VIIa would be the same as the target levels of plasma Factor VIIa indicated for co-infusion of purified Factor VII and coagulation-deficient Tissue Factor. Therefore, other activators of Factor VII could be tested in vivo for generation of Factor VIIa, without undue experimentation, and the dose adjusted to generate the desirable levels of Factor VIIa, using the coagulation-deficient Tissue Factor-based Factor VIIa assay of plasma samples. The proper dose of the Factor VII activator (yielding the desired level of endogenous Factor VIIa) can then be used in combination with the recommended amounts of coagulation-deficient Tissue Factor.

Doses can be timed to provide prolong elevation in Factor VIIa levels. Preferably doses would be administered until the desired anti-tumor effect is achieved, and then repeated as needed to control bleeding. The half-life of Factor VIIa in vivo has been reported to be approximately two hours, although this could vary with different therapeutic modalities and individual patients. Therefore, the half-life of Factor VIIa in the plasma in a given treatment modality should be determined with the coagulation-deficient Tissue Factor-based clotting assay.

The following examples are included to demonstrate certain preferred embodiments of the invention. It will be appreciated by those of skill in the art that the techniques disclosed in the examples that follow represent techniques discovered by the inventor to function well in the practice of the invention, and thus can be considered to constitute certain preferred modes for its practice. However, those of skill in the art should, in light of the present disclosure, appreciate that many changes can be made in the specific embodiments that are disclosed and still obtain a like or similar result without departing from the spirit and scope of the invention.

### EXAMPLE I

### Class II Induction and Immunotoxin Targeting

This example describes successful therapy using an MHC Class II solid tumor model using the anti-tumor endothelial cell immunotoxin, MS/114dgA, and the anti-tumor cell immunotoxin. 11-4.1dgA, alone as well as in combination therapy.

Using a murine model for antibody-directed targeting of vascular endothelial cells in solid tumors, as described in Burrows et al. (1992, specifically incorporated herein by reference), one or both of anti-Class II and anti-Class I immunotoxins were tested. The anti-tumor effects of the anti-tumor endothelial cell immunotoxin, M5-114 dgA, were seen at dosages as low as 20 μg. Sections of the tumor, when H & F-stained. illustrated only surviving “islands” of tumor cells in a “sea” of necrotic cells.

Treatment with 40 μg of M5/115-dgA resulted in dramatic anti-tumor effects. Here, 30 days after tumor inoculation the mean tumor volume equated with day 16 in the controls. 72 hours after treating a 1.2 cm tumor with 100 μg of the anti-Class II immunotoxin M5/114 dgA, the pattern is similar to the 20 μg data, but much more dramatic in that virtually no “islands” of tumor cells remain. This pattern represents a complete necrosis of greater than 95% of the tumor diameter, leaving only a thin cuff of surviving tumor cells, presumably nourished by vessels in overlying skin.

To address this potential source of recurrence, i.e., the potential for a cuff of surviving tumor cells, combined therapy with both an antitumor (anti-Class I) and an anti-endothelial (anti-Class II) immunotoxin was undertaken. The results of this combination therapy demonstrate that both immunotoxins had a transient but noticeable effect in and of themselves, with the anti-tumor immunotoxin showing a slightly greater anti-tumor effect than the anti-tumor endothelial cell immunotoxin, although this might be a dosing effect. Truly dramatic synergistic results were seen when both were used in combination. When 100 μg of the anti-tumor immunotoxin was given on day 14, followed by 20 μg of the anti-tumor endothelial cell immunotoxin on day 16, one out of four cures were observed. When the order of administration was reversed, i.e., the anti-tumor endothelial cell immunotoxin given first, even more dramatic results were observed, with two out of four cures realized. The latter approach is the more logical in that the initial anti-endothelial cell therapy serves to remove tumor mass by partial necrosis, allowing better penetration into the tumor of the anti-tumor immunotoxin.

The findings from this model validate the concept of tumor vascular targeting and, in addition, demonstrate that this strategy is complimentary to that of direct tumor targeting. The theoretical superiority of vascular targeting over the conventional approach was established by comparing the in vivo antitumor effects of two immunotoxins, one directed against tumor endothelium, the other against the tumor cells themselves, in the same model. The immunotoxins were equally potent against their respective target cells in vitro but, while 100 μg of the tumor-specific immunotoxin had practically no effect against large solid C1300(Muγ) tumors, as little as 40 μg of the anti-tumor endothelial cell immunotoxin caused complete occlusion of the tumor vasculature and dramatic tumor regressions.

Despite causing thrombosis of all blood vessels within the tumor mass, the anti-tumor endothelial cell immunotoxin was not curative because a small population of malignant cells at the tumor-host interface survived and proliferated to cause the observed relapses 7-10 days after treatment. The proximity of these cells to intact capillaries in adjacent skin and muscle suggests that they derived nutrition from the extratumoral blood supply, but the florid vascularization and low interstitial pressure in those regions of the tumor rendered the surviving cells vulnerable to killing by the anti-tumor immunotoxin, so that combination therapy produced some complete remissions.

The time course study demonstrated that the anti-Class II immunotoxin exerted its antitumor activity via the tumor vasculature since endothelial cell detachment and diffuse intravascular thrombosis clearly preceded any changes in tumor cell morphology. In contrast with the anti-tumor immunotoxin, the onset of tumor regression in animals treated with the anti-tumor endothelial cell immunotoxin was rapid. Massive necrosis and tumor shrinkage were apparent in 48-72 hours after injection. Focal denudation of the endothelial living was evident within 2-3 hours, in keeping with the fast and efficient in vivo localization of M5/114 antibody and the endothelial cell intoxication kinetics of the immunotoxin (t 1/10=2 hours, t ½=12.6 hours.

As only limited endothelial damage is required to upset the hemostatic balance and initiate irreversible coagulation, many intratumoral vessels were quickly thrombosed with the result that tumor necrosis began within 6-8 hours of administration of the immunotoxin. This illustrates several of the strengths of vascular targeting in that an avalanche of tumor cell death swiftly follows destruction of a minority of tumor vascular endothelial cells. Thus, in contrast to conventional tumor cell targeting, anti-endothelial immunotoxins are effective even if they have short serum half lives and only bind to a subset of tumor endothelial cells.

MHC Class II antigens are also expressed by B-lymphocytes, some bone marrow cells, myeloid cells and some renal and gut epithelia in BALB/c nu/nu mice, however, therapeutic doses of anti-Class II immunotoxin did not cause any permanent damage to these cell populations. Splenic B cells and bone marrow myelocytes bound intravenously injected anti-Class II antibody but early bone marrow progenitors do not express Class II antigens and mature bone marrow subsets and splenic B cell compartments were normal 3 weeks after therapy, so it is likely that any Ia⁺ myelocytes and B cells killed by the immunotoxin were replaced from the stem cell pool. It is contemplated that the existence of large numbers of readily accessible B cells in the spleen prevented the anti-Class II immunotoxin from reaching the relatively inaccessible Ia⁺ epithelial cells but hepatic Kupffer cells were not apparently damaged by M5/114-dgA despite binding the immunotoxin. Myeloid cells are resistant to ricin A-chain immunotoxins, probably due to unique endocytic pathways related to their degradative physiologic function. No severe vascular-mediated toxicity was seen in the studies reported here because mice were maintained on oral antibiotics which minimized immune activity in the small intestine.

The findings described in this example demonstrate the therapeutic potential of the vascular targeting strategy against large solid tumors. As animal models for cancer treatment are widely accepted in the scientific community for their predictive value in regard to clinical treatment, the invention is also intended for use in man.

### EXAMPLE II

### Class II Induction and Coaguligand Targeting

The present example shows the specific coagulation of tumor vasculature in vivo that results following the administration of a tumor vasculature-targeted coagulant (“coaguligand”). In the coaguligand, a bispecific antibody is used as a delivery vehicle for truncated human Tissue Factor. This example also employs a Class II solid tumor model.

To improve the C1300 (Muγ) tumor model, the C1300 (Muγ) cell line was subcloned into a cell line that can grow without being mixed with its parental cell, C1300, but still express the I-A^{d} MHC Class II antigen on the endothelial cells of the tumor. An anti-I-A^{d} antibody (B21-2) was used that has a 5-10 fold higher affinity for its antigen than the initial anti-I-A^{d} antibody (M5/114.15.2) used in this model as determined by FACS. In vivo distribution studies with this new anti-I-A^{d} antibody showed the same tissue distribution pattern as did M5/114.15.2. Intense staining with B21-2 was seen in tumor vascular endothelium, light to moderate staining in Kuppfer cells in the liver, the marginal zones in the spleen and some areas in the small and large intestines. Vessels in other normal tissues were unstained.

TF9/10H10 (referred to as 10H10), a mouse IgG1, is reactive with human TF without interference of TF/factor VIIa activity. The bispecific antibody B21-2/10H10, and appropriate controls, were synthesized.

Intravenous administration of a coaguligand composed of B21-2/10H10 (20 μg) and tTF (16 μg) to mice bearing solid C1300 (Mu□) tumors caused tumors to assume a blackened, bruised appearance within 30 minutes. A histological study of the time course of events within the tumor revealed that 30 minutes after injection of coaguligand all vessels in all regions of the tumor were thrombosed. Vessels contained platelet aggregates, packed red cells and fibrin. At this time, tumor-cells were viable, being indistinguishable morphologically from tumor cells in untreated mice.

By 4 hours, signs of tumor cell distress were evident. The majority of tumor cells had begun to separate from one another and had developed pyknotic nuclei. Erythrocytes were commonly observed in the tumor interstitium. By 24 hours, advanced tumor necrosis was visible throughout the tumor. By 72 hours, the entire central region of the tumor had compacted into morphologically indistinct debris.

These studies indicated that the predominant occlusive effect of the B21-2/10H10-tTF coaguligand on tumor vessels is mediated through binding to Class II antigens on tumor vascular endothelium. In one of three of the tumors examined, a viable rim of tumor cells 5-10 cell layers thick was visible on the outskirts of the tumor where it was infiltrating into surrounding normal tissues. Immunohistochemical examination of serial sections of the same tumor revealed that the vessels in the regions of tumor infiltration lacked class II antigens.

Tumors from control mice which had received B21-2/10H10 bispecific antibody (20 μg) alone 30 minutes or 24 hours earlier showed no signs of infarction. No thrombi or morphological abnormalities were visible in paraffin sections of liver, kidney, lung, intestine, heart, brain, adrenals, pancreas and spleen taken from tumor-bearing mice 30 minutes, 4 hours and 24 hours after administration of coaguligand.

In anti-tumor studies in which a coaguligand composed of B21-2/10H10 and tTF was administered to mice with 0.8 cm diameter tumors, the tumors regressed to approximately half their pretreatment size. Repeating the treatment on the 7th day caused the tumors to regress further, usually completely. In 5/7 animals, complete regressions were obtained. Two of the mice subsequently relapsed four and six months later. These anti-tumor effects are statistically highly significant (P<0.001) when compared with all other groups.

At the end of the study, two mice which had been treated with diluent alone and which had very large tumors of 2.0 cm³ and 2.7 cm³ (i.e. 10-15% of their body weight) were given coaguligand therapy. Both had complete remissions although their tumors later regrew at the original site of tumor growth.

The present studies show that soluble human tTF became a powerful thrombogen for tumor vasculature when targeted by means of a bispecific antibody to tumor endothelial cells. In vitro coagulation studies showed that the restoration of thrombotic activity of tTF is mediated through its cross-linking to antigens on the cell surface.

Administration of a coaguligand directed against class II to mice having tumors with class II-expressing vasculature caused rapid thrombosis of blood vessels throughout the tumor. This was followed by infarction of the tumor and complete tumor regressions in a majority of animals. In those animals where complete regressions were not obtained, the tumors grew back from a surviving rim of tumor cells on the periphery of the tumor where it had infiltrated into the surrounding normal tissues. The vessels at the growing edge of the tumor lacked class II antigens, thus explaining the lack of thrombosis of these vessels by the coaguligand. It is likely that these surviving cells would have been killed by coadministering a drug acting on the tumor cells themselves, as was found previously (Example I).

The anti-tumor effects of the coaguligand were similar in magnitude to those obtained in the same tumor model with an immunotoxin composed of anti-class II antibody and deglycosylated ricin A-chain (Example I). One difference between the two agents is their rapidity of action. The coaguligand induced thrombosis of tumor vessels in less than 30 minutes whereas the immunotoxin took 6 hours to achieve the same effect. The immunotoxin acts more slowly because thrombosis is secondary to endothelial cell damage caused by the shutting down of protein syntheses.

A second and important difference between the immunotoxin and the coaguligand is that they have different toxic side effects. The immunotoxin caused a lethal destruction of class II-expressing gastrointestinal epithelium unless antibiotics were given to suppress class II induction by intestinal bacteria. The coaguligand caused no gastrointestinal damage, as expected because of the absence of clotting factors outside of the blood, but caused coagulopathies in occasional mice when administered at high dosage.

The findings described herein demonstrate the therapeutic potential of targeting human coagulation-inducing proteins to tumor vasculature. The induction of tumor infarction by targeting coagulation-inducing proteins to tumor endothelial cell markers is a valuable approach to the treatment of solid tumors. The coupling of human (or humanized) antibodies to human coagulation proteins to produce wholly human coaguligands is particularly contemplated, thus permitting repeated courses of treatment to be given to combat both the primary tumor and its metastases.

### EXAMPLE III

### Synthesis of Truncated Tissue Factor

tTF is herein designated as the extracellular domain of the mature Tissue Factor protein (amino acid 1-219 of the mature protein; as in SEQ ID NO: 1 of U.S. Pat. Nos. 6,156,321, 6,132,729 and 6,132,730, and WO 98/31394), all specifically incorporated herein by reference.

A. H₆[tTF]

H₆ Ala Met Ala[tTF]. The tTF complimentary DNA (cDNA) was prepared as follows: RNA from J-82 cells (human bladder carcinoma) was used for the cloning of tTF. Total RNA was isolated using the GlassMax™ RNA microisolation reagent (Gibco BRL). The RNA was reverse transcribed to cDNA using the GeneAmp RNA PCR kit (Perkin Elmer).

tTF cDNA was amplified using the same kit. PCR amplification was performed as suggested by the manufacturer. Briefly, 75 μM dNTP; 0.6 μM primer, 1.5 mM MgCl₂ were used and 30 cycles of 30″ at 95° C., 30″ at 55° C. and 30″ at 72° C. were performed.

The tTF was expressed as a fusion protein in a non-native state in E coli inclusion bodies using the expression vector H₆pQE-60 (Qiagen). The E. coli expression vector H₆ pQE-60 was used for expressing tTF (Lee et tat., 1994). The PCR amplified tTF cDNA was inserted between the NcoI and HindIII site. H₆ pQE-60 has a built-in (His)₆ encoding sequence such that the expressed protein has the sequence of (His)₆ at the N terminus, which can be purified on a Ni—NTA column. In addition, the fusion protein has a thrombin cleavage site and residues 1-219 of TF.

To purify tTF, tTF containing H₆ pQE-60 DNA was transformed to E. coli TG-1 cells. The cells were grown to OD₆₀₀=0.5 and IPTG was added to 30 μM to induce the tTF production. The cells were harvested after shaking for 18 h at 30° C. The cell pellet was denatured in 6 M Gu-HCl and the lysate was loaded onto a Ni—NTA column (Qiagen). The bound tTF was washed with 6 M urea and tTF was refolded with a gradient of 6 M−1 M urea at room temperature for 16 h. The column was washed with wash buffer (0.05 Na H₂ PO₄, 0.3 M NaCl, 10% glycerol) and tTF was eluted with 0.2 M Imidozole in wash buffer. The eluted tTF was concentrated and loaded onto a G-75 column. tTF monomers were collected.

B. tTF

Gly[tTF]. The GlytTF complimentary DNA (cDNA) was prepared the same way as described in the previous section except using a different 5′ primer.

The H6 pQE60 expression vector and the procedure for protein purification is identical to that described above except that the final protein product was treated with thrombin to remove the H₆ peptide. This was done by adding 1 part of thrombin (Sigma) to 500 parts of tTF (w/w), and the cleavage was carried out at room temperature for 18 h. Thrombin was removed from tTF by passage of the mixture through a Benzamidine Sepharose 6B thrombin affinity column (Pharmacia). The resultant tTF, designated tTF₂₁₉, consisted of residues 1-219 of TF plus an additional glycine at the N-terminus. It migrated as a single band of molecular weight 26 kDa when analyzed by SDS-PAGE, and the N-terminal sequence was confirmed by Edman degradation.

C. Cysteine-Modified tTFs

(His)₆-N′-cys'tTF₂₁₉-tTF, hereafter abbreviated to H₆-N′-cys-tTF₂₁₉, was prepared by mutating tTF₂₁₉ by PCR with a 5′ primer encoding a Cys in front of the N′-terminus of mature tTF. H₆-tTF₂₁₉-cys-C′ was prepared likewise using a 3′ primer encoding a Cys after amino acid 219 of tTF. Expression and purification were as for tTF₂₁₉ except that Ellman's reagent (5′5′-dithio-bis-2-nitrobenzoic acid) was applied after refolding to convert the N′- or C′-terminal Cys into a stable activated disulfide group. Thrombin cleavage removed the (His)₆ tag and converted the proteins into N′-cys-tTF₂₁₉ and tTF₂₁₉-cys-C′. The products were >95% pure as judged by SDS-polyacrylamide gel electrophoresis.

H₆-tTF₂₂₀-cys-C′ and H₆-tTF₂₂₁-cys-C′ were prepared by mutating tTF₂₁₉ by PCR with 3′ primers encoding Ile-Cys and Ile-Phe-Cys after amino acid 219 of tTF. Expression, refolding and purification were as for H₆-tTF₂₁₉-cys-C′.

### EXAMPLE IV

### Synthesis of Dimeric, Truncated Tissue Factor

The inventors reasoned that Tissue Factor dimers may be more potent than monomers at initiating coagulation. It is possible that native Tissue Factor on the surface of J82 bladder carcinoma cells may exist as a dimer (Fair et al., 1987). The binding of one Factor VII or Factor VIIa molecule to one Tissue Factor molecule may also facilitate the binding of another Factor VII or Factor VIIa to another Tissue Factor (Fair et al., 1987; Bach et al., 1986). Furthermore, Tissue Factor shows structural homology to members of the cytokine receptor family (Edgington et al., 1991) some of which dimerize to form active receptors (Davies and Wlodawer, 1995). The inventors therefore synthesized TF dimers, as follows. While the synthesis of dimers hereinbelow is described in terms of chemical conjugation, recombinant and other means for producing the dimers of the present invention are also contemplated by the inventors.

A. [tTF] Linker [tTF]

The Gly [tTF] Linker [tTF] with the structure Gly[tTF] (Gly)₄ Ser (Gly)₄ Ser (Gly)₄ Ser [tTF] was made. Two pieces of DNA were PCR amplified separately and were ligated and inserted into the vector.

PCR 1: Preparation of tTF and the 5′ half of the linker DNA. Gly[tTF] DNA was used as the DNA template. Further PCR conditions were as described in the tTF section. PCR 2: Preparation of the 3′ half of the linker DNA and tTF DNA. tTF DNA was used as the template in the PCR. The product from PCR 1 was digested with NcoI and BamH. The product from PCR 2 was digested with HindIII and BamH1. The digested PCR1 and PCR2 DNA were ligated with Ncol and HindIII-digested H₆ pQE 60 DNA.

For the vector constructs and protein purification, the procedures were the same as described in the Gly [tTF] section.

B. Cys [tTF] Linker [tTF]

The Cys [tTF] Linker [tTF] with the structure Ser Gly Cys [tTF 2-219] (Gly)₄ Ser (Gly)₄ Ser(Gly)₄ Ser [tTF] was also constructed. DNA was made by PCR. [tTF] linker [tTF] DNA was used as the template. The remaining PCR conditions were the same as described in the tTF section. The vector constructs and protein purification were all as described in the purification of H₆C[tTF].

C. [tTF] Linker [tTF]cys

The [tTF] Linker [tTF]cys dimer with the protein structure [tTF] (Gly)₄ Ser (Gly)₄ Ser (Gly)₄ Ser [tTF] Cys was also made. The DNA was made by PCR. [tTF] linker [tTF] DNA was used as the template. The remaining PCR conditions were the same as described in the tTF section. The vector constructs and protein purification were again performed as described in the purification of [tTF]cys section.

D. Chemically Conjugated Dimers

[tTF] Cys monomer, which had been treated with Ellman's reagent to convert the free Cys to an activated disulfide group, was reduced with half a molar equivalent of dithiothreitol. This generated free Cys residues in half of the molecules. The monomers are conjugated chemically to form [tTF] Cys-Cys [tTF] dimers. This is done by adding an equal molar amount of DTT to the protected [tTF] Cys at room temperature for 1 hr to deprotect and expose the cysteine at the C-terminus of [tTF] Cys. An equal molar amount of protected [tTF] Cys is added to the DTT/[tTF] Cys mixture and the incubation is continued for 18 h at room temperature. The dimers are purified on a G-75 gel filtration column. Dimers of H₆-tTF₂₂₀-cys-C′, H₆-tTF₂₂₁-cys-C′ and H₆-N′-cys-tTF₂₁₉ were prepared likewise. The Cys [tTF] monomer is conjugated chemically to form dimers using the same method.

### EXAMPLE V

### Synthesis of Truncated Tissue Factor Mutants

Three tTF mutants are described that lack the capacity to convert tTF-bound Factor VII to Factor VIIa. There is 300-fold less Factor VIIa in the plasma compared with Factor VII (Morrissey et al., 1993). Therefore, circulating mutant tTF should be less able to initiate coagulation and hence exhibit very low toxicity. However, once the mutant tTF has localized to the tumor site, as is surprisingly demonstrated herein, Factor VIIa may be injected to exchange with the tTF-bound Factor VII. The mutated proteins have the sequences shown in SEQ ID NO: 8 and SEQ ID NO: 9 of co-pending U.S. Pat. Nos. 6.156,321, 6,132.729 and 6,132,730, and WO 98/31394, all specifically incorporated herein by reference, and are active in the presence of Factor VIIa.

A. [tTF]G164A

The “[tTF]G164A∞ has the mutant protein structure with the amino acid 164 (Gly) of tTF₂₁₉ being replaced by Ala. The Chameleon double-stranded site directed mutagenesis kit (Stratagene) was used for generating the mutant. The DNA template is Gly[tTF] DNA. The G164A mutant is represented by SEQ ID NO: 9 of U.S. Pat. Nos. 6,156,321, 6,132,729 and 6,132,730, and WO 98/31394.

B. [tTF]W158R

The tryptophan at amino acid 158 of tTF₂₁₉ was mutated to an arginine by PCR with a primer encoding this change. Expression, refolding and purification was as for tTF₂₁₉. The mutated protein has the sequences shown in SEQ ID NO: 8 of U.S. Pat. Nos. 6,156,321, 6,132,729 and 6,132,730, and WO 98/31394.

C. [tTF]W158R S162A

The [tTF]W158R S162A is a double mutant in which amino acid 158 (Trp) of tTF₂₁₉ is replaced by Arg and amino acid 162 (Ser) is replaced by Ala. The same mutagenizing method is used as described for [tTF] G164A and [tTF]W158R using a mutagenizing primer. The foregoing vector constructs and protein purification procedures are the same as used for purifying Gly[tTF].

### EXAMPLE VI

### Preparation of tTF-Bispecific Antibody Adducts and Synthesis of Truncated Tissue Factor Conjugates

A. Preparation of tTF-Bispecific Antibody Adducts

Bispecific antibodies were constructed that had one Fab′ arm of the 10H10 antibody that is specific for a non-inhibitory epitope on tTF linked to one Fab′ arm of antibodies (OX7. Mac51, CAMPATH-2) of irrelevant specificity. When mixed with tTF, the bispecific antibody binds the tTF via the 10H10 arm, forming a non-covalent adduct. The bispecific antibodies were synthesized according to the method of Brennan et al. (1985; incorporated herein by reference) with minor modifications.

In brief, F(ab′)₂ fragments were obtained from the IgG antibodies by digestion with pepsin (type A; EC 3.4.23.1) and were purified to homogeneity by chromatography on Sephadex G100. F(ab′)₂ fragments were reduced for 16 h at 20° C. with 5 mM 2-mercaptoethanol in 0.1 M sodium phosphate buffer, pH 6.8, containing 1 mM EDTA (PBSE buffer) and 9 mM NaAsO₂. Ellman's reagent (ER) was added to give a final concentration of 25 mM and, after 3 h at 20° C. the Ellman's derivatized Fab′ fragments (Fab′-ER) were separated from unreacted ER on columns of Sephadex G25 in PBSE.

To form the bispecific antibody, Fab′-ER derived from one antibody was concentrated to approximately 2.5 mg/ml in an Amicon ultrafiltration cell and was reduced with 10 mM 2-mercaptoethanol for 1 h at 20° C. The resulting Fab′-SH was filtered through a column of Sephadex G25 in PBSE and was mixed with a 1:1-fold molar excess of Fab′-ER prepared from the second antibody. The mixtures were concentrated by ultrafiltration to approximately 3 mg/ml and were stirred for 16 h at 20° C. The products of the reaction were fractionated on columns of Sephadex G100 in PBS. The fractions containing the bispecific antibody (110 kDa) were concentrated to 1 mg/ml, and stored at 4° C. in 0.02% sodium azide.

To form the tTF-bispecific antibody adducts, the bispecific antibody was mixed with a molar equivalent of tTF or derivatives thereof for 1 hour at 4° C. The adduct eluted with a molecular weight of approximately 130 kDa on gel filtration columns, corresponding to one molecule of bispecific antibody linked to one molecule of tTF.

1. Preparation of IgG-H₆-N′-cys-tTF₂₁₉ and IgG-H₆-tTF₂₁₉-cys-C′

To 26 mg IgG at a concentration of 10 mg/ml in N₂-flushed phosphate-saline buffer was added 250 μg SMPT (Pharmacia) in 0.1 ml dry DMF. After stirring for 30 minutes at room temperature, the solution was applied to a column (1.6 cm diameter×30 cm) of Sephadex G25(F) equilibrated in the same buffer. The derivatized IgG was collected in a volume of 10 to 12 ml and concentrated to about 3.5 ml by ultrafiltration (Amicon, YM2 membrane). The H₆-N′-cys-tTF₂₁₉ or H₆-tTF₂₁₉-cys-C′ (15 mg) was reduced by incubation at room temperature in the presence of 0.2 mM DTT until all Ellman's agent was released (i.e. OD at 412 nm reached a maximum). It was then applied to the Sephadex G25(F) column (1.6 cm diameter×30 cm) equilibrated with N₂-flushed buffer.

The Cys-tTF (˜15 ml) was added directly to the derivatized IgG solution. The mixture was concentrated to about 5 ml by ultrafiltration and incubated at room temperature for 18 hours before resolution by gel filtration chromatography on Sephacryl S200. The peak containing material having a molecular weight of 175,000-200,000 was collected. This component consisted of one molecule of IgG linked to one or two molecules of tTF. The conjugates have the structure:

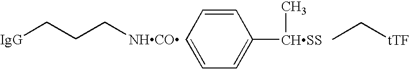

2. Preparation of Fab′-H6-N′-cys-tTF219

Fab′ fragments were produced by reduction of F(ab′)₂ fragments of IgG with 10 mM mercaptoethylamine. The resulting Fab′ fragments were separated from reducing agent by gel filtration on Sephadex G25. The freshly-reduced Fab′ fragment and the Ellman's modified H₆-N′-cys-tTF₂₁₉ were mixed in equimolar amounts at a concentration of 20 μM. The progress of the coupling reaction was followed by the increase in absorbance at 412 nm due to the 3-carboxylato-4-nitrothiophenolate anion released as a result of conjugation. The conjugate has the structure:

Fab′-SS-tTF

B. Synthesis of Tissue Factor Conjugates

1. Chemical Derivatization and Antibody Conjugation

Antibody tTF conjugates were synthesized by the linkage of chemically derivatized antibody to chemically derivatized tTF via a disulfide bond.

Antibody was reacted with a 5-fold molar excess of succinimidyl oxycarbonyl-α-methyl α-(2-pyridyldithio)toluene (SMPT) for 1 hour at room temperature to yield a derivatized antibody with an average of 2 pyridyldisulphide groups per antibody molecule. Derivatized antibody was purified by gel permeation chromatography.

A 2.5-fold molar excess of tTF over antibody was reacted with a 45-fold molar excess of 2-iminothiolane (2IT) for 1 hour at room temperature to yield tTF with an average of 1.5 sulfhydryl groups per tTF molecule. Derivatized tTF was also purified by gel permeation chromatography and immediately mixed with the derivatized antibody.

The mixture was left to react for 72 hours at room temperature and then applied to a Sephacryl S-300 column to separate the antibody-tTF conjugate from free tTF and released pyridine-2-thione. The conjugate was separated from free antibody by affinity chromatography on a anti-tTF column. The predominant molecular species of the final conjugate product was the singly substituted antibody-tTF conjugate (Mr approx. 176,000) with lesser amounts of multiply substituted conjugates (Mr≧approx. 202,000) as assessed by SDS-PAGE.

2. Conjugation of Cysteine-Modified tTF to Derivatized Antibody

Antibody-C[TF] and [tTF]C conjugates were synthesized by direct coupling of cysteine-modified tTF to chemically derivatized antibody via a disulfide bond.

Antibody was reacted with a 1 2-fold molar excess of 21T for 1 hour at room temperature to yield derivatized antibody with an average of 1.5 sulfhydryl groups per antibody molecule. Derivatized antibody was purified by gel permeation chromatography and immediately mixed with a 2-fold molar excess of cysteine-modified tTF. The mixture was left to react for 24 hours at room temperature and then the conjugate was purified by gel permeation and affinity chromatography as described above.

The predominant molecular species of the final conjugate was the singly substituted conjugate (Mr approx. 176,000) with lesser amounts of multiple substituted conjugates (Mr≧approx. 202.000) as assessed by SDS-PAGE.

3. Conjugation of Cysteine-Modified tTF to Fab′ Fragments

Antibody Fab′-C[tTF] and [tTF]C conjugates are prepared. Such conjugates may be more potent in vivo because they should remain on the cell surface for longer than bivalent conjugates due to their limited internalization capacity. Fab′ fragments are mixed with a 2-fold molar excess of cysteine-modified tTF for 24 hours and then the conjugate purified by gel permeation and affinity chromatography as described above.

### EXAMPLE VII

### Tumor Infarction by Truncated Tissue Factor

A. Methods

1. In Vitro Coagulation Assay

This assay was used to verify that tTF, various derivatives and mutants thereof, and immunoglobulin-tTF conjugates acquire coagulation inducing activity once localized at a cell surface. A20 lymphoma cells (1-A^{d} positive) (2×10⁶ cells/ml, 50 μl ) were incubated for 1 h at room temperature with a bispecific antibody (50 μg/ml, 25 μl ) consisting of a Fab′ arm of the B21-2 antibody directed against I-A^{d} linked to a Fab′ arm of the 10H10 antibody directed against a non-inhibitory epitope on tTF. The cells were washed at room temperature and varying concentrations of tTF, derivatives or mutants thereof, or immunoglobulin-tTF conjugates were added for 1 hour at room temperature. The bispecific antibody captures the tTF or tTF linked to immunoglobulin, bringing it into close approximation to the cell surface, where coagulation can proceed.

The cells were washed again at room temperature, resuspended in 75 μl of PBS and warmed to 37° C. Calcium (12.5 mM) and citrated mouse or human plasma (30 μl) were added. The time for the first fibrin strands to form was recorded. Clotting time was plotted against tTF concentration and curves compared with standard curves prepared using standard tTF₂₁₉ preparations.

In some studies, varying concentrations of recombinant human Factor VIIa were added together with tTF₂₁₉ and mutants thereof, to determine whether coagulation rate was enhanced by the presence of Factor VIIa.

2. Factor Xa Production Assays

This assay is useful in addition to or as an alternative to the in vitro coagulation assay to demonstrate that tTF and immunoglobulin-tTF conjugates acquire coagulation inducing activity once localized at a cell surface. The assay measures factor X to Xa conversion rate by means of a chromophore-generating substrate (S-2765) for factor Xa.

A20 cells (2×10⁷ cells) were suspended in 10 ml medium containing 0.2% w/v sodium azide. To 2.5 ml cell suspension were added 6.8 μg of B21-2/10H10 “capture” bispecific antibody for 50 minutes at room temperature. The cells were washed and resuspended in 2.5 ml medium containing 0.2% w/v sodium azide. The tTF and immunoglobulin-tTF conjugates dissolved in the same medium were distributed in 100 μl volumes at a range of concentrations into wells of 96-well microtiter plates. To the wells was then added 100 μl of the cell/bispecific antibody suspension. The plates were incubated for 50 minutes at room temperature.

The plates were centrifuged, the supernatants were discarded and the cell pellets were resuspended in 250 μl of Wash Buffer (150 mM NaCl; 50 mM Tris-HCl, pH 8; 0.2% w/v bovine serum albumin). The cells were washed again and cells resuspended in 100 μl of a 12.5-fold dilution of Proplex T (Baxter, Inc.) containing Factors II, VII, IX and X in Dilution Buffer (Wash Buffer supplemented with 12.5 mM calcium chloride). Plates were incubated at 37° C. for 30 minutes. To each well was added Stop Solution (12.5 mM sodium ethylenediaminetetracetic acid (EDTA)) in wash buffer. Plates were centrifuged. 100 μl of supernatant from each well were added to 11 μl of S-2765 (N-α-benzyloxycarbonyl-D-Arg-L-Gly-L-Arg-p-nitroanilide dihydrochloride, Chromogenix AB, Sweden). The optical density of each solution was measured at 409 nm. Results were compared to standard curves generated from standard tTF₂₁₉.

3. In Vivo Tumor Thrombosis

This model was used to demonstrate that tTF and immunoglobulin-tTF conjugates induced thrombosis of tumor blood vessels and caused tumor infarction in vivo.

Tumor test systems were of four types: i) 3LL mouse lung carcinoma growing subcutaneously in C57BL/6 mice; ii) C1300 mouse neuroblastoma growing subcutaneously in BALB/c nu/nu mice; iii) HT29 human colorectal carcinoma growing subcutaneously in BALB/c nu/nu mice; and iv) C1300 Muγ mouse neuroblastoma growing subcutaneously in BALB/c nu/nu mice. The C1300 Muγ tumor is an interferon-γ secreting transfectant derived from the C1300 tumor (Watanabe et al, 1989).

Further, the C1 300 (Muγ) tumor model of (Burrows, et al., 1992; incorporated herein by reference) was employed and modified as follows: (i) antibody B21-2 was used to target I-A^{d}; (ii) C1300(Muγ) tumor cells, a subline of C1300(Muγ)12 tumor cells, that grew continuously in BALB/c nu/nu mice were used; and (iii) tetracycline was omitted from the mice's drinking water to prevent gut bacteria from inducing I-A^{d} on the gastrointestinal epithelium. Unlike immunotoxins, coaguligands and Tissue Factor constructs do not damage I-A^{d}-expressing intestinal epithelium.

4. Tumor Establishment

To establish tumors, 10⁶ to 1.5×10⁷ tumor cells were injected subcutaneously into the right anterior flank of the mice. When tumors had grown to various sizes, mice were randomly assigned to different study groups. Mice then received an intravenous injection of 0.5 mg/kg of tTF alone or linked to IgG. Fab′, or bispecific antibody. Other mice received equivalent quantities IgG, Fab′ or bispecific antibody alone. The injections were performed slowly into one of the tail veins over approximately 45 seconds, usually followed by 200 μl of saline.

In some studies, the effect of administering cancer chemotherapeutic drugs on the thrombotic action of tTF on tumor blood vessels was investigated. Mice bearing subcutaneous HT29 human colorectal tumors of 1.0 cm diameter were given intraperitoneal injections of doxorubicin (1 mg/kg/day), camptothecin (1 mg/kg/day), etoposide (20 mg/kg/day) or interferon gamma (2×10⁵ units/kg/day) for two days before the tTF injection and again on the day of the tTF injection.

Twenty-four hours after being injected with tTF or immunoglobulin-tTF conjugates, the mice were anesthetized with metophane and were exsanguinated by perfusion with heparinized saline. Tumors and normal tissues were excised and immediately fixed in 3% (v/v) formalin. Paraffin sections were cut and stained with hematoxylin and eosin. Blood vessels having open lumens containing erythrocytes and blood vessels containing thrombi were counted. Paraffin sections were cut and stained with hematoxylin and eosin or with Martius Scarlet Blue (MSB) trichrome for the detection of fibrin.

5. Anti-Tumor Effects

Accepted animal models were used to determine whether administration of tTF or immunoglobulin-tTF conjugates suppressed the growth of solid tumors in mice. The tumor test systems were: i) L540 human Hodgkin's disease tumors growing in SCID mice; ii) C1300 Muγ (interferon-secreting) neuroblastoma growing in nu/nu mice; iii) H460 human non-small cell lung carcinoma growing in nu/nu mice. To establish solid tumors, 1.5×10⁷ tumor cells were injected subcutaneously into the right anterior flank of SCID or BALB/c nu/nu mice (Charles River Labs., Wilmingham, Mass.). When the tumors had grown to various diameters, mice were assigned to different experimental groups, each containing 4 to 9 mice.

Mice then received an intravenous injection of 0.5 mg/kg of tTF alone or linked to bispecific antibody. Other mice received equivalent quantities of bispecific antibody alone. The injections were performed over ˜45 seconds into one of the tail veins, followed by 200 μl of saline. The infusions were repeated six days later. Perpendicular tumor diameters were measured at regular intervals and tumor volumes were calculated.

B. Results

1. In vitro Coagulation by tTF and Variants

To target tTF to I-A^{d} on tumor vascular endothelium, the inventors prepared a bispecific antibody with the Fab′ arm of the B21-2 antibody, specific for I-A^{d}, linked to the Fab′ arm of the 10H10 antibody, specific for a non-inhibitory epitope on the C-module of tTF This bispecific antibody, B21-2/10H10, mediated the binding of tTF in an antigen-specific manner to I-A^{d} on A20 mouse B-lymphoma cells in vitro. When mouse plasma was added to A20 cells to which tTF had been bound by B21-2/10H10, it coagulated rapidly. Fibrin strands were visible 36 seconds after the addition of plasma to antibody-treated cells, as compared with 164 seconds when plasma was added to untreated cells. Only when tTF was bound to the cells was this enhanced coagulation observed: no effect on coagulation time was seen with cells incubated with tTF alone, with homodimeric F(ab′)₂, with Fab′ fragments, or with tTF plus bispecific antibodies that had only one of the two specificities needed for binding tTF to A20 cells.

There was a linear relationship between the logarithm of the number of tTF molecules bound to the cells and the rate of plasma coagulation by the cells. In the presence of cells alone, plasma coagulated in 190 seconds, whereas at 300,000 molecules of tTF per cell coagulation time was 40 seconds. Even with only 20,000 molecules per cell, coagulation was faster (140 seconds) than with untreated cells. These in vitro studies showed that the thrombogenic potency of tTF is enhanced by cell surface proximity mediated through antibody-directed binding to Class II antigens on the cell surface.

H₆-N′-cys-tTF₂₁₉ and H₆-tTF₂₁₉-cys-C′ were as active as tTF at inducing coagulation of plasma once bound via the bispecific antibody to A20 cells. Plasma coagulated in 50 seconds when H₆-N′-cys-tTF₂₁₉ and H₆-tTF₂₁₉-cys-C′ were applied at 3×10⁻⁹ M, the same concentration as for tTF. Thus, mutation of tTF to introduce a (His)₆ sequence and a Cys residue at the N′ or C′ terminus does not reduce its coagulation-inducing activity.

H₆-tTF₂₂₀-cys-C′, tTF₂₂₀-cys-C′, H₆-tTF₂₂₁-cys-C′ and tTF₂₂₁-cys-C′ were as active as tTF₂₁₉ at inducing coagulation of plasma once localized on the surface of A20 cells via the bispecific antibody, B21-2/10H10. With all samples at 5×10⁻¹⁰ M, plasma coagulated in 50 seconds.

2. In Vitro Coagulation by tTF Dimers

H₆-N′cys-tTF₂₁₉ dimer was as active as tTF₂₁₉ itself at inducing coagulation of plasma once localized on-the surface of A20 cells via the bispecific antibody, B21-2/10H10. At a concentration of 1-2×10⁻¹⁰ M. both samples induced coagulation in 50 seconds. In contrast, H₆-tTF₂₂₁-cys-C′ dimer was 4-fold less active than H₆-tTF₂₂₁-cys-C′ monomer or tTF₂₁₉ itself. At a concentration of 4×10⁻⁹M, H₆-tTF₂₂₁-cys-C′ dimer induced coagulation of plasma in 50 seconds, whereas the corresponding monomer needed to be applied at 1×10⁻⁹ M for the same effect on coagulation.

3. In vivo Tumor Thrombosis

In Example II, it was demonstrated that intravenous administration of the B21-2/10H10-tTF coaguligand induced selective thrombosis of tumor vasculature in mice bearing subcutaneous C1300(Muγ) neuroblastomas.

Surprisingly, it was also observed that there was a non-specific thrombotic action of tTF discernible in tumor vessels at later times: In tumors from mice which had been injected 24 hours previously with tTF alone or tTF mixed with the control bispecific antibody, OX7/10H10, the tumors assumed a blackened, bruised appearance starting within 30 minutes and becoming progressively more marked up to 24 hours. A histological study revealed that 24 hours after injection of tTF₂₁₉ practically all vessels in all regions of the tumor were thrombosed. Vessels contained platelet aggregates, packed red cells and fibrin. The majority of tumor cells had separated from one another and had developed pyknotic nuclei and many regions of the tumors were necrotic. These were most pronounced in the tumor core. Erythrocytes were commonly observed in the tumor interstitium.

Similar results were obtained when tTF₂₁₉ was administered to mice bearing large C1300 tumors (>1000 mm³). Again, virtually all vessels were thrombosed 24 hours after injection. Thus, the effects observed on C1300 Muγ tumors were not related to the interferon-γ secretion by the tumor cells.

Further studies were performed in C57BL/6 mice bearing large (>800 mm³) 3LL tumors. Again, thrombosis of tumor vessels was observed, though somewhat less pronounced than with the C1300 and C1300 Muγ tumor. On average 62% of 3LL tumor vessels were thrombosed.

Vessels in small (<500 mm³) C1 300 and C1300 Muγ were largely unaffected by tTF₂₁₉ administration. Thus, as the tumors grow, their susceptibility to thrombosis by tTF₂₁₉ increases. This is possibly because cytokines released by tumor cells or by host cells that infiltrate the tumor activate the tumor vascular endothelium, inducing procoagulant changes in the vessels.

Coaguligand treatment was well tolerated, mice lost no weight and retained normal appearance and activity levels. At the treatment dose of 0.6 mg/kg B21-2/10H10 plus 0.5 mg/kg tTF, toxicity was observed in only two of forty mice (thrombosis of tail vein). It is important to note that neither thrombi, nor histological or morphological abnormalities were visible in paraffin sections of liver, kidney, lung, intestine, heart, brain, adrenals, pancreas, or spleen from the tumor-bearing mice 30 minutes or 24 hours after administration of coaguligand or free tTF. Furthermore, no signs of toxicity (behavioral changes, physical signs, weight changes) were observed in treated animals.

4. Anti-Tumor Effects in C1300 Muγ Tumors

Intravenous administration of the B21-2/10H10-tTF coaguligand inhibited the growth of large (0.8 to 1.0 cm diameter) tumors in mice. The pooled results from three separate studies indicate that mice receiving B21-2/10H10-tTF coaguligand had complete tumor regressions lasting four months or more. These anti-tumor effects were significantly greater than for all other treatment groups (Example II).

Surprisingly, the inventors found that the anti-tumor effect of the B21-2/10H20-tTF coaguligand was attributable, in part, to a non-targeted effect of tTF. Tumors in mice receiving tTF alone or mixed with control bispecific antibodies (CAMPATH II/10H10 or B21-2/OX7) grew significantly more slowly than tumors in mice receiving antibodies or saline alone.

Mice bearing small (300 mm³) C1300 Muγ tumors were injected intravenously with 16-20 μg tTF₂₁₉. The treatment was repeated one week later. The first treatment with tTF₂₁₉ had a slight inhibitory effect on tumor growth, consistent with the lack of marked thrombosis observed with small tumors above. The second treatment had a substantially greater. statistically significant (P<0.01), effect on tumor growth, probably because the tumors had increased in size. One week after the second treatment with tTF₂₁₉, tumors were 60% of the size of tumors in mice receiving diluent alone. The greater effectiveness of the second injection probably derives from the greater thrombotic action of tTF₂₁₉ on vessels in large tumors, observed above.

5. Anti-Tumor Effects in other Systems

In addition to the effects in mice bearing C1300 Muγ tumors, similar anti-tumor effects were observed using other tumor types. In mice bearing H460 human lung carcinomas, the first treatment with tTF₂₁₉ was given when the tumors were small (250 mm³) and had little effect on growth rate. The second treatment with tTF₂₁₉ was given when the tumors were larger (900 mm³) and caused the tumors to regress to 550 mm³ before regrowing.

Anti-tumor effects were also observed in mice bearing HT29 human colorectal carcinomas. Nu/nu mice bearing large (1200 mm³) tumors on their flanks were injected intravenously with tTF₂₁₉ or PBS (control), and growth of the tumors was monitored each day for 10 days. The tumors in the tTF₂₁₉ treated mice discontinued growth for about 7 days after treatment, whereas the tumors in mice treated with PBS continued to grow unchecked.

### EXAMPLE VIII

Inhibition of Tumor Growth by Immunoglobulin-tTF Conjugate

1. Coagulation of Mouse Plasma by Immunoglobulin-TF Conjugates

IgG-H₆-N′-cys-tTF₂₁₉ was active at inducing coagulation of mouse plasma when localized on the surface of A20 cells by means of the bispecific antibody, B21-2/10H10. It induced coagulation in 50 seconds when applied at a tTF concentration of 5×10⁻⁹ M as compared with 1×10⁻⁹ M for non-conjugated tTF₂₁₉ and H₆-N′-cys-tTF₂₁₉. The coagulation inducing activity of IgG-H₆-N′-cys-tTF₂₁₉ is therefore reduced 5-fold relative to unconjugated H₆-N′-cys-tTF₂₁₉ or tTF₂₁₉ itself.

The slight reduction upon IgG conjugation could be because the IgG moiety of IgG-H₆. N′-cys-tTF₂₁₉ impedes access of the B21-2/10H10 bispecific antibody to the tTF moiety (i e., an artifactual reduction related to the assay method). It is probably not because the IgG moiety of IgG-H₆-N′-cys-tTF₂₁₉ interferes with formation of the coagulation initiation complexes because, in prior work, the inventors have found that the tTF moiety in an analogous construct. B21-2 IgG-H₆-N′-cys-tTF₂₁₉, is as active as tTF bound via B21-2/10H10 to I-A^{d} antigens on A20 cells. Similarly. B21-2 IgG-H₆-tTF₂₁₉-cys-C′ was as active at inducing coagulation as was the N′-linked conjugation.

IgG-H₆-N′-cys-tTF₂₁₉ and Fab′-H₆-N′-cys-tTF₂₁₉ were tested for their ability to convert Factor X to Xa in the presence of Factors II, VII and IX, once localized on the surface of A20 lymphoma cells by means of the bispecific antibody, B21-2/10H10. The Fab′-tTF construct was as active as H₆-N′-cys-tTF₂₁₉ itself at inducing Xa formation. The IgG-tTF construct was slightly (2-fold) less active than H₆-N′-cys-tTF₂₁₉ itself.

2. Inhibition of Tumor Growth

Mice bearing small (300 mm³) subcutaneous C1300 Muγ tumors were treated with tTF₂₁₉ or with a complex of tTF₂₁₉ and a bispecific antibody, OX7 Fab′/10H10 Fab′, not directed to a component of the tumor environment. The treatment was repeated 6 days later. The bispecific antibody was simply designed to increase the mass of the tTF₂₁₉ from 25 kDa to 135 kDa, and thus prolong its circulatory half life, and was not intended to impart a targeting function to tTF.

Tumors in mice treated with the immunoglobulin-tTF conjugate grew more slowly than those in mice receiving tTF₂₁₉ alone. Fourteen days after the first injection, tumors were 55% of the size of those in controls receiving diluent alone. In mice receiving tTF₂₁₉ alone, tumors were 75% of the size in controls receiving diluent alone.

### EXAMPLE IX

### Anti-Tumor Activity of Activation Mutants and Factor VIIa

1. Enhancement of Plasma Coagulation by VIIa

The ability of cell-associated tTF₂₁₉ to induce coagulation of mouse or human plasma was strongly enhanced in the presence of free Factor VIIa. In the absence of Factor VIIa, A20 cells treated with B21-2/10H10 bispecific antibody and 10⁻¹⁰ M tTF₂₁₉ coagulated plasma in 60 seconds, whereas in the presence of 13.5 nM Factor VIIa, it coagulated plasma in 20 seconds. This represents approximately a 100-fold enhancement in the coagulation-inducing potency of tTF in the presence of Factor VIIa. Even in the presence of 0.1 nM Factor VIIa, a 2-5 fold increase in coagulation-inducing potency of tTF was observed.

This finding leads to the aspects of the invention that concern the coadministration of Factor VIIa along with tTF or derivatives thereof, or with immunoglobulin-tTF conjugates, in order to enhance tumor vessel thrombosis in vivo.

2. Reduced Coagulation of Mouse Plasma by tTF Factor VII Activation Mutants

Mutations in W158 and G164 of tTF₂₁₉ have been reported to reduce markedly the ability of TF to induce coagulation of recalcified plasma (Ruf et al., 1992; Martin et al, 1995). Residues 157-167 of TF appear to be important in accelerating activation of Factor VII to Factor VIIa, but not the binding of Factor VII to TF. The inventors mutated W158 to R and G164 to A and determined whether the mutants acquired the ability to coagulate plasma once localized by means of a bispecific antibody, B21/2-10H10, on the surface of A20 cells. It was found that the mutants were 30-50-fold less effective than was tTF₂₁₉ at inducing coagulation of plasma.

3. Restoration of Coagulating Ability of Factor VII Activation Mutants by Factor VIIa

Mutant tTF₂₁₉ (G164A) is a very weakly coagulating mutant of tTF₂₁₉ (Ruf, et al, 1992). The mutation is present in a region of TF (amino acids 157-167) thought to be important for the conversion of Factor VII to Factor VIIa. Thus, addition of Factor VIIa to cells coated with bispecific antibody and tTF₂₁₉ (G164A) would be reasoned to induce the coagulation of plasma. In support of this. A20 cells coated with B21-2/10H10 followed by tTF₂₁₉ (G164A) had increased ability to induce coagulation of plasma in the presence of Factor VIIa. Addition of Factor VIIa at 1 nM or greater produced only marginally slower coagulation times than observed with tTF₂₁₉ and Factor VIIa at the same concentrations.

Mutant tTF₂₁₉ (W158R) gave similar results to tTF₂₁₉ (G164A). Again, addition of Factor VIIa at 1 nM or greater to A20 cells coated with B21-2/10H10 followed by tTF₂₁₉ gave only marginally slower coagulation times than did tTF₂₁₉ and Factor VIIa at the same concentrations.

These results support those aspects of the invention that provide that tTF₂₁₉ (G164A) or tTF₂₁₉ (W158R), when coadministered with Factor VIIa to tumor-bearing animals, will induce the thrombosis of tumor vessels. This approach is envisioned to be advantageous because tTF (G164A). tTF (W158R) or Factor VIIa given separately are practically non-toxic to mice, and the same is reasonably expected in humans. Coadministration of the mutant tTF and Factor VIIa is expected not to cause toxicity, yet to cause efficient thrombosis of tumor vessels. Giving mutant tTF together with Factor VIIa is thus contemplated to result in an improved therapeutic index relative to tTF₂₁₉ plus Factor VIIa.

4. Enhanced Anti-Tumor Activity of Activation Mutants and Factor VIIa

For these studies, the inventors chose the HT29 (human colorectal carcinoma) xenograft tumor model. HT29 cells (10⁷ cells/mouse) were subcutaneously injected into BALB/c nu/nu mice. Tumor dimensions were monitored and animals were treated when the tumor size was between 0.5 and 1.0 cm³. Animals were given an intravenous injection of one of the following: tTF₂₁₉ (16 μg). tTF₂₁₉ (16 μg)+Factor VIIa (1 μg), tTF₂₁₉(GI614A) (64 μg), tTF₂₁₉(G164A) (64 μg)+Factor VIIa (1 μg), Factor VIIa alone (1 μg), or saline.

Animals were sacrificed 24 hours after treatment, perfused with saline and heparin and exsanguinated. Tumors and organs were collected, formalin fixed and histological sections were prepared. The average area of necrosis in sections of the tumors was quantified and calculated as a percentage of the total area of tumor on the section.

In these small HT29 tumors, analysis of tumor sections from animals treated with saline, Factor VIIa, tTF₂₁₉ or tTF₂₁₉(G164A) showed some necrosis. The tTF-induced tumor necrosis was the most developed, although this was not as striking, on this occasion, as results from earlier studies using different tumor models and/or large tumors. An analysis of tumor sections from animals treated with tTF₂₁₉+Factor VIIa or tTF₂₁₉(G164A)+Factor VIIa revealed considerable necrosis (12.5% and 17.7% respectively) and a strong correlation between newly thrombosed blood vessels and areas of necrosis. The combined use of Factor VIIa with TF, even a TF construct with particularly deficient in vitro coagulating activity, is therefore a particularly advantageous aspect of the present invention. As the HT29 tumor model is difficult to thrombose in general and these tumors were small in size, these results are likely to translate to even further striking results in other systems and in humans.

### EXAMPLE X

### Enhancement of Anti-Tumor Activity of Truncated Tissue Factor by Endotoxin

The present example shows that low dose endothelial cell activators sensitize tumor blood vessels, but not vessels in normal tissues, to thrombosis and thus enhance the effects of procoagulant tumor therapies.

A. Materials and Methods

1. Reagents, Cell Lines and Animals

Endotoxin, also known as “LPS” (lipopolysaccharides) from E. coli serotype 055:B5 was from Sigma-Aldrich (St. Louis, Mo.). L540rec is a human tumor cell line originally derived from a Hodgkin's lymphoma patient (Diehl et al., 1981) and passaged in vivo for increased metastatic potential. bEnd 3 cells are murine endothelial cells, which can be activated upon stimulation with cytokines (obtained from Dr. B. Engelhardt. Max-Planck-Institute, Bad Nauheim, Germany). 2F2B mouse endothelial cells, constitutively expressing VCAM-1, were purchased from ATCC/LGC (Middlesex, UK). Human umbilical vein endothelial cells (HUVEC) were from Biowhittaker (Walkersville, Md.).

Tissue culture reagents were from Invitrogen/Gibco Life Technologies (Karlsruhe, Germany). Molecular biology reagents were from Roche (Mannheim, Germany). Fox Chase SCID mice^{R} were from M&B (Ry, Denmark).

2. Generation of Recombinant Tissue Factor Mutant

Cloning of the gene encoding the first 219 amino acids of Tissue Factor and the generation of an expression vector (pswc7) for secretion of tTF into the periplasm of E. coli has been described (Gottstein et al. 2001; specifically incorporated herein by reference). E. coli were freshly transformed with pswc7 via heat shock transformation. Single colonies were cultured to a density of A₆₀₀=0.6 and the proteins were recovered from the periplasmic space via osmotic shock as described previously (Gottstein et al., 2001).

Recombinant proteins were purified on a Ni—NTA-affinity column (Qiagen, Hilden, Germany). As a second purification step, a gel filtration on a Superdex™ size exclusion column was performed (Amersham-Pharmacia, Braunschweig, Germany). To remove endotoxin, an affinity resin specific for endotoxins was used (Dimaco, Isnef, Belgium) and the flowthrough was collected in endotoxin-free glassware. Concentration and purity of the recombinant protein were assessed by SDS-PAGE and scanning UV-spectrophotometry.

3. Endotoxin Assay

Endotoxin concentrations were measured by a standard LAL assay (Biowhittaker. Walkersville, Md.) according to the manufacturer's instructions.

4. Coagulation Assay

In vitro coagulation activity was tested in a cell free two-stage coagulation assay. Negatively charged phospholipids at a final concentration of 50 μM (phosphatidylserine and phosphatidylcholine from Sigma, Taufkirchen, Germany) in calcium buffer (50 mM Tris pH=8.1, 150 mM NaCl, 2 mg/ml BSA, 5 mM Ca⁺⁺) were mixed with Factor VIIa (Sigma, Taufkirchen, Germany) at 10 nM and with samples or controls and incubated for five min at 37° C. Factor X was added to a final concentration of 30 nM and samples were incubated for 5 min at room temperature. Finally, the chromogenic substrate S2765 (Haemochrom, Essen, Germany) was added in a 100 mM EDTA solution. Factor Xa generation as a measure of Tissue Factor activity was determined by the increase in the absorption at 405 nm.

5. Cell Free Coagulation Assays

For the quality control of recombinant tTF, in vitro coagulation activity was tested in a cell free two-stage coagulation assay. Negatively charged phospholipids at a final concentration of 50 μM (phosphatidylserine and phosphatidylcholine from Sigma, St. Louis, Mo.) in calcium buffer (50 mM Tris pH=8.1, 150 mM NaCl, 2 mg/ml BSA, 5 mM Ca⁺⁺) were mixed with Factor VIIa (Sigma, St. Louis, Mo.) at 10 nM and with samples or controls and incubated for five minutes at 37° C. Factor X (Sigma, St. Louis, Mo.) was added to a final concentration of 30 nM and samples were incubated for 5 minutes at room temperature. Finally, the chromogenic substrate S2765 (Haemochrom, Essen, Germany) was added in a solution of 100 mM EDTA. pH=8.0. Factor Xa generation as a measure of tissue factor activity was determined by the increase in the absorption at 405 nm.

To assay the influence of endotoxin on the coagulation cascade in the absence of cells, the assay was performed as described above with 100 nM tTF in the presence or absence of 10 μg/ml LPS.

6. Cell Bound Coagulation Assays

To assay the binding of tTF to endothelial cells, 2F2B mouse endothelial cells were seeded in 48 well tissue culture plates at a density of 5×10⁴ cells per well and allowed to adhere overnight. tTF with or without LPS (10 μg/ml) was added and incubated at 4° C. overnight. Cells were washed and coagulation factor mix (as described above) was added. S2765 substrate was added and Factor Xa generation was measured as described above.

To assay the coagulation induction of stimulated versus unstimulated endothelial cells, bEnd 3 cells were seeded in 48 well tissue culture plates at a density of 1×10⁴ cells per well and allowed to adhere overnight. Cells were stimulated with endotoxin (0.5 μg/ml and 10 μg/ml) or TNFα (500 U/ml) for 4 h at 37° C. Then the cells were washed and subsequently incubated with 100 nM tTF or with 100 nM tTF-VIIa equimolar complex. After incubation for 45 min at room temperature, cells were washed and incubated with various coagulation factor mixes as follows: (1) 0.5 μg/ml factor VIIa in a mix containing 2.8 μg/ml factor IX, 3.4 μg/ml factor X, 50 μM phospholipids, in calcium buffer (as specified above); (2) 0.01 μg/ml factor VIIa in a mix as in (1); (3) 2 μg/ml factor VII (Calbiochem-Novabiochem, San Diego, Calif.) in a mix as in (1); (4) 2 μg/ml factor VII 0.01 μg/ml factor VIIa in a mix as in (1). The supernatant of wells was transferred into a 96 well ELISA plate. Substrate S2765 was added and Factor Xa generation measured alongside different concentrations of Factor Xa standard (7 nkat_{S2222}; 0.7 nkat_{S2222}; 0.07 nkat_{S2222}). OD₄₀₅ₙₘ values were calculated as nkat_{S2222} Factor Xa from the Factor Xa standard curve.

7. FACS (Fluorescence Activated Cell Stain) Analyses

To analyze tissue factor expression on the surface of endothelial cells, HUVEC cells were incubated with TNFα (500 U/ml), LPS (10 μg/ml) or vascular endothelial growth factor (VEGF, 1 nM) alone or in combination for 6 h at 37°. Cells were then detached and stained for surface expression of human tissue factor with a sheep-anti-human tissue factor antibody (Haemochrom, Essen, Germany) and an appropriate FITC-conjugated secondary antibody. Fluorescent cells were detected on a flow cytometer (Becton Dickinson, San Jose, Calif.).

To analyze binding of tTF to tissue factor upregulated upon stimulation of endothelial cells. 2F2B cells were stimulated with LPS (20 μg/ml) or TNFα (500 U/mi) for 4 h at 37° C. Cells were then incubated with tTF for 30 minutes at room temperature, washed and bound tissue factor antigen was detected with a sheep-anti-human tissue factor antibody (Haemochrom, Essen, Germany) and an appropriate FITC-conjugated secondary antibody. Fluorescent cells were detected on a flow cytometer (Becton Dickinson, San Jose, Calif.).

8. Real Time Binding Studies of tTF to Immobilized tTF

For real time binding analysis, using surface plasmon resonance (Biacore™), tTF was immobilized on a CM5 sensor chip (Biacore, Uppsala, Sweden) either directly by amine coupling, or captured by a covalently linked anti-human tissue factor antibody. Directly coupled tTF was immobilized at a surface density of 700 RU, the capturing antibody was immobilized at a surface density of 700 RU, and the captured tTF was bound at a density of 300 RU. tTF was then injected at a concentration of 30 μg/ml at a flow speed of 30 μl/min, either alone or after preincubation with LPS (10 μg/ml) or factor VIIa (50 μg/ml).

9. Animal Model

For in vivo studies, a metastasizing mouse model for human Hodgkin's lymphoma was used. 1×10⁷ L540rec cells were injected subcutaneously into the right flank of SCID mice resulting in a subcutaneous tumor with lymph node metastases in the regional lymph node stations. Subcutanous tumors were measured with a caliper in three perpendicular directions a, b, and c, and volumes calculated according to the formula V=π/6×a×b×c.

10. Treatment Studies

Treatment was initiated when subcutaneous tumors reached a size of 150 to 300 μl. Reagents were administered into the lateral tail vein. The mice were divided into eight different treatment groups: (1) diluent (0.9% NaCI-solution, clinical grade); (2) recombinant, depyrogenated tTF (“endotoxin-free tTF”) at 4 μg total dose; (3) endotoxin at 0.01 μg total dose; (4) endotoxin at 0.5 μg total dose; (5) endotoxin at 20 μg total dose; (6) tTF as in (2) spiked with 0.01 μg endotoxin total dose; (7) tTF as in (2) spiked with 0.5 μg endotoxin total dose: and (8) tTF as in (2) spiked with 20 μg endotoxin total dose.

Mice were closely observed after treatment for clinical signs of toxicity and clinical status was documented at defined time points (5 minutes. 10 minutes, 15 minutes, 30 minutes. 1 hour, 2 hours, 24 hours, 48 hours, 72 hours). Blood samples were taken from the tail vein at 1 hour, 2 hours and 24 hours to measure TNFα blood levels. Three days after treatment, the mice were anesthetized, blood samples were taken from the vena cava for coagulation tests, and an autopsy was performed to document any changes in gross pathology. Tumors, lymph node metastases and the major normal organs (heart, lung, brain, liver, kidney, colon, spleen, pancreas) were harvested and prepared for histological analysis.

11. Assessment of Coagulation Parameters

At the time of autopsy, citrated blood was sampled from the vena cava and thrombocyte-free plasma was prepared by centrifugation. The plasma was stored at −80° C. until further analysis. Thrombin-Antithrombin-complexes were detected with the Enzygnost® TAT micro-assay (Dade-Behring, Marburg, Germany) according to the manufacturer's instructions. ATIII levels were determined using the Coamatic® antithrombin-assay (Haemochrom, Essen. Germany) following the manufacturer's instructions. Changes in the blood levels of thrombin and plasmin were detected by mixing citrated plasma with the respective chromogenic substrates S2238 and S2403 (Haemochrom, Essen, Germany) and measuring the increase of the absorption at 405 nm by an ELISA reader.

12. Histological Evaluation

Tissue samples harvested at the time of autopsy were fixed in 3% NBF (normal buffered formalin) and embedded in paraffin wax. Tissue blocs were cut, dewaxed and stained with hematoxilin and eosin (H&E). Tissue sections were analyzed on a light microscope by two independent investigators and histological findings were documented. Tumor sections with necrotic areas were scanned with a GS-700 imaging densitometer (Biorad, Hercules, Calif.) and areas of necrosis were calculated as % of total section area. Statistical Analysis was performed using SPSS software (SPSS Science Software, Erkrath, Germany) applying the Mann-Whitney-U-test for ungrouped data.

13. TNFα Serum Levels in Treated Animals

Blood from mice treated with 0.5 μg/ml LPS, tTF or a combination treatment, was sampled at the time points indicated above, and serum was prepared. TNFα levels in serum were determined using the Quantikine-M kit (R&D Systems, Minneapolis, Minn.) according to the manufacturer∝s instructions.

B. Results

1. Recombinant, Depyrogenated, Truncated Tissue Factor

Recombinant soluble Tissue Factor protein (amino acids 1-219) was extracted from the periplasmic space of transformed E. coli and purified near to homogeneity. After the last endotoxin removal step, no endotoxin was detected in a 1:10 dilution of the final product. The detection limit of the LAL assay was determined to be approximately 1 pg/ml (1 IU corresponds to 30 to 100 pg).

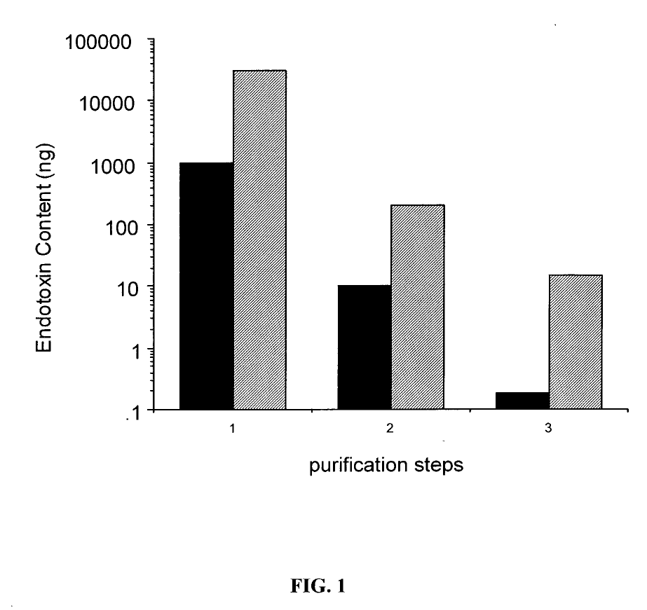

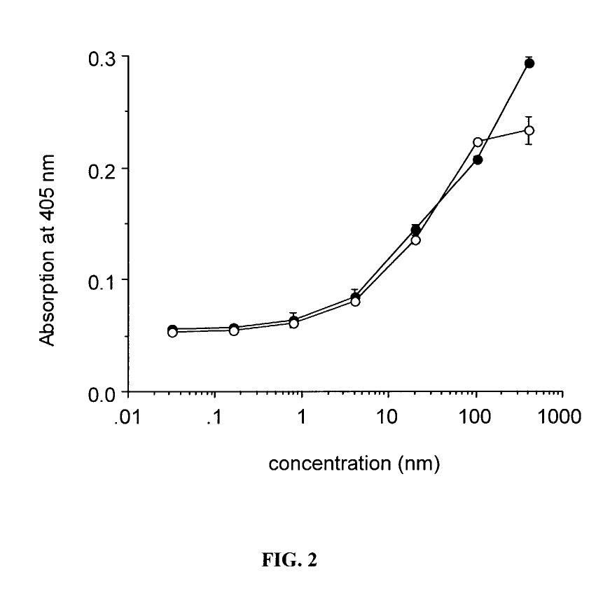

Amounts of endotoxin in the recombinant protein preparation after three subsequent purification steps are shown in FIG. 1. Both the concentration of endotoxin in ng/ml solution (black bars in FIG. 1) and the endotoxin content per mg protein (gray bars in FIG. 1) are shown. Functional activity was verified in a cell free two-stage coagulation assay. The coagulation activities before and after endotoxin removal (depyrogenation) were the same (FIG. 2).

2. Clinical Signs and Macroscopic Evidence in Treated Animals

Table 1 gives an overview on symptoms of toxicity and on the time of onset. Mice given diluent, endotoxin-free tTF, 10 ng endotoxin or tTF with 10 ng endotoxin showed no clinical signs of toxicity. Mice with 0.5 μg endotoxin or tTF plus 0.5 μg endotoxin had only mild toxicity symptoms, whereas mice with high dose endotoxin (20 μg) or the combination of tTF and 20 μg endotoxin showed typical signs of endotoxin related toxicity: hypoactivity beginning 15 minutes after i.v. injection, diarrhea beginning 30 to 60 minutes after injection, and general signs such as ruffled fur, elaborated breathing and haunched posture. Clinical signs of toxicity were alleviated after 48 hours and most mice appeared normal after 72 hours. Some tumors darkened and eventually turned black one day after injection (black tumors are tumor necrotic, as opposed to pink tumors, which are viable). Importantly, at time of autopsy, no gross abnormalities were detected in any of the normal organs.

TABLE 1
Clinical Signs in Tumor Bearing Animals After tTF
and/or Endotoxin Treatment

| | | Onset of symptoms after |
|---|---|---|
| Treatment | Symptoms | treatment |
| Diluent | None | |
| TTF | None | |
| 0.5 μg endotoxin | slightly hypoactive | 15 min |
| 20 μg endotoxin | Hypoactive | 15 min |
| | Diarrhea | 30-60 min |
| 0.01 μg endotoxin + | None | |
| tTF | | |
| 0.5 μg endotoxin + tTF | slightly hypoactive | 15 min |
| 20 μg endotoxin + tTF | Hypoactive | 15 min |
| | Diarrhea | 30-60 min |

3. Histology in Tumors and Normal Organs of Treated Animals

The appearance, thrombosis and necrotic tissue in the tumors of treated mice was examined, representing a macroscopic and microscopic analysis. In viable tumors, open vessels were oberserved. In the treatment groups, sections of damaged tumor tissue was seen with fragmented or pyknotic nuclei; thrombosed vessels were also observed, surrounded by discohesive tumor cells with signs of necrosis.

Tumor tissues treated with the combination of endotoxin and tTF as well as with high dose endotoxin showed thrombotic vessels and necrotic tumor tissue. Tissue necrosis was quantified after densitometry of several representative tissue sections. In these analyses, viable tumor tissue shows dark blue, and necrotic areas within the tumor appear in pink. Percentages of tumor tissue necrosis in the eight treatment groups were as follows: (1) 0% for mice treated with diluent (n=5); (2) 0% for mice treated with 4 μg or 16 μg endotoxin-free tTF (n=8); (3) 11% for mice treated with 0.01 μg endotoxin (n=4); (4) 12% for mice treated with 0.5 μg endotoxin (n=9); (5) 51% for mice treated with 20 μg endotoxin (n=2); (6) 48% for mice treated with the combination of 4 μg tTF and 0.01 μg endotoxin (n=5); (7) 28% for mice treated with the combination of 4 μg tTF and 0.5 μg endotoxin (n=8); and (8) 78% for mice treated with 4 μg tTF and 20 μg endotoxin (n=2).

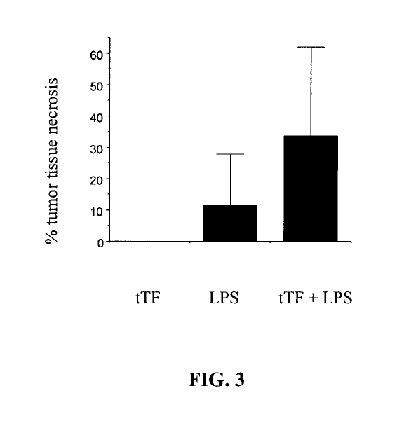

FIG. 3 demonstrates, as an example, average amounts of thrombosis and standard deviations in tumors of mice treated with 0.5 μg LPS, 4 μg tTF or the combination thereof. The amounts of necrosis generally followed the same pattern in lymph node metastases.

In normal organs, there were no necrotic areas in any of the treatment groups. No significant thrombosis or bleeding was detected by light microscopy. Out of 59 mice evaluated for toxicity, single microfocal thrombi were found only in rare cases, in the liver or lung of mice treated with an endotoxin containing regimen. No dose dependency was observed for endotoxin. No histological abnormalities were seen in mice treated with endotoxin-free tTF (n=13) or diluent (n=5).

4. Changes in Coagulation Parameters in Treated Animals

The plasma levels of the following coagulation parameters were analyzed three days after treatment: thrombin-antithrombin-complexes (TAT), antithrombin III (ATIII), thrombin and plasmin. Comparing tumor bearing with non-tumor bearing mice, TAT-levels and ATIII-levels were comparable, whereas thrombin levels and, to a slight extent, plasmin levels were elevated in tumor bearing mice. Table 2 demonstrates that TAT-levels were elevated when mice were treated with tTF, corresponding to a slight decrease of active ATIII. There was also a trend to elevated plasmin levels in tTF treated mice.

TABLE 2
Coagulation Parameters After tTF and/or Endotoxin Tumor Treatment

| Treatment | % tumor necrosis | TAT (ng/ml) | ATIII (%) |
|---|---|---|---|
| Non tumor bearing mice, | n/a | 7.9 | 100 |
| Treated with diluent | | | |
| Diluent | 1 | 4.4 | 89 |
| TTF | 4 | 25.4 | 79 |
| 0.5 μg endotoxin | 7 | 8.0 | 82 |
| 20 μg endotoxin | 47 | 18.0 | 85 |
| 0.5 μg endotoxin + tTF | 25 | 9.4 | 85 |
| 20 μg endotoxin + tTF | 78 | 32.0 | 72 |

Plasma levels of thrombin-antithrombin-complexes (TAT) or antithrombin III (ATIII) were determined in tumor bearing mice three days after i.v. treatment. ATIII-levels measured in non-tumor bearing mice were defined as 100%.

5. TNFα Serum Levels in Treated Animals

TNFα serum levels were increased in all but one mouse, treated with a regimen containing 0.5 μg/ml endotoxin (n=14). One hour after injection. TNFα levels rose to an average of 2.8 ng/ml (range: 0.5-7.6 ng/ml). After 2 hours, average TNFα levels were 0.3 ng/ml (range 0-0.8 ng/ml) and after 24 hours, no TNFα was detectable. In mice treated with tTF containing no endotoxin (n=6). TNF(X could not be detected in the serum at any of the time points investigated.

6. Tissue Factor Expression on the Surface of Endothelial Cells

The expression of tissue factor on the surface of HUVEC cells was measured by FACS analysis. Both VEGF and TNFα upregulated tissue factor expression on the surface of endothelial cells, and the combination of the two substances was highly synergistic, similar to what has been described by Clauss et al (1996) and Camera et al. (1999). In this assay, endotoxin alone or in combination with VEGF did not cause tissue factor upregulation on HUVEC.

7. Effects of Endotoxin on Cell Free Coagulation

Addition of endotoxin to tTF in a cell free coagulation assay did not result in a statistically significant increase of coagulation activity, although a marginal increase of Xa production was observed. That marginal increase of Xa activity was not dose dependant. Endotoxin seems therefore not to function as a direct cofactor in the coagulation cascade.

8. Binding of tTF to Cell-Surface or Immobilized Tissue Factor

The binding of tTF to tissue factor on the surface of cells or immobilized on a carbohydrate matrix was analyzed using a real time binding study. tTF alone or preincubated with either endotoxin or factor VIIa did not bind to or homodimerize with immobilized tTF, as measured by surface plasmon resonance. Moreover, no binding of tTF to endothelial cells that expressed tissue factor on their surface was detected by FACS analysis or by a cell bound coagulation assay.

9. Endotoxin Effect on the Coagulation Activity of Mouse Endothelial Cells

The results of cell bound coagulation assays investigating the effect of endotoxin (LPS) or TNFα on the coagulation activity of mouse endothelial cells are summarized in Table 3. This table concerns the ability of tTF-VIIa complex to increase factor Xa production directly or indirectly via factor VIIa production on the surface of endothelial cells.

When bEnd3 cells were stimulated with either LPS (0.5 μg/ml; 10 μg/ml) or TNFα (500 U/ml), and not further incubated with tTF (Table 3. left side, line 1), the net procoagulant effect was somewhat increased. This was probably due to an upregulation of endogenous tissue factor after stimulation. Stimulation of endothelial cells, followed by the incubation with either tTF or tTF-VIIa complex (Table 3, left side, lines 2 and 3), resulted in a further enhancement of the coagulability.

Incubation with tTF alone (Table 3, left side, line 2), resulted in increased coagulability to the same extent in stimulated and unstimulated cells and decreased when cells were washed more vigorously. It was assumed that this increase in coagulability was a background effect due to unspecific adherence of tTF to the wells. Incubation with tTF-VIIa complex however, (Table 3. left side, line 3), showed a marked increase of procoagulant activity in cells stimulated with TNFα or LPS, but not in unstimulated cells. Therefore, it seems, that stimulation of endothelial cells with TNFα or LPS promotes the ability of the tTF-VIIa complex to adhere and cause procoagulant changes. Table 3. left side, line 4 shows the amount of factor Xa generation by the tTF-VIIa complex after subtraction of the background (Table 3, left side, line 2).

TABLE 3
Ability of tTF-VIIa Complex to Increase Factor Xa Production on Cell Surfaces

| | | | | | Factor Xa generation due to | | |
|---|---|---|---|---|---|---|---|
| | Factor Xa generation* | | | | Factor VIIa production # | | |
| | Stimulation with: | | | | Stimulation with: | | |
| Incubation | | | 0.5 μg/ml | 10 μg/ml | | | 0.5 μg/ml |
| with: | Medium | TNFα | LPS | LPS | Medium | TNFα | LPS |
| Medium | 0.04 | 0.35 | 1.16 | 2.59 | 0.84 | 1.23 | 2.63 |
| (negative | | | | | | | |
| control) | | | | | | | |
| tTF | 0.40 | 0.67 | 2.00 | 2.91 | 0.70 | 1.91 | 3.00 |
| (background | | | | | | | |
| after wash) | | | | | | | |
| tTF-VIIa | 0.56 | 1.51 | 3.35 | 4.46 | 0.95 | 2.93 | 6.25 |
| tTF-VIIa | 0.16 | 0.84 | 1.35 | 1.55 | 0.25 | 1.02 | 3.25 |
| minus sTF | | | | | | | |

*Left side of table: Endothelial cells stimulated with medium (negative control).
TNFα or LPS were incubated with medium (negative control), tTF (background) or tTF-VIIa complex. The coagulation factor mix contained 0.5 μg/ml factor VIIa (VIIa not limiting). Net procoagulant effect was measured as factor Xa generation in nkat_{S2222}.
# Right side of table: The assay was performed analogous, with the exception that the coagulation factor mix contained 2 μg/ml factor VII and 0.01 μg/ml factor VIIa. Although the readout is also factor Xa generation, the values represent de novo formation of factor VIIa.

To analyze whether, in this system, activation of factor VII to VIIa takes place, and whether stimulation of endothelial cells has an impact on this, the following study was conducted. After incubating stimulated vs. unstimulated cells with either medium (negative control), tTF or tTF-VIIa complex, different coagulation factor mixes were added: one mix contained factor VIIa at a concentration of 0.01 μg/ml. At this low concentration of VIIa, no factor Xa production was observed. When a coagulation factor mix containing 2 μg/ml factor VII was used, there was some background activity of factor Xa generation. This activity was markedly increased when factor VII was given together with the per se ineffective dose of 0.01 μg/ml factor VIIa, indicating that the additional coagulation activity was due to de novo factor VIIa generation.

Values of the samples in which the coagulation mix contained 2 μg/ml factor VII (considered as background), were subtracted from those, in which factor VII plus a small amount of VIIa (0.01 μg/ml) was used, and the differential values, reflecting de novo generation of factor VIIa from VII are shown in the right half of Table 3. Final readout was again factor Xa generation. In stimulated cells, which were not incubated with tTF or tTF-VIIa complex (Table 3. right side, line 1), there was a slight increase of VIIa production vs. unstimulated cells. This is most likely due to a higher surface density of tissue factor. When these cells were incubated with tTF-VIIa complex, the additional VIIa production was markedly increased in stimulated cells, but not in unstimulated cells (Table 3, right side, line 4).

In summary, factor VIIa seems to mediate the adhesion or binding of tTF to the surface of activated endothelial cells. This results in an increase of the net procoagulant effect due to both factor Xa production (when VIIa is not a limiting factor) and to generation of additional factor VIIa from factor VII (where factor VIIa is limited).

Incubation of tumor cells with high amounts of endotoxin showed no direct toxicities on the tumor cells when assessed in an XTT assay. Stringent adjustment of endotoxin levels in all treatment groups is thus necessary in in vivo studies where effects of vascular targeting agents in tumor bearing mice are assessed.

Importantly, the present example shows that low dose endothelial cell activators render tumor blood vessels, but not vessels in normal tissues, sensitive to thrombosis induction. This provides the basis for improved human tumor treatment using sensitizing agents in combination with targeted or non-targeted coagulants.

### EXAMPLE XI

### TNFα or Endotoxin Enhance Net Procoagulant Effects

This example describes the enhancement of the net procoagulant effect of truncated tissue factor on endothelial cells in vitro by incubation with TNFα or endotoxin.

bEnd 3 cells were seeded in 48 well tissue culture plates at a density of 1×10⁴ cells per well and allowed to adhere overnight. Cells were stimulated with endotoxin (0.5 μg/ml and 10 μg/ml) or TNFα (500 U/ml) for 4 h at 37° C. Then the cells were washed and subsequently incubated with 100 nM tTF or with 100 nM tTF-VIIa equimolar complex. After incubation for 45 min at room temperature, cells were washed and incubated with coagulation factor mix as follows: 0.5 μg/ml factor VIIa in a mix containing 2.8 μg/ml factor IX, 3.4 μg/ml factor X, 50 μM phospholipids, in calcium buffer; supernatant of wells was transferred into a 96 well ELISA plate. Substrate S2765 was added and Factor Xa generation measured alongside different concentrations of Factor Xa standard (7 nkat_{S2222}; 0,7 nkat_{S2222}; 0,07 nkat_{S2222}). OD405 nm values were calculated as nkat_{S2222} Factor Xa from the Factor Xa standard curve.

The amount of factor Xa generation by the tTF-VIIa complex was plotted after subtraction of the background. The results of these studies showed that the stimulation of endothelial cells, followed by the incubation with either tTF or tTF-VIIa complex, resulted in an enhancement of the coagulability.

### EXAMPLE XII

### Enhanced tTF Coagulation by Endotoxin in Sarcoma Tumors

In this example, the enhanced coagulation effects of tTF by endotoxin are shown using a sarcoma mouse model.

1×10⁷ F9 sarcoma cells were injected subcutaneously into the right flank of balb/c nude mice. Subcutanous tumors were measured with a caliper in three perpendicular directions a, b, and c, and volumes calculated according to the formula V=π/6×a×b×c. Treatment was initiated when subcutaneous tumors reached a size of 150 to 300 μl.

Reagents were administered into the lateral tail vein. Mice were divided in four different treatment groups: (1) diluent (0.9% NaCl-solution, clinical grade); (2) recombinant, depyrogenated tTF at 4 μg total dose; (3) endotoxin at 0.5 μg total dose; and (4) tTF as in (2) spiked with 0.5 μg LPS.

Mice were closely observed after treatment for clinical signs of toxicity and clinical status was documented at defined time points. Three days after treatment, mice were sacrificed and tumors were harvested. Paraffin embedded tissues were stained with hematoxilin and eosin (H&E). Tissue sections were analyzed on a light microscope by two independent investigators and histological findings were documented. Tumor sections with necrotic areas were scanned with a GS-700 imaging densitometer (Biorad. Hercules, Calif.) and areas of necrosis were calculated as % of total section area.

These results showed that the average tumor tissue necrosis was enhanced in the mice treated with the combination of endotoxin and tTF: mice treated with diluent showed 40-50% spontaneous necrosis. Treatment with endotoxin-free tTF resulted in 45% tumor tissue necrosis on average; and treatment with the combination of tTF and endotoxin resulted in 80% average tumor tissue necrosis.

### EXAMPLE XIII

### TNFα-Upregulation of Adhesion Molecules and Procoagulant Effects

Studies were conducted to analyze the different doses of TNFα required for upregulation of adhesion molecules and for enhanced procoagulant effects, which are reported in the present example.

Mouse endothelial cells were seeded in 48 well tissue culture plates and allowed to adhere overnight. Cells were stimulated with TNFα at the following concentrations: 500 U/ml 100 U/ml; 20 U/ml; 4 U/ml; 0.8 U/ml 0.16 U/ml; and with medium only.

Endothelial cells were then investigated for upregulation of the adhesion molecule VCAM-1 by fluorescence activated cell stain (FACS). To this end, cells were stained with an antibody against murine VCAM-1. followed by an appropriate FITC-conjugated secondary antibody. Fluorescent cells were detected on a flow cytometer (Becton Dickinson, San Jose, Calif.).

Endothelial cells were also tested for coagulant activity in a cell based two stage coagulation assay. After incubation with TNFα, cells were washed and incubated with coagulation factor mix (0.5 μg/ml factor VIIa in a mix containing 2.8 μg/ml factor IX, 3.4 μg/ml factor X, 50 μM phospholipids in calcium buffer). The supernatant of wells was transferred into a 96 well ELISA plate. Substrate S2765 was added and Factor Xa generation measured.

The results showed that a measurable increase of VCAM-1 expression required 20 U/ml of TNFα. In the coagulation assay, an increase in comparison to the negative control could be detected at 0.16 U/ml, i.e., at a 125-fold lower dose.

### EXAMPLE XIV

### Enhancement of Anti-Tumor Activity of Immunoglobulin-tTF Conjugate By Etoposide

Mice bearing L540 human Hodgkin's disease tumors were treated with a complex of tTF₂₁₉ and a bispecific antibody together with the anti-cancer drug, etoposide, at a conventional dose. Standard dose etoposide treatment greatly enhanced the action of the immunoglobulin-tTF conjugate.

In this tumor model alone, mice receiving the antibody-tTF complex alone showed little reduction in tumor growth relative to tumors in mice receiving diluent alone. In contrast, tumors in mice receiving both a conventional dose of etoposide and the immunoglobulin-tTF conjugate regressed in size and did not recommence growth for seventeen days. At the end of the study (day 20), tumors in mice receiving etoposide plus immunoglobulin-tTF were an average of 900 mm³ in volume as compared with 2300 mm³ in mice treated with diluent and 2000 mm³ in mice treated with immunoglobulin-tTF alone. In mice receiving etoposide alone, tumors averaged 1400 mm³ on day 14.

### EXAMPLE XV

### Tumor Treatment With Anti-Endoplin-tTF Coaguligand

The present example shows that antibodies directed to endoglin are effective in tumor-targeting and that anti-endoglin antibodies in combination with truncated Tissue Factor exert significant anti-tumor effects in vivo.

The TEC-4 and TEC-11 antibodies are directed against endoglin, an antigen that is upregulated on vascular endothelial cells in a broad range of malignant tumors. As the TEC-4 and TEC-11 antibodies are directed to human endoglin, a SCID mouse model was chosen in which human skin is first grafted onto the animal (human/SCID animals), and then breast cancer cells are injected into the graft. Administering TEC-11 to a human/SCID animal bearing a human skin graft containing a palpable tumor results in the antibody localizing to 84% of blood vessels in the tumor periphery and 46% of blood vessels throughout the tumor, following an overnight treatment period.

A hybridoma producing an antibody directed to mouse endoglin, termed MJ 7/18 (Eugene Butcher, Stanford University), was used to prepare a bispecific antibody construct that binds to endoglin and truncated tissue factor (tTF). This bispecific antibody is termed MJ 7/18-10H10. Mixing the bispecific antibody with human tTF results in a preparation of bispecific antibody bound to tTF, which also includes free tTF (MJ 7/18-10H10-tTF).

The MJ 7/18-10H10-tTF preparation was tested using a mouse model of Hodgkin's tumor. In this model, a human Hodgkin's disease tumor xenograft is established by growing L540 tumor cells in SCID mice. Administration of the bispecific antibody-coagulant mixture resulted in significant anti-tumor effects within 48 hours. In animals with 0-500 mm³ and 500-1,000 mm³ tumors, 33% and 25%, respectively, of animals treated with the bispecific antibody-coagulant mixture alone respond with at least 45% necrosis. This figure rises to 63% and 83% of animals with 1,000-1,500 mm³ and 1.500-3.500 mm³ tumors, respectively. This effect of the anti-endoglin bispecific antibody-coagulant is consistent with its function in collapsing the tumor vasculature rather than simply slowing or inhibiting the growth of new vessels.

### EXAMPLE XVI

### Tumor Treatment with Anti-VCAM-1-tTF Coaguligand

This example presents further successful in vivo tumor treatment data using targeted coagulants in the form of a coaguligand comprising a VCAM-1 targeting agent.

The blood vessels of the major organs and a tumor from mice bearing subcutaneous L540 human Hodgkin's tumors were examined immunohistochemically for VCAM-1 expression using an anti-VCAM-1 antibody. Overall, VCAM-1 expression was observed on 20-30% of total tumor blood vessels stained by the anti-endoglin antibody, MJ 7/18. Constitutive vascular expression of VCAM-1 was found in heart and lungs in both tumor-bearing and normal animals. Strong stromal staining was observed in testis where VCAM-1 expression was strictly extravascular.

Mice bearing subcutaneous L540 tumors were injected intravenously with anti-VCAM-1 antibody and, two hours later, the mice were exsanguinated. The tumor and normal organs were removed and frozen sections were prepared and examined immunohistochemically to determine the location of the antibody. Anti-VCAM-1 antibody was detected on endothelium of tumor, heart and lung. Staining was specific as no staining of endothelium was observed in the tumor and organs of mice injected with a species isotype matched antibody of irrelevant specificity, R187. No localization of anti-VCAM-1 antibody was found in testis or any normal organ except heart and lung.

An anti-VCAM-1●tTF conjugate or “coaguligand” was prepared using truncated tissue factor (tTF). Intravenous administration of the anti-VCAM-1●tTF coaguligand induces selective thrombosis of tumor blood vessels, as opposed to vessels in normal tissues, in tumor-bearing mice.

The anti-VCAM-1●tTF coaguligand was administered to mice bearing subcutaneous L540 tumors of 0.4 to 0.6 cm in diameter. Before coaguligand injection, tumors were viable, having a uniform morphology lacking regions of necrosis. The tumors were well vascularized and had a complete absence of spontaneously thrombosed vessels or hemorrhages. Within four hours of coaguligand injection, 40-70% of blood vessels were thrombosed, despite the initial staining of only 20-30% of tumor blood vessels. The thrombosed vessels contained occlusive platelet aggregates, packed erythrocytes and fibrin. In several regions, the blood vessels had ruptured, spilling erythrocytes into the tumor interstitium.

By 24 h after coaguligand injection, the blood vessels were still occluded and extensive hemorrhage had spread throughout the tumor. Tumor cells had separated from one another, had pyknotic nuclei and were undergoing cytolysis. By 72 h, advanced necrosis was evident throughout the tumor. It is likely that the initial coaguligand-induced thrombin deposition results in increased induction of the VCAM-1 target antigen on central vessels, thus amplifying targeting and tumor destruction.

The thrombotic action of anti-VCAM-1●tTF on tumor vessels was antigen specific. None of the control reagents administered at equivalent quantities (tTF alone, anti-VCAM-1 antibody alone, tTF plus anti-VCAM-1 antibody or the control coaguligand of irrelevant specificity) caused thrombosis.

In addition to the thrombosis of tumor blood vessels, this study also shows that intravenous administration of the anti-VCAM-1●tTF coaguligand does not induce thrombosis of blood vessels in normal organs. Despite expression of VCAM-1 on vessels in the heart and lung of normal or L540 tumor-bearing mice, thrombosis did not occur after anti-VCAM-1●tTF coaguligand administration. No signs of thrombosis, tissue damage or altered morphology were seen in 25 mice injected with 5 to 45 μg of coaguligand 4 or 24 h earlier. There was a normal histological appearance of the heart and lung from the same mouse that had major tumor thrombosis. All other major organs (brain, liver, kidney, spleen, pancreas, intestine, testis) also had unaltered morphology.

Frozen sections of organs and tumors from coaguligand-treated mice gave coincident staining patterns when developed with either the anti-TF antibody, 10H10, or an anti-rat IgG antibody and confirmed that the coaguligand had localized to vessels in the heart, lung and tumor. The intensity of staining was equal to that seen when coaguligand was applied directly to the sections at high concentrations followed by development with anti-TF or anti-rat IgG, indicating that saturation of binding had been attained in vivo.

These studies show that binding of coaguligand to VCAM-1 on normal vasculature in heart and lung is not sufficient to induce thrombosis, and that tumor vasculature provides additional factors to support coagulation.

The anti-tumor activity of anti-VCAM-1●tTF coaguligand was determined in SCID mice bearing 0.3-0.4 cm³ L540 tumors. The drug was administered i.v. 3 times at intervals of 4 days. Mean tumor volume of anti-VCAM-1●tTF treated mice was significantly reduced at 21 days of treatment (P<0.001) in comparison to all other groups. Nine of a total of 15 mice treated with the specific coaguligand showed more than 50% reduction in tumor volume. This effect was specific since unconjugated tTF, control IgG coaguligand and mixture of free anti-VCAM-1 antibody and tTF did not affect tumor growth.

### EXAMPLE XVII

### Phosphatidylserine Expression on Tumor Blood Vessels

To explain the lack of thrombotic effect of anti-VCAM-1●tTF on VCAM-1 positive vasculature in heart and lungs, the inventors developed a concept of differential PS localization between normal and tumor blood vessels. Specifically, they hypothesized that endothelial cells in normal tissues segregate PS to the inner surface of the plasma membrane phospholipid bilayer, where it is unable to participate in thrombotic reactions; whereas endothelial cells in tumors translocate PS to the external surface of the plasma membrane, where it can support the coagulation action of the coaguligand. PS expression on the cell surface allows coagulation because it enables the attachment of coagulation factors to the membrane and coordinates the assembly of coagulation initiation complexes (Ortel et al, 1992).

The inventors' model of PS translocation to the surface of tumor blood vessel endothelial cells, as developed herein, is surprising in that PS expression does not occur after, and does not inevitably trigger, cell death. PS expression at the tumor endothelial cell surface is thus sufficiently stable to allow PS to serve as a targetable entity-for therapeutic intervention.

To confirm the hypothesis that tumor blood vessel endothelium expresses PS on the luminal surface of the plasma membrane, the inventors used the following immunohistochemical study to determine the distribution of anti-PS antibody after intravenous injection into L540 tumor bearing mice.

A. Methods

1. Antibodies

Anti-phosphatidylserine (anti-PS) and anti-cardiolipin antibodies, both mouse monoclonal IgM antibodies, were produced as described by Rote (Rote et al., 1993). Details of the characterization of the anti-PS and anti-cardiolipin antibodies were also reported by Rote et al. (1993, incorporated herein by reference).

2. Detection of PS Expression on Vascular Endothelium

L540 tumor-bearing mice were injected i.v. with 20 μg of either anti-PS or anti-cardiolipin mouse IgM antibodies. After 10 min., mice were anesthetized and their blood circulations were perfused with heparinized saline. Tumors and normal tissues were removed and snap-frozen. Serial sections of organs and tumors were stained with either HRP-labeled anti-mouse IgM for detection of anti-PS antibody or with anti-VCAM-1 antibody followed by HRP-labeled anti-rat Ig.

To preserve membrane phospholipids on frozen sections, the following protocol was developed. Animals were perfused with DPBS containing 2.5 mM Ca²⁺. Tissues were mounted on 3-aminopropyltriethoxysilane-coated slides and were stained within 24 h. No organic solvents, formaldehyde or detergents were used for fixation or washing of the slides. Slides were re-hydrated by DPBS containing 2.5 mM Ca²⁺ and 0.2% gelatin. The same solution was also used to wash sections to remove the excess of reagents. Sections were incubated with HRP-labeled anti-mouse IgM for 3.5 h at room temperature to detect anti-PS IgM.

B. Results

This immunohistochemical study showed that anti-PS antibody localized within 10 min. to the majority of tumor blood vessels, including vessels in the central region of the tumor that can lack VCAM-1. Vessels that were positive for VCAM-1 were also positive for PS. Thus, there is coincident expression of PS on VCAM-1-expressing vessels in tumors.

In the in vivo localization studies, none of the vessels in normal organs, including VCAM-1-positive vasculature of heart and lung, were stained, indicating that PS is absent from the external surface of the endothelial cells. In contrast, when sections of normal tissues and tumors were directly stained with anti-PS antibody in vitro, no differences were visible between normal and tumor, endothelial or other cell types, showing that PS is present within these cells but only becomes expressed on the surface of endothelial cells in tumors.

The specificity of PS detection was confirmed by two independent studies. First, a mouse IgM monoclonal antibody directed against a different negatively charged lipid, cardiolipin, did not home to tumor or any organs in vivo. Second, pretreatment of frozen sections with acetone abolished staining with anti-PS antibody, presumably because it extracted the lipids together with the bound anti-PS antibody.

### EXAMPLE XVIII

### Annexin V Blocks Coaguligand Activity

1. Annexin V Blocks Coaguligand Activation of Factor X In Vitro

The ability of Annexin V to affect Factor Xa formation induced by coaguligand was determined by a chromogenic assay. IL-1α-stimulated bEnd.3 cells were incubated with anti-VCAM-●tTF and permeabilized by saponin. Annexin V was added at concentrations ranging from 0.1 to 10 μg/ml and cells were incubated for 30 min. before addition of diluted Proplex T. The amount of Factor Xa generated in the presence or absence of Annexin V was determined. Each treatment was performed in duplicate and repeated at least twice.

The need for surface PS expression in coaguligand action is further indicated by the inventors' finding that annexin V, which binds to PS with high affinity, blocks the ability of anti-VCAM-1●tTF bound to bEnd.3 cells to generate factor Xa in vitro.

Annexin V added to permeabilized cells preincubated with anti-VCAM-1●tTF inhibited the formation of factor Xa in a dose-dependent manner. In the absence of Annexin V, cell-bound coaguligand produced 95 ng of factor Xa per 10,000 cells per 60 min. The addition of increasing amounts of Annexin V (in the μg per ml range) inhibited factor Xa production. At 10 μg per ml. Annexin V inhibited factor Xa production by 58%. No further inhibition was observed by increasing the concentration of Annexin V during the assay, indicating that annexin V saturated all available binding sites at 10 μg per ml.

2. Annexin V Blocks Coaguligand Activity In Vivo

The ability of Annexin V to inhibit coaguligand-induced thrombosis in vivo was examined in L540 Hodgkin-bearing SCID mice. Tumors were grown in mice and two mice per group (tumor size 0.5 cm in diameter) were injected intravenously via the tail vein with one of the following reagents: a) saline; b) 100 μg of Annexin V; c) 40 μg of anti-VCAM-1[tTF; d) 100 μg of Annexin V followed 2 hours later by 40 μg of anti-VCAM-1●tTF.

Four hours after the last injection mice were anesthetized and perfused with heparinized saline. Tumors were removed, fixed with 4% formalin, paraffin-embedded and stained with hematoxylene-eosin. The number of thrombosed and non-thrombosed blood vessels were counted and the percentage of thrombosis was calculated.

Annexin V also blocks the activity of the anti-VCAM-1●tTF coaguligand in vivo. Groups of tumor-bearing mice were treated with one of the control or test reagents. The mice were given (a) saline; (b) 100 μg of Annexin V; (c) 40 μg of anti-VCAM-1●tTF coaguligand; or (d) 100 μg of Annexin V followed 2 hours later by 40 μg of anti-VCAM-1●tTF coaguligand. Identical results were obtained in both mice per group.

No spontaneous thrombosis, hemorrhages or necrosis were observed in tumors derived from saline-injected mice. Treatment with Annexin V alone did not alter tumor morphology.

In accordance with other data presented herein, 40 μg of anti-VCAM-1●tTF coaguligand caused thrombosis in 70% of total tumor blood vessels. The majority of blood vessels were occluded with packed erythrocytes and clots, and tumor cells were separated from one another. Both coaguligand-induced anti-tumor effects, i e., intravascular thrombosis and changes in tumor cell morphology, were completely abolished by pre-treating the mice with Annexin V.

These findings confirm that the anti-tumor effects of coaguligands are mediated through the blockage of tumor vasculature. These data also demonstrate that PS is essential for coaguligand-induced thrombosis in vivo.

### EXAMPLE XIX

### Externalized Phosphatidylserine is a Global Marker of Tumor Blood Vessels

A. Methods

PS exposure on tumor and normal vascular endothelium was examined in three animal tumor models: L540 Hodgkin lymphoma, NCI-H358 non-small cell lung carcinoma, and HT 29 colon adenocarcinoma (ATCC). To grow the tumors in vivo, 2×10⁶ cells were injected into the right flank of SCID mice and allowed to reach 0.8-1.2 cm in diameter. Mice bearing large tumors (volume above 800 mm³) were injected intravenously via the tail vein with 20 μg of either anti-PS or anti-cardiolipin antibodies. The anti-cardiolipin antibody served as a control for all studies since both antibodies are directed against negatively charged lipids and belong to the same class of immunoglobulins (mouse IgM).

One hour after injection, mice were anesthetized and their blood circulation was perfused with heparinized saline. Tumors and normal organs were removed and snap-frozen. Frozen sections were stained with anti-mouse IgM-peroxidase conjugate (Jackson Immunoresearch Labs) followed by development with carbazole.

B. Results

The anti-PS antibodies specifically homed to the vasculature of all three tumors (HT 29, L540 and NCI-H358) in vivo, as indicated by detection of the mouse IgM. The average percentages of vessels stained in the tumors were 80% for HT 29, 30% for L540 and 50% for NCI-H358. Vessels in all regions of the tumors were stained and there was staining both of small capillaries and larger vessels.

No vessel staining was observed with anti-PS antibodies in any normal tissues. In the kidney, tubules were stained both with anti-PS and anti-CL, and this likely relates to the secretion of IgMs by this organ. Anti-cardiolipin antibodies were not detected in any tumors or normal tissues, except kidney. These findings indicate that only tumor endothelium exposes PS to the outer site of the plasma membrane.

To estimate the time at which tumor vasculature loses the ability to segregate PS to the inner side of the membrane, the inventors examined anti-PS localization in L540 tumors ranging in volume from 140 to 1.600 mm³. Mice were divided into 3 groups according to their tumor size: 140-300, 350-800 and 800-1.600 mm³. Anti-PS Ab was not detected in three mice bearing small L540 tumors (up to 300 mm³). Anti-PS Ab localized in 3 animals of 5 in the group of intermediate size L540 tumors and in all mice (4 out of 4) bearing large L540 tumors. Percent of PS-positive blood vessels from total (identified by pan endothelial marker Meca 32) was 10-20% in the L540 intermediate group and 20-40% in the group of large L540 tumors.

### EXAMPLE XX

Anti-Tumor Effects of Unconjugated Anti-Phosphatidylserine Antibodies

A. Methods

The effects of anti-PS antibodies were examined in syngeneic and xenogeneic tumor models. For the syngeneic model, x10⁷ cells of murine colorectal carcinoma Colo 26 (obtained from Dr. Ian Hart, ICRF, London) were injected subcutaneously into the right flank of Balb/c mice. In the xenogeneic model, a human Hodgkin's lymphoma L540 xenograft was established by injecting 1×10⁷ cells subcutaneously into the right flank of male CB17 SCID mice. Tumors were allowed to grow to a size of about 0.6-0.9 cm³ before treatment.

Tumor-bearing mice (4 animals per group) were injected i.p. with 20 μg of naked anti-PS antibody (IgM). control mouse IgM or saline. Treatment was repeated 3 times with a 48 hour interval. Animals were monitored daily for tumor measurements and body weight and tumor volume was calculated. Mice were sacrificed when tumors had reached 2 cm³ or earlier if tumors showed signs of necrosis or ulceration.

B. Results

The growth of both syngeneic and xenogeneic tumors was effectively inhibited by treatment with naked anti-PS antibodies. Anti-PS antibodies caused tumor vascular injury, accompanied by thrombosis, and tumor necrosis. The presence of clots and disintegration of tumor mass surrounding blocked blood vessels was evident.

Quantitatively, the naked anti-PS antibody treatment inhibited tumor growth by up to 60% of control tumor volume in mice bearing large Colo 26 and L540 tumors. No retardation of tumor growth was found in mice treated with saline or control IgM. No toxicity was observed in mice treated with anti-PS antibodies, with normal organs preserving unaltered morphology, indistinguishable from untreated or saline-treated mice.

Tumor regression started 24 hours after the first treatment and tumors continue to decline in size for the next 6 days. This was observed in both syngeneic and immunocompromised tumor models, indicating that the effect was mediated by immune status-independent mechanism(s). Moreover, the decline in tumor burden was associated with the increase of alertness and generally healthy appearance of the animals, compared to control mice bearing tumors larger than 1500 mm³. Tumor re-growth occurred 7-8 days after the first treatment.

The results obtained with anti-PS treatment of L540 tumors are further compelling for the following reasons. Notably, the tumor necrosis observed in L540 tumor treatment occurred despite the fact that the percentage of vessels that stained positive for PS in L540 tumors was less than in HT 29 and NCI-H358 tumors. This implies that even more rapid necrosis would likely result when treating other tumor types. Furthermore, L540 tumors are generally chosen as an experimental model because they provide clean histological sections and they are, in fact, known to be resistant to necrosis.

### EXAMPLE XXI

### Phosphatidylserine Induction by Hydrogen Peroxide

The discovery of PS as an in vivo surface marker unique to tumor vascular endothelial cells prompted the inventors to further investigate the effect of a tumor environment on PS translocation and outer membrane expression. The present example shows that exposing endothelial cells in vitro to certain conditions that mimic those in a tumor duplicates the observed PS surface expression in intact, viable cells.

A. Methods

Mouse bEnd.3 endothelial cells were seeded at an initial density of 50,000 cells/well. Twenty-fours later cells were incubated with increasing concentrations of H₂O₂ (from 10 μM to 500 μM) for 1 hour at 37° C. or left untreated. At the end of the incubation, cells were washed 3 times with PBS containing 0.2% gelatin and fixed with 0.25% glutaraldehyde. Identical wells were either stained with anti-PS IgM or trypsinized and evaluated for viability by the Trypan Blue exclusion test. For the anti-PS staining, after blocking with 2% gelatin for 10 min., cells were incubated with 2 μg/ml of anti-PS antibody, followed by detection with anti-mouse IgM-HRP conjugate.

Wells seeded with mouse bEnd.3 endothelial cells were also incubated with different effectors and compared to control, untreated wells after the same period of incubation at 37° C. After incubation, cells were washed and fixed and were again either stained with anti-PS IgM or evaluated for viability using the Trypan Blue exclusion test.

B. Results

Exposing endothelial cells to H₂O₂ at concentrations higher than 100 μM caused PS translocation in ˜90% cells. However, this was accompanied by detachment of the cells from the substrate and cell viability decreasing to about 50-60%. The association of surface PS expression with decreasing cell viability is understandable, although it is still interesting to note that ˜90% PS translocation is observed with only a 50-60% decrease in cell viability.

Using concentrations of H₂O₂ lower than 100 μM resulted in significant PS expression without any appreciable reduction in cell viability. For example. PS was detected at the cell surface of about 50% of cells in all H₂O₂ treated wells using H₂O₂ at concentrations as low as 20 μM. It is important to note that, under these low H₂O ₂ concentrations, the cells remained firmly attached to the plastic and to each other, showed no morphological changes and had no signs of cytotoxicity. Detailed analyses revealed essentially 100% cell-cell contact, retention of proper cell shape and an intact cytoskeleton.

The 50% PS surface expression induced by low levels of H₂O₂ was thus observed in cell populations in which cell viability was identical to the control, untreated cells (i.e., 95%). The PS expression associated with high H₂O, concentrations was accompanied by cell damage, and the PS-positive cells exposed to over 100 μM H₂O₂ were detached, floating and had disrupted cytoskeletons.

The maintenance of cell viability in the presence of low concentrations H₂O₂ is consistent with data from other laboratories. For example, Schorer et al. (1985) showed that human umbilical vein endothelial cells (HUVEC) treated with 15 μM H₂O₂ averaged 90 to 95% viability (reported as 5% to 10% injury), whilst those exposed to 1500 μM H₂O₂ were only 0%-50% viable (50% to 100% injured).

The use of H₂O₂ to mimic the tumor environment in vitro is also appropriate in that the tumor environment is rich in inflammatory cells, such as macrophages. PMNs and granulocytes. which produce H₂O₂ and other reactive oxygen species. Although never before connected with stable tumor vascular markers, inflammatory cells are known to mediate endothelial cell injury by mechanisms involving reactive oxygen species that require the presence of H₂O₂ (Weiss et al., 1981; Yamada et al, 1981; Schorer et al., 1985). In fact. studies have shown that stimulation of PMNs in vitro produces concentrations of H₂O₂ sufficient to cause sublethal endothelial cell injury without causing cell death (measured by chromium release assays) or cellular detachment; and that these H₂O₂ concentrations are attainable locally in vivo (Schorer et al., 1985).

The present in vitro translocation data correlates with the earlier results showing that anti-PS antibodies localize specifically to tumor vascular endothelial cells in vivo, and do not bind to cells in normal tissues. The finding that in vivo-like concentrations of H₂O₂ induce PS translocation to the endothelial cell surface without disrupting cell integrity has important implications in addition to validating the original in vivo data and the inventors' therapeutic approaches.

Human, bovine and murine endothelial cells are all known to be PS-negative under normal conditions. Any previously documented PS expression has always been associated with cell damage and/or cell death. This is simply not the case in the present studies, where normal viability is maintained. This shows that PS translocation in tumor vascular endothelium is mediated by biochemical mechanisms unconnected to cell damage. This is believed to be the first demonstration of PS surface expression in morphologically intact endothelial cells and the first indication that PS expression can be disconnected from the apoptosis pathway(s). Returning to the operability of the present invention, these observations again confirm that PS is a sustainable, rather than transient, marker of tumor blood vessels and a suitable candidate for therapeutic intervention.

### EXAMPLE XXII

### Anti-Tumor Effects of Annexin-tTF Conjugates

The present example details the use of non-antibody-based targeting regions in delivering coagulants for targeted cancer treatment.

In this example, annexins (aminophospholipid-binding proteins) are used to specifically deliver therapeutic agents to tumor vasculature. The following data shows the anti-tumor effects that result from the in vivo administration of annexin-TF constructs.

An annexin V-tTF conjugate was prepared and administered to nu/nu mice with solid tumors. The tumors were formed from human HT29 colorectal carcinoma cells that formed tumors of at least about 1.2 cm³. The annexin V-tTF coaguligand (10 μg) was administered intravenously and allowed to circulate for 24 hours. Saline-treated mice were separately maintained as control animals. After the one day treatment period, the mice were sacrificed and exsanguinated and the tumors and major organs were harvested for analysis.

The annexin V-tTF conjugate was found to induce specific tumor blood vessel coagulation in HT29 tumor bearing mice. Approximately 55% of the tumor blood vessels in the annexin V-tTF conjugate treated animals were thrombosed following a single injection. In contrast, there was minimal evidence of thrombosis in the tumor vasculature of the control animals.

### EXAMPLE XXIII

### Generation and Unique Characteristics of Anti-VEGF Antibody 2C3

A. Materials and Methods

1. Immunogens

Peptides corresponding to the N-terminal 26 amino acids of human VEGF (huVEGF) and the N-terminal 25 amino acids of guinea pig VEGF (gpVEGF) were synthesized by the Biopolymers Facility of the Howard Hughes Medical Institute at UT Southwestern Medical Center at Dallas. The peptides had the sequences as disclosed in Example I of U.S. Pat. Nos. 6,342.219, 6,342,221 and 6,416,758, each specifically incorporated herein by reference.

Peptides were conjugated via the C-terminal cysteine to thyroglobulin using succinimidyl 4-(N-maleimidomethyl) cyclohexane-1-carboxylate (SMCC) linker (Pierce, Rockford, Ill.). Control conjugates were also prepared that consisted of L-cysteine linked to thyroglobulin. Conjugates were separated from free peptide or linker by size exclusion chromatography.

Recombinant human VEGF was also separately used as an immunogen (obtained from Dr. S. Ramakrishnan, University of Minnesota, Minneapolis, Minn.).

2. Hybridomas

For the production of anti-gpVEGF antibody producing hybridomas, C57/Bl-6 mice were immunized with the gpVEGF-peptide-thyroglobulin conjugate in TiterMax adjuvant (CytRX Co., Norcross, Ga.). For the production of anti-human VEGF antibodies, BALB/c mice were immunized with either the huVEGF-peptide-thyroglobulin conjugate or recombinant human VEGF in TiterMax. Three days after the final boost spleenocytes were fused with myeloma P3X63AG8.653 (American Type Culture Collection, Rockville, Md.) cells and were cultured.

3. Antibody Purification

IgG antibodies (2C3, 12D7, 3E7) were purified from tissue culture supernatant by ammonium sulfate precipitation and Protein A chromatography using the Pierce ImmunoPure Binding/Elution buffering system (Pierce).

IgM antibodies (GV39M, 11B5, 7G3) were purified from tissue culture supernatant by 50% saturated ammonium sulfate precipitation, resuspension of the pellet in PBS (pH 7.4) and dialysis against dH₂O to precipitate the euglobulin. The dH₂O precipitate was resuspended in PBS and fractionated by size-exclusion chromatography on a Sepharose S300 column (Pharmacia). The IgM fraction was 85-90% pure, as judged by SDS-PAGE.

4. Control Antibodies

Various control antibodies have been used throughout these studies including mAb 4.6.1 (mouse anti-human VEGF from Genentech, Inc.), Ab-3 (mouse anti-human VEGF from OncogeneScience, Inc.), A-20 (rabbit anti-human VEGF from Santa Cruz Biotechnology, Inc., Santa Cruz, Calif.), OX7 (mouse anti-rat Thy1.1 from Dr. A. F. Williams, MRC Cellular Immunology Unit, Oxford, UK), MTSA (a mouse myeloma IgM of irrelevant specificity from Dr. E. S. Vitetta, UT-Southwestern, Dallas, Tex.), 1A8 (mouse anti-mouse Flk-1, Philip E. Thorpe and colleagues), MECA 32 (rat anti-mouse endothelium from Dr. E. Butcher, Stanford University, Stanford, Calif.), and TEC 11 (mouse anti-human endoglin; U.S. Pat. No. 5,660,827).

5. Initial Screening

For the initial screening, 96-well ELISA plates (Falcon, Franklin Lakes, N.J.) were coated with 250 ng of either the VEGF peptide or VEGF-Cys-thyroglobulin conjugate and blocked with 5% casein acid hydrolysate (Sigma, St. Louis, Mo.). Supernatants from the anti-gpVEGF hybridomas and the initial anti-human VEGF hybridomas were screened on the antigen coated plates through a dual indirect ELISA technique.

Hybridomas that showed preferential reactivity with VEGF peptide-thyroglobulin but no or weak reactivity with Cys-thyroglobulin were further screened through immunohistochemistry (described below) on frozen sections of tumor tissue.

6. Immunohistochemistry

Guinea pig line 10 hepatocellular carcinoma tumor cells (obtained from Dr. Ronald Neuman, NIH, Bethesda, Md.) were grown in strain 2 guinea pigs (NCI, Bethesda, Md.). The human tumors NCI-H358 non-small cell lung carcinoma (NSCLC), NCI-H460 NSCLC (both obtained from Dr. Adi Gazdar, UT Southwestern, Dallas, Tex.), HT29 colon adenocarcinoma (American Type Culture Collection), and L540CY Hodgkin's lymphoma (obtained from Professor V. Diehl, Cologne. Germany) were grown as xenografts in CB17 SCID mice (Charles River, Wilmington, Mass.).

Tumors were snap frozen in liquid nitrogen and stored at −70° C. Frozen samples of tumor specimens from patients were obtained from the National Cancer Institute Cooperative Human Tissue Network (Southern Division. Birmingham, Ala.).

7. ELISA Analysis

Hybridoma supernatants from animals immunized with VEGF were screened through a differential indirect ELISA technique employing three different antigens: human VEGF alone, VEGF:Flk-1/SEAP complex, and Flk-1/SEAP alone. For the human VEGF alone, certain ELISA plates were coated with 100 ng of VEGF.

For Flk-1/SEAP alone, other ELISA plates were coated with 500 ng of Flk-1/SEAP, a soluble form of the mouse VEGF receptor (cells secreting Flk-1/SEAP were obtained from Dr. Ihor Lemischka Princeton University, Princeton, N.J.). The Flk-1/SEAP protein was produced and purified using the extracellular domain of Flk-1 (sFlk-1) produced in Spodoptera frugiperda (Sf9) cells and purified by immunoaffinity techniques utilizing a monoclonal anti-Flk-1 antibody (1A8). sFlk-1 was then biotinylated and bound on avidin-coated plates.

To prepare plates coated with VEGF:Flk-1/SEAP complex, purified sFlk-1 was biotinylated and reacted with VEGF overnight at 4° C. in binding buffer (10 mM HEPES. 150 mM NaCl, 20 μg/ml bovine serum albumin and 0.1 μg/ml heparin) at a molar ratio of sFlk-1 to VEGF of 2.5:1 to encourage dimer formation. The VEGF:sFlk-1 complex was then incubated in avidin coated wells of a 96 well microtiter plate to produce plates coated with VEGF associated with its receptor.

The reactivity of the antibodies with VEGF alone, biotinylated sFlk-1 and VEGF:sFlk-1 complex was then determined in controlled studies using the three antigens on avidin-coated plates. The reactivity was determined as described above for the initial screening.

A capture ELISA was also developed. In the capture ELISA, microtiter plates were coated overnight at 4° C. with 100 ng of the indicated antibody. The wells were washed and blocked as above, then incubated with various concentrations of biotinylated VEGF or VEGF:sFlk-1-biotin. Streptavidin conjugated to peroxidase (Kirkegaard & Perry Laboratories, Inc.). diluted 1:2000. was used as a second layer and developed.

Competition ELISA studies were performed by first labeling the antibodies with peroxidase according to the manufacturer's instructions (EZ-Link Activated Peroxidase, Pierce). The antigen used for the competition studies with 12D7, 3E7, 2C3. and 7G3 was VEGF-biotin captured by avidin on an ELISA plate. Approximately 0.5-2.0 μg/ml of peroxidase labeled test antibody was incubated on the plate in the presence of either buffer alone, an irrelevant IgG, or the other anti-VEGF competing antibodies in a 10-100 fold excess.

The binding of the labeled antibody was assessed by addition of 3,3′5,5′-tetramethylbenzidine (TMB) substrate (Kirkegaard and Perry Laboratories, Inc). Reactions were stopped after 15 min with 1 M H₃PO₄ and read spectrophotometrically at 450 nM. The assay was done in triplicate at least twice for each combination of labeled and competitor antibody. Two antibodies were considered to be in the same epitope group if they cross-blocked each other's binding by greater than 80%.

GV39M and 11B5 did not retain binding activity after peroxidase labeling but tolerated biotinylation. GV39M and 11B5 were biotinylated and tested against VEGF:sFlk-1 that had either been captured by the anti-Flk-1 antibody (1A8) or coated directly on an ELISA plate.

8. Western Blot Analysis

Purified recombinant VEGF in the presence of 5% fetal calf serum was separated by 12% SDS-PAGE under reducing and non-reducing conditions and transferred to nitrocellulose. The nitrocellulose membrane was blocked using Sea-Block PP82-41 (East Coast Biologics, Berwick, Me.), and probed with primary antibodies using a mini-blotter apparatus (Immunetics, Cambridge, Mass.). The membranes were developed after incubation with the appropriate peroxidase-conjugated secondary antibody by ECL enhanced chemiluminescence.

B. RESULTS

1. 2C3 has a Unique Epitope Specificity

Table 1 of U.S. Pat. Nos. 6,342,219, 6,342,221 and 6,416,758 (see also WO 00/64946), each specifically incorporated herein by reference, summarizes information on the class/subclass of different anti-VEGF antibodies, the epitope groups that they recognize on VEGF, and their preferential binding to VEGF or VEGF:receptor (VEGF:Flk-1) complex. In all instances the antibodies bound to VEGF121 and VEGF165 equally well and produced essentially the same results. The results are for VEGF165 unless stipulated otherwise.

Competitive binding studies using biotinylated or peroxidase-labeled test antibodies and a 10-100-fold excess of unlabeled competing antibodies showed that 2C3 binds to a unique epitope. These studies first revealed that GV39M and 11B5 cross-blocked each other's binding to VEGF:Flk-1, and that 3E7 and 7G3 cross-blocked each other's binding to VEGF-biotin captured onto avidin. GV39M and 11B5 were arbitrarily assigned to epitope group 1, while 3E7 and 7G3 were assigned to epitope group 2. 2C3 and the remaining antibody, 12D7, did not interfere significantly with each other's binding or the binding of the rest of the antibodies to VEGF or VEGF:receptor. 12D7 was assigned to epitope group 3, and 2C3 was assigned to epitope group 4.

2C3 thus sees a different epitope to the antibody A4.6.1. The inventors' competition studies showed that 2C3 and A4.6.1 are not cross-reactive. The epitope recognized by A4.6.1 has also been precisely defined and is a continuous epitope centered around amino acids 89-94 (Kim et al., 1992; Wiesmann et al., 1997; Muller et al.,1998; Keyt et al., 1996; each incorporated herein by reference). There are also a number known differences between 2C3 and A4.6.1 (see below).

2. 2C3 Binds to Free, not Receptor Bound, VEGF

There were marked differences in the ability of the antibodies to bind to soluble VEGF in free and complexed form. These studies provide further evidence of the unique nature of 2C3, GV39M and 11B5 display a strong preference for the VEGF:receptor complex, with half-maximal binding being attained with VEGF:Flk-1 at 5.5 and 2nM respectively as compared with 400 and 800 nM respectively for free VEGF in solution.

In contrast, 2C3 and 12D7 displayed a marked preference for free VEGF, with half-maximal binding being attained at 1 and 20 nM respectively as compared with 150 and 250 nM respectively for the VEGF:Flk-1 complex. 3E7 bound equally well to free VEGF and the VEGF:Flk-1 complex, with half-maximal binding being attained at 1 nM for both.

3. 2C3 Recognizes a Non-Conformationally-Dependent Epitope

Western blot analysis shows that 12D7, 2C3 and 7G3 react with denatured VEGF121 and VEGF165 under reducing and non-reducing conditions. These antibodies therefore appear to recognize epitopes that are not conformationally-dependent.

In contrast, GV39M, 11B5, and 3E7 did not react with VEGF on western blots, possibly because they recognize an epitope on the N-terminus of VEGF that is conformationally-dependent and is distorted under denaturing conditions. A typical western blot for the different antibodies shows that dimeric VEGF is a large band at approximately 42 kd and a multimer of VEGF is evident with 12D7, 7G3, and a positive control antibody at approximately 130 kd.

4. Tumor Immunohistochemistry

Tumors examined through immunohistochemistry were human tumors of various types from cancer patients, transplantable human tumor xenografts of various types grown in mice, guinea pig Line 10 tumor grown in guinea pig, and mouse 3LL tumor grown in mice.

GV39M and 11B5. which recognize epitope group 1 on VEGF, stained vascular endothelial cells strongly and perivascular connective tissue moderately in all tumors examined. The epitope group 1 antibodies differed in their reactivity with tumor cells, in that GV39M reacted only weakly with tumor cells while 11B5 reacted more strongly. Approximately 80% of endothelial cells that were stained by MECA 32 (mouse) or TEC 11 (human) were also stained by GV39M and 11B5.

3E7 and 7G3. which recognize VEGF epitope group 2, showed reactivity with vascular endothelial cells, connective tissue, and tumor cells in all tumors examined. The intensity of endothelial cell staining was typically stronger than the tumor cell or connective tissue staining, especially when the antibodies were applied at low (1-2 μg/ml) concentrations where there was a noticeably increased selectivity for vascular endothelium. 12D7 and 2C3 did not stain frozen sections of any tumor tissues, probably because acetone fixation of the tissue destroyed antibody binding. However, 2C3 localized to tumor tissue after injection in vivo (see below).

GV39M, 11B5, 3E7 and 7G33 reacted with rodent vasculature on frozen sections of guinea pig line 10 tumor grown in guinea pigs and mouse 3LL tumor grown in mice. GV39M. 11 B5, and 7G33 reacted as strongly with guinea pig and mouse tumor vasculature as they did with human vasculature in human tumor specimens. 3E7 stained the mouse 3LL tumor less intensely than it did the guinea pig or human tumor sections, suggesting that 3E7 has a lower affinity for mouse VEGF. These results accord with analysis by indirect ELISA, which has shown that all the antibodies except 2C3 react with mouse VEGF.

5. Advantages of 2C3 Over A4.6.1

There are a number differences between 2C3 and A4.6.1. The antibodies recognize distinct epitopes on VEGF based upon ELISA cross-blocking studies. Mutagenesis and X-ray crystallographic studies have earlier shown that A4.6.1 binds to an epitope on VEGF that is centered around amino acids 89-94 (Muller et al., 1998).

Of particular interest is the fact that A4.6.1 blocks VEGF from binding to both VEGFR1 and VEGFR2 (Kim et al., 1992; Wiesmann et al., 1997; Muller et al.,1998; Keyt et al., 1996), while 2C3 only blocks VEGF from binding to VEGFR2 (Example IV). Compelling published evidence that A4.6.1 inhibits VEGF binding to VEGFR2 and VEGFR1 comes from detailed crystallographic and structural studies (Kim et al., 1992; Wiesmann et al., 1997; Muller et al.,1998; Keyt et al., 1996; each incorporated herein by reference). The published data indicate that A4.6.1 inhibits VEGF binding to VEGFR2 by competing for the epitope on VEGF that is critical for binding to VEGFR2, and blocks binding of VEGF to VEGFR1 most probably by steric hindrance (Muller et al.,1998; Keyt et al, 1996).

A humanized version of A4.6.1 is currently in clinical trials (Brem, 1998; Baca et al., 1997; Presta et al., 1997; each incorporated herein by reference). Macrophage/monocyte chemotaxis and other endogenous functions of VEGF that are mediated through VEGFR1 will most likely be impaired in the A4.6.1 trials. In contrast, 2C3 is envisioned to be superior due its ability to specifically block VEGFR2-mediated effects. 2C3 is thus potentially a safer antibody, particularly for long-term administration to humans. The benefits of treatment with 2C3 include the ability of the host to mount a greater anti-tumor response, by allowing macrophage migration to the tumor at the same time it is blocking VEGF-induced tumor vasculature expansion. Also, the many systemic benefits of maintaining macrophage chemotaxis and other effects mediated by VEGFR1 should not overlooked.

### EXAMPLE XXIV

### 2C3 Specifically Localizes to Tumors In Vivo

A. Materials and Methods

In Vivo Localization to Human Tumor Xenografts

Tumors were grown subcutaneously in immunocompromised mice (NCI-H358 NSCLC in nu\nu mice and HT29 colon adenocarcinoma in SCID mice) until the tumor volume was approximately 1 cm³. 100 μg of unlabeled antibody for studies using SCID mice, or 100 μg of biotinylated antibody for studies using nude mice, was injected intravenously via a tail vein. Twenty four hours later, the mice were anesthetized, perfused with PBS, and tumor and organs including heart, lungs, liver, kidneys, intestines and spleen were collected and snap frozen in liquid nitrogen.

The tumor and organs from each mouse were sectioned on a cryostat and stained for antibody immunohistochemically as above, with the exception that sections from the nude mice were developed using peroxidase labeled streptavidin-biotin complex (Dako, Carpinteria, Calif.) and the sections from the SCID mice were developed using two peroxidase-conjugated secondary antibodies, a goat anti-mouse IgG+IgM followed by a rabbit anti-goat IgG.

B. Results

In Vivo Localization in Tumor-Bearing Mice

100 μg of 3E7, GV39M, 2C3, and isotype matched control antibodies were injected intravenously into nu/nu mice bearing NCI-H358 human NSCLC and SCID mice bearing HT29 human colonic adenocarcinoma. Twenty four hours later, the mice were exsanguinated and the tumors and tissues were analyzed immunohistochemically to determine the binding and distribution of the antibodies.

3E7 specifically localized to vascular endothelium within the tumors. Approximately 70% of MECA 32 positive blood vessels were stained by 3E7 injected in vivo. The larger blood vessels that feed the microvasculature were 3E7-positive. Small microvessels in both the tracks of stroma and in the tumor nests were also positive for 3E7. The intensity of the staining by 3E7 was increased in and around areas of focal necrosis. In necrotic areas of the tumor, extravascular antibody was evident, but in viable regions of the tumor there was little evidence of extravascular staining. Vascular endothelium in all normal tissues examined. including the kidney, was unstained by 3E7.

GV39M also specifically localized to vascular endothelium of the tumors. Approximately 80% of the MECA 32 positive blood vessels in the tumor were stained by GV39M. The GV39M positive vessels were distributed evenly throughout the tumor, including large blood vessels, but also small capillaries. As with 3E7, the staining intensity of the GV39M positive blood vessels was increased in areas of focal necrosis in the tumor. However, unlike 3E7, endothelial cells or mesangial cells in the kidney glomeruli were also stained. It appears that the staining of the glomeruli by GV39M is antigen-specific since a control IgM of irrelevant specificity produced no staining of the glomeruli. Vascular endothelium in tissues other than the kidney was not stained by GV39M.

Biotinylated 2C3 produced intense staining of connective tissue surrounding the vasculature of the H1358 human NSCLC tumor after i.v. injection. The large tracks of stromal tissue that connect the tumor cell nests were stained by 2C3, with the most intense localization being observed in the largest tracks of stroma. It was not possible to distinguish the vascular endothelium from the surrounding connective tissue in these regions. However, the endothelial cells in vessels not surrounded by stroma, such as in vessels running through the nests of tumor cells themselves, were stained in some cases. There was no detectable staining by 2C3 in any of the normal tissues examined.

In the HT29 human tumor model, 2C3 also localized strongly to the connective tissue but the most intense staining was observed in the necrotic regions of the tumor.

### EXAMPLE XXV

### 2C3 Inhibits VEGF Binding to VEGFR2, but not VEGFR1

A. Materials and Methods

1. Cell Lines and Antibodies

Porcine aortic endothelial (PAE) cells transfected with either VEGFR1 (PAE/FLT) or VEGFR2 (PAE/KDR) were obtained from Dr. Johannes Waltenberger (Ulm, Germany) and were grown in F-12 medium containing 5% FCS, L-glutamine. penicillin, and streptomycin (GPS). bEND.3 cells were obtained from Dr. Werner Risau (Bad Nauheim, Germany) and were grown in DMEM medium containing 5% FCS and GPS. NCI-H358 NSCLC (obtained from Dr. Adi Gazdar, UT-Southwestern, Dallas, Tex.), A673 human rhabdomyosarcoma, and HT1080 human fibrosarcoma (both from American Type Culture Collection) were grown in DMEM medium containing 10% FCS and GPS.

2C3 and 3E7, anti-VEGF monoclonal antibodies, and 1A8, monoclonal anti-Flk-1 antibody, and T014, a polyclonal anti-Flk-1 antibody are as described above. A4.6.1. mouse anti-human VEGF monoclonal antibody, was obtained from Dr. Jin Kim (Genentech Inc., Calif.) and has been described previously (Kim et al., 1992). Negative control antibodies used were OX7, a mouse anti-rat Thy1.1 antibody, obtained from Dr. A. F. Williams (MRC Cellular Immunology Unit, Oxford, UK) and C44, a mouse anti-colchicine antibody (ATCC).

2. ELISA Analysis

The extracellular domain of VEGFR1 (Flt-1/Fc, R&D Systems, Minneapolis) or VEGFR2 (sFlk-1-biotin) was coated directly on wells of a microtiter plate or captured by NeutrAvidin (Pierce. Rockford, Ill.) coated wells, respectively. VEGF at a concentration of 1 nM (40 ng/ml) was incubated in the wells in the presence or absence of 100-1000 nM (15 μg-150 μg/ml) of control or test antibodies. The wells were then incubated with 1 μg/ml of rabbit anti-VEGF antibody (A-20, Santa Cruz Biotechnology, Santa Cruz, Calif.).

The reactions were developed by the addition of peroxidase-labeled goat anti-rabbit antibody (Dako, Carpinteria, Calif.) and visualized by addition of 3,3′5,5′-tetramethylbenzidine (TMB) substrate (Kirkegaard and Perry Laboratories, Inc.). Reactions were stopped after 15 min with 1 M H₃PO₄ and read spectrophotometrically at 450 nM.

The assay was also performed by coating wells of a microtiter plate with either control or test IgG. The wells were then incubated with VEGF:Flt-1/Fc or VEGF:sFlk-1-biotin and developed with either peroxidase-labeled goat anti-human Fc (Kirkegaard and Perry Laboratories. Inc.) or peroxidase-labeled streptavidin. respectively and visualized as above.

B. Results

ELISA Reactivity of VEGFR1 and VEGFR2 with VEGF:IgG Complex

The anti-VEGF antibody 2C3 blocked VEGF from binding to VEGFR2 (KDR/Flk-1) but not to VEGFR1 (FLT-1) in the ELISA assay. In the presence of a 100-fold and 1000-fold molar excess of 2C3, the amount VEGF that bound to VEGFR2-coated wells was reduced to 26% and 19%, respectively, of the amount that bound in the absence of 2C3. In contrast, in the presence of a 100 fold and 1000 fold molar excess of 2C3, the amount VEGF that bound to VEGFR1-coated wells was 92% and 105%, respectively, of the amount that bound in the absence of 2C3.

The amounts of VEGF that bound to VEGFR1 or VEGFR2 were unaffected by the presence of a 100-1000 fold excess of the non-blocking monoclonal anti-VEGF antibody 3E7 or of a control IgG of irrelevant specificity.

A4.6.1 blocked VEGF binding to both VEGFR2 (KDR/Flk-1) and VEGFR1 (FLT-1).

### EXAMPLE XXVI

### Anti-Tumor Effects of 2C3

A. Materials and Methods

1. In Vivo Tumor Growth Inhibition

Nu/nu mice were injected subcutaneously with either 1×10⁷ NCI-H358 NSCLC cells or 5×10⁶ A673 rhabdomyosarcoma cells on day 0. On day 1 and subsequently twice per wk the mice were given i.p. injections of 2C3 at 1, 10. or 100 μg or controls as indicated. The tumors were then measured twice per wk for a period of approximately six wk for the NCI-H358 bearing mice and four wk for the A673 bearing mice. Tumor volume was calculated according to the formula: volume=L×W×H, where L=length, W=width, H=height.

2. In Vivo Tumor Therapy

Nu/nu mice bearing subcutaneous NCI-H358 tumors or HT1080 fibrosarcoma 200-400 mm³ in size were injected i.p. with test or control antibodies. The NCI-H358 bearing mice were treated at 100 μg per injection three times per wk during the first wk and twice per wk during the second and third wk. The mice were then switched to 50 μg per injection every five days. The HT1080 bearing mice were treated with 100 μg of the indicated antibody or saline every other day throughout the duration of the study. In both studies mice were sacrificed if they appeared sick or if their tumors reached 2500 mm³ in size.

B. Results

1. Growth Inhibition of Newly-implanted Human Tumor Xenografts

2C3 inhibits the in vivo growth of both NCI-H358 NSCLC and A673 rhabdomyosarcoma in nu/nu mice in a dose dependent manner. 100 μg of 2C3 given i.p. 2 times per wk to mice that had been injected with tumor cells subcutaneously one day earlier inhibited the growth of both human tumor types. The final tumor volume in the 2C3 recipients was approximately 150 mm³ in both tumor systems, as compared with approximately 1000 mm³ in the recipients of controls. Treatment with either 10 or 1 μg of 2C3 twice per wk was less effective at preventing tumor growth. However, both lower doses of 2C3 did slow the growth of A673 tumors to a similar degree compared to the untreated mice.

In contrast the 10 μg dose of 2C3 only marginally slowed the growth of the NCI-H358 tumors and mice given 1 μg of 2C3 showed no tumor growth retardation. The differences between these two tumor models and their response to inhibition of VEGFR2 activity by 2C3 correlates with the aggressiveness of the two types of tumors in vivo. NCI-H358 grows in vivo much more slowly than does A673 and appears to be less sensitive to low doses of 2C3, whereas, A673 tumors grow more quickly and aggressively and appear to be more sensitive to lower doses of 2C3.

3E7, which binds to VEGF but does not block its activity, had no effect on the growth of NCI-H358 tumors. However. 3E7 given at a dose of 100 μg twice per wk stimulated the growth of A673 tumors, suggesting that it increases the efficiency of VEGF signaling in the tumor.

2. Treatment of Established Human Tumor Xenografts with 2C3

Mice bearing subcutaneous NCI-H358 NSCLC tumors that had grown to a size of approximately 300 mm³ were injected i.p. with 2C3, A4.6.1, 3E7, or an IgG of irrelevant specificity. Doses were 100 μg twice weekly for 4 wk and 50 μg weekly thereafter. A4.6.1 was used as a positive control because it has been shown by other investigators to block VEGF activity in vivto resulting in an inhibition of tumor growth.

Treatment with either 2C3 or A4.6.1 led to a slow regression of the tumors over the course of the study. The mean tumor volume at the end of the study was 34% or 35% of the initial mean tumor volume, respectively. Representative mice from each treatment group were studied. However, these results are complicated by the fact that spontaneous tumor regressions were seen in all groups of mice, beginning at approximately 40 days after tumor cell injection. These spontaneous regressions contributed to the tumor regressions in the 2C3 and A4.6.1 treated groups. The results up to 40 days, before the spontaneous regressions are evident, show that both 2C3 and A4.6.1 treatment prevent tumor growth.

A further study was conducted in which mice bearing NCI H358 were treated for a prolonged period with 100 μg of either 2C3 or 3E7. In this study, spontaneous regressions were less pronounced. The mean tumor volume of the. 2C3 treated mice at the start of treatment was 480 mm³ and after approximately 14 wk of treatment the mean tumor volume dropped to 84 mm³, a decrease of approximately 80% in volume. The 3E7 treated mice began treatment with a mean tumor volume of 428 mm³ and rose to a volume of 1326 mm³ after approximately 14 wk, an increase of 300% in volume.

The tumor growth curves of mice bearing a human fibrosarcoma, HT1080, that were every treated every two days with 100 μg of 2C3, 3E7, or a control IgG, or saline were generated. 2C3 arrested the growth of the tumors. 50% of which began to slowly regress in size. The mice treated with 3E7, control IgG, or saline bore tumors that grew identically and to a size that led to sacrifice of the mice in less that 4 wk after tumor cell injection.

### EXAMPLE XXVII

### 2C3-Tissue Factor Conjugates

2C3 was modified with SMPT as follows. 4-Succinimidyloxycarbonyl-α-methyl-α-(2-pyridyldithio)-toluene (SMPT) in N′N-dimethylformamide (DMF) was added to 2C3 IgG at a molar ratio of 5:1 (SMPT:2C3) and incubated at room temperature (RT) for 1 hr in PBS with 5 mM EDTA (PBSE). Free SMPT was removed by G25 size exclusion chromatography run in PBSE and the peak (2C3-SMPT) was collected under nitrogen. 600 μl of 2C3-SMPT was removed to quantitate thiopyridyl groups after addition of dithiothreitol (DTT) to 50 mM. An average of 3 MPT groups were introduced per IgG. Human truncated tissue factor (tTF) having a cysteine residue introduced at the N-terminus was reduced with 5 mM β 2-ME. β 2-ME was removed by G25 chromatography.

Reduced N-Cys-tTF was pooled with the 2C3-SMPT and incubated at a molar ratio of 2.5:1 (tTF:IgG) for 24 hours at RT. The reaction was concentrated to 1-2 ml using an Amicon with a 50,000 molecular weight cut off (MWCO) membrane. Unconjugated tTF and IgG were separated from conjugates using Superdex 200 size exclusion chromatography, thus providing 2C3-tTF.

All of the compositions and methods disclosed and claimed herein can be made and executed without undue experimentation in light of the present disclosure. While the compositions and methods of this invention have been described in terms of certain preferred embodiments, it will be apparent to those of skill in the art that variations may be applied to the compositions and methods, and in the steps or in the sequence of steps of the methods, described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

Abrams and Oldham, In: Monoclonal Antibody Therapy of Human Cancer, Foon and Morgan (Eds.), Martinus Nijhoff Publishing, Boston, pp. 103-120, 1985.

Aiello, Pierce, Foley, Takagi, Chen, Riddle, Ferrara, King, Smith, “Suppression of retinal neovascularization in vivo by inhibition of vascular endothelial growth factor (VEGF) using soluble VEGF-receptor chimeric proteins,” Proc. Natl. Acad. Sci. USA, 92:10457-10461, 1995.

Anderson, Croyle, Lingrel, “Primary structure of a gene encoding rat T-kininogen,” Gene. 81(1):119:28, 1989.

Antibodies: A Laboratory Manual, Cold Spring Harbor Laboratory, 1988.

Asano, Yukita, Matsumoto, Kondo, Suzuki, “Inhibition of tumor growth and metastasis by an immunoneutralizing monoclonal antibody to human vascular endothelial growth factor/vascular permeability factor,” Cancer Res., 55:5296-5301, 1995.

Baca et al., “Antibody humanization using monovalent phage display,” J. Biol. Chem., 272(16): 10678-84, 1997.

Bach et al., Biochemistry. 25: 4007-4020, 1986.

Bannermann, Goldblum, “Direct effects of endotoxin on the endothelium: Barrier function and injury,” Lab Invest., 79:1181-1199, 1999.

Barbas, Kang, Lerner, Benkovic, “Assembly of combinatorial antibody libraries on phage surfaces: the gene III site,” Proc. Natl. Acad Sci. USA, 88(18):7978-7982, 1991.

Bauer, tenCate, Barzegar, Spriggs, and Rosenberg, Blood, 74:165, 1989.

Bauer, et al., Vox Sang, 61:156-157, 1991.

Baxter, et al., Micro. Res., 41(1):5-23. 1991.

Becker, Rudbach, “Potentiation of endotoxin toxicity by carrageenan”, Infect Immun, 19:1099-1100. 1978.

Bennett et al., “Endogenous Production of Cytotoxic Factors in Serum of BCG-Primed Mice by Monophosphoryl Lipid A, a Detoxified Form of Endotoxin,” Journal of Biological Response Modifiers, 7:65-76, 1988.

Berman, Mellis, Pollock, Smith, Suh, Heinke, Kowal, Surti, Chess, Cantor, et al., “Content and organization of the human Ig VH locus: definition of three new VH families and linkage to the Ig CH locus,” EMBO J., 7(3):727-738. 1988.

Bernier and Jolles. “Purification and characterization of a basic 23 kDa cytosolic protein from bovine brain,” Biochim. Biophys. Acta, 790(2):1 74-181, 1984.

Bernier, Tresca, Jolles, “Ligand-binding studies with a 23 kDa protein purified from bovine brain cytosol,” Biochim. Biophys. Acta, 871(1):19-23, 1986.

Beutler, Mahoney, Le Trang, Pekala, Cerami, “Purification of cachectin. a lipoprotein lipase-suppressin hormone secreted by endotoxin-induced RAW 264.7 cells”, J. Exp Med., 161:984-995. 1985.

Bevilacqua, Pober, Majeau, Fiers, Cotran, Gimbrone, Jr., “Recombinant tumor necrosis factor induces procoagulant activity in cultured human vascular endothelium: characterization and comparison with the actions of interleukin 1,” Proc. Natl. Acad. Sci., USA., 83:4533-4537, 1986.

Bevilacqua, “Endothelial-leukocyte adhesion molecules.” Ann. Rev. Immunol., 11:767-804, 1993.

Bierhaus, Zhang, Deng, Mackman, Quehenberger, Haase, Luther, Muller, Bohrer, Greten, Martin, Baeuerle, Waldherr, Kisiel, Ziegler, Stern, Nawroth, “Mechanism of the tumor necrosis factor □-mediated induction of endothelial tissue factor,” J. Biol. Chem., 270:26419-26432, 1995.

Bjorck et al, “Antibodies to distinct epitopes on the CD40 molecule co-operate in stimulation and can be used for the detection of soluble CD40.” Immunology 83:430-437. 1994.

Borgstrom et al., “Complete inhibition of angiogenesis and growth of microtumors by anti-vascular endothelial growth factor neutralizing antibody: novel concepts of angiostatic therapy from intravital videomicroscopy,” Cancer Res., 56(17):4032-1439. 1996.

Borgstrom et al . “Neutralizing anti-vascular endothelial growth factor antibody completely inhibits angiogenesis and growth of human prostate carcinoma micro tumors in vivo,” Prostate. 35(1):1-10, 1998.

Bornstein, “Thrombospondins: structure and regulation of expression.” FASEB J, 6(14):3290-3299,1992.

Brem. “Angiogenesis antagonists: current clinical trials.” Angiogenesis, 2:9-20, 1998.

Brennan et al, Science, 229:81-83. 1985.

Bruijn and Dinklo. “Distinct patterns of expression of intercellular adhesion molecule-1, vascular cell adhesion molecule-1, and endothelial-leukocyte adhesion molecule-1 in renal disease.” Lab. Invest., 69:329-335. 1993.

Burke et al., “Cloning of large segments of exogenous DNA into yeast by means of artificial chromosome vectors”, Science, 236: 806-812, 1987.

Burrows, Watanabe, Thorpe, “A murine model for antibody-directed targeting of vascular endothelial cells in solid tumors.” Cancer Res., 52:5954-5962, 1992.

Burrows and Thorpe, “Eradication of large solid tumors in mice with an immunotoxin directed against tumor vasculature,” Proc. Natl. Acad. Sci. USA, 90:8996-9000, 1993.

Buske et al., “In vitro activation of low-grade non-Hodgkin's lymphoma by murine fibroblasts, IL-4. anti-CD40 antibodies and the soluble CD40 ligand,” Leukemia 11:1862-1867, 1997.

Byers and Baldwin Immunol., 65:329-335, 1988.

Camera, Giesen, Fallon, Aufiero, Taubman, Tremoli, Nemerson, “Cooperation between VEGF and TNF-□ is necessary for exposure of active tissue factor on the surface of human endothelial cells,” Arterioscler. Thromb. Vasc. Biol., 19:531-537, 1999.

Campbell, In: Monoclonal Antibody Technology, Laboratory Techniques in Biochemistry and Molecular Biology, Vol. 13, Burden and Von Knippenberg (Eds.), Elseview, Amsterdam, pp. 75-83, 1984.

Campos, Almeida, Takeuchi, Akira, Valente, Procopio, Travassos, Smith, Golenbock, and Gazinelli, J. Immunol., 167:416,2001.

Carswell, Old, Kassel, Green, Fiore, Williamson, “An endotoxin-induced serum factor that causes necrosis of tumor,” Proc. Natl. Acad. Sci., U.S.A., 72:3666, 1975.

Chang, Reuter, Heston, Bander, Grauer, Gaudin. “Five different anti-prostate-specific membrane antigen (PSMA) antibodies confirm PSMA expression in tumor-associated neovasculature”, Cancer Res., 59(13):3192-3198, 1999

Chedid, Parant, Audibert, Riveau, Parant, Lederer, Choay, and Lefrancier, Infection and Immunity 35:417, 1982.

Cheng, Huang. Nagane, Ji, Wang, Shih, Arap, Huang, Cavenee, “Suppression of glioblastoma angiogenicity and tumorigenicity by inhibition of endogenous expression of vascular endothelial growth factor.” Proc. Natl. Acad Sci. USA, 93:8502-8507, 1996.

Ching, Cao, Kieda, Zwain, Jameson, and Baguley, British Journal of Cancer 17:1937, 2002.

Chomel, Simon-Lavoine, Thouvenot, Valette, Choay, Chedid, and Aymard, Journal of Biological Response Modifiers 7:581, 1987.

Chu and Prasad, Journal of Surgical Research 80:80, 1998.

Clapp et al . “The 16-kilodalton N-terminal fragment of human prolactin is a potent inhibitor of angiogenesis,38 Endocrinology. 133(3): 1292-1299, 1993.

Clauss, Grell, Fangmann, Fiers, Scheurich, Risau, “Synergistic induction of endothelial tissue factor by tumor necrosis factor and vascular endothelial growth factor: functional analysis of the tumor necrosis factor receptors,” FEBS Lett, 390:334-338,1996.

Coley, “The treatment of malignant tumor by repeated inoculation of erysipelas, with a report of 10 original cases.” Am. J. Med Sci.,105:487, 1893.

Coughlin et al., “Interleukin-12 and interleukin-18 synergistically induce murine tumor regression which involves inhibition of angiogenesis,” J. Clin. Invest., 101(6):1441-1452, 1998.

D'Amato et al., “Thalidomide is an inhibitor of angiogenesis.” Proc. Natl. Acad Sci. USA, 91(9):4082-4085, 1994.

D'Angelo et al., “Activation of mitogen-activated protein kinases by vascular endothelial growth factor and basic fibroblast growth factor in capillary endothelial cells is inhibited by the antiangiogenic factor 16-kDa N-terminal fragment of prolactin,” Proc. Natl. Acad Sci. USA, 92(14):6374-6378, 1995.

Davies and Wiodawer, FASEB J., 9:50-56, 1995.

DeVore et al., “Phase I Study of the Antineovascularization Drug CM101,” Clin. Cancer Res., 3(3):365-372, 1997.

Diehl, Kirchner, Schaadt, Fonatsch, Stein, Gerdes, Boie, “Establishment and characterization of four in vitro cell lines,” J. Cancer Res. Clin. Oncol., 101: 111-124, 1981.

Donate, Kelly, Ruf, Edgington, “Dimerization of tissue factor supports solution-phase autoactivation of factor VII without influencing proteolytic activation of factor X,” Biochemistry 39:11467-11476. 2000.

Donati, “Cancer and thrombosis: from Phlegmasia alba dolens to transgenic mice,” Thromb. Haemost., 74:278-281, 1995.

Drake, Cheng. Chang, Taylor Jr., “Expression of tissue factor, thrombomodulin, and E-selectin in baboons with lethal Escherichia coli sepsis,” Am. J. Pathol., 142:1458-1470, 1993.

Droz, Patey, Paraf, Chretien, Gogusev, “Composition of extracellular matrix and distribution of cell adhesion molecules in renal cell tumors.” Lab. Invest., 71:710-718, 1994.

Dvorak et al., J. Exp. Med., 174:1275-1278, 1991.

Dvorak, Nagy, Dvorak, “Structure of Solid Tumors and Their Vasculature: Implications for Therapy with Monoclonal Antibodies,” Cancer Cells, 3(3):77-85, 1991.

Edgington, Mackman. Brand, Ruf. “The Structural Biology of Expression and Function of Tissue Factor.” Thromb. Haemost., 66(1):67-79, 1991.

Edwards, Geng, Tan, Onishko, Donely, and Hallahan, Cancer Research 62:4671, 2002.

Elsayed, Nakagawa, Kamikubo, Enjyoji, Kato, Sueishi, “Effects of recombinant tissue factor pathway inhibitor on thrombus formation and its in vivo distribution in a rat DIC model.” Am. J. Clin. Pathol., 106:574-583, 1996.

Epenetos et al., Cancer Res., 46:3183-3191, 1986.

Fair, Blood, 62:784-791, 1983.

Fair et al., J. Biol. Chem., 262:11692-11698, 1987.

Ferrara, Clapp, Weiner, “The 16K fragment of prolactin specifically inhibits basal or fibroblast growth factor stimulated growth of capillary endothelial cells.” Endocrinology. 129(2):896-900, 1991.

Ferrara, J. Cell. Biochem., 47:211-218, 1991.

Fisher, Gorman, Vehar, O'Brien, Lawn, “Cloning and expression of human tissue factor cDNA,” Thromb. Res., 48:89-99, 1987.

Folkman et al., “Angiogenesis inhibition and tumor regression caused by heparin or a heparin fragment in the presence of cortisone.” Science, 221:719-725, 1983.

Fotsis et al., “The endogenous oestrogen metabolite 2-methoxyoestradiol inhibits angiogenesis and suppresses tumour growth,” Nature, 368(6468):237-239, 1994.

Franco, de Jonge, Dekkers, Timmermann, Pek, an Deventer, an Deursen, van Kerkhoff, van Gemen, ten Cate, van der Poll, and Reitsma, Hemostasis, Thrombosis, and Vascular Biology 96:554, 2000.

Frater-Schroder et al., “Tumor necrosis factor type alpha, a potent inhibitor of endothelial cell growth in vitro, is angiogenic in vivo,” Proc. Natl. Acad Sci USA, 84(15):5277-5281, 1987.

Frazier, “Thrombospondins,” Curr Opin. Cell Biol, 3(5):792-799, 1991.

Fries, Williams, Atkins, Newman, Lipscomb, Collins, “Expression of VCAM-1 and E-selectin in an in vivo model of endothelial activation,” Am. J. Pathol., 143:725-737. 1993.

Gabrilovac, Tomasic, Boranic, Martin-Kleiner, and Osmak, Research in Experimental Medicine 189:265, 1989.

Gagliardi et al., “Antiangiogenic and antiproliferative activity of suramin analogues,” Cancer Chemother. Pharmacol., 41(2):117-124, 1998.

Gagliardi, Hadd, Collins, “Inhibition of angiogenesis by suramin,” Cancer Res., 52(18):5073-5075, 1992.

Gagliardi and Collins. “Inhibition of angiogenesis by antiestrogens,” Cancer Res., 53(3):533-535, 1993.

Galanos, Freudenberg, Reutter, “Galactosamine-induced sensitization to lethal effects of endotoxin.” Proc. Natl. Acad. Sci, U.S.A., 76:5939-5943, 1979.

Gefter et al., “A simple method for polyethylene glycol-promoted hybridization of mouse myeloma cells,” Somatic Cell Genet, 3:23 1-236, 1977.

Gems, Ferguson, Robertson, Nieves, Page, Blaxter, Maizels, “An abundant, trans-spliced mRNA from Toxocara canis invective larvae encodes a 26-kDa protein with homology to phosphatidylethanolamine-binding proteins,” J. Biol. Chem., 270(31):18517-18522, 1995.

Giles et al., Brit. J. Haematol., 69:491-497, 1988.

Glauser, Zanetti, Baumgartner, Cohen, “Septic shock: Pathogenesis,” Lancet, 338:732-736, 1991.

Goding, In: Monoclonal Antibodies: Principles and Practice, 2nd Edition, Academic Press, Orlando, Fla., pp. 60-61, 65-66, 71-74, 1986.

Good et al., “A tumor suppressor-dependent inhibitor of angiogenesis is immunologically and functionally indistinguishable from a fragment of thrombospondin,” Proc. Natl. Acad. Sci. USA, 87(17):6624-6628, 1990.

Gottstein, Wels, Ober, Thorpe, “Generation and characterization of recombinant vascular targeting agents from hybridoma cell lines,” BioTechniques, 30:190-200, 2001.

Grant et al., “Fibronectin fragments modulate human retinal capillary cell proliferation and migration,” Diabetes, 47(8):1335-1340, 1998.

Gratia, Linz, “Le phenoméne de Shwartzman dans le sarcome du Cobaye,” C. R. Séances Soc. Biol. Ses. Fil., 108:427-428, 1931.

Hagen et al., Proc. Natl. Acad. Sci. U S.A., 83:2412-2416, 1986.

Haran et al., “Tamoxifen enhances cell death in implanted MCF7 breast cancer by inhibiting endothelium growth,” Cancer Res., 54(21):5511-5514, 1994.

Harlos, Martin, O'Brien, Jones, Stuart, Polikarpov, Miller, Tuddenham, Boys, “Crystal structure of the extracellular region of human tissue factor,” Nature, 370:662-666, 1994.

Hasselaar and Sage, “SPARC antagonizes the effect of basic fibroblast growth factor on the migration of bovine aortic endothelial cells,” J. Cell Biochem., 49(3):272-283, 1992.

Hellerqvist et al., “Antitumor effects of GBS toxin: a polysaccharide exotoxin from group B beta-hemolytic streptococcus,” J Cancer Res. Clin. Oncol., 120(1-2):63-70, 1993.

Hiscox and Jiang, “Interleukin-12, an emerging anti-tumour cytokine.” In Vivo, 11(2):125-132, 1997.

Hori, Chae, Murakawa, Matoba, Fukushima, Okubo, Matsubara, “A human cDNA sequence homologue of bovine phosphatidylethanolamine-binding protein,” Gene, 140(2):293-294, 1994.

Horn et al., “Differential effects of angiostatic steroids and dexamethasone on angiogenesis and cytokine levels in rat sponge implants,” Br. J Pharmacol., 118(7):1584-1591, 1996.

Huang, Molema, King, Watkins, Edgington, Thorpe, “Tumor infarction in mice by antibody-directed targeting of tissue factor to tumor vasculature,” Science, 275:547-550, 1997.

Huse, Sastry, Iverson, Kang, Alting-Mees, Burton, Benkovic, Lerner, Science, “Generation of a large combinatorial library of the immunoglobulin repertoire in phage lambda.” 246(4935):1275-1281, 1989.

Ingber el al., “Angioinhibins: Synthetic analogues of fumagillin which inhibit angiogenesis and suppress tumor growth,” Nature, 48:555-557, 1990.

Iwamoto et al., “Inhibition of angiogenesis, tumour growth and experimental metastasis of human fibrosarcoma cells HT1080 by a multimeric form of the laminin sequence Tyr-Ile-Gly-Ser-Arg (YIGSR),” Br. J. Cancer, 73(5):589-595, 1996.

Jackson et al., “Stimulation and inhibition of angiogenesis by placental proliferin and proliferin-related protein,” Science. 266(5190):1581-1584, 1994.

Jain, Cancer Meta. Rev., 9(3):253-266, 1990.

Jendraschak and Sage, “Regulation of angiogenesis by SPARC and angiostatin: implications for tumor cell biology,” Semin. Cancer Biol., 7(3):139-146, 1996.

Jersmann, Hii, Hodge, and Ferrante, Infection and Immunity 69:479, 2001.

Jones, Dear, Foote, Neuberger, Winter, “Replacing the complementarity-determining regions in a human antibody with those from a mouse,” Nature, 321(6069):522-525, 1986.

Jones and Hall, “A 23 kDa protein from rat sperm plasma membranes shows sequence similarity and phospholipid binding properties to a bovine brain cytosolic protein,” Biochim. Biophys. Acta 1080(1):78-82, 1991.

Juweid et al, Cancer Res., 52:5144-5153, 1992.

Kabat et al., “Sequences of Proteins of Immunological Interest,” 5th Ed. Public Health Service, National Institutes of Health, Bethesda. Md., pp. 647-669 in particular, 1991.

Kang, Barbas, Janda, Benkovic, Lerner, “Linkage of recognition and replication functions by assembling combinatorial antibody Fab libraries along phage surfaces,” Proc Natl. Acad Sci., US.A, 88(10):4363-4366, 1991.

Kellermann. Lottspeich, Henschen. Muller-Esterl. “Completion of the primary structure of human high-molecular-mass kininogen. The amino acid sequence of the entire heavy chain and evidence for its evolution by gene triplication,” Eur. J. Biochem., 154(2):471-478, 1986.

Kendall and Thomas, “Inhibition of vascular endothelial cell growth factor activity by an endogenously encoded soluble receptor,” Proc. Natl Acad. Sci. USA, 90:10705-10709, 1993.

Kengatharan, de Kimpe, Foster, and Thiemermarn, Journal of Experimental Medicine 188:305, 1998.

Kenyon, Browne, D'Amato, “Effects of thalidomide and related metabolites in a mouse corneal model of neovascularization,” Exp Eye Res., 64(6):971-978, 1997.

Keyt et al., “Identification of vascular endothelial growth factor determinants for binding KDR and FLT-1 receptors. Generation of receptor-selective VEGF variants by site-directed mutagenesis,” J. Biol. Chem., 271(10):5638-46, 1996.

Kim, Li, Houck, Winer, Ferrara, “The vascular endothelial growth factor proteins: identification of biologically relevant regions by neutralizing monoclonal antibodies.” Growth Factors. 7:53-64, 1992.

Kim et al., “Inhibition of vascular endothelial growth factor-induced angiogenesis suppresses tumour growth in vivo,” Nature, 362:841-844, 1993.

Kitamura, Takagaki, Furuto, Tanaka, Nawa, Nakanishi, “A single gene for bovine high molecular weight and low molecular weight kininogens,” Nature, 305(5934):545-549, 1983.

Kitamura, Kitagawa, Fukushima, Takagaki, Miyata, Nakanishi, “Structural organization of the human kininogen gene and a model for its evolution,” J. Biol. Chem., 260(14):8610-8617, 1985.

Kitamura, Ohkubo, Nakanishi, “Molecular biology of the angiotensinogen and kininogen genes,” J. Cardiovasc. Pharmacol., 10(Suppl 7):S49-S53, 1987.

Kitamura, Nawa, Takagaki, Furuto-Kato, Nakanishi, “Cloning of cDNAs and genomic DNAs for high-molecular-weight and low-molecular-weight kininogens,” Methods Enzymol, 163:230-240, 1988.

Kleinman et al., “The laminins: a family of basement membrane glycoproteins important in cell differentiation and tumor metastases,” Vitam. Horm., 47:161-186, 1993.

Kohler and Milstein, “Continuous cultures of fused cells secreting antibody of predefined specificity,” Nature, 256:495-497, 1975.

Kohler and Milstein, “Derivation of specific antibody-producing tissue culture and tumor lines by cell fusion,” Eur. J. Immunol., 6:511-519, 1976.

Kondo, Asano, Suzuki, “Significance of vascular endothelial growth factor/vascular permeability factor for solid tumor growth. and its inhibition by the antibody,” Biochem Biophys. Res. Commun., 194:1234-1241, 1993.

Konieczny, Bobrzecka, Laidler, Rybarska, “The combination of IgM subunits and proteolytic IgG fragment by controlled formation of interchain disulphides,” Haematologia, 14(1):95-99, 1981.

Krosnick, Mule, McIntosh, and Rosenberg, Cancer Research 49, 3729, 1989.

Kyte and Doolittle. “A simple method for displaying the hydropathic character of a protein.” J. Mol. Biol., 157(1):105-132, 1982.

Lane, Iruela-Arispe, Sage, “Regulation of gene expression by SPARC during angiogenesis in vitro. Changes in fibronectin, thrombospondin-1, and plasminogen activator inhibitor-1,” J. Biol. Chem., 267(23):16736-16745, 1992.

Ledbetter et al., “Agonistic and antagonistic properties of CD40 mAb G28-5 are dependent on binding valency,” Circ. Shock 44:67-72, 1994.

Ledbetter et al., “Agonistic activity of a CD40-specific single-chain Fv constructed from the variable regions of mAb G28-5,” Crit. Rev. Immunol. 17:427-435, 1997.

Lee et al., Methods in Enymology, 237:146-164, 1994.

Lee et al., “Inhibition of urokinase activity by the antiangiogenic factor 16K prolactin: activation of plasminogen activator inhibitor 1 expression,” Endocrinology, 139(9):3696-3703, 1998.

Lehmann, Freudenberg, and Galanos, Journal of Experimental Medicine 165:657, 1987.

Lew et al., Cancer Res., 59:6033-6037, 1999.

Li, Touyz, and Schiffrin, Hypertension 31, 487, 1997.

Lin, Buxton, Acheson, Radziejewski, Maisonpierre, Yancopoulos, Channon, Hale, Dewhirst, George, Peters, “Anti-angiogenic gene therapy targeting the endothelium-specific receptor tyrosine kinase Tie2”, Proc. Natl. Acad Sci, USA, 95(15):8829-34, 1998a.

Lin, Sankar, Shan, Dewhirst, Polverini, Quinn, Peters, “Inhibition of tumor growth by targeting tumor endothelium using a soluble vascular endothelial growth factor receptor,” Cell Growth Differ., 9:49-58, 1998b.

Lindner and Borden, “Effects of tamoxifen and interferon-beta or the combination on tumor-induced angiogenesis,” Int. J. Cancer. 71(3):456-461, 1997.

Lingen, Polverini, Bouck, “Inhibition of squamous cell carcinoma angiogenesis by direct interaction of retinoic acid with endothelial cells,” Lab. Invest., 74(2):476-483, 1996.

Lingen, Polverini, Bouck, “Retinoic acid and interferon alpha act synergistically as antiangiogenic and antitumor agents against human head and neck squamous cell carcinoma.” Cancer Res., 58(23):5551-5558, 1998.

Lip, Chin, Blann, “Cancer and the prothrombotic state,” Lancet Oncol., 3:27-34, 2002.

Liu, Moy, Kim, Xia, Rajasekaran, Navarro, Knudsen, Bander, “Monoclonal antibodies to the extracellular domain of prostate-specific membrane antigen also react with tumor vascular endothelium”, Cancer Res., 57:3629-3634, 1997.

Lowder et al. Blood, 69:199-210, 1987.

Luo, Toyoda, Shibuya, “Differential inhibition of fluid accumulation and tumor growth in two mouse ascites tumors by an antivascular endothelial growth factor/permeability factor neutralizing antibody,” Cancer Res., 58(12):2594-2600, 1998a.

Luo et al., “Significant expression of vascular endothelial growth factor/vascular permeability factor in mouse ascites tumors,” Cancer Res., 58(12):2652-2660, 1998b.

Maisonpierre, Suri Jones, Bartunkova, Wiegand, Radziejewski, Compton, McClain, Aldrich, Papadopoulos, Daly, Davis, Sato, and Yancopoulos, Science 277:55, 1997.

Majewski et al., “Vitamin D3 is a potent inhibitor of tumor cell-induced angiogenesis,” J. Invest. Dermatol. Symp. Proc., 1(1):97-101, 1996.

Martin, Silverman, “Gram-negative sepsis and the adult respiratory distress syndrome,” Clin. Infect. Dis., 14:1213-1228, 1992.

Martin, Reutelingsperger, McGahon, Rader, van Schie, LaFace, Green, “Early redistribution of plasma membrane phosphatidylserine is a general feature of apoptosis regardless of the initiating stimulus: inhibition by overexpression of Bcl-2 and Abl,” J. Exp. Med., 182(5):1545-1556, 1995.

McKay and Shapiro, “Alterations in the blood coagulation system induced by bacterial endotoxin, I. In vivo (generalized Shwartzman reaction),” J. Exp. Med., 107:353-367, 1958.

McKay, “Vessel Wall and Thrombogenesis—Endotoxin,” Thrombos. Diathes. Haemorrh., 29:11-26, 1973.

Mesiano, Ferrara, Jaffe, “Role of vascular endothelial growth factor in ovarian cancer: inhibition of ascites formation by immunoneutralization,” Am. J Pathol., 153(4):1249-1256, 1998.

Millauer, Longhi, Plate, Shawver, Risau, Ullrich, Strawn, “Dominant-negative inhibition of Flk-1 suppresses the growth of many tumor types in vivo,” Cancer Res., 56:1615-1620, 1996.

Mills, Brooker, Camerini-Otero, “Sequences of human immunoglobulin switch regions: implications for recombination and transcription.” Nucl. Acids Res., 18:7305-7316, 1990.

Moll, Czyz, Holzmuller, Hofer-Warbinek, Wagner, Bach, Hofer, “Regulation of the tissue factor promoter in endothelial cells. Binding of NF kappa B-, AP-1-, and Sp1-like transcription factors.” J. Biol Chem., 270:3849-3857, 1995.

Moon, Geczy, “Recombinant IFN-□ synergizes with lipopolysaccharide to induce macrophage membrane procoagulants.” J. Immunol., 141:1536-1542, 1988.

Moore et al. “Tumor angiogenesis is regulated by CXC chemokines,” J. Lab. Clin. Med. 132(2):97-103, 1998.

Morrison, Johnson, Herzenberg, Oi, “Chimeric human antibody molecules: mouse antigen-binding domains with human constant region domains,” Proc. Natl. Acad. Sci USA, 81(21):6851-6855, 1984.

Morrison, Wims, Kobrin, Oi, “Production of novel immunoglobulin molecules by gene transfection,” Mt. Sinai J. Med., 53(3):175, 1986.

Morrissey, Fakhrai, Edgington, “Molecular cloning of the cDNA for tissue factor, the cellular receptor for the initiation of the coagulation protease cascade,” Cell, 50:129-135, 1987.

Morrissey et al., Blood, 81:734-744, 1993.

Morrissey, “Tissue Factor: An enzyme cofactor and a true receptor.” Thromb. Haemost., 86:66-74, 2001.

Muller, et al., “VEGF and the Fab fragment of a humanized neutralizing antibody: crystal structure of the complex at 2.4 A resolution and mutational analysis of the interface,” Structure, 6(9): 1153-67, 1998.

Muller, Ultsch, Kelley. De Vos, “Structure of the extracellular domain of human tissue factor: Location of the tissue factor VIIa binding site,” Biochemistry, 33:10864-10870, 1994.

Munro, “Endothelial-leukocyte adhesive interactions in inflammatory diseases,” European Heart Journal. 14:72-77, 1993.

Murphy, Greene, Tino, Boynton, Holmes, “Isolation and characterization of monoclonal antibodies specific for the extracellular domain of prostate specific membrane antigen”, J. Urology. 160(6 Pt 2):2396-401, 1998.

Nagler, Feferman, Shoshan, “Reduction in basic fibroblast growth factor mediated angiogenesis in vivo by linomide,” Connect Tissue Res., 37(1-2):61-68, 1998.

Nakanishi, Ohkubo, Nawa, Kitamura, Kageyama, Ujihara, “Angiotensinogen and kininogen: closing and sequence analysis of the cDNAs,” Clin. Exp. Hypertens., 5(7-8):997-1003, 1983.

Nawroth, Handley, Matsueda, De Waal, Gerlach, Blohm, Sterm, “Tumor necrosis factor/cachectin-induced intravascular fibrin formation in meth A fibrosarcomas,” J. Exp. Med, 168:637-647, 1988.

Nowotny, “Molecular aspects of endotoxic reactions,” Bacteriol. Rev., 33:72-98, 1969.

Ohizumi, Tsunoda, Taniguchi, Saito, Esaki, Makimoto, Wakai, Tsutsumi, Nakagawa, Utoguchi, Kaiho, Ohsugi, Mayumi, “Antibody-based therapy targeting tumor vascular endothelial cells suppresses solid tumor growth in rats,” Biochem. Biophys. Res Comm., 236:493-496, 1997.

Olkawa et al., “A highly potent antiangiogenic activity of retinoids,” Cancer Lett., 48(2):157-162, 1989.

Old, Boyse, “Current enigmas in cancer research,” Harvey Lect. 67:273-315, 1973.

O'Reilly et al., “Angiostatin: a novel angiogenesis inhibitor that mediates the suppression of metastases by a Lewis lung carcinoma.” Cell, 79:315-328, 1994.

O'Reilly et al., “Endostatin: an endogenous inhibitor of angiogenesis and tumor growth.” Cell, 88(2):277-285, 1997.

Ortel, Devore-Carter, Quinn-Allen, Kane, “Deletion analysis of recombinant human factor V. Evidence for a phosphatidylserine binding site in the second C-type domain,” J. Biol. Chem., 267:4189-4198, 1992.

Paborsky et al., J. Biol. Chem., 266(32):21911-21916, 1991.

Parkins et al., Brit. J. Cancer, 83:811-816, 2000.

Parmley and Smith, “Antibody-selectable filamentous fd phage vectors: affinity purification of target genes,” Gene, 73(2):305-318, 1988.

Parr, Wheeler, Alexander, “Similarities of the anti-tumour actions of endotoxin, lipid A and double-stranded RNA,” Br. J. Cancer, 27:370-389, 1973.

Parry, Mackman, “Transcriptional regulation of tissue factor expression in human endothelial cells,” Arterioscler. Thromb. Vasc. Biol., 15:612-621, 1995.

Patey, Vazeux, Canioni, Potter, Gallatin, Brousse, “Intercellular adhesion molecule-3 on endothelial cells: Expression in tumors but not in inflammatory responses,” Am. J. Pathol., 148:465-472, 1996.

Pepper et al., “Leukemia inhibitory factor (LIF) inhibits angiogenesis in vitro,” J Cell Sci., 108(Pt 1):73-83, 1995.

Perry, Hall, Bell, Jones, “Sequence analysis of a mammalian phospholipid-binding protein from testis and epididymis and its distribution between spermatozoa and extracellular secretions,” Biochem. J, 301(Pt 1):235-242, 1994.

Philpott, Joseph, Crosier, Baguley, and Ching, British Journal of Cancer 76:1586, 1997.

Pieroni, Broderick, Bundeally, Levine, “A simple method for the quantitation of submicrogram amounts of bacterial endotoxin,” Proc. Soc. Exp. Biol. Med. 133:790-794, 1970.

Pradier, Willems, Abramowicz, Schandene, de Boer, Thielemans, Capel, and Goldman, European Journal of Immunology 26:3048, 1996.

Presta. Chen, O'Connor, Chisholm, Meng, Krummen, Winkler, Ferrara, “Humanization of an anti-vascular endothelial growth factor monoclonal antibody for the therapy of solid tumors and other disorders,” Cancer Res., 57:4593-4599, 1997.

Quinn et al., CM101. a polysaccharide antitumor agent, does not inhibit wound healing in murine models,” J. Cancer Res. Clin. Oncol, 121(4):253-256, 1995.

Ran, Gao, Duffy, Watkins, Rote, Thorpe, “Infarction of solid Hodgkin tumors in mice by antibody-directed targeting of tissue factor to tumor vasculature,” Cancer Res., 58:4646-4653, 1998.

Ravlic-Gulan, Radosevic-Stasic, Trobonjaca, Petkovic, Cuk, and Rukavina, International Archives ofAllergy and Immunology 119:13, 1999.

Remington's Pharmaceutical Sciences, 16th Ed., Mack Publishing Company, 1980.

Richer and Lo, “Introduction of human DNA into mouse eggs by injection of dissected human chromosome fragments”, Science 245, 175-177, 1989.

Riechmann, Clark, Waldmann, Winter, “Reshaping human antibodies for therapy,” Nature, 332(6162):323-327, 1988.

Rietschel, Kirikae, Schade, Ulmer, Hoist, Brade, Schmidt, Mamat, Grimmecke, Kusumoto, Zaahringer, “The chemical structure of bacterial endotoxin in relation to bioactivity,” Immunobiol., 187:169-190, 1993.

Roboz et al., Blood, 96:1525-1530, 2000.

Rote, Ng, Dostal-Johnson, Nicholson, Siekman, “Immunologic detection of phosphatidylserine externalization during thrombin-induced platelet activation,” Clin. Immunol. Immunopathol., 66:193-200, 1993.

Ruf and Edgington, Proc Natl. Acad. Sci. USA., 88:8430-8434, 1991a.

Ruf and Edgington, Thrombosis and Haemostasis. 66(5):529-533, 40, 1991b.

Ruf, Rehemtulla, Edgington, “Phospholipid-independent and -dependent interactions required for tissue factor receptor and cofactor function.” Biol Chem., 266:2158-2166, 1991.

Ruf et al., J. Biol. Chem., 267:22206-22210, 1992a.

Ruf et al., J. Biol. Chem., 267:6375-6381. 1992b.

Ruf et al., J Biol. Chem., 267(31):22206-22210. 1992c.

Sakai and Kisiel, Thrombosis Res., 60:213-222, 1990.

Sakamoto et al., “Heparin plus cortisone acetate inhibit tumor growth by blocking endothelial cell proliferation,” Canc J. 1:55-58, 1986.

Saleh, Stacker, Wilks, “Inhibition of growth of C6 glioma cells in vivo by expression of antisense vascular endothelial growth factor sequence,” Cancer Res., 56:393-401, 1996.

Sambrook, Fritsch, Maniatis, Molecular Cloning: A Laboratory Manual, 2nd Ed., Cold Spring Harbor Press, Cold Spring Harbor, N.Y., 1989.

Sands, Immunoconjugates and Radiopharmaceuticals, 1:213-226, 1988.

Sang, “Complex role of matrix metalloproteinases in angiogenesis,” Cell Res., 8(3):171-177, 1998.

Sanlioglu, Williams, Samavati, Butler, Wang, McCray, Ritchie, Hunninghake, Zandi, and Engelhardt, Journal of Biological Chemistry 32:30188, 2001.

Santucci, Erlich, Labriola, Wilson, Kao, Kickler, Spillert, Mackman, “Measurement of tissue factor activity in whole blood,” Thromb. Haemost., 83:445-454, 2000.

Scarpati, Wen, Broze Jr., Miletich, Flandermeyer, Siegel, Sadler, “Human tissue factor: cDNA sequence and chromosome localization of the gene,” Biochemistry, 26:5234-5238, 1987.

Schoentgen, Saccoccio, Jolles, Bernier, Jolles, “Complete amino acid sequence of a basic 21-kDa protein from bovine brain cytosol,” Eur J. Biochem., 166(2):333-338, 1987.

Schorer, Rick, Swaim, Moldow, “Structural features of endotoxin required for stimulation of endothelial cell tissue factor production; exposure of preformed tissue factor after oxidant-mediated endothelial cell injury.” J. Lab. Clin. Med., 106:38-42, 1985.

Sersa, Cemazar, Parkins, and Chaplin, European Journal of Cancer 35:672, 1999.

Shear, “Chemical treatment of tumors. IX. Reactions of mice with primary subcutaneous tumors to injection of a hemorrhage-producing bacterial polysaccharide,” J. Nat. Cancer Inst., 4:461-476, 1944.

Sheibani and Frazier. “Thrombospondin 1 expression in transformed endothelial cells restores a normal phenotype and suppresses their tumorigenesis,” Proc. Natl. Acad. Sci. USA, 92(15):6788-6792. 1995.

Sheu et al., “Inhibition of angiogenesis in vitro and in vivo: comparison of the relative activities of triflavin, an Arg-Gly-Asp-containing peptide and anti-alpha(v)beta3 integrin monoclonal antibody,” Biochim. Biophys. Acta, 1336(3):445-454, 1997.

Shôckley et al., Ann. N.Y Acad. Sci., 617:367-382, 1991.

Sideras, Mizuta, Kanamori, Suzuki, Okamoto, Kuze, Ohno, Doi, Fukuhara, Hassan, et al., “Production of sterile transcripts of C gamma genes in an IgM-producing human neoplastic B cell line that switches to IgG-producing cells.” Intl. Immunol., 1(6):631-642, 1989.

Siebahn. “Cellular consequences upon factor VIIa binding to tissue factor,” Haemostasis 30(suppl 2):41-47, 2001.

Siemeister, Martiny-Baron, Marme, “The pivotal role of VEGF in tumor angiogenesis:

molecular facts and therapeutic opportunities,” Cancer Metastasis Rev., 17(2):241-248., 1998.

Silver, Pellicer, Fair, Heston, Cordon-Cardo, “Prostate-specific membrane antigen expression in normal and malignant human tissues, Clinical Cancer Research, 3(1):81-5, 1997.

Sioussat, Dvorak, Brock, Senger, “Inhibition of vascular permeability factor (vascular endothelial growth factor) with antipeptide antibodies.” Arch. Biochem. Biophys., 301:15-20, 1993.

Sipos et al., “Inhibition of tumor angiogenesis,” Ann. NY Acad. Sci., 732:263-272, 1994.

Sluiter, Pietersma, Lamers, Koster, “Leukocyte adhesion molecules on the vascular endothelium: their role in the pathogenesis of cardiovascular disease and the mechanisms underlying their expression,” J. Cardiol. Pharmacol., 22:S37-S44. 1993.

Soff et al., “Expression of plasminogen activator inhibitor type 1 by human prostate carcinoma cells inhibits primary tumor growth, tumor-associated angiogenesis, and metastasis to lung and liver in an athymic mouse model,” J. Clin. Invest., 96(6):2593-2600, 1995.

Spicer, Horton, Bloem, Bach, Williams, Guha, Kraus, Lin, Nemerson, Konigsberg, “Isolation of cDNA clones coding for human Tissue Factor: Primary structure of the protein and cDNA,” Proc. Natl. Acad. Sci. USA, 84:5148-5152, 1987.

Spiegelberg and Weigle, J. Exp. Med., 121 :323-338, 1965.

Staal-van den Brekel, Thunnissen, Buurmnan, Wouters, “Expression of E-selectin, intercellular adhesion molecule (ICAM)-1 and vascular cell adhesion molecule (VCAM)-1 in non-small-cell lung carcinoma.” Virchows Arch., 428:21-27, 1996.

Stone, Ruf, Miles, Edgington, Wright, “Recombinant soluble human tissue factor secreted by Saccharomyces cerevisiae and refolded from E. coli inclusion bodies: glycosylation of mutants, activity, and physical characterization,” Biochem. J., 310(2):605-614, 1995.

Tada et al., “Inhibition of tubular morphogenesis in human microvascular endothelial cells by co-culture with chondrocytes and involvement of transforming growth factor beta: a model for avascularity in human cartilage,” Biochim. Biophys. Acta, 1201(2):135-142, 1994.

Takano et al. “Suramin, an anticancer and angiosuppressive agent, inhibits endothelial cell binding of basic fibroblast growth factor, migration. proliferation, and induction of urokinase-type plasminogen activator,” Cancer Res., 54(10):2654-2660, 1994.

Tanaka et al., “Viral vector-mediated transduction of a modified platelet factor 4 cDNA inhibits angiogenesis and tumor growth,” Nat. Med., 3(4):437-442, 1997.

ten Cate “Pathophysiology of disseminated intravascular coagulation in sepsis,” Crit. Care Med., 28(Suppl):S9-S11, 2000.

Thornhill, Kyan-Aung, Haskard, “IL-4 increases human endothelial cell adhesiveness for T cells but not for neutrophils.” J. Immunol., 144:3060-3065, 1990.

Thorpe et al. “Heparin-Steroid Conjugates: New Angiogenesis Inhibitors with Antitumor Activity in Mice,” Cancer Res., 53:3000-3007, 1993.

Thorpe and Ran, “Tumor infarction by targeting tissue factor to tumor vasculature”, Cancer J Sci Am, 6(Suppl 3):S237-S244, 2000.

Tolsma et al., “Peptides derived from two separate domains of the matrix protein thrombospondin-1 have anti-angiogenic activity,” J. Cell Biol., 122(2):497-511, 1993.

Trousseau. “Phlegmasia alba dolens,” Lectures on clinical medicine, delivered at the Hôtel-Dieu, Paris, London, New Sydenham Society, 281-295, 1872.

Tryggvason, “The laminin family,” Curr. Opin. Cell Biol., 5(5):877-882. 1993.

Valinger, Ladesic, Hrsak, and Tomasic, International Journal of Immunopharmacology 9:325, 1987.

van Dijk, Wamaar, van Eendenburg, Thienpont, Braakman, Boot, Fleuren, Bolhuis, “Induction of tumor-cell lysis by bi-specific monoclonal antibodies recognizing renal-cell carcinoma and CD3 antigen,” Int. J. Cancer, 43:344-349, 1989.

Verbon, Dekkers, ten Hove, Hack, Pribble, Turner, Souza, Axtelle, Hoek, FJ; van Deventer, van der Poll, “IC14, an anti-CD14 antibody, inhibits endotoxin-mediated symptoms and inflammatory responses in humans,” J. Immunol., 166(5):3599-605, 2001.

Vitetta et al., “Phase I immunotoxin trial in patients with B-cell lymphoma,” Cancer Res., 15:4052-4058. 1991.

Volpert, Lawler, Bouck, “A human fibrosarcoma inhibits systemic angiogenesis and the growth of experimental metastases via thrombospondin-1,” Proc Natl. Acad. Sci. USA, 95(11):6343-6348, 1998.

Vukanovic et al., “Antiangiogenic effects of the quinoline-3-carboxamide linomide,” Cancer Res., 53(8):1833-1837, 1993.

Waltenberger et al . “Suramin is a potent inhibitor of vascular endothelial growth factor. A contribution to the molecular basis of its antiangiogenic action.” J. Mol. Cell Cardiol., 28(7):1523-1529, 1996.

Wamil et al., “Soluble E-selectin in cancer patients as a marker of the therapeutic efficacy of CM101, a tumor-inhibiting anti-neovascularization agent, evaluated in phase I clinical trail,” J. Cancer Res. Clin. Oncol., 123(3):173-179, 1997.

Warr, Rao, Rapaport, “Disseminated intravascular coagulation in rabbits induced by administration of endotoxin or tissue factor: effect of anti-tissue factor antibodies and measurement of plasma extrinsic pathway inhibitor activity,” Blood, 75:1481-1489, 1990.

Watanabe, Niitsu. Umeno, Kuriyama. Neda. Yamauchi, Maeda, Urushizaki, “Toxic effect of tumor necrosis factor on tumor vasculature in mice,” Cancer Res., 48:2179-2183, 1988.

Watanabe et al., Proc Natl. Acad. Sci USA. 86:9456-9460, 1989.

Weiss, Young, LoBuglio, Slivka, Nimeh, “Role of Hydrogen Peroxide in Neutrophil-Mediated Destruction of Cultured Endothelial Cells,” J. Clin. Invest., 68:714-721, 1981.

Wells, “Starving cancer into submission” Chem. Biol., 5(4):R87-88, 1998.

Wiesmann, et al., “Crystal structure at 1.7 A resolution of VEGF in complex with domain 2 of the Flt-1 receptor,” Cell. 91(5):695-704, 1997.

Winter and Milstein, “Man-made antibodies,” Nature, 349:293-299, 1991.

Wolff et al., “Dexamethasone inhibits glioma-induced formation of capillary like structures in vitro and angiogenesis in vivo,” Klin. Padiatr., 209(4):275-277, 1997.

Woodlock, Sahasrabude, Marquis, Greene, Pandya, and McCune, Journal of Immunotherapy 22:251, 1999.

Yamada, Moldow, Sacks, Craddock, Boogaens, Jacob, “Deleterious Effects of Endotoxin on Cultured Endothelial Cells: An in vitro Model of Vascular injury,” Inflammation, 5:115-116, 1981.

Yamamura et al., “Effect of Matrigel and laminin peptide YIGSR on tumor growth and metastasis,” Semin. Cancer Biol., 4(4):259-265, 1993.

Yoon et al., “Inhibitory effect of Korean mistletoe (Viscum album coloratum) extract on tumour angiogenesis and metastasis of haematogenous and non-haematogenous tumour cells in mice,” Cancer Lett, 97(1):83-91, 1995.

Yoshida et al., “Suppression of hepatoma growth and angiogenesis by a fumagillin derivative TNP470: possible involvement of nitric oxide synthase,” Cancer Res., 58(16):3751-3756, 1998.

Zhang, Deng, Wendt, Liliensiek, Bierhaus, Greten, He, Chen, Hach-Wunderle, Waldherr, Ziegler, Männel, Stern, Nawroth, “Intravenous somatic gene transfer with antisense tissue factor restores blood flow by reducing tumor necrosis factor-induced tissue factor expression and fibrin deposition in mouse Meth-A sarcomas,” J. Clin. Invest., 97:2213-2224, 1996.

Zapata et al., Protein Eng., 8(10):1057-1062, 1995.

Ziche et al., “Linomide blocks angiogenesis by breast carcinoma vascular endothelial growth factor transfectants,” Br. J. Cancer, 77(7): 1123-1129, 1998.

Zuckerman, Surprenant, “Induction of endothelial cell/macrophage procoagulant activity:

synergistic stimulation by gamma-interferon and granulocyte-macrophage colony stimulating factor, “Thromb. Haemostasis., 61:178-182, 1989.

What is claimed is:

1. A method for treating an animal having a vascularized tumor, comprising subjecting said animal to a sensitizing treatment in a manner effective to enhance the procoagulant status of the vasculature of said vascularized tumor; and administering to said animal a non-targeted coagulation-deficient Tissue Factor compound in an amount effective to induce coagulation in the vasculature of said tumor.

2. The method of claim 1, wherein said sensitizing treatment is performed at a biologically effective time prior to administration of said coagulation-deficient Tissue Factor compound.

3. The method of claim 1, wherein said sensitizing treatment and the administration of said coagulation-deficient Tissue Factor compound are performed essentially simultaneously.

4. The method of claim 1, wherein said sensitizing treatment comprises administering a sensitizing dose of a sensitizing agent to said animal.

5. The method of claim 4, wherein said sensitizing agent is endotoxin or a detoxified endotoxin derivative.

6. The method of claim 5, wherein said sensitizing agent is monophosphoryl lipid A (MPL).

7. The method of claim 4, wherein said sensitizing agent is an activating antibody that binds to the cell surface activating antigen CD14 and that does not bind to a tumor antigen on the cell surface of a tumor cell.

8. The method of claim 4, wherein said sensitizing agent is a cytokine selected from the group consisting of monocyte chemoattractant protein-1 (MCP-1), platelet-derived growth factor-BB (PDGF-BB) and C-reactive protein (CRP).

9. The method of claim 4, wherein said sensitizing agent is tumor necrosis factor-α (TNFα) or an inducer of TNFα.

10. The method of claim 9, wherein said sensitizing agent is an inducer of TNFα selected from the group consisting of endotoxin, a Rac1 antagonist, DMXAA, CM101 or thalidomide.

11. The method of claim 4, wherein said sensitizing agent is muramyl dipeptide (MDP), threonyl-MDP or MTPPE.

12. The method of claim 4, wherein said sensitizing agent is a sensitizing dose of an anti-angiogenic agent.

13. The method of claim 12, wherein said sensitizing agent is a sensitizing dose of an anti-angiogenic agent selected from the group consisting of vasculostatin, canstatin and maspin.

14. The method of claim 12, wherein said sensitizing agent is a sensitizing dose of a VEGF inhibitor.

15. The method of claim 14, wherein said sensitizing agent is a sensitizing dose of an anti-VEGF blocking antibody.

16. The method of claim 14, wherein said sensitizing agent is a sensitizing dose of a soluble VEGF receptor construct (sVEGF-R), a tyrosine kinase inhibitor, an antisense VEGF construct, an anti-VEGF RNA aptamer or an anti-VEGF ribozyme.

17. The method of claim 4, wherein said sensitizing agent is an activating antibody that binds to the cell surface activating antigen CD40.

18. The method of claim 4, wherein said sensitizing agent is sCD40-Ligand (sCD153).

19. The method of claim 4, wherein said sensitizing agent is a sensitizing dose of a combretastatin, or a prodrug or tumor-targeted form thereof.

20. The method of claim 19, wherein said sensitizing agent is a sensitizing dose of combretastatin A-1, A-2, A-3, A-4, A-5, A-6, B-1, B-2, B-3, B-4, D-1 or D-2, or a prodrug or tumor-targeted form thereof.

21. The method of claim 4, wherein said sensitizing agent is a sensitizing dose of thalidomide.

22. The method of claim 4, wherein a single composition comprising said sensitizing agent and said coagulation-deficient Tissue Factor compound is administered to said animal.

23. The method of claim 4, wherein distinct compositions comprising said sensitizing agent and said coagulation-deficient Tissue Factor compound are administered to said animal.

24. The method of claim 1, wherein said non-targeted coagulation-deficient Tissue Factor compound is between about 100-fold and about 1,000,000-fold less active in coagulation than full length, native Tissue Factor.

25. The method of claim 24, wherein said non-targeted coagulation-deficient Tissue Factor compound is at least about 1,000-fold less active in coagulation than full length, native Tissue Factor.

26. The method of claim 25, wherein said non-targeted coagulation-deficient Tissue Factor compound is at least about 10,000-fold less active in coagulation than full length, native Tissue Factor.

27. The method of claim 26, wherein said non-targeted coagulation-deficient Tissue Factor compound is at least about 100,000-fold less active in coagulation than full length, native Tissue Factor.

28. The method of claim 1, wherein said non-targeted coagulation-deficient Tissue Factor compound is a human Tissue Factor compound.

29. The method of claim 1, wherein said non-targeted coagulation-deficient Tissue Factor compound is prepared by recombinant expression.

30. The method of claim 1, wherein said non-targeted coagulation-deficient Tissue Factor compound is deficient in binding to a phospholipid surface.

31. The method of claim 1, wherein said non-targeted coagulation-deficient Tissue Factor compound is a truncated Tissue Factor.

32. The method of claim 31, wherein said non-targeted coagulation-deficient Tissue Factor compound is about 219 amino acids in length.

33. The method of claim 1, wherein said non-targeted coagulation-deficient Tissue Factor compound is a dimeric or polymeric Tissue Factor.

34. The method of claim 1, wherein said non-targeted coagulation-deficient Tissue Factor compound has been modified to increase its biological half life, other than by attachment to a binding region that binds to a component of a tumor cell, tumor vasculature or tumor stroma.

35. The method of claim 34, wherein said non-targeted coagulation-deficient Tissue Factor compound is operatively linked to an inert carrier molecule that increases the biological half life of said coagulation-deficient Tissue Factor compound.

36. The method of claim 35, wherein said inert carrier molecule is an inert protein carrier molecule.

37. The method of claim 36, wherein said inert carrier molecule is an albumin or a globulin.

38. The method of claim 36, wherein said inert carrier molecule is an antibody or portion thereof, wherein the antibody does not specifically bind to a component of a tumor cell, tumor vasculature or tumor stroma.

39. The method of claim 38, wherein said inert carrier molecule is an Fc portion of an antibody.

40. The method of claim 35, wherein said inert carrier molecule is a polysaccharide or synthetic polymer carrier molecule.

41. The method of claim 1, wherein said animal is a human patient.

42. A method for treating an animal having a vascularized tumor, comprising administering to said animal a sensitizing dose of a sensitizing agent effective to enhance the procoagulant status of the vasculature of said vascularized tumor; and administering to said animal a non-targeted coagulation-deficient Tissue Factor compound in an amount effective to induce coagulation in the vasculature of said tumor.

43. A method for treating an animal having a vascularized tumor, comprising administering to said animal a sensitizing dose of endotoxin or a detoxified endotoxin derivative effective to enhance the procoagulant status of the vasculature of said vascularized tumor; and administering to said animal a non-targeted, truncated, coagulation-deficient Tissue Factor compound in an amount effective to induce coagulation in the vasculature of said tumor.
